SEQUENCE LISTING (1688 sequences)

SEQ ID NO: 1 - DNA, 100 bp, Artificial
  Hairpin sequence Hsd_1, human
ggttgggcaaggtgcggggctagggctaacagcagtcttactgaaggtttcctggaaaccacgcacatgctgttgccactaacctcaaccttactcggtc

SEQ ID NO: 2 - DNA, 96 bp, Artificial
  Hairpin sequence Hsd_10, human
ttctccatgcctggcatatagcaggtcctcaataagtatttgttgaaagaataaataaaccaacaagtcttatttgagcacctgttatgtggagga

SEQ ID NO: 3 - DNA, 93 bp, Artificial
  Hairpin sequence Hsd_100, human
gacctgggcttgggtggtaggaggaattggtgctggtctttcattttggatttgactccagccccacagcctcagccaccccagccaattgtc

SEQ ID NO: 4 - DNA, 92 bp, Artificial
  Hairpin sequence Hsd_101, human
ggctatgaggctgctggcctttttcctgtgctgtgctgccccttttggcttctttgagtgtgctagcaggagagccagctgcttttcatccc

SEQ ID NO: 5 - DNA, 81 bp, Artificial
  Hairpin sequence Hsd_102, human
cccagccctgcagtgggtgctggagacattcacacacagtgaattacaccagcatggttcctatcctgctggagcttaggg

SEQ ID NO: 6 - DNA, 90 bp, Artificial
  Hairpin sequence Hsd_103, human
agaattctgtctcttaggctttctcttcccagatttcccaaagttgggaaaagctgggttgagagggcaaaaggaaaaaaaaagaattct

SEQ ID NO: 7 - DNA, 98 bp, Artificial
  Hairpin sequence Hsd_104, human
gaatggttacaaggagagaacactggacttggagtcagaaaactttcatccaagtcattccctgctctaagtcccatttctgttccatgagattgttt

SEQ ID NO: 8 - DNA, 72 bp, Artificial
  Hairpin sequence Hsd_105, human
agccatatctgggctgctgtctcagaatatctaagcgagtagagaagaggaggaaaggagtttggaatggct

SEQ ID NO: 9 - DNA, 95 bp, Artificial
  Hairpin sequence Hsd_106, human
tgtcctggttccccctctctaatccttgctacctgggtgagagtgctttctgaatgcagtgcacccaggcaaggattctgcaagggggagtgaca

SEQ ID NO: 10 - DNA, 85 bp, Artificial
  Hairpin sequence Hsd_107, human
tgtctgccagtgactgcccaggtgtctgctggttcctccccaggagtagggaggaaccaggtgggctggctgggatgggtggata

SEQ ID NO: 11 - DNA, 98 bp, Artificial
  Hairpin sequence Hsd_108, human
gagccactctttggtacttgaagagaggataccctttgtatgttcacttgattaatggcgaatatacagggggagactcttatttgcgtatcaaactc

SEQ ID NO: 12 - DNA, 71 bp, Artificial
  Hairpin sequence Hsd_109, human; Hsd_56, human; Hsd_68, human
tcacagtcatcaggtaagtgagtcaagagaaggaatctggagactctgacttgcccgaggtgggcatgtgg

SEQ ID NO: 13 - DNA, 96 bp, Artificial
  Hairpin sequence Hsd_11, human
tgctctcctggcccatgaaatcaagcgtgggtgagacctggtgcagaacgggaaggcgacccatacttggtttcagaggctgtgagaataactgca

SEQ ID NO: 14 - DNA, 98 bp, Artificial
  Hairpin sequence Hsd_110, human
atgaactgctccttctcccatacccattgcatatcggagttgtgaattctcaaaacacctcctgtgtgcatggattacaggagggtgagccttgtcat

SEQ ID NO: 15 - DNA, 81 bp, Artificial
  Hairpin sequence Hsd_111, human
ttcacacacaacacactgagaggcatcgtggttgaataacccatctgaggccaatagctactgagtagtgtagtgggtgaa

SEQ ID NO: 16 - DNA, 81 bp, Artificial
  Hairpin sequence Hsd_12, human
gttggcatggggaggagggaggagatgggccaagttccctctggctggaacgcccttcccccccttcttcacctggcgaac

SEQ ID NO: 17 - DNA, 98 bp, Artificial
  Hairpin sequence Hsd_13, human
tggcaggccaggaagaggaggaagccctggaggggctggaggtgatggatgttttcctccggttctcagggctccacctctttcgggccgtagagcca

SEQ ID NO: 18 - DNA, 99 bp, Artificial
  Hairpin sequence Hsd_14, human
gatgcacagatctcagacatctcggggatcatcatgtcacgagataccagtgtgcacttgtgacagattgataactgaaaggtctgggagccactcatc

SEQ ID NO: 19 - DNA, 93 bp, Artificial
  Hairpin sequence Hsd_15, human
ggtaactgccctcaaggagcttacaatctagctgggggtaaatgacttgcacatgaacacaactagactgtgagcttctagagggcagggacc

SEQ ID NO: 20 - DNA, 96 bp, Artificial
  Hairpin sequence Hsd_16, human
gctgtttcatacttgaggagaaattatccttggtgtgttcgctttatttatgatgaatcatacaaggacaatttctttttgagtatcaaatcttgc

SEQ ID NO: 21 - DNA, 66 bp, Artificial
  Hairpin sequence Hsd_17, human
ggtaagtgcgcctcgggtgagcatgcacttaatgtgggtgtatgtcactcggctcggcccactacc

SEQ ID NO: 22 - DNA, 95 bp, Artificial
  Hairpin sequence Hsd_18, human
gatgctttgctggctggtgcagtgcctgagggagtaagagtcctgttgttgtaagatagtgtcttactccctcaggcacatctccaacaagtctc

SEQ ID NO: 23 - DNA, 103 bp, Artificial
  Hairpin sequence Hsd_19, human
agccctccagggtaagtggaaagatggtgggccgcagaacatgtgctgagttcgtgccatatgtctgctgaccatcacctttagaagccccctttcgctgagg

SEQ ID NO: 24 - DNA, 95 bp, Artificial
  Hairpin sequence Hsd_2, human
gaagaaatcctactcggatggatataatacaacctgctaagtgtcctagcacttagcaggttgtattatcattgtccgtgtctatggctctcgtc

SEQ ID NO: 25 - DNA, 96 bp, Artificial
  Hairpin sequence Hsd_20, human
atattgtaggtttttgctcatgcaccatggttgtctgagcatgcagcatgcttgtctgctcataccccatggtttctgagcaggaaccttcattgt

SEQ ID NO: 26 - DNA, 96 bp, Artificial
  Hairpin sequence Hsd_21, human
gcccttggtgaggactcgggaggtggagggtggtgccgccggggccgggcgctgtttcagctcgcttctccccccacctcctctctcctcaggtgc

SEQ ID NO: 27 - DNA, 95 bp, Artificial
  Hairpin sequence Hsd_22, human
ggagggtaagtcattattagtgaaagccatggattcaggagccagatagcccaggttcaagtcctggcttcaagtatgtgactttgggcacttct

SEQ ID NO: 28 - DNA, 98 bp, Artificial
  Hairpin sequence Hsd_23, human
ctgtagccagtcagaaatgagcttattcataaaagtgcagtatggtgaagtcaatctgtaattttatgtataagctagtctctgattgaaacatgcag

SEQ ID NO: 29 - DNA, 98 bp, Artificial
  Hairpin sequence Hsd_24, human
cggtgcggtttgataagtggtcggttcctggagctgcggtcttctgctagcctgatccgcagcttcagcctccgccgcctcctcagccctcggcgccg

SEQ ID NO: 30 - DNA, 97 bp, Artificial
  Hairpin sequence Hsd_25, human
tgaagaaggggaggtggagctcgttcttgtgtgcaaactacagggttaaggctcagaactgtggcttgtccaagaacgttcataccactgcttctca

SEQ ID NO: 31 - DNA, 99 bp, Artificial
  Hairpin sequence Hsd_26, human
cactggctgggccagtgtgtggtgcaggcagtctgtctacactggtggggccagtgcatggggcaggcagtccgcctacactgacggagccagcacgtg

SEQ ID NO: 32 - DNA, 100 bp, Artificial
  Hairpin sequence Hsd_27, human
ggctcgtgcctccatccttccaggaggaaacaaggacacaaaggtggcacacctgcctcccaagttcctccctgtcctccaggagctggatctcagagct

SEQ ID NO: 33 - DNA, 97 bp, Artificial
  Hairpin sequence Hsd_28, human
gaacttgagaaataaggcttctgtctactactggagacactggtagtataaaacccagagtctccagtaatggacgggagccttatttctatcactc

SEQ ID NO: 34 - DNA, 92 bp, Artificial
  Hairpin sequence Hsd_29, human
ttcccccatcctccttacgtcccaccccccactcctgtttctggtgaaatattcaaacaggagtgggggtgggacataaggaggatagggaa

SEQ ID NO: 35 - DNA, 100 bp, Artificial
  Hairpin sequence Hsd_3, human
cttcaaagtggcagactggaaaatctctgcaggcaaatgtgatgtcactgaggaaatcacacacttacccgtagagattctacagtctgacatcttcaag

SEQ ID NO: 36 - DNA, 96 bp, Artificial
  Hairpin sequence Hsd_30, human
acctctgatgtgtcagtctctcttcagggctcccgagacacagaaacagacacctgccctcgaggagctcacagtctagacaaacaaacccagggt

SEQ ID NO: 37 - DNA, 91 bp, Artificial
  Hairpin sequence Hsd_31, human
taaggagcagagcagcatggaaagaagggaggctttgggatcaaccctgggcttaattcccagctcagctgcttcctggatgtgtttctta

SEQ ID NO: 38 - DNA, 96 bp, Artificial
  Hairpin sequence Hsd_32, human
ggggaggtggggttgcgggggttagagctgtgggtgatcagaagggaagggcttcatttctacgctctgcctccgctacctcttccccaccacccc

SEQ ID NO: 39 - DNA, 83 bp, Artificial
  Hairpin sequence Hsd_33, human
tcctcaatgtactaagcgttgggaatgtagagactgaccaaagtatggtctctaccttcaaggagctcacagtctattgggga

SEQ ID NO: 40 - DNA, 79 bp, Artificial
  Hairpin sequence Hsd_34, human
aattaatccctctctttctagttcttcctagagtgaggaaaagctgggttgagagggcaaacaaattaactaattaatt

SEQ ID NO: 41 - DNA, 99 bp, Artificial
  Hairpin sequence Hsd_35, human
tgctgggggcgtgggggcggggacggcgtccgcgccttgcccctggtccattttggacgccgaggctaaggtctgtgcgcgcccccgctgcgtctagca

SEQ ID NO: 42 - DNA, 88 bp, Artificial
  Hairpin sequence Hsd_36, human
tttgcattaaaaatgaggccttctcttcccagttcttcccagagtcaggaaaagctgggttgagagggtagaaaaaaaatgatgtagg

SEQ ID NO: 43 - DNA, 95 bp, Artificial
  Hairpin sequence Hsd_37, human
gctgggtgggaaaagtggctaggagttagcttgggagccgctggtgttctgagcgctcccctctctggccactttttctccacgcccacccccgc

SEQ ID NO: 44 - DNA, 92 bp, Artificial
  Hairpin sequence Hsd_38, human
cacctacctaactgggttagggccctggctccatctcctttaggaaaaccttctgtggggagtggggcttcgaccctaacccaggtgggctg

SEQ ID NO: 45 - DNA, 99 bp, Artificial
  Hairpin sequence Hsd_39, human
agtctgctcaacagcttgttatttggtggattgtggcagtaaatcggggcgagtggggaacccggcgcaggaactgcagccgcggctgggagtggtgct

SEQ ID NO: 46 - DNA, 99 bp, Artificial
  Hairpin sequence Hsd_4, human
tgatgatattatgccatgacattgtgtcaatatgcgatgatgtgttgtgatggcacagcgtcatcacgtggtgacgcaacatcatgacgtaagacgtca

SEQ ID NO: 47 - DNA, 86 bp, Artificial
  Hairpin sequence Hsd_40, human
ggtgggtggtttctccgtttgcctgtttcgctgatgtgcattcaactcattctcagcaaaataagcaaatggaaaattcgtccatc

SEQ ID NO: 48 - DNA, 92 bp, Artificial
  Hairpin sequence Hsd_41, human
gggggtccaggaggctccagggtgagcccactcctccccctagatacaagggagggccctgggaccctgtctgctgttcctcctggactcct

SEQ ID NO: 49 - DNA, 93 bp, Artificial
  Hairpin sequence Hsd_42, human
gacctgacccaggttttagtggctccctctgcctgcagaatggaagaccacgtgcaggtgagggtggagccaccgatacctgggtgatgggtc

SEQ ID NO: 50 - DNA, 98 bp, Artificial
  Hairpin sequence Hsd_43, human
cggtgcggtttgctaagtggtcggttcctggagctgcggtcttctgctagcctgatccgcagcttcagcctccgccgcctcctcagccctcggcgccg

SEQ ID NO: 51 - DNA, 98 bp, Artificial
  Hairpin sequence Hsd_44, human
atgacattcagactgaggtcctcaaaactgaggggcattttctgtggtttgaaaggaaagtgcacccagttttggggatgtcaattgtgaatcctcat

SEQ ID NO: 52 - DNA, 94 bp, Artificial
  Hairpin sequence Hsd_45, human
gataacagcacaatcctattctagagtccatatcaacaataaggtttacgacctcgatgttggatcaggacatcctaatggtgtagctgctatt

SEQ ID NO: 53 - DNA, 99 bp, Artificial
  Hairpin sequence Hsd_46, human
ttcccctgcagcaaggggcggggccaccccaacgccgcttctgcggccaaagtaggttgggagtggaaggtggtggctgctgctccgcagtgtcgggaa

SEQ ID NO: 54 - DNA, 98 bp, Artificial
  Hairpin sequence Hsd_47, human
tctaaacacaactcactgtgcccccaggacatttggagacttctggagacatctttggttatctcagctgggggtagtgggtgcatctagtgggtaga

SEQ ID NO: 55 - DNA, 92 bp, Artificial
  Hairpin sequence Hsd_48, human
gggagtccagctcatctacagagtcatttttattcctggccatggagcccttgaatgccagggtcagggatggctcagttcagccgggcccc

SEQ ID NO: 56 - DNA, 97 bp, Artificial
  Hairpin sequence Hsd_49, human
ctgctactcggcccgcactctctccattacactaccctgcctcttctccatgagaggcagcggggtgtagtggatagagcacgggttcaagtcccgg

SEQ ID NO: 57 - DNA, 99 bp, Artificial
  Hairpin sequence Hsd_5, human
gctgacgtgcggtacttaatgagaagttgcccgtgtttttttcgctttatttgtgacgaaacattcgcggtgcacttctttttcagtatcctattctgc

SEQ ID NO: 58 - DNA, 85 bp, Artificial
  Hairpin sequence Hsd_50, human
gagggaatagtgagggcgagggcacggacagccaggaaggtccggggatgcccggaaccatcatcccgttccttgtccgtccttt

SEQ ID NO: 59 - DNA, 61 bp, Artificial
  Hairpin sequence Hsd_51, human
gctgttccccagcctccttggtattccagagtgtgtggaatacccaagggctattgacagc

SEQ ID NO: 60 - DNA, 99 bp, Artificial
  Hairpin sequence Hsd_52, human
gccggcgggggcgcgggcggcagtggcgggagcggcccctcggccatcctccgtctgcccagttaccgcttccgctaccgccgccgctcccgctctagc

SEQ ID NO: 61 - DNA, 62 bp, Artificial
  Hairpin sequence Hsd_53, human
ggtggtgaagggtgcaggctctaaaaccagaaaacccaggttcaagtcctggccctaccacc

SEQ ID NO: 62 - DNA, 93 bp, Artificial
  Hairpin sequence Hsd_54, human
gtctgtacagataagtcggcttcaggggcgctctgtctctgttgctctaacagaccagacttgctcctgccgttgactctctgctcccagcgc

SEQ ID NO: 63 - DNA, 95 bp, Artificial
  Hairpin sequence Hsd_55, human
agagttggccatagcagccgaggggaccccggcagatctgcccagctggctgtgatctgctgccaacccctggggtcctgcccacccctggctct

SEQ ID NO: 64 - DNA, 91 bp, Artificial
  Hairpin sequence Hsd_57, human
ttctgatttcttcttcatttaaagaagtccttgttactttatattcacactctgtaactgcacaggacttcagaggagggagatatgggaa

SEQ ID NO: 65 - DNA, 97 bp, Artificial
  Hairpin sequence Hsd_58, human
ggcaagggtagccattgtttgggttggggtggtcggccctggagggggtttgtttgcttattcccctctgtgcttcacccctacccagcttcccgcc

SEQ ID NO: 66 - DNA, 54 bp, Artificial
  Hairpin sequence Hsd_59, human
gccgttgcccgcatcccccacctctcgcggcgagagtggatgcgtggccccggc

SEQ ID NO: 67 - DNA, 85 bp, Artificial
  Hairpin sequence Hsd_6, human
cttgctttctctcctccatgccttgagtgtaggaccgttggcatcttaattaccctcccacacccaaggcttgcaaaaaagcgag

SEQ ID NO: 68 - DNA, 91 bp, Artificial
  Hairpin sequence Hsd_60, human
gccccggggtgcagatccttgggagccctgttagactctggattttacacttggagtgaacgggcgccatcccgaggctttgcacaggggc

SEQ ID NO: 69 - DNA, 98 bp, Artificial
  Hairpin sequence Hsd_61, human
aggcacggtccctgccctcgaggagctcacagtctagtaaggaagacatatggggaaccgattccccaaagatgcggggagtaggggaggcagtgctt

SEQ ID NO: 70 - DNA, 98 bp, Artificial
  Hairpin sequence Hsd_62, human
gaagaaaggaaacggggaaaatcaaaaggggttcaaaccccacctcagtaggtggaggggagcgcctgccattggttgtatttttgttctgagttttc

SEQ ID NO: 71 - DNA, 100 bp, Artificial
  Hairpin sequence Hsd_63, human
ctcttcggcccacgcctcctcccctccccaggttcctcattaaaccagcaagaagccctatgcttcaaggggcggggagaatctggtggctgcctgggag

SEQ ID NO: 72 - DNA, 48 bp, Artificial
  Hairpin sequence Hsd_64, human
cttctcttcctggttcttcctgaagtcaggaaaagctgggttgagagg

SEQ ID NO: 73 - DNA, 100 bp, Artificial
  Hairpin sequence Hsd_65, human
gcaggcagccactggggtgtccctgcttactcagtcacagaggaggccctggctggagtggggctgcgggaagagctgggatcactcccggggctcctgc

SEQ ID NO: 74 - DNA, 47 bp, Artificial
  Hairpin sequence Hsd_66, human
ctgccatccctgggagcctggaaccaggcaatgttcgggggaggcag

SEQ ID NO: 75 - DNA, 82 bp, Artificial
  Hairpin sequence Hsd_67, human
cctgaatcaggtaggcagtgtattgttagctggctgcttgggtcaagtcagcagccacaactaccctgccacttgcttctgg

SEQ ID NO: 76 - DNA, 97 bp, Artificial
  Hairpin sequence Hsd_69, human
agctgaagagcctgtgggaaagagaagagcagggcagggtgaaggcccggcggagacactctgcccaccccacaccctgcctatgggccacacagct

SEQ ID NO: 77 - DNA, 87 bp, Artificial
  Hairpin sequence Hsd_7, human
ggctatgcactgcacaaccctaggagagggtgccattcacatagactataattgaatggcgccactagggttgtgcagtgcacaacc

SEQ ID NO: 78 - DNA, 95 bp, Artificial
  Hairpin sequence Hsd_70, human
gatgctttgctggctggtgcagtgcctgagggagtaagagccctgttgttgtaagatagtgtcttactccctcaggcacatctccaacaagtctc

SEQ ID NO: 79 - DNA, 97 bp, Artificial
  Hairpin sequence Hsd_71, human
cagggtcttgtcttgtccctgcggggacaggccagggcatctaggctgtgcacagtgacgcccctcctgcccccacagaagcgcagaccctactctg

SEQ ID NO: 80 - DNA, 94 bp, Artificial
  Hairpin sequence Hsd_72, human
ctgttcccccccaggagggatgcactggggaactggggttggcacggggacttggagttggtgacctcactgctgccttgtgggtggggggcag

SEQ ID NO: 81 - DNA, 85 bp, Artificial
  Hairpin sequence Hsd_73, human
ggccgctgggcgcacccgtcccgttcgtccccggacgttgctctctaccccgggaacgtcgagactggagcgcccgaactgagcc

SEQ ID NO: 82 - DNA, 80 bp, Artificial
  Hairpin sequence Hsd_74, human
gctgtgcagagttattgcctctgttctaacacaagactaggcttccctgtgttagaataggggcaataactctgcactgc

SEQ ID NO: 83 - DNA, 98 bp, Artificial
  Hairpin sequence Hsd_75, human
ctgcagcacccagatcagtgcttggcacctagcaagcactcagtaaatatttgttgagtgcctgctatgtgccaggcattgtgctgagggctttgtgg

SEQ ID NO: 84 - DNA, 94 bp, Artificial
  Hairpin sequence Hsd_76, human
tagaatggggattgtggggggtcgctctaggcaccgcagcactgtgctggggatgttgcagctgcctgggagtgacttcacacagtcctctctg

SEQ ID NO: 85 - DNA, 98 bp, Artificial
  Hairpin sequence Hsd_77, human
agcctcccgcttcgctctctgctcctcctgttcgacagtcagccgcatcttcttttgcgtcgccaggtgaagacgggcggagagaaacccgggaggct

SEQ ID NO: 86 - DNA, 92 bp, Artificial
  Hairpin sequence Hsd_78, human
gggtctgcgggcaagaggcgcacgggggtcgaccagggctccctagggctagatgtgcggccaagcacccccggggcgtcgtccgcagaccc

SEQ ID NO: 87 - DNA, 95 bp, Artificial
  Hairpin sequence Hsd_79, human
ggcaggcgtctgcggaactggagcgcggctgaagaagctgctcttggcagaggaaggggcaggctggcaaagtggattggctgtgggcgcctgcc

SEQ ID NO: 88 - DNA, 88 bp, Artificial
  Hairpin sequence Hsd_8, human
gcctggatacatgagatggttgaccagagagcacacgctttatttgtgccgtttgtgacctggtccactaaccctcagtatctaatgc

SEQ ID NO: 89 - DNA, 99 bp, Artificial
  Hairpin sequence Hsd_80, human
tgtatcacctgccacttgcacctgtatcacacctgtgtgttacacctgtcactcctgtgtgtcactcttgtgtgttgcagatgtgggggcaggtgagca

SEQ ID NO: 90 - DNA, 87 bp, Artificial
  Hairpin sequence Hsd_81, human
ccagatcctagaaccctatcaatattgtctctgctgtgtaaatagttctgagtagtgcaatattgcttatagggttttggtgtttgg

SEQ ID NO: 91 - DNA, 96 bp, Artificial
  Hairpin sequence Hsd_82, human
gctttgctggctggtgcagtgcctgagggagtaagagccctgttgttgtcagatagtgtcttactccctcaggcacatctccagcgagtctcttgc

SEQ ID NO: 92 - DNA, 99 bp, Artificial
  Hairpin sequence Hsd_83, human
ccaccctttggtacttaaagagaggataccctttgtatgttcacttgattaatggcgaatatacagggggagactctcatttgcgtatcaaactgtcgg

SEQ ID NO: 93 - DNA, 92 bp, Artificial
  Hairpin sequence Hsd_84, human
ttagggtgaccagccattatggtttgcctgggactgaggaatttgctgggatatgtcagttccaggccaaccaggctggttggtctccctga

SEQ ID NO: 94 - DNA, 72 bp, Artificial
  Hairpin sequence Hsd_85, human
tgtggcctaaggtggtcaaaggagccccatcaggcctgaggtctgacccttggctctgccacctggaagaca

SEQ ID NO: 95 - DNA, 92 bp, Artificial
  Hairpin sequence Hsd_86, human
gctgggatgtgtcaggtaggcagtgtattgctagcggctgttaatgattttaacagttgctagttgcactcctctctgttgcattcagaagc

SEQ ID NO: 96 - DNA, 54 bp, Artificial
  Hairpin sequence Hsd_87, human
aggtttgaatcctactgggtggcccagcaggggccccctacagggctcagggct

SEQ ID NO: 97 - DNA, 89 bp, Artificial
  Hairpin sequence Hsd_88, human
agtactgaggacaacaggatatgggctctaatgcttggaaaattatagtctctctgcatttagagccctgggaccctgtctgtttggct

SEQ ID NO: 98 - DNA, 98 bp, Artificial
  Hairpin sequence Hsd_89, human
gcaaacattaggagagtatcttctctgttttggccatgtgtgtactcacagcccctcacacatggccgaaacagagaagttactttcctaatatttgc

SEQ ID NO: 99 - DNA, 100 bp, Artificial
  Hairpin sequence Hsd_9, human
gtgctaacctttggtacttggagagtggttatccctgtcctgttcgttttgctcatgtcgaatcgtacagggtcatccactttttcagtatcaagagcgc

SEQ ID NO: 100 - DNA, 99 bp, Artificial
  Hairpin sequence Hsd_90, human
agcataaaccaaaagccccagggggagaaggtgtaagacttgtctgagagccagtggactcgtttggcacaaagggtatctgggggaattggtgtagtt

SEQ ID NO: 101 - DNA, 97 bp, Artificial
  Hairpin sequence Hsd_91, human
ggaagttttttgggaatgcagattccagggcccctccttcagataggcttatttgatgggtccaaaaaagggggtctgaatttgtaataaacgcgcc

SEQ ID NO: 102 - DNA, 74 bp, Artificial
  Hairpin sequence Hsd_92, human
gacacagatggcgcgcgggctcttgggttctgtagttttctcgcgatccaaaaggctccgtgcccaagtgagtc

SEQ ID NO: 103 - DNA, 97 bp, Artificial
  Hairpin sequence Hsd_93, human
taaggtcagagctggtcagtgacgaagccagacccagagcccaggtcaccaggccatggggctttttactgcctcagtgatcgtgagcctggtttta

SEQ ID NO: 104 - DNA, 100 bp, Artificial
  Hairpin sequence Hsd_94, human
tcccccctgctggccgcaggtgctctgacgaggttgcactactgtgctctgagaagcagtgcaatgatattgtcaaagcatctgggaccagccttgggga

SEQ ID NO: 105 - DNA, 83 bp, Artificial
  Hairpin sequence Hsd_95, human
ttctcctctcagggaagctttttgctcgaattatgtttctgatccgaatataagacgaacaaaaggtttgtctgagggcagag

SEQ ID NO: 106 - DNA, 87 bp, Artificial
  Hairpin sequence Hsd_96, human
tctcaagctatgagtctacaaaggaaagcgctttctgttgtcagaaagaagagaaagcgcttcccttttgagggttacggtttgaga

SEQ ID NO: 107 - DNA, 95 bp, Artificial
  Hairpin sequence Hsd_97, human
cgaggcactgcggcccagggtctggtgcggagagggcccacagtggacttggtgacgctgtatgccctcaccgctcagcccctggggctggcttg

SEQ ID NO: 108 - DNA, 95 bp, Artificial
  Hairpin sequence Hsd_98, human
gattcctgggctctgacctgagacctctgggttctgagctgtgatgttgctctcgagctgggatctccggggtcttggttcagggccggggcctc

SEQ ID NO: 109 - DNA, 82 bp, Artificial
  Hairpin sequence Hsd_99, human
aggtggtacaccctttcctctctgccccatagggtgtagctctaactaccctctagggaagagaaggttgggtgaacagcct

SEQ ID NO: 110 - DNA, 96 bp, Artificial
  Hairpin sequence Hsd_from_Mmd_100, human
tcatgggggagggcgggcaagtcaggtgaccgggtgagaatcagagctatgctgaggtggcctggttaccttgacattcctgtgattctgccaagg

SEQ ID NO: 111 - DNA, 99 bp, Artificial
  Hairpin sequence Hsd_from_Mmd_101, human
tggtgaggtggtgagaaattttgtcttcaagcaaaggtcattggaccagacttttggatttagtgacggtggaggctataatttttcctgctggcagca

SEQ ID NO: 112 - DNA, 89 bp, Artificial
  Hairpin sequence Hsd_from_Mmd_105, human
tagggttgatgcaaaagaaaggaaaacatctttgaagttctgaaaaataggacttcaaagatgttttcctttcttctccatcgaatcta

SEQ ID NO: 113 - DNA, 100 bp, Artificial
  Hairpin sequence Hsd_from_Mmd_107, human
tatgaaaaaaatgtctgtaatatgcagtaaatgaggcccataactcattggaacttccaatgagttatgtgttaaattcatatttcacaacatggtcatg

SEQ ID NO: 114 - DNA, 99 bp, Artificial
  Hairpin sequence Hsd_from_Mmd_112, human
gttcctgtttcctcctgtggcccagagtttgatgtgtagggcagtgatgcccagctccttgcacctctgggccacatcctgggtggccagcatggcagc

SEQ ID NO: 115 - DNA, 99 bp, Artificial
  Hairpin sequence Hsd_from_Mmd_115, human
gagaacatttctgtgccgggccagcaggtgttacctgagtaagattgagattctctcaccaggcgacaaggcttgttgtgcccgagacaggcacagctc

SEQ ID NO: 116 - DNA, 98 bp, Artificial
  Hairpin sequence Hsd_from_Mmd_117, human
aggtgctgggcgaggccatgactggcatctcccaaaatgccaagaacggaaacctgccagagtttggagatgccatttccacagcctcaaaggcactt

SEQ ID NO: 117 - DNA, 98 bp, Artificial
  Hairpin sequence Hsd_from_Mmd_119, human
ggcttgtcagaatgaattctagggtgccaagagacattttgcatatttaagagtatgcaaagtgtctcatggcacattagaattcattctgacaagcc

SEQ ID NO: 118 - DNA, 75 bp, Artificial
  Hairpin sequence Hsd_from_Mmd_120, human
ggggtgggactggtcatgcagccgtgtgtcaggagtaatgattgtagaggggcggggcatgaagagtgccgttct

SEQ ID NO: 119 - DNA, 91 bp, Artificial
  Hairpin sequence Hsd_from_Mmd_123, human
ttcctggggattaatgaatagctgggtggagttaattgcctaaagtatagtctcaggccattaacctcagttggtcactaatccctaggaa

SEQ ID NO: 120 - DNA, 77 bp, Artificial
  Hairpin sequence Hsd_from_Mmd_126, human
ggaattagctctctaaatgccctacaatgactggctgccttgtcagtcctctgagggcgatttgagtacctaactcc

SEQ ID NO: 121 - DNA, 100 bp, Artificial
  Hairpin sequence Hsd_from_Mmd_128, human
gagcttttaaaaaaattactggtgtccaagctccaccctggaccaattaaatgagctctctaggggtggggcctggacatcagtaattttttaaaagctc

SEQ ID NO: 122 - DNA, 99 bp, Artificial
  Hairpin sequence Hsd_from_Mmd_129, human
ctgacctccacctccactgactccctcttttccttcttccctcagccaatgctgaggccttgaaggagatgctgggggacagtgaaggagaagggacag

SEQ ID NO: 123 - DNA, 93 bp, Artificial
  Hairpin sequence Hsd_from_Mmd_133, human
tttgcaaggtccagctattaattacactaagtgacaaagattctccttgttgcatgatggacacaaggatgaatctttgttactgactgcaaa

SEQ ID NO: 124 - DNA, 98 bp, Artificial
  Hairpin sequence Hsd_from_Mmd_136, human
cattactagcagttaatgattggtttgttagttaatggcccaaggcaaagctgagggcggttaagactctaacaaacgagttgtgaattgtagcaatg

SEQ ID NO: 125 - DNA, 83 bp, Artificial
  Hairpin sequence Hsd_from_Mmd_137, human
tgtgccatatcatattcttaccaaaacatttgctttgattggtggctcggtcaacaaagactttggctgagatgacgtggcca

SEQ ID NO: 126 - DNA, 96 bp, Artificial
  Hairpin sequence Hsd_from_Mmd_138, human
tcttattgtgtttggttgaggcctagtaattttcgataaagcccttaatccaagtacttgactgaaaattaccaggcttctctgtggaggatgagg

SEQ ID NO: 127 - DNA, 93 bp, Artificial
  Hairpin sequence Hsd_from_Mmd_141, human
gggctgcccaagagaagggcttattttgactcaggatgctgcagaagctgtggcaggcttgtcaaaatacatctgcctcttcttgagcggccc

SEQ ID NO: 128 - DNA, 79 bp, Artificial
  Hairpin sequence Hsd_from_Mmd_145, human
gtccactccatcaaagggtctaatggtcagcagagggggctaaatgtgtcccccgtgacctgacattaacggagtggac

SEQ ID NO: 129 - DNA, 84 bp, Artificial
  Hairpin sequence Hsd_from_Mmd_147, human
ggagagaggcgcagacaagagaaaataagcctgcccagcaatcatgtctggggctgctgggagggctctctctgttctctttcc

SEQ ID NO: 130 - DNA, 81 bp, Artificial
  Hairpin sequence Hsd_from_Mmd_148, human
tggcagctaatgacctactggaggacttatgtgcctaaggcaaagctgaaggccattagcccaacaggtcattaactgaca

SEQ ID NO: 131 - DNA, 100 bp, Artificial
  Hairpin sequence Hsd_from_Mmd_149, human
gctgaattgatttctgtgcataaacggtggcctagattcttgccctgtggaaatccatggaattaagaactctaggttcctggagtgataagaggtaagc

SEQ ID NO: 132 - DNA, 91 bp, Artificial
  Hairpin sequence Hsd_from_Mmd_150, human
ccagatacaagtgaatttaagcccacatgtattgaagctgcaagcttagtttgcaatttatgtgcaagtggttttgtatttattgatctgg

SEQ ID NO: 133 - DNA, 96 bp, Artificial
  Hairpin sequence Hsd_from_Mmd_154, human
gtgaccatcgttgttcaaggtgttgtgttcagggcccttccaggtaattgaggctttggggcggccacagactcgacatctaagaacaacggtctc

SEQ ID NO: 134 - DNA, 77 bp, Artificial
  Hairpin sequence Hsd_from_Mmd_155, human
tggtgtgcctgcccccttccgtcattgctgtgaatgggctggacggaggaggggccggcgaaaatgatgatgaacca

SEQ ID NO: 135 - DNA, 95 bp, Artificial
  Hairpin sequence Hsd_from_Mmd_156, human
ttttcacttccaggtaatctaattccaaggcatcaggagctttttaataaaaatcctgtgtttgctggagtaaggttgcctgaaatcaaggaaag

SEQ ID NO: 136 - DNA, 79 bp, Artificial
  Hairpin sequence Hsd_from_Mmd_157, human
gctgagagcagcaggatgagggttgacctatatctgtcaggtgtaggtcaaaccctgaatttttgttgcaaacttcagt

SEQ ID NO: 137 - DNA, 89 bp, Artificial
  Hairpin sequence Hsd_from_Mmd_159, human
agtaagtggtcagagagcaggaaatggcttctaattagtagtgtcctcacaggatacaactgcagaagataatttcctgtccccttatt

SEQ ID NO: 138 - DNA, 98 bp, Artificial
  Hairpin sequence Hsd_from_Mmd_161, human
gcttgatgatgctgctgatgctggcggtgatcccgatggtgtgagctggaaatggggtgctacgtcatcgttgtcatcgtcatcatcatcatccgagc

SEQ ID NO: 139 - DNA, 72 bp, Artificial
  Hairpin sequence Hsd_from_Mmd_162, human
aaatgagaaatttttagggccattgagcaccatgcattttcagtgttcagtagccctgaaaatttctcattt

SEQ ID NO: 140 - DNA, 86 bp, Artificial
  Hairpin sequence Hsd_from_Mmd_163, human
gctaaatcattgccagtagtttccaggacattccgctaggctaagcaaaattgccctaagcggtagtcagaaaccactggagtagt

SEQ ID NO: 141 - DNA, 88 bp, Artificial
  Hairpin sequence Hsd_from_Mmd_166, human
tgttaaaataaatggctcatgacttatgaagtgttaaaataaacgtttatttcaacactatacatgtcataagccatttatttcaaca

SEQ ID NO: 142 - DNA, 94 bp, Artificial
  Hairpin sequence Hsd_from_Mmd_167, human
ggcggtagcccaagccttgcaggccccagcggaatgaccagggcgatggaaggaagtcgtgctcgttgcgggcgtggggaatgggcttaccggc

SEQ ID NO: 143 - DNA, 93 bp, Artificial
  Hairpin sequence Hsd_from_Mmd_169, human
gtcaatatctgtgcctcactgtttgttaaatattttgacccagttgccatgggtcagggtgagtgacctgaagggtgggcaggaaggagtgat

SEQ ID NO: 144 - DNA, 94 bp, Artificial
  Hairpin sequence Hsd_from_Mmd_170, human
tttgctgtgaaaattgcaaatgtttgcctggagcaaatacgtgaaaatttgcaatttgttgtaggtgtggaattttgctatttttcacagtgga

SEQ ID NO: 145 - DNA, 97 bp, Artificial
  Hairpin sequence Hsd_from_Mmd_172, human; Hsd_from_Mmd_96, human
ttttcaagtagtcaaactctggttcatggctctggaaggtgctgtaaacattatattctcctctgctatgagactggattttgttctaaaatgaaaa

SEQ ID NO: 146 - DNA, 89 bp, Artificial
  Hairpin sequence Hsd_from_Mmd_173, human
ggtatggattttctttctcaattgctctggagtcaccccgaggctcttaatttgggctattagccgtgggtgatttcaaaatccatatt

SEQ ID NO: 147 - DNA, 81 bp, Artificial
  Hairpin sequence Hsd_from_Mmd_174, human
gaaaacagctttaaagtttagtcattctgatggaatgtaaaagcttgtatcagtgacaggttgacagaaaaagctgttttc

SEQ ID NO: 148 - DNA, 81 bp, Artificial
  Hairpin sequence Hsd_from_Mmd_176, human
gagtctccaaagacaattgtgaaggggattgtgtttgactgatcaatcaaaagcaacccccttgcttgtcttctgagactc

SEQ ID NO: 149 - DNA, 85 bp, Artificial
  Hairpin sequence Hsd_from_Mmd_178, human
cactggaattatgttttatcttaagtccacactggatcctcaggatggatccagtgaggattttgaaagaagcataattcctttg

SEQ ID NO: 150 - DNA, 94 bp, Artificial
  Hairpin sequence Hsd_from_Mmd_180, human
gaggaagggagcggcagcagtgaaggatttgacccccctgccactgataggcagttctctggggcccggaatcagctgcgccgcccccagtatc

SEQ ID NO: 151 - DNA, 94 bp, Artificial
  Hairpin sequence Hsd_from_Mmd_181, human; Hsd_from_Mmd_276, human
gagacttcgaaaatgaacgagaacgagatcttgatgcagatcttgagcgagaagagcgagatccagacttggatttgtttgtttttgacatctc

SEQ ID NO: 152 - DNA, 98 bp, Artificial
  Hairpin sequence Hsd_from_Mmd_182, human
gtttaggggtggacctgatgtccctgagtgtatgtggtgaacctgaatttgccttgggtttcctcatattcattcaggagtgtcagttgccccttcac

SEQ ID NO: 153 - DNA, 94 bp, Artificial
  Hairpin sequence Hsd_from_Mmd_183, human
ggattccactcttgaatctgaacacaggaaataagcttaggaaccatctatgtctgttggttcctgggcattcttgggactggagagtgtttct

SEQ ID NO: 154 - DNA, 87 bp, Artificial
  Hairpin sequence Hsd_from_Mmd_184, human
gaatgactaaacagtcttgcatatatttctggaatacttggaaatgcctctgttatttttggaaatgtgtgcttgttcaggttattc

SEQ ID NO: 155 - DNA, 75 bp, Artificial
  Hairpin sequence Hsd_from_Mmd_185, human
gaccgaccaccagacagacagaggggacgctgctgccaccgcagggactaatctgtctgtggggatgggagagtc

SEQ ID NO: 156 - DNA, 99 bp, Artificial
  Hairpin sequence Hsd_from_Mmd_186, human
ctgcctgctctgcaaaacttaggaggctacttttgattggaataaaagtgcacacactgactttttaaaatgtggctttaattaatggtgggtgggcag

SEQ ID NO: 157 - DNA, 100 bp, Artificial
  Hairpin sequence Hsd_from_Mmd_187, human; Hsd_from_Mmd_525, human
gaggagttaagagttcattcggctgtccagatgtatccaagtaccctgtgttatttggcaataaatacatctgggcaactgactgaacttttcacttttc

SEQ ID NO: 158 - DNA, 91 bp, Artificial
  Hairpin sequence Hsd_from_Mmd_189, human
ttttcaaagcaatgtgtgacaggtacagggacaaatcccgttaataagtaagaggatttgtgcttggctctgtcacatgccactttgaaaa

SEQ ID NO: 159 - DNA, 93 bp, Artificial
  Hairpin sequence Hsd_from_Mmd_19, human
cgcagccgctgctgctgccggcccccctagccttgagggttccgcccacggccgggccccctccgggccggcagcagccgccgaacagctccg

SEQ ID NO: 160 - DNA, 97 bp, Artificial
  Hairpin sequence Hsd_from_Mmd_190, human
ctggccgtgctggcggggctggctctgggcctcagacactctggaatcgccaaggtctgagctcagggcctggcccccttcccaggctaacagccgg

SEQ ID NO: 161 - DNA, 59 bp, Artificial
  Hairpin sequence Hsd_from_Mmd_193, human
ggaggagcagcagggtgaaactgacacagttctggtgagtttcactttgctgctcctcc

SEQ ID NO: 162 - DNA, 99 bp, Artificial
  Hairpin sequence Hsd_from_Mmd_195, human
ctgtaaagcctttattgccacttgttaaggaagacctgtatagctcactcgtgagtaataaggcggtcaacccaaactaaggcactaaaaattttatag

SEQ ID NO: 163 - DNA, 96 bp, Artificial
  Hairpin sequence Hsd_from_Mmd_196, human
agaaacatctcaaatcatgctgacagcattttcactattagcggcaaacaaatcaactataattaaaatgctgtcaaagtgatttgagatttttct

SEQ ID NO: 164 - DNA, 85 bp, Artificial
  Hairpin sequence Hsd_from_Mmd_197, human
aatctaggaggcaggtgctcacttgtcctcctccatgcttggaaaatgcagggaggaggccatagtggcaactgttaccatgatt

SEQ ID NO: 165 - DNA, 85 bp, Artificial
  Hairpin sequence Hsd_from_Mmd_198, human
gcgggggattcctttctggcctcattcctgtaccatttgtttgcaggcttgtagggggagaggaggacattttggaatcctttgt

SEQ ID NO: 166 - DNA, 79 bp, Artificial
  Hairpin sequence Hsd_from_Mmd_20, human
tcctcctcaggtgctcatgtgggagagattagcttgtgtttataagccgatctgttcattaagcgcctgctggtgtgga

SEQ ID NO: 167 - DNA, 100 bp, Artificial
  Hairpin sequence Hsd_from_Mmd_203, human
gcagaaataatcatgtaatcatcttgtaattatatggtacttactatgcaattacatagtaaatacattgtaaattcaaaataattgcatggttatttgc

SEQ ID NO: 168 - DNA, 98 bp, Artificial
  Hairpin sequence Hsd_from_Mmd_204, human
ccctggcagcgggctctgcttcggaaactgttgactcagagggtggccgggcggccagcgtctgatgtcagcctggagctgggccagggagtgcaggg

SEQ ID NO: 169 - DNA, 94 bp, Artificial
  Hairpin sequence Hsd_from_Mmd_208, human
agatgatcccttaccctacctgtctttctatcagtgtagctgaggctaattcagtgcattgatgtaagattattatggggttgagcatttatct

SEQ ID NO: 170 - DNA, 89 bp, Artificial
  Hairpin sequence Hsd_from_Mmd_211, human
gcatcaaacggcttgttttgggcctcaatccgctccttttctgattttcgaaggtagggagcagcttcgccaaaaacagccatctcagc

SEQ ID NO: 171 - DNA, 85 bp, Artificial
  Hairpin sequence Hsd_from_Mmd_212, human
ggtagaggagatggcgcaggggacacgggcaaagacttgggggttcctgggaccctcagacgtgtgtcctcttctccctcctccc

SEQ ID NO: 172 - DNA, 99 bp, Artificial
  Hairpin sequence Hsd_from_Mmd_215, human
ctggagtccagccctccaggggttaagggggcgggaccagctagttgccatggtgccagactgtttgggtctgctctctgacccttgggggctccccag

SEQ ID NO: 173 - DNA, 95 bp, Artificial
  Hairpin sequence Hsd_from_Mmd_217, human
ttgagctgtaattccattgagggtttctggtgactccagcttcgtacttgtggttatagtattcccagaacccctcaatggagtgatttcctgaa

SEQ ID NO: 174 - DNA, 83 bp, Artificial
  Hairpin sequence Hsd_from_Mmd_219, human
catgagtggggtctcctgaacatagtgttcaggtggacttttgggtgtctgagcaccacttttggagggagatttcgctcctg

SEQ ID NO: 175 - DNA, 99 bp, Artificial
  Hairpin sequence Hsd_from_Mmd_222, human
tgggctgcagaggcaccagccctgatggtgtgtcagccaagacatggaagtgagaggtaggcggaggccccattggggttggtcgactctcagcatcca

SEQ ID NO: 176 - DNA, 95 bp, Artificial
  Hairpin sequence Hsd_from_Mmd_223, human
tgtaattccatcttggtgttatggtgacatgaaaatccactcaagcttttctgtcagggtttatgaagttatcaaagccccttgatggaattaca

SEQ ID NO: 177 - DNA, 92 bp, Artificial
  Hairpin sequence Hsd_from_Mmd_225, human; Mmd_225, mouse
tgcatttcagagaatatgcattttacctttgggaatatgttaatttcaggcagcattccctatgggaaaggtgataccagctctgatatgca

SEQ ID NO: 178 - DNA, 91 bp, Artificial
  Hairpin sequence Hsd_from_Mmd_226, human
ttaatgtatccatttttctcagttagtgtcaggtttcattaggaaacatgagaggcctgacattaatcgtatgaaaaatgggtctcaggga

SEQ ID NO: 179 - DNA, 96 bp, Artificial
  Hairpin sequence Hsd_from_Mmd_227, human
cagcgagaggcactttgtacttctgccaggagaccatatgatatctctcagtgactctcctgcatgccaggaacacggacccatgcctcttagctg

SEQ ID NO: 180 - DNA, 99 bp, Artificial
  Hairpin sequence Hsd_from_Mmd_228, human; Mmd_228, mouse
gcctcctcactgcactcttgtctcagttcttgtttcgttagcaggattgactcagtcttgcagcgaggctcggagcagcccagggtcgcaggtagaggt

SEQ ID NO: 181 - DNA, 98 bp, Artificial
  Hairpin sequence Hsd_from_Mmd_229, human
ttgcagcccagtgaagtggggtccggtcattccagtctacatctttgtagtttgggtcacagagacctttcttcaaaatcttcttcactaagctgtag

SEQ ID NO: 182 - DNA, 94 bp, Artificial
  Hairpin sequence Hsd_from_Mmd_23, human
atttttcattccttcagtgttgaaacaatctctactgaaccagcttcaaacaagttcactggagtttgtttcaatattgcaagaatgataagat

SEQ ID NO: 183 - DNA, 93 bp, Artificial
  Hairpin sequence Hsd_from_Mmd_231, human
tagccagtaggtactgatttatgaaaagctgtaaaagatcaggtggagctctgtaggacatcgtttacagctgcatggattgaaacctatcta

SEQ ID NO: 184 - DNA, 98 bp, Artificial
  Hairpin sequence Hsd_from_Mmd_232, human
tgttttcttcacttctgtttatttgcagtcactcaaactgacacgtgctgtcaatatctcctcactgtaagtgtaagcagaagtatttaaagaaagcg

SEQ ID NO: 185 - DNA, 99 bp, Artificial
  Hairpin sequence Hsd_from_Mmd_235, human
gtgtgcttgttgtcgaagatgagggcctcctggatgagctggtgctgctgctccagcaggtccaggttgggcttgtagtccacgatgctgcgttcatac

SEQ ID NO: 186 - DNA, 91 bp, Artificial
  Hairpin sequence Hsd_from_Mmd_236, human
tttctggagattaatagtggactgaagctaatagcctttggctgactctccagccatcagcttttcccagctggtcattaattcccagaaa

SEQ ID NO: 187 - DNA, 77 bp, Artificial
  Hairpin sequence Hsd_from_Mmd_237, human
cgctggcggaggagcagacggaggtggcggtcaagctagagcctgagggaccgccaacgctgctacctccgcaggcg

SEQ ID NO: 188 - DNA, 100 bp, Artificial
  Hairpin sequence Hsd_from_Mmd_243, human
gaaaagtgaaaagttcagtcagttgcccagatgtatttattgccaaataacacagggtacttggatacatctggacagccgaatgaactcttaactcctc

SEQ ID NO: 189 - DNA, 100 bp, Artificial
  Hairpin sequence Hsd_from_Mmd_245, human
caggagcagctggagtcggggattaccccctgctgctgacgtgaggacggtgaacaatcgggaacgttcggtggaagggggaattccccgccgctccctg

SEQ ID NO: 190 - DNA, 98 bp, Artificial
  Hairpin sequence Hsd_from_Mmd_246, human; Hsd_from_Mmd_266, human
tgaccccaagaaaccaaagggcaagatgtctgcttatgccttctatgtgcagacgtgcagagaagaacataggaagaaaaacccagaggtccctgtca

SEQ ID NO: 191 - DNA, 96 bp, Artificial
  Hairpin sequence Hsd_from_Mmd_247, human
ccactttcagctttcagtttttatttaaaagacttaattggaagtttttattactcccaattaggctttttaattaaaaggaaaagttgaaagagg

SEQ ID NO: 192 - DNA, 92 bp, Artificial
  Hairpin sequence Hsd_from_Mmd_249, human
taactctttgattgccatggcaagaaccatgctgatgattctagcttgtgacatttgtgtggttaaaggttgccgcagcagtcaaagggtta

SEQ ID NO: 193 - DNA, 99 bp, Artificial
  Hairpin sequence Hsd_from_Mmd_250, human
tggaccaaaggaacttttctggtctattgggtaaatatgtattaaagtaatttttaaaatacttacatacttaataggctggaagtggtcaatggttta

SEQ ID NO: 194 - DNA, 79 bp, Artificial
  Hairpin sequence Hsd_from_Mmd_253, human; Hsd_from_Mmd_319, human; Mmd_253, mouse
ccttgatttcaatgtttcccactttggtggttgatctgataataggtcttccaaccaaagctgggaagatgtgttctgg

SEQ ID NO: 195 - DNA, 93 bp, Artificial
  Hairpin sequence Hsd_from_Mmd_255, human
tggtgctaaagttggtatggcagcctgcaccattccagttcccaggaatgggcttaggatcaaaggttgctatcactccaaagtcttcacaca

SEQ ID NO: 196 - DNA, 99 bp, Artificial
  Hairpin sequence Hsd_from_Mmd_256, human
gaaaggcctcagtctgcgggccccgaggagggttgtgggcccttttttgtgaatgaagcgccacggaacagccctcctggggtccccacgagccgcgtc

SEQ ID NO: 197 - DNA, 98 bp, Artificial
  Hairpin sequence Hsd_from_Mmd_258, human
ggggtgtttgtttctttctcggtttgctggttggaagacgaagatgaggaggagctggtgctggccctcgaatcgtcatccgacatagcgaacccccc

SEQ ID NO: 198 - DNA, 99 bp, Artificial
  Hairpin sequence Hsd_from_Mmd_259, human
tcccctgctgcactaaattgacacttgggcaagcaatggctctcgtcagtcactccgaagctgctacttccagaagtgtcgatttagtgcagcggggga

SEQ ID NO: 199 - DNA, 96 bp, Artificial
  Hairpin sequence Hsd_from_Mmd_26, human; Mmd_26, mouse
ctctgcttcacctttcttttagcaaagatgaggatttctaccttcaacatacattgttggtcttgtcttctgtttttgttaggagggacagcagtg

SEQ ID NO: 200 - DNA, 96 bp, Artificial
  Hairpin sequence Hsd_from_Mmd_260, human
tatctcttttctgattagagaggtggcatggagcctggctcattaggcagaagtaatgagccagtctatacgccatctctaattagagaaaagata

SEQ ID NO: 201 - DNA, 94 bp, Artificial
  Hairpin sequence Hsd_from_Mmd_261, human
agaatcttctgacaatgaaggtaggcggacaacgaggagattgctgtcacgcatagcttctagcatgtcatctacttttttttcacgaagatct

SEQ ID NO: 202 - DNA, 98 bp, Artificial
  Hairpin sequence Hsd_from_Mmd_262, human
ttacacctgaaacttctcacttgggggtacaattagcctctggcgcctttagaatgagttgctggtgactaattatgccctcaaggaggcatttgtaa

SEQ ID NO: 203 - DNA, 98 bp, Artificial
  Hairpin sequence Hsd_from_Mmd_263, human
gagtacagctggcagaaagtgttacagatataatccttattttccttgtttttgttggtattgtaggagggatctgtaggcctttctgtccagcgctc

SEQ ID NO: 204 - DNA, 97 bp, Artificial
  Hairpin sequence Hsd_from_Mmd_264, human
caaatgctgtgactcgaactcggggaaattgttcctgtaatggagttctcaatttattggtcaatttcaccttgattttgtgtcactgttagctttg

SEQ ID NO: 205 - DNA, 93 bp, Artificial
  Hairpin sequence Hsd_from_Mmd_270, human
tggttgggtgtggggatgggggcacttctgttcctgacaacctcattatgtggttatcaggagcagacactgcccaggttgtcacgacaacca

SEQ ID NO: 206 - DNA, 92 bp, Artificial
  Hairpin sequence Hsd_from_Mmd_271, human
cttggaggctttgcctcatccacaggcatttggcatccgagatggaaaatttcacttgtcaggtcgccatggaaaccagcagagccactgag

SEQ ID NO: 207 - DNA, 98 bp, Artificial
  Hairpin sequence Hsd_from_Mmd_272, human
tattagcatgttaataggtctttgtgcccagaggctttggattataaattatgaagactgcattagccagatgggcaggattaattagcatgctagta

SEQ ID NO: 208 - DNA, 99 bp, Artificial
  Hairpin sequence Hsd_from_Mmd_274, human
caggatggctggggtcgtacatctgtgggtaaggataagcatgcaagtactgtatgtatgactgatgctgactcatgggtgaggagacagccactcttg

SEQ ID NO: 209 - DNA, 98 bp, Artificial
  Hairpin sequence Hsd_from_Mmd_275, human; Hsd_from_Mmd_389, human
ggcaccagctcctccagctccagccccaggtcctgcgcgatgcggcccacgaaggtgccgtgtttggcctcctcggggacggagtagcggagctggcc

SEQ ID NO: 210 - DNA, 78 bp, Artificial
  Hairpin sequence Hsd_from_Mmd_277, human
tagctaattgcaggtgcaattggccacttgcaccatcagttgaagggcaagtggttaattgagcccacaagtgaccta

SEQ ID NO: 211 - DNA, 99 bp, Artificial
  Hairpin sequence Hsd_from_Mmd_279, human
tctaggcttgttttgagttcattgttcagcagatgagcttgttcaagatttcactttcagcagttcatttgattgaacttgggaccaataaaagcgaga

SEQ ID NO: 212 - DNA, 96 bp, Artificial
  Hairpin sequence Hsd_from_Mmd_28, human
tggctttgctccttttctgcatcacttccattaatgcacccacaattcctgaagtgggtgcaggtgttggtggtgtagactcttggccatcagcca

SEQ ID NO: 213 - DNA, 93 bp, Artificial
  Hairpin sequence Hsd_from_Mmd_280, human
ggctacagtaggcttgctgcccagtccaaggccagcagtgttgtgtctggcctgtaagaggcaggtagcaagccatgaatatgtccaaaggcc

SEQ ID NO: 214 - DNA, 80 bp, Artificial
  Hairpin sequence Hsd_from_Mmd_284, human
acgttagtccatttgctcagcggcccttccatgatggacgccatcttgggagccgccaatgacaacaaacggcctgacgt

SEQ ID NO: 215 - DNA, 98 bp, Artificial
  Hairpin sequence Hsd_from_Mmd_286, human
agaaactaatctggattcactccctctggttgatacccactcaaaaaggacacttctgattaagacggttgaaactagagatggacaggttggtatct

SEQ ID NO: 216 - DNA, 100 bp, Artificial
  Hairpin sequence Hsd_from_Mmd_291, human
cccagcgcgtggccgaccgcctgggcctggagctgggcaaggtggtcacgaagaagttcagcaaccaggagaccaggtgggggccgcgccggcggccggg

SEQ ID NO: 217 - DNA, 100 bp, Artificial
  Hairpin sequence Hsd_from_Mmd_293, human
gcttgagctaggggggtaacctcaagctatggaaaaaattagggttagacatgaagtattttttcatagcctgaggaaggccccatgggcagcagcaggc

SEQ ID NO: 218 - DNA, 91 bp, Artificial
  Hairpin sequence Hsd_from_Mmd_294, human
attttcatcacctagggatcttgttaaaaagcagattctgattcagggaccaagattctgcatttttagcaagttctcaagtgatgctaat

SEQ ID NO: 219 - DNA, 99 bp, Artificial
  Hairpin sequence Hsd_from_Mmd_295, human
ggtcagagtgttttgttgtcaggattcttagttgcttgcattggcttatgaaaaggtcattatgagagaagggatctgaatagcaacgagattccagct

SEQ ID NO: 220 - DNA, 87 bp, Artificial
  Hairpin sequence Hsd_from_Mmd_296, human
gtatatttttctcttgcagggttacattttgccatctgcctttccagagtaagtggctctatgtgggcactgaacgaggtaatatac

SEQ ID NO: 221 - DNA, 96 bp, Artificial
  Hairpin sequence Hsd_from_Mmd_297, human
agactaattaatgtttcagagtgtttaaacacaatgcctctacagaagcaatggcattataatgtgttgggacactctagtgttgtaattagtact

SEQ ID NO: 222 - DNA, 98 bp, Artificial
  Hairpin sequence Hsd_from_Mmd_298, human
cttcagcaaccttacagccccatcattcatttgcataatggatgcctttttatccattatgcaaatggtatgagaggaaaattaggcaataagggaag

SEQ ID NO: 223 - DNA, 91 bp, Artificial
  Hairpin sequence Hsd_from_Mmd_299, human
gtgatgaatgtatattggtaaggagggtgaggttgaaggggcagggacctctcaaatgaagtgactttctttaccagcacattcagaacac

SEQ ID NO: 224 - DNA, 95 bp, Artificial
  Hairpin sequence Hsd_from_Mmd_300, human
cagccagaactaccctggcgggtggaaacagcttttaccgcgtgtggctgtcgcatgtggttttggaattttccaacgccccctacgattggctg

SEQ ID NO: 225 - DNA, 99 bp, Artificial
  Hairpin sequence Hsd_from_Mmd_301, human
aggctataattccactgaggggtgctggcggtgacatgtaccatgaagtcagaatctgagtggtttgtcaccagaacccccagggtagaattataggct

SEQ ID NO: 226 - DNA, 96 bp, Artificial
  Hairpin sequence Hsd_from_Mmd_302, human
ggcagtgcctaaggaattaatggcccgctgaggttaatgagtccctcctctgggccatcaactctcccagctggccattaatcccctagaaatgtc

SEQ ID NO: 227 - DNA, 92 bp, Artificial
  Hairpin sequence Hsd_from_Mmd_303, human
gtaacacaggtggtacaccctttcctctctgccccatagggtgtagctctaactaccctctagggaagagaaggttgggtgaacagccttac

SEQ ID NO: 228 - DNA, 94 bp, Artificial
  Hairpin sequence Hsd_from_Mmd_305, human
gcgatgcgttgcatttgggtgacaggcagttgttcgtgcttgaataactcaggatttgaattctacagtttgtggcatccgtgtaatgcttagc

SEQ ID NO: 229 - DNA, 97 bp, Artificial
  Hairpin sequence Hsd_from_Mmd_306, human
tcaacattttccttgccacagaatgattcctctgagaacaaagtaattgttgacttcaaagaactctgaggaagttcagtggatggaaagaatttgg

SEQ ID NO: 230 - DNA, 97 bp, Artificial
  Hairpin sequence Hsd_from_Mmd_31, human
gcggtcccatcagcggaagctcagggcctgcgggaactggagggtgcctctgaccccttgctctcactctttggctctacttctgacaacgacttgc

SEQ ID NO: 231 - DNA, 96 bp, Artificial
  Hairpin sequence Hsd_from_Mmd_311, human
ggattaattggtcaggaaagacactttttatattgtcaagtggcacttaagccattaattcttgactgtgaaactgcttttcctgggcaatgctct

SEQ ID NO: 232 - DNA, 97 bp, Artificial
  Hairpin sequence Hsd_from_Mmd_314, human
aaattaatgaccaaaatgtcagatgtgtccacagctaattattcattagatgcttaattagtgtgggatgatcatgacattttaatgttattaattt

SEQ ID NO: 233 - DNA, 92 bp, Artificial
  Hairpin sequence Hsd_from_Mmd_316, human; Mmd_316, mouse
tcctctttgcaaaatcatatttttgtgggaatgggccctgctttttgtggcaaggcctgttctgattaataaaggatcgtgaaaaagtaggg

SEQ ID NO: 234 - DNA, 82 bp, Artificial
  Hairpin sequence Hsd_from_Mmd_318, human
gatctctaatacccattagtgttagggcctgttacctcaatcatgagtgacagagccctaattattaaggtaattagaagtt

SEQ ID NO: 235 - DNA, 99 bp, Artificial
  Hairpin sequence Hsd_from_Mmd_321, human
agtatatgagcctataactggcttttacagatgattaaatgctcaggttgaagccgaggcttttgataactgctagagccatttatgacctcatatact

SEQ ID NO: 236 - DNA, 99 bp, Artificial
  Hairpin sequence Hsd_from_Mmd_324, human
ggccgcatggaccacgtggaccgctgcttcgacgtgctggctgtggagtactatgtgccggagacggaccagtggaccagcgtgagccccatgcgggcc

SEQ ID NO: 237 - DNA, 82 bp, Artificial
  Hairpin sequence Hsd_from_Mmd_325, human
ggcgtgagggtatgtgcctttggactacatcgtggaagccagcaccatgcagtccatgggcatatacacttgcctcaaggcc

SEQ ID NO: 238 - DNA, 81 bp, Artificial
  Hairpin sequence Hsd_from_Mmd_326, human
tttcctaagtgatgtcagcccctgtgagcttccaataggaaccaggcataaatcagggagctgaccttatccaataggaaa

SEQ ID NO: 239 - DNA, 91 bp, Artificial
  Hairpin sequence Hsd_from_Mmd_328, human
ccaagcagggacttcttgccagcccttggcagcatgaggcctcccatcatcatccaccgagctggcaagaagtatggcttcaccctgcggg

SEQ ID NO: 240 - DNA, 90 bp, Artificial
  Hairpin sequence Hsd_from_Mmd_33, human
ggtcagaatcctgtgcttacgacacctcggtctgttgccatggaaacaggggcggattaaggctggagcgtggagcgcagggatcaggcc

SEQ ID NO: 241 - DNA, 95 bp, Artificial
  Hairpin sequence Hsd_from_Mmd_330, human
gtgccccgcggagtggctgtgatgagcgggatgcggaaggtgtccactgagccgatctgtctcccgctcacccacagcagttgctccatggtcac

SEQ ID NO: 242 - DNA, 100 bp, Artificial
  Hairpin sequence Hsd_from_Mmd_333, human
gggctgatgagcagttcaagttccctgcatagaaatagcctcagaatagttccagtgttgtttctaattgcaatggatgagcagactgtctcattaatcc

SEQ ID NO: 243 - DNA, 99 bp, Artificial
  Hairpin sequence Hsd_from_Mmd_334, human
ctttcacaaagagcaaagggtttcagaatcaagtttgtcttcaagcagcagcaaacgcttgctgacaagatattttggaacaactcgctattaggaagg

SEQ ID NO: 244 - DNA, 95 bp, Artificial
  Hairpin sequence Hsd_from_Mmd_335, human
gaggcgagtgtggatctaatcttcagctgattaaatgtccctcattaatgagtttctttaattaatgaagtttttgggtctgctccacttgcttt

SEQ ID NO: 245 - DNA, 88 bp, Artificial
  Hairpin sequence Hsd_from_Mmd_336, human
gtgggaattacaggctggagagaccttccttctgcaggagaagaaattcttgaagtagaatctgaggtgtatcaagcttaattcctat

SEQ ID NO: 246 - DNA, 77 bp, Artificial
  Hairpin sequence Hsd_from_Mmd_338, human
tccaagttcatgatgaagagtttatcagtgggggtcagttccacaggctgtctggtgaactctttgtcgaagttgga

SEQ ID NO: 247 - DNA, 76 bp, Artificial
  Hairpin sequence Hsd_from_Mmd_34, human
gtcgagaattgattttatattcctggtcaagttgctgaaatgtagcaattgggtcaggaagatcggttccttggac

SEQ ID NO: 248 - DNA, 98 bp, Artificial
  Hairpin sequence Hsd_from_Mmd_340, human
ggcccagccacagtgacattcatcccccggtgggtctggcctcagtcctcacaacccaccgacaacaatgaatgttgattgtgacctcggctgtggcc

SEQ ID NO: 249 - DNA, 100 bp, Artificial
  Hairpin sequence Hsd_from_Mmd_343, human
aatatgcagggtgatggttggggatttggcagcatggccccatgaacattagtgggagttagtctgctaaatcccagacactgaacagaatcctcatgtt

SEQ ID NO: 250 - DNA, 100 bp, Artificial
  Hairpin sequence Hsd_from_Mmd_344, human
gttctcggtagctatggaaatcccagggtttccctagcaacagctccagatgggtcttgttgctagggaacgctgggatttcccaggctgccagatgagc

SEQ ID NO: 251 - DNA, 91 bp, Artificial
  Hairpin sequence Hsd_from_Mmd_346, human
ctcaggcgcgggttggggaggatgttttggatgccgctctgctgacgctggtgccctctgttcttacatcctccttaatttactgcctgag

SEQ ID NO: 252 - DNA, 98 bp, Artificial
  Hairpin sequence Hsd_from_Mmd_347, human
gcggcgggctgagttttgaaaacttaggattctaaatgtataaattgtttggaacatttaacatctagtcattaagcagtcacccccttgccctccgt

SEQ ID NO: 253 - DNA, 83 bp, Artificial
  Hairpin sequence Hsd_from_Mmd_35, human
gggcttggcctggctctgctgggacacttcgcacttttgccatttttggccagaaggcgctccctgctagcccggctctgttc

SEQ ID NO: 254 - DNA, 99 bp, Artificial
  Hairpin sequence Hsd_from_Mmd_353, human
gcattttctgaggattgatgaccagctaggaggagttgatggtccttggctgaaacttgggccattaactctagccagtcattaattgcctggaaatgt

SEQ ID NO: 255 - DNA, 92 bp, Artificial
  Hairpin sequence Hsd_from_Mmd_354, human
ggcctctgtatttaatttggctcagccgggaagatttttggctctgtgcgtgtgtatattctgaaaaatctttccgagtgagcccaggggtt

SEQ ID NO: 256 - DNA, 94 bp, Artificial
  Hairpin sequence Hsd_from_Mmd_355, human
gttgtagcatgtggttgtattaatgaacgttacaggagagctttataatcatttataaatctgtaacattcaataaagaaacacatgttgcaat

SEQ ID NO: 257 - DNA, 98 bp, Artificial
  Hairpin sequence Hsd_from_Mmd_356, human
ttggtgttgtcaatttgcgagaccagtctggaattctcggccttggtacacaagatctagaattaagagattgtacacacaaatgaagcaaacaccaa

SEQ ID NO: 258 - DNA, 88 bp, Artificial
  Hairpin sequence Hsd_from_Mmd_358, human
ggtcggtgcagcagagaaacagaggataatggcgtctgcagtgctgtcgcctggagcgccctcctcctttctcgttcgcgcactcact

SEQ ID NO: 259 - DNA, 93 bp, Artificial
  Hairpin sequence Hsd_from_Mmd_361, human
agaaagatgctgaaaccaatgaagcaatctattgtacaaaggagcctttcattaaggctcgtgttattgtcattcgttggctggtttctttct

SEQ ID NO: 260 - DNA, 97 bp, Artificial
  Hairpin sequence Hsd_from_Mmd_363, human
agacaaaccctaaaaatgtagcagaagcatttccgcacactggtgtccagaatctagtttgtgcagaaatgtttccactagatttatagagtactct

SEQ ID NO: 261 - DNA, 99 bp, Artificial
  Hairpin sequence Hsd_from_Mmd_367, human
gcacggcttggcattggaagttttatttgaatcaggggagaatattatttgagatattttcctaaatgacaaatgaatgatttacctgcagcaatgtgt

SEQ ID NO: 262 - DNA, 98 bp, Artificial
  Hairpin sequence Hsd_from_Mmd_37, human
gagtccgtgaccgggatttgcttgtgcagttgtttattaactacctgggcattttgctgatggtgcaatttgcagagctgttaatggtaatgaaactc

SEQ ID NO: 263 - DNA, 100 bp, Artificial
  Hairpin sequence Hsd_from_Mmd_370, human
atgccatgcaaattggcacagaggcaatcagcctgactctaaaagctgccccactggccgcaggctggctcattgtcttttctgtcaatttgcatctcat

SEQ ID NO: 264 - DNA, 99 bp, Artificial
  Hairpin sequence Hsd_from_Mmd_371, human
gcagctaggagcctgccaagtgctggtggaagtagaggccgaaaaggctggcgagcagctggcaggcagccgtccaccagatgaggtaggccaggacgc

SEQ ID NO: 265 - DNA, 88 bp, Artificial
  Hairpin sequence Hsd_from_Mmd_372, human
cttggcgtccttgtctctctctcccctgcccagtggcctccttgtctggctcactgggcaggagccctaatcggattcgacagctgag

SEQ ID NO: 266 - DNA, 88 bp, Artificial
  Hairpin sequence Hsd_from_Mmd_373, human
tattttctgtttcagaaaattcttccaggaggaaatacatcatttgatgtagtttttcttgcaagagtagtaggaaatgtagaaaata

SEQ ID NO: 267 - DNA, 96 bp, Artificial
  Hairpin sequence Hsd_from_Mmd_376, human
gttatcataaaataatcacagccctcaggtgctgtgaggcactgggtgcagctaagtgtctctaattccccaagggggtgattagctgatgataac

SEQ ID NO: 268 - DNA, 83 bp, Artificial
  Hairpin sequence Hsd_from_Mmd_378, human
cgaacacccaaaataatgtgagatcaggcaccatgcattatgcatgaggtctgatttaacatgaacattatttcttgtgtacg

SEQ ID NO: 269 - DNA, 81 bp, Artificial
  Hairpin sequence Hsd_from_Mmd_382, human
aggactcgatgacgttgaagttgcacagggggctgacctcaaagaaggtcatgcagttcttctctgcgtacgcgcgggcct

SEQ ID NO: 270 - DNA, 93 bp, Artificial
  Hairpin sequence Hsd_from_Mmd_383, human
cttcacctggcacactgggctggaagactttattcagatccaaggaggaggctctggaatgggttttctgtggccgaggtggcggggtgggag

SEQ ID NO: 271 - DNA, 84 bp, Artificial
  Hairpin sequence Hsd_from_Mmd_387, human
tagggaaacccagttgcttgggtggatccagacaaaatggattttcatccttggattttgtgcccacgcctctgggtcaccttg

SEQ ID NO: 272 - DNA, 100 bp, Artificial
  Hairpin sequence Hsd_from_Mmd_388, human
agggtgtgtattatagacgagcaatgatgtgccttggcagttaatggcactcagtccaaactaccagggcatgtcattattaggtcataatacccaccct

SEQ ID NO: 273 - DNA, 94 bp, Artificial
  Hairpin sequence Hsd_from_Mmd_39, human; Mmd_39, mouse
cagctcagaatgtggtaggagctatcagaacttagtgatcaagtgaagtcgtagttactaatttctgatgctcttcccctgcagaagagagctg

SEQ ID NO: 274 - DNA, 82 bp, Artificial
  Hairpin sequence Hsd_from_Mmd_391, human
gcaaagctggcatttcggagcacgtcagaagcgctggctcccagccctggaagtcagcgtctggcctctgagccagcactgc

SEQ ID NO: 275 - DNA, 79 bp, Artificial
  Hairpin sequence Hsd_from_Mmd_396, human
tacaggacgtctgtgcaattcgatggagctcatcacttttcggtctatgagcaattaaattgcaccaggaagccctgta

SEQ ID NO: 276 - DNA, 91 bp, Artificial
  Hairpin sequence Hsd_from_Mmd_398, human
tgcttgattagtcagattgatggaagaggccatatgtggccatgtcaaaacttggctacaaggctctttccctctctatgaaggacaggca

SEQ ID NO: 277 - DNA, 87 bp, Artificial
  Hairpin sequence Hsd_from_Mmd_399, human
gctcaccctggaggctacagatgctgatggaagccgcagccatgccgctgtggactacagcatcatcagtggcaactggggccgagt

SEQ ID NO: 278 - DNA, 100 bp, Artificial
  Hairpin sequence Hsd_from_Mmd_40, human
ctgtcggctcagaagtccatggagctgtgggccaggtagtccttgcgaccgatgttgctgacctgcttggtctgcatagcctcgagtttggggcagtcag

SEQ ID NO: 279 - DNA, 95 bp, Artificial
  Hairpin sequence Hsd_from_Mmd_405, human
ctgccctctgcagcctgccccgagcctgggccctcttcccacccaggtcaggagcgggggagggtagggtcctggggagggaagaggaggggcag

SEQ ID NO: 280 - DNA, 92 bp, Artificial
  Hairpin sequence Hsd_from_Mmd_408, human
gaaaaatcagctttaattaatttgagtgccagctctgtgtataattacacagcatgagagtttgcatgcaaatgcagaaatgctgattgttc

SEQ ID NO: 281 - DNA, 88 bp, Artificial
  Hairpin sequence Hsd_from_Mmd_409, human
ccatcagtcaggaaattgtatgataaataggaggtagccaggtttcgtggcacttccttctgcttcagcagttttcgcctgactttgg

SEQ ID NO: 282 - DNA, 83 bp, Artificial
  Hairpin sequence Hsd_from_Mmd_41, human
gtttgtgtgcatttgcaggaacttgtgagtctcctattgaaaatgaacaggagactgatgagttcccgggaacacccacaaat

SEQ ID NO: 283 - DNA, 91 bp, Artificial
  Hairpin sequence Hsd_from_Mmd_410, human
gtcgttgctgacccaagccgtcaggtagatgtagaaagcgctgggattaatgatgccatctgcatccaccagacgctgtttagtcaactac

SEQ ID NO: 284 - DNA, 88 bp, Artificial
  Hairpin sequence Hsd_from_Mmd_411, human
cttcttgtataagcactgtgctaaaattgcagacactaggaccatgtcttggtttttgcaataatgctagcagagtacacacaagaag

SEQ ID NO: 285 - DNA, 96 bp, Artificial
  Hairpin sequence Hsd_from_Mmd_412, human
ccattagcagtaggctatggattaggagtggtagaaaggatatctttaaatagatttctttgtttgtttgtttggtttgaggcctagtgattgagg

SEQ ID NO: 286 - DNA, 98 bp, Artificial
  Hairpin sequence Hsd_from_Mmd_413, human
gcaggcatttcctgaggattaatgaccagctgggaggaaccagtggcccttggctctgcctcccagccagccattaattctaaggaaatgtcttttgc

SEQ ID NO: 287 - DNA, 100 bp, Artificial
  Hairpin sequence Hsd_from_Mmd_416, human
ggctcggaggtgggggaggcggtttgtgctggtgcctggtgtataatcgggttaatccagtagacaaagaataaaccaatagccctcccacctcacttcc

SEQ ID NO: 288 - DNA, 98 bp, Artificial
  Hairpin sequence Hsd_from_Mmd_418, human
tcactggttctcctcccctcccccaatccaaacttccctactccgtctaccggcagcaacaaccacgtgggggaggggtaagagagtagaaccggtga

SEQ ID NO: 289 - DNA, 96 bp, Artificial
  Hairpin sequence Hsd_from_Mmd_42, human
gaccacaagaaaccaaagggcaagatgtctgcttatgccttctttgcgcagacatgcagagaagaacataagaagaaaaacccagaggtccctgtc

SEQ ID NO: 290 - DNA, 100 bp, Artificial
  Hairpin sequence Hsd_from_Mmd_422, human
tcctcatttgagtcataatcctccatttgtgacatcacaattagccacttgtggtgatgattataatgtcatgaacaaaggattatgactgggagttgga

SEQ ID NO: 291 - DNA, 99 bp, Artificial
  Hairpin sequence Hsd_from_Mmd_424, human
atcccaagagtcactgacacaagctctacaactattgaactagaatgggaacccccagctttcaatggtggtggggaaattgttggctattttgttgat

SEQ ID NO: 292 - DNA, 94 bp, Artificial
  Hairpin sequence Hsd_from_Mmd_426, human
gcctgttgacatgttcagacgcaactccagtagaagtgctgggcctggcagggggccaggcctgagtcacagctgctgggcgtatcaacgtggc

SEQ ID NO: 293 - DNA, 87 bp, Artificial
  Hairpin sequence Hsd_from_Mmd_428, human
tcgatgtgaagatcctgcagcagctggtgaccttgaatgagggcatcgaggcagtgcgctggctgttggaggagcgggggacgttga

SEQ ID NO: 294 - DNA, 86 bp, Artificial
  Hairpin sequence Hsd_from_Mmd_429, human
gttttacagctcagaggggattaggacaaaagatgtatttaaaatatggtatgtgtctctctctgtccctctgcagctgatggagc

SEQ ID NO: 295 - DNA, 78 bp, Artificial
  Hairpin sequence Hsd_from_Mmd_430, human
ccatcgtgcaggattacatttgttctcctcatgctgaaagtatgaggaagagaaaccagattgtgttcaccatggtgg

SEQ ID NO: 296 - DNA, 87 bp, Artificial
  Hairpin sequence Hsd_from_Mmd_431, human
gctggttttggcacttctctcactctagtgagttgcaaaaaaacaagcaggtgctagcaatctgagagaccacatgccagctacagc

SEQ ID NO: 297 - DNA, 98 bp, Artificial
  Hairpin sequence Hsd_from_Mmd_433, human
gttggtgccctgataaagccgagccctggcagataaatgctgaggtgcagccaagccatttatttgaggagcagcagggctcctatcagggcaccaac

SEQ ID NO: 298 - DNA, 88 bp, Artificial
  Hairpin sequence Hsd_from_Mmd_435, human
agcatttgtgtgcataaaatgggtaattgcacacacaaatgggcaattttgtatgcaattatccattttgcatacgcaattatatgct

SEQ ID NO: 299 - DNA, 97 bp, Artificial
  Hairpin sequence Hsd_from_Mmd_436, human
tccaggtgtccaccaaggacgtgccgctggcgctgatggcctgtgccctgcggaagaaggccacagtgttccggcagccgctggtggagcagccgga

SEQ ID NO: 300 - DNA, 91 bp, Artificial
  Hairpin sequence Hsd_from_Mmd_437, human
gcctttcctgggcatacagaaccccctggcagccagtcttgaggcaacacctgccttccgcctggctgacagcaggactaacccagcaggc

SEQ ID NO: 301 - DNA, 97 bp, Artificial
  Hairpin sequence Hsd_from_Mmd_438, human
gttgttattaaatcaccttgaagtgtctcttgtaaaagataactccctgcattgtagaaatgtctttgacaagagaggattagggtgaaactgcaat

SEQ ID NO: 302 - DNA, 89 bp, Artificial
  Hairpin sequence Hsd_from_Mmd_439, human; Mmd_439, mouse
tctagggagccaccagtggtgtaagtgctttggctaagtgatggagacaactgaggaggagtgtgattaccactgggttcctgaatgga

SEQ ID NO: 303 - DNA, 99 bp, Artificial
  Hairpin sequence Hsd_from_Mmd_44, human
ggtgaggagcaggttagctgggtgaaaagttcacagtgaggggagctgtctgttccctcgcttaatttatccactatttggctaaccttgctctgaacc

SEQ ID NO: 304 - DNA, 93 bp, Artificial
  Hairpin sequence Hsd_from_Mmd_443, human
cagagggggaatcacatgagctacagctgagttcacaggaatgatttgtgagcctcagtatttgcacatgtgtggtcatgggggacctctctg

SEQ ID NO: 305 - DNA, 73 bp, Artificial
  Hairpin sequence Hsd_from_Mmd_448, human
ccttctcagccccagctcccgctcacccctgccacgtcaaaggaggcagaaggggagttgggagcagagaggg

SEQ ID NO: 306 - DNA, 83 bp, Artificial
  Hairpin sequence Hsd_from_Mmd_45, human
gggccctgctgattgttaaactcaacggagatagaattttttttcttcctctgtgtgtgtttattgtaatatctggagggctt

SEQ ID NO: 307 - DNA, 90 bp, Artificial
  Hairpin sequence Hsd_from_Mmd_450, human
gcagctggaaagggcttagccctttaatgccggcagctgtatcttcacagtgcggctgctctgctgaggggctggactctgtccagaagc

SEQ ID NO: 308 - DNA, 91 bp, Artificial
  Hairpin sequence Hsd_from_Mmd_452, human; Hsd_from_Mmd_473, human
taataaattaaatgcctaaactggcagagtgcaaacaattttgactcagatctaaatgtttgcactggctgtttaaacatttaatttgtta

SEQ ID NO: 309 - DNA, 97 bp, Artificial
  Hairpin sequence Hsd_from_Mmd_455, human
tctttgtcatggtgtcagaagattaattatgctcgtgtgagaacagctttctcattgggtaaaaagagacattatccttctgacttgtgacagaaga

SEQ ID NO: 310 - DNA, 87 bp, Artificial
  Hairpin sequence Hsd_from_Mmd_456, human
taggcacgcagctttggagacgccgaggggaaaatggatttaattgattcgttacctctggtattgtaatgcctgctgcgatgttta

SEQ ID NO: 311 - DNA, 82 bp, Artificial
  Hairpin sequence Hsd_from_Mmd_459, human
gaccagctcacaggaagtggccttcaatagtccgatagcttcctgtatcctgacattgtgaaggcccacttcctgtgctgtc

SEQ ID NO: 312 - DNA, 98 bp, Artificial
  Hairpin sequence Hsd_from_Mmd_461, human
gccggagggggcggcgcgcgagagcctggtggccctggtcagcacctcggacagggactcgggcgccaacgggcaagtgcgctgcgccctctatgggc

SEQ ID NO: 313 - DNA, 100 bp, Artificial
  Hairpin sequence Hsd_from_Mmd_464, human
ttaggcagcaggagaaaacaatgggccaggttctcattctgtgtaatctcataggatggttccattgtatagcccattgtttcttccatgtactgcctga

SEQ ID NO: 314 - DNA, 87 bp, Artificial
  Hairpin sequence Hsd_from_Mmd_465, human
ccgtgtgagtttgtactgcaaagctccttggcatccttgcctgagttgggtgttgggaagctcaaattgcagctacaaactggctgg

SEQ ID NO: 315 - DNA, 91 bp, Artificial
  Hairpin sequence Hsd_from_Mmd_466, human
gtgtttatttgaatgtgtgatggggaggtcatcaaaatgaacacactcgttgtgtttatttgaatctcacatcgctcataagaatacacgc

SEQ ID NO: 316 - DNA, 79 bp, Artificial
  Hairpin sequence Hsd_from_Mmd_468, human
gtccgcggagtctctaactggcgacagatgggccactttcttctggccacaaaggggccggaatggagcgctccgcggc

SEQ ID NO: 317 - DNA, 85 bp, Artificial
  Hairpin sequence Hsd_from_Mmd_47, human
gcctgccccagaaccagccctttgtcttctcccttggcacaggccaggtcatcaagggctgggaccaggggctgctggggtgagt

SEQ ID NO: 318 - DNA, 92 bp, Artificial
  Hairpin sequence Hsd_from_Mmd_470, human
ttggtcctatttcctcctgtggcccggagttggatgtgtagggcaatgatacccagctccttgcacctctgggccacatcctgggtggtcaa

SEQ ID NO: 319 - DNA, 94 bp, Artificial
  Hairpin sequence Hsd_from_Mmd_471, human
aaaatttttgtaggtggactgggagagcagcagattgcagatgcgtttaacagcagggtattttctgctgccatgaccagttaaggaaaatttt

SEQ ID NO: 320 - DNA, 90 bp, Artificial
  Hairpin sequence Hsd_from_Mmd_472, human
ggcccctctgtgtggctgctgctggctcagacccctgcccaccttgggcgggtgggtctgggtcttggctggcacgcacacccaggggcc

SEQ ID NO: 321 - DNA, 95 bp, Artificial
  Hairpin sequence Hsd_from_Mmd_476, human
ccgggtctcagggaaatcaggcttctggagagtgagtttgtttgtaataataatacgaattattctctcagcctgaggtcaccgggaggtgtagg

SEQ ID NO: 322 - DNA, 98 bp, Artificial
  Hairpin sequence Hsd_from_Mmd_477, human
gtccagcaatattttaagctcccagccagacagcttttatgaagggagggatgtttgggaaacaatgggagtgagagaatgggagagctgtgttgggc

SEQ ID NO: 323 - DNA, 95 bp, Artificial
  Hairpin sequence Hsd_from_Mmd_478, human
tcggtccagtatatggttggttggtcctgagcatgttggaagagtttctgttcaacatgacatgctctgaggccacttggccagaaatgagctga

SEQ ID NO: 324 - DNA, 93 bp, Artificial
  Hairpin sequence Hsd_from_Mmd_48, human
cctttggttcacattaaaatataaattccctgtgggagagaccctattaagccttgcagggaattgctaatttacatcttaatgtgaatgagg

SEQ ID NO: 325 - DNA, 99 bp, Artificial
  Hairpin sequence Hsd_from_Mmd_480, human
gatcacgtcaccatggccaaccgggagttggcatttaaagctggcgacgtcatcaaagtcttggatgcttccaacaaggattggtggtggggccagatc

SEQ ID NO: 326 - DNA, 96 bp, Artificial
  Hairpin sequence Hsd_from_Mmd_481, human
aagtggtttggagggctgcgttcattttgagtgctgttgtaatcaattttagatacagcattccacaatggatcagtttgcatccaaacctgcctt

SEQ ID NO: 327 - DNA, 99 bp, Artificial
  Hairpin sequence Hsd_from_Mmd_482, human
cctcggctccctggagccgctccaccgatggcaccatcttggcgcagaaactcgccgaggaggtgcccatggacgtggcctcttacctctacaccgggg

SEQ ID NO: 328 - DNA, 91 bp, Artificial
  Hairpin sequence Hsd_from_Mmd_483, human
ccagctgcccgggaagcttggcagccaggctgagggaaggaagcatcggctctctttgaaggtcggctgccagtgaatctcaggcagctgg

SEQ ID NO: 329 - DNA, 77 bp, Artificial
  Hairpin sequence Hsd_from_Mmd_485, human
ttatttcctccactgcctgactttattgaatgccatggggcatattaatcagtaaagccaagtagtgcatgaaataa

SEQ ID NO: 330 - DNA, 99 bp, Artificial
  Hairpin sequence Hsd_from_Mmd_486, human
gtcggagctgttacatttggcagcattccttgttagcatttgataaacaattattgccaaatgttagcaaggaaacctgccaaatgttacagctcagat

SEQ ID NO: 331 - DNA, 100 bp, Artificial
  Hairpin sequence Hsd_from_Mmd_487, human; Hsd_from_Mmd_535, human; Hsd_from_Mmd_78, human
tgctgttgtccttggagaagtctgggcctgggacagaggagggatgtaatctgaacactcctttgtcacaggtcaccagggtgtgcttgaggggacggta

SEQ ID NO: 332 - DNA, 91 bp, Artificial
  Hairpin sequence Hsd_from_Mmd_488, human
cagctagctccgttcgtgatccgggagcctggtgccagcgagacctggaatttccggtctggttggtctggggccccgcggagccaggttg

SEQ ID NO: 333 - DNA, 98 bp, Artificial
  Hairpin sequence Hsd_from_Mmd_489, human
gggaaagaggactattatttaaaaggcttttaaatgatgtcactatatattcttttaacaacatcctctaaaagcctttgaaaaaatagtcccttcct

SEQ ID NO: 334 - DNA, 98 bp, Artificial
  Hairpin sequence Hsd_from_Mmd_49, human
gcgagagccatccccgaacaccttgcggtaggaggaggaggagcacaggtagtgctccgagccgaagctcatggtgccgggatcggggccggacgtgc

SEQ ID NO: 335 - DNA, 89 bp, Artificial
  Hairpin sequence Hsd_from_Mmd_490, human
agtcaggtactcgaatggaggttgtccatggtgtgttcattttatttatgatgagtattacatggccaatctcctttcggtactcaatt

SEQ ID NO: 336 - DNA, 93 bp, Artificial
  Hairpin sequence Hsd_from_Mmd_491, human
gaactgcttttcttttctcctttttgcacaaagagtctcatgtctgatatttagacatgatgagctttgtgcaaaaggggagctggctacttc

SEQ ID NO: 337 - DNA, 99 bp, Artificial
  Hairpin sequence Hsd_from_Mmd_492, human
cgtcaaggatttggcatttcaatcgatgggaaatgatgtgtgatcattctaatttattatacatcatttcccatcaatcgacgaactaaagccttgacg

SEQ ID NO: 338 - DNA, 91 bp, Artificial
  Hairpin sequence Hsd_from_Mmd_494, human
gctctgtggccatttcggtttttccagtccgatgcccctgagggggagggtcgggccccttggaaaatccgttttcatggcaacacggagc

SEQ ID NO: 339 - DNA, 95 bp, Artificial
  Hairpin sequence Hsd_from_Mmd_499, human
ttcgagaggtcacacatgtgatgaattgtgggtgcttgcctggcttcccaggcgagaaccataagcaattattaaatgccattgtgcgtcaggaa

SEQ ID NO: 340 - DNA, 81 bp, Artificial
  Hairpin sequence Hsd_from_Mmd_500, human
gattttcccaacattttatggcaatttgtttcatttgccgcaggcccgtaacgatgcaaattgccataaagtgggataatc

SEQ ID NO: 341 - DNA, 96 bp, Artificial
  Hairpin sequence Hsd_from_Mmd_501, human
tctaatggccagataatggaccacaataaaagttatgggtccccagctggggtgaagctcataatggttggcaagatccattatttgaccattagg

SEQ ID NO: 342 - DNA, 89 bp, Artificial
  Hairpin sequence Hsd_from_Mmd_503, human
ggccaaaatctgccctcagttatgcatgcacagcttgcattgacgtcagtaagaactgtgctcatgtaacaggaggcagaaatttggct

SEQ ID NO: 343 - DNA, 98 bp, Artificial
  Hairpin sequence Hsd_from_Mmd_505, human
tcccatgtgcctttaaggaatcattcttgatctcagccataaaattggaaagctgcataactgacaagatgatccctgaaggcagctgaaacatgggg

SEQ ID NO: 344 - DNA, 91 bp, Artificial
  Hairpin sequence Hsd_from_Mmd_506, human
gtcttttaactctatattgctggtggctctgatttcatgatgcattgtgcttatgaaactctagctttcagcactaaaatgagttaaagat

SEQ ID NO: 345 - DNA, 100 bp, Artificial
  Hairpin sequence Hsd_from_Mmd_507, human
gcggcgcggggcgcgtcgcccccctcagtccaccagagcccggatacctcagaaattcggctctgggtctgtggggagcgaaatgcaacccaaaccccgt

SEQ ID NO: 346 - DNA, 80 bp, Artificial
  Hairpin sequence Hsd_from_Mmd_509, human
ggctcactggaggctgcagagaagtgaactgaagaattacagtcggtttcacttccctgccttatgtgccttcaggtgcc

SEQ ID NO: 347 - DNA, 92 bp, Artificial
  Hairpin sequence Hsd_from_Mmd_51, human
cacttttttgaagtgtgaattgcctgctgtcagcctggagttctcacctccgcaatcctggctgcaggcatatttgcatattggatgctgtg

SEQ ID NO: 348 - DNA, 98 bp, Artificial
  Hairpin sequence Hsd_from_Mmd_510, human
catggtcttggtttggtagcgccccaggatggagtaggcagggccaaggtccttgccagtcctcagtatcttggggtttacattgtaacggggccctg

SEQ ID NO: 349 - DNA, 74 bp, Artificial
  Hairpin sequence Hsd_from_Mmd_511, human
cagaaggtactctgtgggagggaggagataatagccaatgattatattcctccttcatacttgcagtacctctg

SEQ ID NO: 350 - DNA, 97 bp, Artificial
  Hairpin sequence Hsd_from_Mmd_515, human; Mmd_515, mouse
gggcatagttggacgagagggcgtcttggcaggtctctgtgtttaaagagcacaggtgtgagacactgggacgcacgctttcggctcaacattgcct

SEQ ID NO: 351 - DNA, 89 bp, Artificial
  Hairpin sequence Hsd_from_Mmd_516, human
aaaacacttgcagctgattggccgaggtcgatatggagcagtatataaaggctccttggatgagcgtccagttgctgtaaaagtgtttt

SEQ ID NO: 352 - DNA, 94 bp, Artificial
  Hairpin sequence Hsd_from_Mmd_517, human
ttgaggcaaacagcacagaagtcctcattctctattggggctggggggcccttcttggctcctggggttcgaggaatgagtctgtgttcttcag

SEQ ID NO: 353 - DNA, 81 bp, Artificial
  Hairpin sequence Hsd_from_Mmd_518, human
gtcacacagccggattgtccctttgctgctgctgtacacgaaggtgttgcaatgatgggggtggaactcggctgctgtgat

SEQ ID NO: 354 - DNA, 71 bp, Artificial
  Hairpin sequence Hsd_from_Mmd_519, human
gaggtgcccaggtcagtgcgctggttcatagaggcggacgtgtctatgaggaacagcaggatgggcatctt

SEQ ID NO: 355 - DNA, 92 bp, Artificial
  Hairpin sequence Hsd_from_Mmd_52, human; Mmd_52, mouse
ccagcagcagcttcaggccgacagcttcaaagctaagcagatggaaaaccaccagcttattaaggaggctgtagaaatgaagtctgtcatgg

SEQ ID NO: 356 - DNA, 98 bp, Artificial
  Hairpin sequence Hsd_from_Mmd_523, human
gagttgtatcgctcaaataaaagattgaaggatgctgtggcccgctgccatgaggcagagaggaacatagctttgcgttcaagtttagagtggaattc

SEQ ID NO: 357 - DNA, 67 bp, Artificial
  Hairpin sequence Hsd_from_Mmd_524, human; Mmd_524, mouse
gggtgtgataggtatacagctggttgttaccatggtgatggccagtgtcatgcagaagattatacct

SEQ ID NO: 358 - DNA, 88 bp, Artificial
  Hairpin sequence Hsd_from_Mmd_526, human
tcctcctgcttggcgttgatgtctgctttcatctccatggcaacatgacggggaaggacagacagcaggagccgttcctgcagaggga

SEQ ID NO: 359 - DNA, 95 bp, Artificial
  Hairpin sequence Hsd_from_Mmd_527, human
cgggtttgtgctgtagacccctcatttactcctttgtggagttaggagcagcttcccattaaaggagatcaggggtaattagcagcattattttg

SEQ ID NO: 360 - DNA, 99 bp, Artificial
  Hairpin sequence Hsd_from_Mmd_529, human
ggtaaatttcctaagcagtgtgtgagaggtgtacgcagaaatcctgttgacaaaatattttgtgcaagtgcctcccacacacagctttaaaatgttacc

SEQ ID NO: 361 - DNA, 99 bp, Artificial
  Hairpin sequence Hsd_from_Mmd_530, human
cccagtgggtgtccagatgcctgacaacattctccaggccaagatacataagagtcctgtggtctggggatgcgaggacagctggacaaacagcttggg

SEQ ID NO: 362 - DNA, 90 bp, Artificial
  Hairpin sequence Hsd_from_Mmd_532, human
agcggcaggggatttgaggggtggttgtgtgatttgtacagctgtcattagggtaattgcatgatattttcccaaatttctcctgcagct

SEQ ID NO: 363 - DNA, 98 bp, Artificial
  Hairpin sequence Hsd_from_Mmd_533, human
agtcacttaagctgagtgcattgtgatttccaataattgaggcagtggttctaaaagctgtctacattaatgaaaagagcaatgtggccagcttgact

SEQ ID NO: 364 - DNA, 92 bp, Artificial
  Hairpin sequence Hsd_from_Mmd_534, human
tctatataccctcagagctgatatgagtgtggagattccattcacttcaaaggaagcttcacgcttggatcttctccacaggggtgcttaga

SEQ ID NO: 365 - DNA, 93 bp, Artificial
  Hairpin sequence Hsd_from_Mmd_536, human; Mmd_536, mouse
ggctggggctgaaccgaatgctggcacccctccaaacccaggggatcccccaaaagtaggagggctgccaaacactggagcagcaccgaagcc

SEQ ID NO: 366 - DNA, 96 bp, Artificial
  Hairpin sequence Hsd_from_Mmd_54, human
gaggagggtctcctcgaggggtctctgcctctacccaggactctttcatgaccaggaggctgaggcccctcacaggcggcttcttactctctcctt

SEQ ID NO: 367 - DNA, 97 bp, Artificial
  Hairpin sequence Hsd_from_Mmd_56, human
gaccaaggtctttggtcttggaggaaggtgtgctactggaagaggccaccgaggcagggctggtgggggcatcttttttcaggctacgggccttgtc

SEQ ID NO: 368 - DNA, 99 bp, Artificial
  Hairpin sequence Hsd_from_Mmd_58, human
ctcttctgcacccaacgaagggttgaccaaagcggccatgatggcaatgtctgagtatgaagatcggttgtcatctggtctggtcagcccggccccgag

SEQ ID NO: 369 - DNA, 100 bp, Artificial
  Hairpin sequence Hsd_from_Mmd_59, human
gccaccgcgtcctgggccaggcgcccgcccggccggcgggaagaggcgcggacacgcgcacccggcccgccgcgggggcgcgcctggctctgggcggggc

SEQ ID NO: 370 - DNA, 83 bp, Artificial
  Hairpin sequence Hsd_from_Mmd_60, human
gccttggcttcctgctcctcctcatactgttcccgcagcaggtcacagtcgtggcgggaggactgcagggcgtgcgccatggc

SEQ ID NO: 371 - DNA, 98 bp, Artificial
  Hairpin sequence Hsd_from_Mmd_67, human
cttcttccagtcatcctgaggtagatatcatacaggaatgctggggccttatggcttacagcaatccagtaatgatataaaagatgattggaggttag

SEQ ID NO: 372 - DNA, 93 bp, Artificial
  Hairpin sequence Hsd_from_Mmd_68, human
tcgtcctgtctaaaggaagaagtttgttctactgtaaacagttatgggaactgtttgctttggatgggcttggtcctcattggctgaggagga

SEQ ID NO: 373 - DNA, 98 bp, Artificial
  Hairpin sequence Hsd_from_Mmd_69, human
tgggtatttctttcaggtagaagccaggtgaaactcaaagggtccaaactgggttttgctttgagtttccttttggttctgcttgaacttgacactca

SEQ ID NO: 374 - DNA, 95 bp, Artificial
  Hairpin sequence Hsd_from_Mmd_70, human
gattaattctacatgcagctaattatacccacaattggctaattgtcggtgcaattagccaattgcaggcccaattggccacttgtcccataatt

SEQ ID NO: 375 - DNA, 94 bp, Artificial
  Hairpin sequence Hsd_from_Mmd_71, human
cactgtaacaacattttactcctgactgattacattacatattgatattagtgagcatcagtatgtagtgcaatcagtcaggagaaaatgagtg

SEQ ID NO: 376 - DNA, 97 bp, Artificial
  Hairpin sequence Hsd_from_Mmd_74, human
catgccctgatatttgtgtagggataattactatctctcaggcctcacttggtggcaagatgagatgaaaacccataaaacaccactcagggctgtg

SEQ ID NO: 377 - DNA, 99 bp, Artificial
  Hairpin sequence Hsd_from_Mmd_75, human
ggggaaaaaaaaggcagacactgtgtttgatgccaataatggttgccagtggcaccgaggttggcatcagaccctctttgtctagttgctgaatgcctt

SEQ ID NO: 378 - DNA, 88 bp, Artificial
  Hairpin sequence Hsd_from_Mmd_76, human
agggcagcaatttgtgtcagatttcaatctattttgagtggaaaccaatgtaagcctggttgaaaaatggtgagtaaattgctgtcct

SEQ ID NO: 379 - DNA, 92 bp, Artificial
  Hairpin sequence Hsd_from_Mmd_80, human
cggacagtgtgttgatgcttattggtgtctcttttctcttctaggaccagcagagagaagagattccatcttccagaggttgccactgtctg

SEQ ID NO: 380 - DNA, 95 bp, Artificial
  Hairpin sequence Hsd_from_Mmd_81, human
ttcctttaactctcagcttctatttaaaaagctaatttgggagcttgtcgatgctcccaattcagacattttaaataggaactgaaagtcaggag

SEQ ID NO: 381 - DNA, 82 bp, Artificial
  Hairpin sequence Hsd_from_Mmd_82, human
ggtccatctttgggccaagatatgtagtcgccaacagtgagttggcgactatattttttgagtcctggcgcaaagtaagatc

SEQ ID NO: 382 - DNA, 93 bp, Artificial
  Hairpin sequence Hsd_from_Mmd_85, human
acaggccactccatctgttcaaacataatgctccaaaccaaggaatatgttccaaaccaaagagcatgatatttgacagatggagaggtctgt

SEQ ID NO: 383 - DNA, 88 bp, Artificial
  Hairpin sequence Hsd_from_Mmd_90, human
gcctccatttaggtgtcaattgtgtaatttccagctgttcctcagttcaacagctggagttcaatatgctcagggcttatttggaggc

SEQ ID NO: 384 - DNA, 99 bp, Artificial
  Hairpin sequence Hsd_from_Mmd_91, human
cgcacaggaagcagcaggtagttctgtctgctgtctctttagataaaatgtatgtctggcaccaagggaacagctgagaactacctgttgttatgtatg

SEQ ID NO: 385 - DNA, 100 bp, Artificial
  Hairpin sequence Hsd_from_Mmd_92, human
caccttgggtgagggcatgacgctgaaggtgttcatgatgcggtctgggtactcttcccggatcttgctgatgagcagggtgcccatcccggaccccgtg

SEQ ID NO: 386 - DNA, 92 bp, Artificial
  Hairpin sequence Hsd_from_Mmd_93, human
ttgtagttgactaaacagcgtctggtggatgcagatggcatcattaatcccagcgctttctacatctacctgacggcttgggtcagcaacga

SEQ ID NO: 387 - DNA, 93 bp, Artificial
  Hairpin sequence Hsd_from_Mmd_94, human
tgacattttctgtagattgtggacaaagccctagaagagccaaagtatagctcactgtatgctcagctatgtctgcgattggcagaagatgca

SEQ ID NO: 388 - DNA, 83 bp, Artificial
  Hairpin sequence Hsd_from_Mmd_95, human
gacactatcaaagttaaggcaggagagcctgtccacatccctgcagatgtgacaggccttccaatgcctaagattgaatggtc

SEQ ID NO: 389 - DNA, 92 bp, Artificial
  Hairpin sequence Hsd_from_Mmd_97, human
agctttctccaactgttacaatctccacttcactgtcagttgaggtaacattaatttcttcattggcagtaacctggggagtggaggaagct

SEQ ID NO: 390 - DNA, 83 bp, Artificial
  Hairpin sequence Hsd_from_Mmd_98, human
ggcccggctccgggtctcggcccgtacagtccggccggccatgctggcggggctggggccggggccgagcccgcggcggggcc

SEQ ID NO: 391 - DNA, 100 bp, Artificial
  Hairpin sequence Mmd_1, mouse
ggctgggcaaggtgcggggctagggctaacagcagtcttactgacggtttcctggaaaccacacacatgctgttgccactaacctcaaccttactcggtc

SEQ ID NO: 392 - DNA, 86 bp, Artificial
  Hairpin sequence Mmd_10, mouse
cctggcatatagcaggtcctcaataagtatttgttgaaaaaataaatgaagcaacaaatcttatttgagcacctgttatgtggagg

SEQ ID NO: 393 - DNA, 95 bp, Artificial
  Hairpin sequence Mmd_100, mouse
tgagtcatggagaagggagggcaagtcaggtgaccaggtgagactcagaactcctgaggtagcctggttaccttgacattcctgtgattatgcca

SEQ ID NO: 394 - DNA, 99 bp, Artificial
  Hairpin sequence Mmd_101, mouse
tggtgcagcactgagaaattttgtctccaagcaaaggtcattggatcggagtcttggatttagtgacggtggaggctataatttttcttgctggcagca

SEQ ID NO: 395 - DNA, 99 bp, Artificial
  Hairpin sequence Mmd_102, mouse
gagatggaggaagccgagtccaggcctccatgcagcggggagatggagaagtcggcctggggctgtggaggcacggcactcgggttgctaaggatgctc

SEQ ID NO: 396 - DNA, 87 bp, Artificial
  Hairpin sequence Mmd_103, mouse
tgctctgctccccaaatcatgtacccccacagagggtccagatggttcttagttcttctggaggcacatggtttgaagagcagagca

SEQ ID NO: 397 - DNA, 95 bp, Artificial
  Hairpin sequence Mmd_104, mouse
ggcagaggaatgtggctgcttttaaaaggggtctcaggtaaggctgtgtgtgccagccttcctgaccatcagtgatagcagcttgggtgtctgct

SEQ ID NO: 398 - DNA, 87 bp, Artificial
  Hairpin sequence Mmd_105, mouse
aggttgatgcaaaagaaaggaaaacatctttgaagttctgaaaaacaagacttcaaaagatgttttcctttcttctttctcgaatct

SEQ ID NO: 399 - DNA, 90 bp, Artificial
  Hairpin sequence Mmd_106, mouse; Mmd_168, mouse
gccgcgggtgcgcgtctctcggggtcgggggtgcgtggcgggggcccgtcccccgcctcccctccgcgcgccgggtttcgcccccgcggc

SEQ ID NO: 400 - DNA, 89 bp, Artificial
  Hairpin sequence Mmd_107, mouse
atgcccgtaatgtacagtaaatgaggcccataactcattggagcctccaatgagttatgtgctaaattcatatttcacaacatggccat

SEQ ID NO: 401 - DNA, 99 bp, Artificial
  Hairpin sequence Mmd_108, mouse
ccttttcttttcttgaagcctgactccccgcgggacttccaaggcagcataacttcgggtccagactgggctgtcaggcttttccaggaaggatccagg

SEQ ID NO: 402 - DNA, 97 bp, Artificial
  Hairpin sequence Mmd_109, mouse
gggggtgtgagcccctttttctgctttccagcatctttggcatttgcgcaaagttgctcagattgcaatgctggggtgcagtgggcgcgcgactctt

SEQ ID NO: 403 - DNA, 98 bp, Artificial
  Hairpin sequence Mmd_11, mouse
gtgctcttgtggcccatgaaatcaagcttgggtgagacctggtgcagaacaggaaggcgacccatacttggtttcagtggctgcaagaatgactgcat

SEQ ID NO: 404 - DNA, 94 bp, Artificial
  Hairpin sequence Mmd_110, mouse
gtggccagggtggggctgggtggtgcaggcaggagagccattgatctattgtgggctctactccctgccccagccagagcaccccattggctac

SEQ ID NO: 405 - DNA, 81 bp, Artificial
  Hairpin sequence Mmd_111, mouse
agatttcatcagtcaggctagtggttatactccaggacttatctggtcctggatataaccactgccagactgaatggtgct

SEQ ID NO: 406 - DNA, 93 bp, Artificial
  Hairpin sequence Mmd_112, mouse
tgtacctgtttcctcctgtggcccggagtttgatgtgcagggcagtgatgcccaactccttgcacctctgggccacatcctgggcagccaaca

SEQ ID NO: 407 - DNA, 97 bp, Artificial
  Hairpin sequence Mmd_113, mouse
ccaaatgagagggagtgtcagctcagaccctggccatctgttgtgtgtgaggagctggcccctcaggtgtttggtgtggatattcccttctattagg

SEQ ID NO: 408 - DNA, 95 bp, Artificial
  Hairpin sequence Mmd_114, mouse
ggccagggcactttaggctaccccagtgtgtcagtgtagagcaatgttgacaagctctgcctaactgagctgtgggtgttctagagggtatgacc

SEQ ID NO: 409 - DNA, 96 bp, Artificial
  Hairpin sequence Mmd_115, mouse
ggacatttctgtcccaggccagcagctgtcaccttagaaagattaagattctcccaccaggcgacaaggcttgttgtgcccgagacagatacggct

SEQ ID NO: 410 - DNA, 87 bp, Artificial
  Hairpin sequence Mmd_116, mouse
agactttattatgttcgccaaacatctggccttcccaagtatgtcattagacattgggccaggtgtgaggcatcagagtggattgct

SEQ ID NO: 411 - DNA, 97 bp, Artificial
  Hairpin sequence Mmd_117, mouse
aggtcctaggtgaggccatgactggcatctcccaaaatgccaagaatggaaatctgccggagtttggagatgccattgccacagcctccaaggctct

SEQ ID NO: 412 - DNA, 95 bp, Artificial
  Hairpin sequence Mmd_118, mouse
acctttgctactggcatttgagagatggggaaataggcacttggagaggtcgggaatcttgttcaaggtcatcagcaaaggtcagttgtgaaggt

SEQ ID NO: 413 - DNA, 98 bp, Artificial
  Hairpin sequence Mmd_119, mouse
ggcttgtcagaatgaattctagggtgccaagagacattttgcatatttaagggtatgcaaagtgtctcatggcacattagaattcattctgacaggcc

SEQ ID NO: 414 - DNA, 98 bp, Artificial
  Hairpin sequence Mmd_12, mouse
cgattcttgtttgcgtggggaggagggaggggatgggccaagttccctatgcctggaacgcccttcccccccttcttcacctggcgaactcctactcg

SEQ ID NO: 415 - DNA, 92 bp, Artificial
  Hairpin sequence Mmd_120, mouse
gtggccacaggggggtgggactggtcatgtagccgtgtgtcaggagtaatgattgtagaggggcggggcatgaagagtgccattctggaaac

SEQ ID NO: 416 - DNA, 95 bp, Artificial
  Hairpin sequence Mmd_121, mouse
gcttaggccagccgcgtcgccgcttcatgaatggaacccacgtgaccaaacatcatgtgacgtctgaggagcggcggcggcggcggattctgagc

SEQ ID NO: 417 - DNA, 95 bp, Artificial
  Hairpin sequence Mmd_122, mouse
cagtgagaaattaggagggatgtaaagtaagctggctttttgcctgagggcttaatttaaaagaatcctttaggtgcctctgtgatttcttactg

SEQ ID NO: 418 - DNA, 91 bp, Artificial
  Hairpin sequence Mmd_123, mouse
ttcctggggattaatgaatggctgggtggagttaattgcctaaagtatagtctcaggccattaacctcagttggtcactaatctcaaggaa

SEQ ID NO: 419 - DNA, 82 bp, Artificial
  Hairpin sequence Mmd_124, mouse
aggcaggatacagagctctttaaataatgcccagcatttcctggaccagcatgatatttaaagatttttgttccacctgcct

SEQ ID NO: 420 - DNA, 94 bp, Artificial
  Hairpin sequence Mmd_125, mouse
gtttttctattcttgaagcctgactccccgcgggacttccaaggcagcataacttcgggtccagactgggctgtcaggcttttccaggaaggat

SEQ ID NO: 421 - DNA, 91 bp, Artificial
  Hairpin sequence Mmd_126, mouse
gaagcaaggaattagctctctaaatgccccacgatgactggctgacttgctggtcctctgagggcgatttgaatatctaactcctctcttc

SEQ ID NO: 422 - DNA, 100 bp, Artificial
  Hairpin sequence Mmd_127, mouse
gggaatggaggaggcattaggggaagaggggactctgtctgatggattttcattctttttaggaattacccttgtgtccctggtggttcctgtggcttct

SEQ ID NO: 423 - DNA, 99 bp, Artificial
  Hairpin sequence Mmd_128, mouse
agctttttttaaaaattactggtgcccaggctccaccctggaccaattaaatgagctctcttggggaggggcctggacatcagtaattttttaaaagct

SEQ ID NO: 424 - DNA, 96 bp, Artificial
  Hairpin sequence Mmd_129, mouse
ccctgacctctacctccactgactccctcttctccccctccatcagccaatgctgaggccttgaaggagatgctaggggacagtgaaggagaaggg

SEQ ID NO: 425 - DNA, 98 bp, Artificial
  Hairpin sequence Mmd_13, mouse
tggcaggccaggaagaggaggaagccctggaggggctggaggtgatggatgttttcctccggttctcagggctccacctctttcgagccgtagagcca

SEQ ID NO: 426 - DNA, 100 bp, Artificial
  Hairpin sequence Mmd_130, mouse
ctccccagagagatctttcagtaactgatcttctagctggcatttttatagattaggaagcaggccagctggaggtcatactaaattaaactctgctgag

SEQ ID NO: 427 - DNA, 60 bp, Artificial
  Hairpin sequence Mmd_131, mouse
aagtcaggttgtgtggttttgtggttagttttctaagcacaaagccaaagatcttgattt

SEQ ID NO: 428 - DNA, 90 bp, Artificial
  Hairpin sequence Mmd_132, mouse
gaaggagaaaagcaagcagggtgtgaggggactcgtcccacacctgtgtaaacaatgtcaccttacaccccgtcaagcaccctcgccttc

SEQ ID NO: 429 - DNA, 98 bp, Artificial
  Hairpin sequence Mmd_133, mouse
gtgaaggctattaattacactgagtgacaaagactctccgtgttgtatgattgacacaaggatgaatctttgttactgattgcaaataataggaccgc

SEQ ID NO: 430 - DNA, 99 bp, Artificial
  Hairpin sequence Mmd_134, mouse
aggagaaaggacatgtggtgtttactgtgtgagtggtatttggtcaaacagtggcgatctaccagttcataaggatttaccacttagcctaaggtccct

SEQ ID NO: 431 - DNA, 98 bp, Artificial
  Hairpin sequence Mmd_135, mouse
gacagtgattgttgctatgggagacagtgtgggtttgtgacacaagaaaaattctcttgctgttgcaaaagtggatccagactgtcccaatggatgtc

SEQ ID NO: 432 - DNA, 83 bp, Artificial
  Hairpin sequence Mmd_136, mouse
gcagttaatgattggtttgttagttaatggcccaaggcaaagctgagggcggttaagactctaacaaacgatttgtgaattgt

SEQ ID NO: 433 - DNA, 97 bp, Artificial
  Hairpin sequence Mmd_137, mouse
tgtcttttggatcatcatattcttaccaaaacatttgctctgattggtggcccggtcaacaaagactttggctgagatgacatgaccaaaagggaca

SEQ ID NO: 434 - DNA, 96 bp, Artificial
  Hairpin sequence Mmd_138, mouse
tcttattgtgtttggctgaggcctagtaattttcaataaagcccttaatccaagtacttcactgaaaattaccaggcttctctgtggaggatgaga

SEQ ID NO: 435 - DNA, 88 bp, Artificial
  Hairpin sequence Mmd_139, mouse
gtatttcctgaggattaatgatcaactggaagagcctgtggtcttagggccattaactccgggtggtcattaattctgttggaaatat

SEQ ID NO: 436 - DNA, 85 bp, Artificial
  Hairpin sequence Mmd_14, mouse
ggatctcagacgtctcggggatcatcatgtcacgagataccactgtgcccttgtgacagattgataactgaaaggtctgggagcc

SEQ ID NO: 437 - DNA, 99 bp, Artificial
  Hairpin sequence Mmd_140, mouse
gtcttgccagcagaattctcagaggcaaaaggagtgaaatattttgctggaggggatactttcctttcaagaagcattcctttgcctcctgaacaagat

SEQ ID NO: 438 - DNA, 93 bp, Artificial
  Hairpin sequence Mmd_141, mouse
gggctgcccaagacaagggcttattttgacttaggatgctgctgaagctgtggcagccttgtcaaaatacatctgcctcttcttacgctgccc

SEQ ID NO: 439 - DNA, 100 bp, Artificial
  Hairpin sequence Mmd_142, mouse
gatggtgatctagccctttagttttgaggttggtgtactgtgtgtgagtatacatatttatcacacacagtcactatcttcgaaagtgagggtgcacatc

SEQ ID NO: 440 - DNA, 97 bp, Artificial
  Hairpin sequence Mmd_143, mouse
gtggccttgaggctggaccatgtgacacccaagcacagtggttccaatatggttgtctggttgtgggtggagttgcagaactctggctgtgagccat

SEQ ID NO: 441 - DNA, 97 bp, Artificial
  Hairpin sequence Mmd_144, mouse
gtgaagggctggggtggggtgtaggagattctgtactgagattgcagtctctgcctccagctcttctccacagtttcctcatccccatgcctgaaat

SEQ ID NO: 442 - DNA, 79 bp, Artificial
  Hairpin sequence Mmd_145, mouse
gtccactccatcaaagggtctaatggtcagcagaggaggctaaatgtctcccctgtgacctgacattaaccgagtggac

SEQ ID NO: 443 - DNA, 78 bp, Artificial
  Hairpin sequence Mmd_146, mouse
ggagaggtgggggaaagaattaatgtgtgctctctgattcctttcactgaggacttacagattaactcccacttctcc

SEQ ID NO: 444 - DNA, 81 bp, Artificial
  Hairpin sequence Mmd_147, mouse
ggaaagacacagacaagagaaaataagcctgcctagcaattacgtctgggctgcttggagggctctctctgttctctttcc

SEQ ID NO: 445 - DNA, 97 bp, Artificial
  Hairpin sequence Mmd_148, mouse
agagcccttctggcagctaatgacctgctggagtcgtgtgtgcccaaggcaaagcttaaggccattagtccaacaggtcattaactgacaaatgcct

SEQ ID NO: 446 - DNA, 96 bp, Artificial
  Hairpin sequence Mmd_149, mouse
ttgatttccgtgcataaacagtggcctagattcttgccctgtggaaatccacggaattaagaactctaggttcctggagtgataagaggtaagcaa

SEQ ID NO: 447 - DNA, 97 bp, Artificial
  Hairpin sequence Mmd_15, mouse
tggggactgccctcaaggagcttacaatctagctgggggtagatgacttgcacttgaacacaactagactgtgagcttctagagggcaggggcctta

SEQ ID NO: 448 - DNA, 99 bp, Artificial
  Hairpin sequence Mmd_150, mouse
gtgtgccagatacaagtgaatttaagcccacatggattgaagctgcaagcttactttgcaatttatgtgcaagtggttttggatttattgatctggacc

SEQ ID NO: 449 - DNA, 100 bp, Artificial
  Hairpin sequence Mmd_151, mouse
ggggtccctgagttcctgtcagttgttgtatctgcatgtctctttcctgtggcctcagggaagtggcatgcggtccagcttgtcttggtttactttctct

SEQ ID NO: 450 - DNA, 100 bp, Artificial
  Hairpin sequence Mmd_152, mouse
ggaaaaggctgtaattccatcttggggtatctggtgaaccaagctgtgggtctgtggtttatgatgtcaccagaacccccagctggaattacagccttcc

SEQ ID NO: 451 - DNA, 98 bp, Artificial
  Hairpin sequence Mmd_153, mouse
gctttgcttggccttctgggagttgtagttctagaggcacccgggttgcctggctggccccggaactgtggactacagctcccaggccttctctaagc

SEQ ID NO: 452 - DNA, 95 bp, Artificial
  Hairpin sequence Mmd_154, mouse
ggtcatcgttgttcaaagtgttgtgttcggggcccttccaggtgattgaggccttggggcggccacagacacgacatctaaaaacaacggtctcc

SEQ ID NO: 453 - DNA, 71 bp, Artificial
  Hairpin sequence Mmd_155, mouse
tgtggtgtgcctgcccccttccgtcattgctgtgaatgggctggacggaggaggggctggcgaaaatgacg

SEQ ID NO: 454 - DNA, 99 bp, Artificial
  Hairpin sequence Mmd_156, mouse
gcttattttcctcctaggtaatttaattccaagacaccaggagctattttataaaaatcctgtgtttgctggagtaaggttgcctgaggtcaaagatgc

SEQ ID NO: 455 - DNA, 91 bp, Artificial
  Hairpin sequence Mmd_157, mouse
ggccacagctgctgcgagcagcaggatgagggttgacctatatctgtcctctgtaggtcaacccccgaatttgtttgtagaagcttcagtc

SEQ ID NO: 456 - DNA, 85 bp, Artificial
  Hairpin sequence Mmd_158, mouse
gtcatttagaatgatgtgcacagggtaaattcattgggcatatgggcagcatgtgttcaaggactccctgtagcattgagatgac

SEQ ID NO: 457 - DNA, 100 bp, Artificial
  Hairpin sequence Mmd_159, mouse
ctgttggaaggtggtcagagagcaggaaatggcttctaattagtagtgtcctcacaggatgcaactgcagaagacaatttcctgtccccttattggatgg

SEQ ID NO: 458 - DNA, 97 bp, Artificial
  Hairpin sequence Mmd_16, mouse
gctactgtttcatacttgaggagaaattatccttggtgtgttggctcttttggatgaatcatacaaggataatttctttttgagtatcaaatactgc

SEQ ID NO: 459 - DNA, 88 bp, Artificial
  Hairpin sequence Mmd_160, mouse; Mmd_240, mouse
tcttttcttgaagcctgactccccgcgggacttccaaggcagcataacttcgggtccagactgggctgtcaggcttttccaggaagga

SEQ ID NO: 460 - DNA, 98 bp, Artificial
  Hairpin sequence Mmd_161, mouse
gatgctgctgatgctggcggtgatgccgatggtgcgagctgaaaatgggctgctacgtcatcgtcgtcatcgttatcatcatcatcagcacagccatc

SEQ ID NO: 461 - DNA, 81 bp, Artificial
  Hairpin sequence Mmd_162, mouse
gtaagaaatgagaaatttttagggccattgaacaccacgtatttgcagtgttcagtagccctgaaaatttctcatttaagc

SEQ ID NO: 462 - DNA, 90 bp, Artificial
  Hairpin sequence Mmd_163, mouse
gctaaatcattgccagtagtttccaagacattccgctaggctaagcaaattgccctaagtggtagtcagaaacgactggcatggtactgt

SEQ ID NO: 463 - DNA, 99 bp, Artificial
  Hairpin sequence Mmd_164, mouse
gggatttaatggatccccatcctgtcttttgctacacattcacaaattattatttactgagtgcagagcaaaaggcatggtgggtatctatttggttac

SEQ ID NO: 464 - DNA, 95 bp, Artificial
  Hairpin sequence Mmd_165, mouse
acctaggagattggctgtaggggcttctgagtctgatgcccttatatcttaagggacatgaggatagtcagtagtccaatgtccctcttgatggt

SEQ ID NO: 465 - DNA, 100 bp, Artificial
  Hairpin sequence Mmd_166, mouse
gaaaagtgttaaaataaatggctcatgacttatgaagtgttaaaataaacgtttatttcaacactactcatgtcataagccatttatttcgacacttttt

SEQ ID NO: 466 - DNA, 93 bp, Artificial
  Hairpin sequence Mmd_167, mouse
ggcggtagcccaaggtttgcaggcctggccgaatgaccagggcaatggaaggaagtcgtggtcgtcgcgggcgtggggaatgggcttaccggc

SEQ ID NO: 467 - DNA, 99 bp, Artificial
  Hairpin sequence Mmd_169, mouse
gttgatgtccgtgcctcactgtttgttaaatattctgacccggtcaccctgggtcagggtgactgacctgaagggtgagcaggaggaaatgataagagc

SEQ ID NO: 468 - DNA, 66 bp, Artificial
  Hairpin sequence Mmd_17, mouse
ggtaagtgtgcctcgggtgagcatgcacttaatgtaggtgtatgtcactcggctcggcccactacc

SEQ ID NO: 469 - DNA, 94 bp, Artificial
  Hairpin sequence Mmd_170, mouse
tttgctgtgaaaattgcaaatgtttgcctcaagcaaatatgtgaaaatttgcaatttgttgtaggtgtggaattttgctatttttcacagtgga

SEQ ID NO: 470 - DNA, 97 bp, Artificial
  Hairpin sequence Mmd_171, mouse
gggacattccctggggctgggccaagggtcaccctctgactctgtggccaagggtagacaggtcagaggtcgatcctgggcctactctggggctcct

SEQ ID NO: 471 - DNA, 94 bp, Artificial
  Hairpin sequence Mmd_172, mouse
tttcaagtagtcaaattctggctcatggctctggaaggtgctgtaaacattgtattctcctctgctatgagactggattttgttctcactgggg

SEQ ID NO: 472 - DNA, 85 bp, Artificial
  Hairpin sequence Mmd_173, mouse
tatggattttctttctcaattgctacggagtcaccctgaggctattaatttgggctattagctgagggtgatttcaaaatccata

SEQ ID NO: 473 - DNA, 83 bp, Artificial
  Hairpin sequence Mmd_174, mouse
ggagaacagctttaaagtttagtcactctgatggaatgtaaaagcttctatcagtgacaggttgacagagaaagctgttttcc

SEQ ID NO: 474 - DNA, 65 bp, Artificial
  Hairpin sequence Mmd_175, mouse
tcagttaggctgctggctatactccaggacttaccttgtcctggatataaccactgccagactga

SEQ ID NO: 475 - DNA, 80 bp, Artificial
  Hairpin sequence Mmd_176, mouse
agtctccaaagacaattgtgaagggattgtgtttgactgatcaatcaaaggcagcccccccttgcttgtcttccgagact

SEQ ID NO: 476 - DNA, 82 bp, Artificial
  Hairpin sequence Mmd_177, mouse
tagaaaaggcaattaaaatccacaactgacttccttcccgtgtccaaggcgagggacagtgcgtgggtgtgtgcccttttta

SEQ ID NO: 477 - DNA, 91 bp, Artificial
  Hairpin sequence Mmd_178, mouse
gtctgccctggaattaggttttatcttaagtccacactggatcctctggatgggtccactgaggattttgaaggaagcataattccttgac

SEQ ID NO: 478 - DNA, 94 bp, Artificial
  Hairpin sequence Mmd_179, mouse
ggtcttggctctgtgtggaccagggcaggtgagggagcctggagatgttctctaggttatcctctgttgctgtgaactgggaggatccaaggac

SEQ ID NO: 479 - DNA, 98 bp, Artificial
  Hairpin sequence Mmd_18, mouse
tcacttgatgggggtattcccagctgtctgcctgaggaggagaggcagctgccctggcaggcagctggggtgtgctggccatgcctgatctgatgtgg

SEQ ID NO: 480 - DNA, 94 bp, Artificial
  Hairpin sequence Mmd_180, mouse
gaggaagggagcggcagcagtgaaggatttgagccccctgccgcagatgggcagttctctggggccaggaatcagctgcgccgcccccagtatc

SEQ ID NO: 481 - DNA, 98 bp, Artificial
  Hairpin sequence Mmd_181, mouse
ccgagactttgaaaacgaacgagaccgagatctggatgcagatcttgagcgagaagagcgagacccagacttggatttgtttgtttttgacatcttgg

SEQ ID NO: 482 - DNA, 100 bp, Artificial
  Hairpin sequence Mmd_182, mouse
ggtttggaggtgggcctgacatccctgagtgtatgtggtgaacctgaacttgccctgggtttcctcatatccattcaggagtgtcagctgcctcttcgct

SEQ ID NO: 483 - DNA, 95 bp, Artificial
  Hairpin sequence Mmd_183, mouse
gggattccacttcagagtctgaatacaggaaataagcttaggaaccacctaggtttgttgattcctgggcattcttaggcctggagagcatttct

SEQ ID NO: 484 - DNA, 85 bp, Artificial
  Hairpin sequence Mmd_184, mouse
gaatgactaaacagccctgcatatatttctggaatacttggaaatgcctgttatttctggaaatgtgtgcttgttcaggttattc

SEQ ID NO: 485 - DNA, 62 bp, Artificial
  Hairpin sequence Mmd_185, mouse
ccaccagacagacagaacggacgctgcagcctctgcagggactaatctgtctgtggggatgg

SEQ ID NO: 486 - DNA, 99 bp, Artificial
  Hairpin sequence Mmd_186, mouse
ctgcctactcctcaaaacttccgaggctacttttgattgggataaaagtgcacatactgattttttaaaatgtggctttaattaatggtgggtgggcag

SEQ ID NO: 487 - DNA, 98 bp, Artificial
  Hairpin sequence Mmd_187, mouse; Mmd_525, mouse
gaggggttaagagttcattcggctgtccagatgtacccaagtaccctgttatttggcaatacatacatctgggcaactgattgaacttttcgcttctc

SEQ ID NO: 488 - DNA, 65 bp, Artificial
  Hairpin sequence Mmd_188, mouse
tcagtcaggctgctggtgattctccaggacttatctggtcctggatataaccactgccagactga

SEQ ID NO: 489 - DNA, 99 bp, Artificial
  Hairpin sequence Mmd_189, mouse
aggtactttcaaagcaatgtttgacaggcacagggacaaatcttgttaacaagtaagaggatttgtgcttggctctgtcacatgccactttgaaaacct

SEQ ID NO: 490 - DNA, 98 bp, Artificial
  Hairpin sequence Mmd_19, mouse
gggcccccgcaactgctgctaccgccggccccccgagctttgaggtttccacccatggccgggcctcctccgggccggcagcagccgccgaacagctc

SEQ ID NO: 491 - DNA, 88 bp, Artificial
  Hairpin sequence Mmd_190, mouse
tggcctggctggcggcgctggctctgggcctcagacactctggaatcgccaaggtctgagctcagggcctggcccccttcccaggcta

SEQ ID NO: 492 - DNA, 92 bp, Artificial
  Hairpin sequence Mmd_191, mouse
ggatggggaggtggatagaattgtgggaggtaggctctgtccctcagaacccctttcctcagacagcttgttatccctgtgtcccttgagcc

SEQ ID NO: 493 - DNA, 86 bp, Artificial
  Hairpin sequence Mmd_192, mouse
gccaggaagaggcacatgaaactgaacacagtgtctgggctagcgccttgaattcagaatcaggaagtcatggttctattcctggc

SEQ ID NO: 494 - DNA, 76 bp, Artificial
  Hairpin sequence Mmd_193, mouse
tggcaagtggaggagcagcagggtgaaactgacacagtgctggtgagtttcactttgctgctcctcctgactccca

SEQ ID NO: 495 - DNA, 91 bp, Artificial
  Hairpin sequence Mmd_194, mouse
tctgcccttttcctgtccgtttcaccccgggtagcaaatggcccccacagccaagaggctacccctccctctgaagcccagactgggctga

SEQ ID NO: 496 - DNA, 99 bp, Artificial
  Hairpin sequence Mmd_195, mouse
ctgtaaagcctttactgccacttgttaaggaagacctgtatagctcactcgtgagtaataaggcggtcaacccaaactcaggcactaaaaattttatag

SEQ ID NO: 497 - DNA, 96 bp, Artificial
  Hairpin sequence Mmd_196, mouse
agaaacatctcaaaacctgctgacagcattttcactattagcggcaaacaaatcaactataattaaaatgctgtcaaagtgattggagatttttct

SEQ ID NO: 498 - DNA, 94 bp, Artificial
  Hairpin sequence Mmd_197, mouse
aatctgaatcttggaggcaggtgctcacatgtcctcctccatgcttataaatacatggaggaggccatagtggcaactgtcaccatgattgatt

SEQ ID NO: 499 - DNA, 97 bp, Artificial
  Hairpin sequence Mmd_198, mouse
ggtgggcgggggttcctggctgacctcactcctgtactgtttgtttgcgggcttgtagggggagaggaggacattttggaatcctttgtgtcaaatc

SEQ ID NO: 500 - DNA, 93 bp, Artificial
  Hairpin sequence Mmd_199, mouse
aagcacttaattgtgggtgcagttagtcgaggcgggcactctctgcagatttcaggggtgaagaaacctaattatacctgcaattgagtgctt

SEQ ID NO: 501 - DNA, 95 bp, Artificial
  Hairpin sequence Mmd_2, mouse
gaagaaatcctactcgggtggatataatacaacctgctaagtgttctagcacttagcaggttgtattatcattgtccgaggttatggctctcgtc

SEQ ID NO: 502 - DNA, 87 bp, Artificial
  Hairpin sequence Mmd_20, mouse
ggtgagtctcctcaggtgctcatgtgggagagattagcctgtgtttataagctggtctgttcattaagcgcctgctggagtggaacc

SEQ ID NO: 503 - DNA, 99 bp, Artificial
  Hairpin sequence Mmd_200, mouse
ttcgggattcttcagtcatgtagcacggggagtgtcttagcttggttgtctacagtgactcaggcaatacaccgtcctaggtgcctgaagagacttgga

SEQ ID NO: 504 - DNA, 98 bp, Artificial
  Hairpin sequence Mmd_201, mouse
ctgggctgtgctgttttgtagagaattgtccagatcaaaaccctcgtctcaggaactgggatccaggacaagattctgcgaagcacattgttatcaag

SEQ ID NO: 505 - DNA, 96 bp, Artificial
  Hairpin sequence Mmd_202, mouse
gtgggaattcgactttgcatctctacatctgtaaactgaggaacactttcagtgctctcagtgatgggtgagatgacatgtttgagccgaagaagc

SEQ ID NO: 506 - DNA, 100 bp, Artificial
  Hairpin sequence Mmd_203, mouse
gcagaaataatcatgtaatcatcttgtaattatatggtacttactatgcaattacatagtaaacacattgtaaattcaaaataattacatggttatttgc

SEQ ID NO: 507 - DNA, 94 bp, Artificial
  Hairpin sequence Mmd_204, mouse
ccctggcagcaagctgcgcttcggaaactgttgactcagagggtggcctggcggccagcgtctgatgtcagcctggagcccggccgcgccgggg

SEQ ID NO: 508 - DNA, 97 bp, Artificial
  Hairpin sequence Mmd_205, mouse
agtggaaggagaggagatagcgcggatcgctggtggggaatgttaatgggcgggagcccaggccgctggccaccgagacgtgggtatctcctccatt

SEQ ID NO: 509 - DNA, 74 bp, Artificial
  Hairpin sequence Mmd_206, mouse
ctggcaaccatgaggcccagatgctaagcactggaagatatttcttccaatagcagcatctggcatgctggcag

SEQ ID NO: 510 - DNA, 97 bp, Artificial
  Hairpin sequence Mmd_207, mouse
tccgctaccagaagcagctgcagcacaactatatccagatgtaccggcgtaaccggcagctggagcaggaactgcagcaactgagcctggagctgga

SEQ ID NO: 511 - DNA, 94 bp, Artificial
  Hairpin sequence Mmd_208, mouse
agatgatccttccccttacctgtctttctatcagtgtagctgaagctaattcagtgcactgatataagattattatcaggttgagcacttatct

SEQ ID NO: 512 - DNA, 84 bp, Artificial
  Hairpin sequence Mmd_209, mouse
atttgggcagaaattcttgactcagctcccaaggaatgtaaaccagaccctgggagtggaggagagttaagaaaaatctcagat

SEQ ID NO: 513 - DNA, 98 bp, Artificial
  Hairpin sequence Mmd_21, mouse
gtgtgtctcatttctgagcctttatataccctcccatattgagaattgagcaatgctgtggtgggaggttaaggatgaaggcctcaggaggctaaagc

SEQ ID NO: 514 - DNA, 94 bp, Artificial
  Hairpin sequence Mmd_210, mouse
ggtgagacagtcaacagcccagtgtccctcttgatggcactttggagatggctggagtgctctgtaaggcctgggctcaatgaccctcctcatc

SEQ ID NO: 515 - DNA, 89 bp, Artificial
  Hairpin sequence Mmd_211, mouse
gcgtcaaaaggcttattttgggcctcaatcctctccttctccgattttcggaggtaaggagcagcttcgccaaaaatagccatctccgc

SEQ ID NO: 516 - DNA, 84 bp, Artificial
  Hairpin sequence Mmd_212, mouse
ggtagaggagatggcgcaggggacacaaggtaggccttgcgggtctgtggacccttggacatgtgtcctcttctccctcctccc

SEQ ID NO: 517 - DNA, 99 bp, Artificial
  Hairpin sequence Mmd_213, mouse
gtcttcctgtagcaacagtggcagcagctctctggtttcctgtggtgtaaacaggaagccagactgtgtcaaaacggtagtctacagctgtgagaaggc

SEQ ID NO: 518 - DNA, 99 bp, Artificial
  Hairpin sequence Mmd_214, mouse
ctaggagataagttggagggttatctaggagtgatgtccccctgttttgtgtaacatgggggcaatcaacagcctaatgtccctcttgatggcacctag

SEQ ID NO: 519 - DNA, 99 bp, Artificial
  Hairpin sequence Mmd_215, mouse
ctggagtccagccctccaggggttaagggggcgggaccagttggttgctatggtgccagactgtttgggtctgctctctgacccttgggggctccctag

SEQ ID NO: 520 - DNA, 87 bp, Artificial
  Hairpin sequence Mmd_216, mouse
tcagcatggaatgaactgttagactatagccagggcctctggattctattagaggctcctggaatttctgaaccactcctaagttga

SEQ ID NO: 521 - DNA, 95 bp, Artificial
  Hairpin sequence Mmd_217, mouse
ttgagctgtaattccactgagggtttctagtgactccagcttcacacttgtggttatagtattcccagaacccctcggtggagtgatttcctgaa

SEQ ID NO: 522 - DNA, 98 bp, Artificial
  Hairpin sequence Mmd_218, mouse
cggattcttctctgttttagggttcttgtggtgcctgctgggctttcagtgctgtgggggcccttgaagggcagctgaagctgaaaacggggaagctg

SEQ ID NO: 523 - DNA, 77 bp, Artificial
  Hairpin sequence Mmd_219, mouse
gagcggagtctcctggacatagtgttctgggggacttttgggtgtctgagcaccacttttggagggagacttggctc

SEQ ID NO: 524 - DNA, 89 bp, Artificial
  Hairpin sequence Mmd_22, mouse
gtatttgaggtttaggaaggcgtgataatgaggctgtgaagattcatggcctcttttctatggcttaatttaggctctatatcaaatat

SEQ ID NO: 525 - DNA, 98 bp, Artificial
  Hairpin sequence Mmd_220, mouse
ggtaaaacctacttgaaaggagtgtttgtaggttctagtaaagctttgtgacggagtattgcagggcctgcagacacattctcctcagaagtgtgact

SEQ ID NO: 526 - DNA, 96 bp, Artificial
  Hairpin sequence Mmd_221, mouse
ctctgcccctgctttgtctttcagaataactgccatggaaggatgctcctcctgccaattggcacttatcaatgacatcgagggagagggacagag

SEQ ID NO: 527 - DNA, 91 bp, Artificial
  Hairpin sequence Mmd_222, mouse
gctgaagaggaaccagccctgatggtgtgtccgccaagacatggaagtgagaggtgggtggaggccccattggggttggtcggctctcagc

SEQ ID NO: 528 - DNA, 99 bp, Artificial
  Hairpin sequence Mmd_223, mouse
gctgtaattccatcttggtgttatggtgacatgaaaatccactcaagctgggttgtcagggtttatgaagttatcaaagccccttgatggaattatagc

SEQ ID NO: 529 - DNA, 97 bp, Artificial
  Hairpin sequence Mmd_224, mouse
tcattgtcatcgtcttggagggacatcattgtgtcactagtctgccaggttgttcagacagtccgacctgatgtcaagcaccaggatgatgacatgg

SEQ ID NO: 530 - DNA, 91 bp, Artificial
  Hairpin sequence Mmd_226, mouse
ttaatgtatccatttttctcagttagtgtcaggtttcattaggaaacacgggaggcctgacattaatcgcatgaaaaatgtgtctcaggga

SEQ ID NO: 531 - DNA, 96 bp, Artificial
  Hairpin sequence Mmd_227, mouse
cagcaagaggcactttgtacttctgccaggagaccatatgatatctctcagcgactctcctgcatgccaggaatacggaaccatgcctctcagctg

SEQ ID NO: 532 - DNA, 94 bp, Artificial
  Hairpin sequence Mmd_229, mouse
gcagcccagtgaagtggagttctgtcgttccagtctgcatctttgtagtttgggtcacagagacctttctttaaaatcttcttcactgaattgc

SEQ ID NO: 533 - DNA, 83 bp, Artificial
  Hairpin sequence Mmd_23, mouse
cattctttcagtgttgaaacaatctctactgaaccaagctccaaagcgagttcactggagtttgtttcagtattgcaggagtg

SEQ ID NO: 534 - DNA, 93 bp, Artificial
  Hairpin sequence Mmd_230, mouse
tattctcatcttgatgtgtggggctgtacagacagaaatgccattagcattaatggtaattatgcgcgtgagtccccgtgctccatgagaata

SEQ ID NO: 535 - DNA, 84 bp, Artificial
  Hairpin sequence Mmd_231, mouse
gtaggtactgatttatgaaaagctgtaaaagatcaggtggaactccgtaggacatcgtttacagctgcatggattgaaacctat

SEQ ID NO: 536 - DNA, 96 bp, Artificial
  Hairpin sequence Mmd_232, mouse
gctttcctcacttctgtttattttcagtcactcaaactgacgtgtgctgtcagtatctcctcactgtaagtgtaagcaggagtatttaaagaaagc

SEQ ID NO: 537 - DNA, 97 bp, Artificial
  Hairpin sequence Mmd_233, mouse
gctgcagtggcagcttggttgtcatatgtgtgatgacactttctaaagtcttccagaatgacaccacatatatggcagctaaactgttacatggaac

SEQ ID NO: 538 - DNA, 92 bp, Artificial
  Hairpin sequence Mmd_234, mouse
tcctggaaatgccctttaggtcagagtctgggatgcagcttctgactaacaatgaagtccactcccacttcctccagacctggcaaccaggg

SEQ ID NO: 539 - DNA, 95 bp, Artificial
  Hairpin sequence Mmd_235, mouse
gtgtgcttgttgtcgaagatgagggcctcctggatgagctggtgctgctgctccagcaggtccaggctgggcttgtagtccacgatgctgcgctc

SEQ ID NO: 540 - DNA, 98 bp, Artificial
  Hairpin sequence Mmd_236, mouse
gcatttcaggagattaatggtagaccgaagctaatagcctttggctgactctccagccatcagcttttcccagctgctcattaattcccagaaaatgt

SEQ ID NO: 541 - DNA, 98 bp, Artificial
  Hairpin sequence Mmd_237, mouse
gcaagatggcggcgctggcggaggagcagacggaggtggcggtcaagctagagcctgagggaccgccgacgctgctacctccgcaggctggcgacggc

SEQ ID NO: 542 - DNA, 94 bp, Artificial
  Hairpin sequence Mmd_238, mouse
cccgccttgcccgctggtagggcctactaggccgccgggacatcccgggtctcaagtaggcctagtctgccagcaagggcgtcccaaaggcggg

SEQ ID NO: 543 - DNA, 85 bp, Artificial
  Hairpin sequence Mmd_239, mouse
gcctgaatgcatgactctacaaccttaggacttgcagaattaaaggaatgccgtcctgaggttgttgagctgtgcgtttctgggc

SEQ ID NO: 544 - DNA, 95 bp, Artificial
  Hairpin sequence Mmd_24, mouse
ttctcaacagtgatgtgatatgtgatttccccttccaagccatggtgcagtttcatcggcaccatggccaagagggctccatcttggtaagagaa

SEQ ID NO: 545 - DNA, 97 bp, Artificial
  Hairpin sequence Mmd_241, mouse
gggagaatgattggaaagattatctaaatctcttctctgggctctcaggagtacgtgagctggtggtttgggtggtttgggttttacaggttctccc

SEQ ID NO: 546 - DNA, 97 bp, Artificial
  Hairpin sequence Mmd_242, mouse
ttctgcaatgttttgcagccaggcacacacactacaggtgctctcagagattgagctgatggccctgctgtgtgcactgtaaacaattattatagaa

SEQ ID NO: 547 - DNA, 98 bp, Artificial
  Hairpin sequence Mmd_243, mouse
gagaagcgaaaagttcaatcagttgcccagatgtatgtattgccaaataacagggtacttgggtacatctggacagccgaatgaactcttaacccctc

SEQ ID NO: 548 - DNA, 69 bp, Artificial
  Hairpin sequence Mmd_244, mouse
gtgaaacatagcctcagtattttcaggtgattttaatgaacacctgataaaactgaggtatagtaccac

SEQ ID NO: 549 - DNA, 100 bp, Artificial
  Hairpin sequence Mmd_245, mouse
caggagcagctggagccggggattaccccctgctgctgacgtgaggacggtgaacaatccggaacgttcggtggaagggggaattcccgtccgttccctg

SEQ ID NO: 550 - DNA, 98 bp, Artificial
  Hairpin sequence Mmd_246, mouse; Mmd_266, mouse
tgaccccaagaaaccaaagggcaagatgtctgcttatgccttctttgtgcagacatgcagggaagaacataagaagaaaaacccagaggttcccgtca

SEQ ID NO: 551 - DNA, 99 bp, Artificial
  Hairpin sequence Mmd_247, mouse
gccactttgagagcatcggtttttatttaaaaggcttaattggaagtttttattactcccaattaggctttttaattaaaaggaaaagttgagagaggc

SEQ ID NO: 552 - DNA, 99 bp, Artificial
  Hairpin sequence Mmd_248, mouse
ccattttcaagtgcggtggatttacacttcaaaaagtcaggcgcgacttcataattgaattgcacctgatcttttcaagtgccaggcaccggagccggg

SEQ ID NO: 553 - DNA, 91 bp, Artificial
  Hairpin sequence Mmd_249, mouse
taactctttgactgccatggcacaaaccatgctgatgattctagattgtgaccgttgtatggttaaggttgccacagcagtcaaagggtta

SEQ ID NO: 554 - DNA, 86 bp, Artificial
  Hairpin sequence Mmd_25, mouse
gggaggtggtaatggtaggagattggcctagtaggactgtcaccggtggtgtgtccttgtggctgtgtcttagctggactcttcct

SEQ ID NO: 555 - DNA, 93 bp, Artificial
  Hairpin sequence Mmd_250, mouse
accaaaggagcttttctggtctattgggtaaatatgtattaaagtaatttttaaaatacttatgtactcaataggctggaagtggtcaatggt

SEQ ID NO: 556 - DNA, 88 bp, Artificial
  Hairpin sequence Mmd_251, mouse
tcttttcttgaagcctgactccccgcgggacttccaaggcagcataacttcgggtccagactgggctgtcaggcgtttccaggaagga

SEQ ID NO: 557 - DNA, 93 bp, Artificial
  Hairpin sequence Mmd_252, mouse
gggtgatccgggtctgatgtccccatgccatgagggacatggtgagtccagtgtccctcttgatggcacttttggacatggccagggtggcct

SEQ ID NO: 558 - DNA, 84 bp, Artificial
  Hairpin sequence Mmd_254, mouse
gcaatgagccatgggccaacaatgtgagtaatggctagttttgttattttgaacttgggtagttggctgatgggtgtttattgc

SEQ ID NO: 559 - DNA, 90 bp, Artificial
  Hairpin sequence Mmd_255, mouse
gtgctgaagttggtatggcagcctgcaccatcccagttccctggaatgggcttggggtcaaaggttgctatcaccccaaagtcttcgcat

SEQ ID NO: 560 - DNA, 97 bp, Artificial
  Hairpin sequence Mmd_256, mouse
gcggcccgagccggtaggccccgaggagggttgtgggcccttctttttgtgaatgaagctccacggaacatccctcctgggggtccccgggagccgc

SEQ ID NO: 561 - DNA, 86 bp, Artificial
  Hairpin sequence Mmd_257, mouse
gtgtgctcagctggagtaattagaggaaaaacctgagggaagtctaccccagaagactcactcagccttctccagctgagtctcac

SEQ ID NO: 562 - DNA, 98 bp, Artificial
  Hairpin sequence Mmd_258, mouse
ggggtgctgccttctttctcggtctgctggttggaggacgaagatgacgaggagctggtgctggccctcgaatcgtcatccgacatagcgaacccccc

SEQ ID NO: 563 - DNA, 99 bp, Artificial
  Hairpin sequence Mmd_259, mouse
tcccctgctgcactaaattgacacttgtgcaagcaatggctctcgtcagtcactctgaagctgctgcctccagaagtgtcgatttagtgcagcgggaga

SEQ ID NO: 564 - DNA, 96 bp, Artificial
  Hairpin sequence Mmd_260, mouse
tatctcttttttgattagagaggtggcagggagcctggctcattaggcagaagtaatgagccaggctacatgccatctctaattagagaaaagatg

SEQ ID NO: 565 - DNA, 94 bp, Artificial
  Hairpin sequence Mmd_261, mouse
agaatcttctgacaatgaaggtaggcgtacaactaggagattgctgtcacggatagcttctagcatgtcatctacttttttctcacgaagatct

SEQ ID NO: 566 - DNA, 98 bp, Artificial
  Hairpin sequence Mmd_262, mouse
ttacacctgaaacttctcacttgggggtacaattagcctctggcgcctttagaatgagttgccagtgactaatggtgccctcaaggaagcatttgtaa

SEQ ID NO: 567 - DNA, 98 bp, Artificial
  Hairpin sequence Mmd_263, mouse
gaggacagctggcagaaagtgttacaggtataaccctaattctccttgtttctgttggtgttgtaggagagatctgtaggcctttctgtccagcgctc

SEQ ID NO: 568 - DNA, 94 bp, Artificial
  Hairpin sequence Mmd_264, mouse
aaacgctgtgactcaaactcggggaaattgttcctgtaatggagttctcaatttattggtcaatttcaccttgattttgtgtcactgttggttt

SEQ ID NO: 569 - DNA, 99 bp, Artificial
  Hairpin sequence Mmd_265, mouse; Mmd_463, mouse; Mmd_504, mouse
ctttttcttttcttgaagcctgactccccgcgggacttccaaggcagcataacttcgggtccagactgggctgtcaggcttttccaggaaggatccagg

SEQ ID NO: 570 - DNA, 96 bp, Artificial
  Hairpin sequence Mmd_267, mouse
ggacctgaaagatggtgaactatgactgggcagggcaaagccagaggaaactctggtggaagtctgtagcagtcctgacctgcaaatcggtggtcc

SEQ ID NO: 571 - DNA, 54 bp, Artificial
  Hairpin sequence Mmd_268, mouse; Mmd_307, mouse; Mmd_467, mouse
gcctctcggcgccccctcgatgctcttagctgagtgtcccgcggggcccgaagc

SEQ ID NO: 572 - DNA, 82 bp, Artificial
  Hairpin sequence Mmd_269, mouse
tctcgggctctgggggactatgactacaagtgtgtggatggcaagggccctacagagcagcttgtttctccagagcctgagg

SEQ ID NO: 573 - DNA, 92 bp, Artificial
  Hairpin sequence Mmd_27, mouse
ggggaatttctggccacaatttgtaacactaaaaggctccccaaagggagggggtgcaaaatgatacaaattgggtacaaaggatagccact

SEQ ID NO: 574 - DNA, 91 bp, Artificial
  Hairpin sequence Mmd_270, mouse
ttgggtgtggggataggggcacttctgttcctgataaccttattatgtgtttatcaggagcagacactgcccaggttgtcacgacaaccag

SEQ ID NO: 575 - DNA, 92 bp, Artificial
  Hairpin sequence Mmd_271, mouse
cttggaggctttgcctcatccacaggcatttggcatcggagatggaaaatttcacttgtcaggtcaccatggaaacaagcagagccactgag

SEQ ID NO: 576 - DNA, 94 bp, Artificial
  Hairpin sequence Mmd_272, mouse
ttagcatgttaataggtctttgtgcccaaaggctttggattataaattatcaagactgcattagccagatgggcaggattaattagcatgctag

SEQ ID NO: 577 - DNA, 85 bp, Artificial
  Hairpin sequence Mmd_273, mouse
ctggacacctgcgtccctctcttgtttcctctcctcttcttatgccccctcctggggaggaggctactggatgtaagctgtgcag

SEQ ID NO: 578 - DNA, 94 bp, Artificial
  Hairpin sequence Mmd_274, mouse
gggtggctggggtcatacatctgtgggtaaggataagcatgcaagtattgtatgtaggactggtgctgactcatgggtgaggacacggccactc

SEQ ID NO: 579 - DNA, 98 bp, Artificial
  Hairpin sequence Mmd_275, mouse
ggcaccagctccgtcagctccagccccaggtcctgcgcgatgcggcccacaaaggtgccgtgtttggcctcctctgggacggagtagcggagctggcc

SEQ ID NO: 580 - DNA, 97 bp, Artificial
  Hairpin sequence Mmd_276, mouse
ccgagactttgaaagcaaacgagaccgagatctggatgcagatcttgagcgagaagagcgagacccagacttggtttgtttgtttttgacatcttgg

SEQ ID NO: 581 - DNA, 82 bp, Artificial
  Hairpin sequence Mmd_277, mouse
tagttagctaattgcaggtgcaattggccacttgctccatcagatgaagggtaagtggttaattgaactcatgagtgaccta

SEQ ID NO: 582 - DNA, 98 bp, Artificial
  Hairpin sequence Mmd_278, mouse
cgccttgcctagtcgggccgccaacaccagatccttctccttctggcggatcaccgacagcagctcgggatcctgggtaggcggcggcggcagcggcg

SEQ ID NO: 583 - DNA, 99 bp, Artificial
  Hairpin sequence Mmd_279, mouse
ggcttgttttgagttcattgttcagcagatgagctagttcaggttttagctttcagcagttcatttgattgaacttgggaccaataaaagtgagaggcc

SEQ ID NO: 584 - DNA, 96 bp, Artificial
  Hairpin sequence Mmd_28, mouse
tgaatggctttgctccttttctgcatcacttccatcagcgcacccacaattcctgaagtgggtgcgggtgttggtggtgtggactcttggccatca

SEQ ID NO: 585 - DNA, 94 bp, Artificial
  Hairpin sequence Mmd_280, mouse
cctggctgcaagaggcttgctgcctagtccgaggccagcagtgttgtgtctggcctgtaagaggcaggtagcaagccgagactatgtccaaagg

SEQ ID NO: 586 - DNA, 91 bp, Artificial
  Hairpin sequence Mmd_281, mouse
tctgaaggccacgcccattcgggactggagggatgggcttcctaacggaagcgcacccttcaagtcctggaacaccgactctatcttctga

SEQ ID NO: 587 - DNA, 88 bp, Artificial
  Hairpin sequence Mmd_282, mouse
ctgttctgctaagacctgcgctgtggtgtaggaccaagggaatgtgcccctggcttttgtgtcagcatgcttggttactacagaacag

SEQ ID NO: 588 - DNA, 99 bp, Artificial
  Hairpin sequence Mmd_283, mouse
gttagcgcagagcttcaagctaagcaaggcatctcagcacgaggtgtcaaacataagacagctaatgctggaagccagagcaagaaggctcttcttgac

SEQ ID NO: 589 - DNA, 80 bp, Artificial
  Hairpin sequence Mmd_284, mouse
acgttagtccatttgctcagcggcccttccacgatggacgccatcttgggagccgccaatgacaacaaacggcctgacgt

SEQ ID NO: 590 - DNA, 94 bp, Artificial
  Hairpin sequence Mmd_285, mouse
attaattgagccaagccaagctcaaccttgaagatgctactttggtgtgttcccgaaatagggctgagcttatggctggttcttacttgttagt

SEQ ID NO: 591 - DNA, 90 bp, Artificial
  Hairpin sequence Mmd_286, mouse
actaacctggagtcacttcctctggttgacacccactcaaaaagaacactcctgattaagacggttgagaccagagatggacaggtcagt

SEQ ID NO: 592 - DNA, 99 bp, Artificial
  Hairpin sequence Mmd_287, mouse
ttcctttatcagcttccctgcaggactgcagggcaatggcagagtgtaagctgccatgggtagagcttcggggatggcagggcagaggtgaggagggag

SEQ ID NO: 593 - DNA, 98 bp, Artificial
  Hairpin sequence Mmd_288, mouse
ttggctgttttcctattagcatatgaggaacctaggactgtcccttgttgtcaaggtaacagctattccttgggtagctgcaatagaaaagaagccaa

SEQ ID NO: 594 - DNA, 99 bp, Artificial
  Hairpin sequence Mmd_289, mouse
ggcctgtttttcaacctggctatttctttgatattagtctcaatagtagatgggatacagtgccagaaaagtagcactggacttggagtcagaaaagcc

SEQ ID NO: 595 - DNA, 99 bp, Artificial
  Hairpin sequence Mmd_29, mouse
ccttttcttttcttgaagcctgactccccgcgggacttccaaggcagcataacttcaggtccagactgggctgtcaggcttttccaggaaggatccagg

SEQ ID NO: 596 - DNA, 96 bp, Artificial
  Hairpin sequence Mmd_290, mouse
gctggcctcactgacccaggaattcatagcaggaaattaggcaggattaaggaaggctagttttcttgtgtgagaagggtaaaggtggaggaaagc

SEQ ID NO: 597 - DNA, 97 bp, Artificial
  Hairpin sequence Mmd_291, mouse
gtcgcagcgcgtggccgatcgcctgggcctggagctgggcaaggtggtcaccaagaagtttagcaaccaggagaccaggtgggagcctcgctggggc

SEQ ID NO: 598 - DNA, 88 bp, Artificial
  Hairpin sequence Mmd_292, mouse
agcttcttttcccgggcgagctgcctacgggtccgctgctggcacacagccagctgggtcttggcggctttgttgctgggatagagct

SEQ ID NO: 599 - DNA, 94 bp, Artificial
  Hairpin sequence Mmd_293, mouse
gcttgagctgaggggcctacctcaagctatggaaaaaattagggttagacaagaagcatttttttatagcctgaggaaggcccatggcaagagc

SEQ ID NO: 600 - DNA, 81 bp, Artificial
  Hairpin sequence Mmd_294, mouse
cgtcacctagggatcttgttaaaaagcagagtctgattgaggggccaagattctgcatttttagcaagctctcaagtgatg

SEQ ID NO: 601 - DNA, 99 bp, Artificial
  Hairpin sequence Mmd_295, mouse
ggtcaggatgctttgttgtctggattctcagttgcttgtattggcttttgaaaaggtcattatgagaaaagggatctgaatagcaaaaggattccagct

SEQ ID NO: 602 - DNA, 92 bp, Artificial
  Hairpin sequence Mmd_296, mouse
tgtctgtgtttcctcttgcagggttacattttgccatctgcctttccagagtaagtggctctatgtgggcactgagcgaggaaacatacaca

SEQ ID NO: 603 - DNA, 96 bp, Artificial
  Hairpin sequence Mmd_297, mouse
tagactaattaatgtttgagtgtttaaacacaatgcctttacagaagcaatggcattataatgtgttgggacactctagcattgtaattagcacta

SEQ ID NO: 604 - DNA, 98 bp, Artificial
  Hairpin sequence Mmd_298, mouse
cttcagcaaccttgcagccccatcgctcatttgcataatggatgcctttttatccattatgcaaatggtatgagaggaaaattaggcaataagggaag

SEQ ID NO: 605 - DNA, 94 bp, Artificial
  Hairpin sequence Mmd_299, mouse
ggtgatgaatgtatattggtaaggagggtgaagttgaaggggcagagacctctcaaatgaattgacttttctttaccaacatattcagaacacc

SEQ ID NO: 606 - DNA, 86 bp, Artificial
  Hairpin sequence Mmd_3, mouse
ttggcagactgggaaatctctgcaggcaaatgtgatgtcactgaagaaaccacacacttacctgtagagattcttcagtctgacaa

SEQ ID NO: 607 - DNA, 97 bp, Artificial
  Hairpin sequence Mmd_30, mouse
attggggtgcaccagtgagagaaccttccggaaagactcttgggcttggaacagggcccttgagtcttggtgctctcttccctttctgggcctcagt

SEQ ID NO: 608 - DNA, 90 bp, Artificial
  Hairpin sequence Mmd_300, mouse
cagctagaactcccctggcgagtggaaacagcttttaccgcggctgtagctgtggttttggaattttccaacgccccctgtgattggctg

SEQ ID NO: 609 - DNA, 95 bp, Artificial
  Hairpin sequence Mmd_301, mouse
ggctatgattccactgaggggtgctggcggtgacatgtgccatgaagtcaatctgagtggtttatcaccagaactcccagggtggaattataacc

SEQ ID NO: 610 - DNA, 99 bp, Artificial
  Hairpin sequence Mmd_302, mouse
gcagtgcctaaggaattaatgaccagctgaggttaatgagtccccactcagctccgggccatcaactctcccagctggccattaatcccctggaaatgc

SEQ ID NO: 611 - DNA, 82 bp, Artificial
  Hairpin sequence Mmd_303, mouse
aggtggtacgccctttcctctctgccccatagggtgtagctctaactaccctctagggaagagaaggttgggtgaacagcct

SEQ ID NO: 612 - DNA, 83 bp, Artificial
  Hairpin sequence Mmd_304, mouse
ttccagccaggccacctctcctttatctgcacagtctattttaagagtactctactgcaaggagaatggggcctaggcaggaa

SEQ ID NO: 613 - DNA, 94 bp, Artificial
  Hairpin sequence Mmd_305, mouse
gcgaagcattgcatttgggtgacaggcagttgttcgtgcttgaataactcaggatttgaattctacagtttgtggcacccgtgtaatgcttagc

SEQ ID NO: 614 - DNA, 96 bp, Artificial
  Hairpin sequence Mmd_306, mouse
ccacattttcttcgccacagaatgactcctctgagaacaaagtcattgttgacttcaaagaactctgaggaagttcagtggatggaaagaatttgg

SEQ ID NO: 615 - DNA, 97 bp, Artificial
  Hairpin sequence Mmd_308, mouse
gaggttgaggtctcctattcctggctcccagcttcttgcctccaggttcctccaccatccttgagctgtgaccttgacttggtgacctatgaacctc

SEQ ID NO: 616 - DNA, 64 bp, Artificial
  Hairpin sequence Mmd_309, mouse
ggccttttccatgttgggtaactgctgtgcatgctacccagttacccaagccaggaagcaggcc

SEQ ID NO: 617 - DNA, 99 bp, Artificial
  Hairpin sequence Mmd_31, mouse
agctgtgtcatcaatagaagctcagggccttagggaactggagggcacttctgacccactgctctcactctttggctctacttctgacaatgacttgct

SEQ ID NO: 618 - DNA, 98 bp, Artificial
  Hairpin sequence Mmd_310, mouse
ttctccagcgttggagtcagccacggcttacctggaagattgaatgtctacttggctttcttcctgtcagccgtgggtgcctcttgctactggaagag

SEQ ID NO: 619 - DNA, 85 bp, Artificial
  Hairpin sequence Mmd_311, mouse
attggtcaggaaagacactttttatattgtcaagtggcacttaagccactaattcttgactgtgaaactgcttttcctgggcaat

SEQ ID NO: 620 - DNA, 71 bp, Artificial
  Hairpin sequence Mmd_312, mouse
gggaggaggcggaaagccgacattgtttacttaattaaaagtaaacaacaatttgccgctgccagcctccc

SEQ ID NO: 621 - DNA, 89 bp, Artificial
  Hairpin sequence Mmd_313, mouse
gtacatctaggaagcagctgaaagggtttctctagtctgatatctcagagagtggactgagaaatccctctctttacctgaagacgtac

SEQ ID NO: 622 - DNA, 98 bp, Artificial
  Hairpin sequence Mmd_314, mouse
aaattaatgaccaaaaatgtcagatgtgtccacagctaattattcattagatgcttaattagtgtgggatgatcatgacattttaatgttattaattt

SEQ ID NO: 623 - DNA, 98 bp, Artificial
  Hairpin sequence Mmd_315, mouse
tcattgagcaggtggggctcaggaggtcctggggccgccctgaaggcagaacatgagaattctgggccctccagacctcatgcttcctgcctgtgtgg

SEQ ID NO: 624 - DNA, 88 bp, Artificial
  Hairpin sequence Mmd_317, mouse
aggaaggatctgggccaattttctggttgcttcttggcactgagttcttgtgtttttacagcagtcacacaggccctacatccttcct

SEQ ID NO: 625 - DNA, 82 bp, Artificial
  Hairpin sequence Mmd_318, mouse
gacggctaatgcccattagtgttggggactgttacctcgatcaggagtgacagagccctaattattaaggtaattagaagtt

SEQ ID NO: 626 - DNA, 96 bp, Artificial
  Hairpin sequence Mmd_319, mouse
aggtccttgatttcaatgtttcccactttggtggttgatctgataataggtcttccaaccaaagctgggaagatgtgttctggaaaattagagcct

SEQ ID NO: 627 - DNA, 95 bp, Artificial
  Hairpin sequence Mmd_32, mouse
aagctgacgctacccatcccctccacgtgccccgagccctttgcccgcctactggagggtgagccagggccctgtggagagggtaggctcagctt

SEQ ID NO: 628 - DNA, 89 bp, Artificial
  Hairpin sequence Mmd_320, mouse
aggtgcctgtgactcatctggtacctattgtgctctctgcaaaggcgcggaggcttgcttgtgtgatgggtgccatttgagagccactt

SEQ ID NO: 629 - DNA, 97 bp, Artificial
  Hairpin sequence Mmd_321, mouse
agtatatgacgctataacttgtttttacagatgattaaatgctcggttgaagctgaggctttgatgactgttggagccatttatgacctcatatact

SEQ ID NO: 630 - DNA, 88 bp, Artificial
  Hairpin sequence Mmd_322, mouse
tcttttcttgaagcctgactccccgcgggacttccaaggcagcataacctcgggtacagactgggctgtcaggcttttccaggaagga

SEQ ID NO: 631 - DNA, 99 bp, Artificial
  Hairpin sequence Mmd_323, mouse
ccttttcttttcttgaagcctgactccccgcgggacttccaaggcaccataacttcaggtccagactgggctgtcaggcttttccaggaaggatccagg

SEQ ID NO: 632 - DNA, 99 bp, Artificial
  Hairpin sequence Mmd_324, mouse
ggccgcatggaccacgtggaccgctgcttcgatgtgctggctgtggagtactacgtgccggatgccgaccagtggaccagtgtgacacccatgcgagct

SEQ ID NO: 633 - DNA, 83 bp, Artificial
  Hairpin sequence Mmd_325, mouse
ccctggtgtgagcgtatgtgcctttggactacatcgtgaacgcagcaccatgcagtccacgggcatatacacttgcctcaagg

SEQ ID NO: 634 - DNA, 99 bp, Artificial
  Hairpin sequence Mmd_326, mouse
cctagctttcctaagtgatgtcagcccctgcgagcttccaataggaaccaggcataaatcagggagctgaccttatccaataggaaactttattatagg

SEQ ID NO: 635 - DNA, 98 bp, Artificial
  Hairpin sequence Mmd_327, mouse
aggaattagggtcaggacccagaaactttgctctatcagctggtgtttgtttcagctcaggtggggtgggtttcccctttgatgggtcatttcctcct

SEQ ID NO: 636 - DNA, 91 bp, Artificial
  Hairpin sequence Mmd_328, mouse
ccaagcagggacttcttgccagcccttggcagcttgaggcctcccatcatcatccaccgagctggcaagaagtatggcttcaccctgcggg

SEQ ID NO: 637 - DNA, 94 bp, Artificial
  Hairpin sequence Mmd_329, mouse
tccttgatcttcaagccaggcgtctggaagtcgccgtaaagagggaacacgtcctctttggggtcgccttccagagggtaggtgcagtctggga

SEQ ID NO: 638 - DNA, 97 bp, Artificial
  Hairpin sequence Mmd_33, mouse
gctgtgaggtcagaatcctgtgcttacgacacctcggtctgttgccatggaaacaggggcggattaaggctggagcgcagggatcaggcccctccgc

SEQ ID NO: 639 - DNA, 95 bp, Artificial
  Hairpin sequence Mmd_330, mouse
gtgccccgaggggtggctgtgatgagcgggatgcggaaagtgtctacagagccgatctgcttcccgctcacccacagcagctgctccatggtcac

SEQ ID NO: 640 - DNA, 97 bp, Artificial
  Hairpin sequence Mmd_331, mouse
ggaaagctgtcacatttggaaaggagcaaccaggcagggagaaactaaattgtttttctttgtctgcctgctgttccaagtcgttgcaggcactgcc

SEQ ID NO: 641 - DNA, 95 bp, Artificial
  Hairpin sequence Mmd_332, mouse
tactccacgggtagcatgttggagatggtcaggtggaataataaggagagtccacagaccaggagcttctccgtaactgctgcctggaacgggtg

SEQ ID NO: 642 - DNA, 100 bp, Artificial
  Hairpin sequence Mmd_333, mouse
gggctgatgagcagttcaagctccctgcatagaaatagcctcagaatagttgtggtgttgtttctaattgcaatgggtgagcagactggctcattaatcc

SEQ ID NO: 643 - DNA, 99 bp, Artificial
  Hairpin sequence Mmd_334, mouse
tccttcacaaagtacaaagggtttcagaatcaagtgtattccagcagcagcccaggcttgctgacaagatattttgaaacaacttgctattaggaagga

SEQ ID NO: 644 - DNA, 95 bp, Artificial
  Hairpin sequence Mmd_335, mouse
gaggcgagtgtggatctaatcttcagctgattaaatgtccctcattaatgagtttctttaattaatgaagttttggggcctgctccacttgcttt

SEQ ID NO: 645 - DNA, 99 bp, Artificial
  Hairpin sequence Mmd_336, mouse
gcatgccagtgggaatcataggctggagagaccttccttctgcaggagatgaaattcttgaagtagaatctgaggtattcaagcctaattccctaatgt

SEQ ID NO: 646 - DNA, 97 bp, Artificial
  Hairpin sequence Mmd_337, mouse
aatttattttcgctaggagaggaatgacccataactcacgaacggtgatgcttaaagtgagttatggaggttactctcctgtgagaggaaatggatt

SEQ ID NO: 647 - DNA, 77 bp, Artificial
  Hairpin sequence Mmd_338, mouse
tccaagttcatgatgaagagtttgtcagtgggagtcagttccacaggctgcctggtgaactctttgtcgaagttgga

SEQ ID NO: 648 - DNA, 100 bp, Artificial
  Hairpin sequence Mmd_339, mouse
ggcccccatctgggaggatgatgtcatctatctccccaaagaaaaggccacactttcctttgtggaagtccatggtgacatcacctcctgatcacatgcc

SEQ ID NO: 649 - DNA, 96 bp, Artificial
  Hairpin sequence Mmd_34, mouse
gagcaatctagaattgattttatattcctggtcaagttgctgaaatgtagcaattgggtcaggaagatcagttccttggacatgtatcttgtgctt

SEQ ID NO: 650 - DNA, 97 bp, Artificial
  Hairpin sequence Mmd_340, mouse
ggcccagccacagtgacattcatcccccggtgggtctggctgcctcctcacaacccaccgacaacaatgaatgttaattgtgacctttgctgtggcc

SEQ ID NO: 651 - DNA, 95 bp, Artificial
  Hairpin sequence Mmd_341, mouse
gcaatgtggagaagcctagggcttgccctggctctctgtctcctcccctatggaggagcagagagccaaggccaaagctctgcttgttacaaagc

SEQ ID NO: 652 - DNA, 89 bp, Artificial
  Hairpin sequence Mmd_342, mouse
ttattcacctggaaactgctgaggagcacaaatgtcacaagataaatcaaacctgtgagattggcatctctcagcagcaggatgggtag

SEQ ID NO: 653 - DNA, 89 bp, Artificial
  Hairpin sequence Mmd_343, mouse
cagggtgatggttagggatttggcagcatggccccattaacattagtgggagttagtcagctaaatcccagacatggaacagaatcctg

SEQ ID NO: 654 - DNA, 95 bp, Artificial
  Hairpin sequence Mmd_344, mouse
tctcagtaactatggaaatcccagggtttccctagcaacagctccaaatgggtcttgttgctagggaacacagggatttcccaggctaccaaaga

SEQ ID NO: 655 - DNA, 95 bp, Artificial
  Hairpin sequence Mmd_345, mouse
aagctgcggagtcaaccatgcagacaaatgagaagctgtcttttgtctgcagaaggcagcaatcctacatcaacatggaggtctctgtctgactt

SEQ ID NO: 656 - DNA, 90 bp, Artificial
  Hairpin sequence Mmd_346, mouse
ctcaggcggggtgggggaggatgttttggatgccgctctgctgacgttggtgccctctgttcttacatcctccttaatttactgcctgag

SEQ ID NO: 657 - DNA, 98 bp, Artificial
  Hairpin sequence Mmd_347, mouse
gcggcgggctgagttttgaaaacttaggattctaaatgtataaattgtttggaacatttaacatctagtcattaagcagtcaccctcttgccctccgc

SEQ ID NO: 658 - DNA, 94 bp, Artificial
  Hairpin sequence Mmd_348, mouse
ggccttcttagctgctctaattaggatccccttagtccctcccggtttctccggagaggacagggggctttttaattgaggctattaggagttc

SEQ ID NO: 659 - DNA, 96 bp, Artificial
  Hairpin sequence Mmd_349, mouse
agccattaacatgtcacccctgcttgaaacaatgactcagcttggttctgcataataggacaagtgagggagacaagcagaggtgacaggaaggtt

SEQ ID NO: 660 - DNA, 83 bp, Artificial
  Hairpin sequence Mmd_35, mouse
gggcttggcctggctctgctgggacacttcacacttttgccatttttggccagaaggctctccctgctagccgggctctgttc

SEQ ID NO: 661 - DNA, 97 bp, Artificial
  Hairpin sequence Mmd_350, mouse
ttccaaattttatgggccaaattctgccctcaattatgcacatgcaactaattgcacacatataacagagggcagaatttcgcttttggaactggaa

SEQ ID NO: 662 - DNA, 98 bp, Artificial
  Hairpin sequence Mmd_351, mouse
caaaagcagatattttcccaagcagacaccagccacagccttcttggctgaaaggctaaatggctggctactcatgtactgttgaatgtctgagtatg

SEQ ID NO: 663 - DNA, 99 bp, Artificial
  Hairpin sequence Mmd_352, mouse
gtttaagtacctgtgggcagcatcagggtctgccttgcggtgggaccttttgggtttcagcgcagggtctctggtgtttcctgacaggcgatctgaagc

SEQ ID NO: 664 - DNA, 99 bp, Artificial
  Hairpin sequence Mmd_353, mouse
gcattttctgcagattaataattagctagaaggagttgatggtccttggctaaaacgtgggtcattaactccagccagtcattaattgcctggaaatgt

SEQ ID NO: 665 - DNA, 94 bp, Artificial
  Hairpin sequence Mmd_354, mouse
gactctgtatttaatttggctcagccgggaagattttttggctctgtgtgtgtatgtgtattctgaaaaatctttccgagcaagccccagggtc

SEQ ID NO: 666 - DNA, 92 bp, Artificial
  Hairpin sequence Mmd_355, mouse
ttgtagcatgtggttgtattaatgaacgttacaggacagctttataatcatttataaatctgtaacattcaataaagaagcacatgtggcaa

SEQ ID NO: 667 - DNA, 98 bp, Artificial
  Hairpin sequence Mmd_356, mouse
ttggtgttgtcaatttgtgagaccagtctggaattttcagccttggtacacaagatctagaattaagagattgcacacacaaatgagacaaacaccag

SEQ ID NO: 668 - DNA, 90 bp, Artificial
  Hairpin sequence Mmd_357, mouse
ttcaggtattatgggctatctagctgtggtgaaattgggaaggatgatatcttagaaatctttcctgactgcagagacttgtaacctgaa

SEQ ID NO: 669 - DNA, 95 bp, Artificial
  Hairpin sequence Mmd_358, mouse
gcgacgtccgtgcagcggagaagcagaggataatggcgtctgcagcgctgtcgcctgtagcgccctcctcctttctcgttcgcgcactctgtggt

SEQ ID NO: 670 - DNA, 90 bp, Artificial
  Hairpin sequence Mmd_359, mouse
ttggggtaccgagttgtgggtgagtctttttccccagttcccacggttctcccttgctggggtcctggcttctccgctctttgtccccga

SEQ ID NO: 671 - DNA, 99 bp, Artificial
  Hairpin sequence Mmd_36, mouse
gctgatatacagcatttggcaggattccttgctagcatttggtaataattccataccaaatgctagggagaaacccacccaaatggaaccgcttcaagc

SEQ ID NO: 672 - DNA, 88 bp, Artificial
  Hairpin sequence Mmd_360, mouse
ggcactgggggcagaaggccatttgaaaattttccacattgaaattttgattaattaaatttgtctcaagtggctggctttcagtgtc

SEQ ID NO: 673 - DNA, 93 bp, Artificial
  Hairpin sequence Mmd_361, mouse
agaaggatgctgaaaccaatgaaacaatatattgtacaaaagagcctttcatccaggctcgggttattgtcattcgttggctggtttctttct

SEQ ID NO: 674 - DNA, 99 bp, Artificial
  Hairpin sequence Mmd_362, mouse; Mmd_386, mouse
ctttttcttttcttgaagcctgactccccgcgggacttccaaggcagcataacttcaggtccagactgggctgtcaggcttttccaggaaggatccagg

SEQ ID NO: 675 - DNA, 97 bp, Artificial
  Hairpin sequence Mmd_363, mouse
aggcaaaccctacaaatgtagcagaagcatttccgcacactggcatcaacatctagtttgtgcagaaatgtttccactagattcatagagtgttctt

SEQ ID NO: 676 - DNA, 91 bp, Artificial
  Hairpin sequence Mmd_364, mouse
ccatggctgctgctgtagcagaagggttgagccagtccaggctggaaggatctgtcacccgatcccaggtctgtgctacagccagtcttgg

SEQ ID NO: 677 - DNA, 95 bp, Artificial
  Hairpin sequence Mmd_365, mouse
ggaaggatatctgaggactggatgtgttcatgttttacaccaaatgctttcaacatgagaatccgcccagtcgtatctaataaaaatatccttcc

SEQ ID NO: 678 - DNA, 79 bp, Artificial
  Hairpin sequence Mmd_366, mouse
gtcacctcttcaaggtgtttgggattcgctttgatatccttgtggatggcaaggtaagtgtcctatgtgaagggatggc

SEQ ID NO: 679 - DNA, 97 bp, Artificial
  Hairpin sequence Mmd_367, mouse
cacagcttggcactggaaattttatttgaatcaggggagaatattatttgcgatattttcctaaatgacaaatgaatgatttaccggcagcaatgtg

SEQ ID NO: 680 - DNA, 82 bp, Artificial
  Hairpin sequence Mmd_368, mouse
ccgcatttgctccagtggacactggtgcgcacccacttcaagatacactgggctagtgtcagtctccaaggagcgcgtgcgg

SEQ ID NO: 681 - DNA, 93 bp, Artificial
  Hairpin sequence Mmd_369, mouse
tgagacctttctgctctcgctgtggttatgtgtctcactgtttctctacaagggtgagacaaagcaatttcacagttatcagaagggttatca

SEQ ID NO: 682 - DNA, 98 bp, Artificial
  Hairpin sequence Mmd_37, mouse
ccttctggaatttgcttgtggcgctgtttattacctaccaggcattttgctgaaggtgcaatttgcagagccattaatggcaatgaagttccagcggg

SEQ ID NO: 683 - DNA, 100 bp, Artificial
  Hairpin sequence Mmd_370, mouse
atgccatgcaaattggcacgaaggcaatcagtctgactctgaaggctgccccaccggccgcaggctggctcattgtcttttctgtcaatttgcatctcat

SEQ ID NO: 684 - DNA, 96 bp, Artificial
  Hairpin sequence Mmd_371, mouse
agaggctgccaggtgctggtggaaatagaggccgaagaggctggagagcagctggcaggcagctgtccaccagatgatgtaagccagcatgcctct

SEQ ID NO: 685 - DNA, 99 bp, Artificial
  Hairpin sequence Mmd_372, mouse
gtccccgttgtctgcgtccttgcctgtctcttccctgcccagtggcctccttgtccggctcactgggcaggagccctaatcggattggacagctgagat

SEQ ID NO: 686 - DNA, 84 bp, Artificial
  Hairpin sequence Mmd_373, mouse
ttttctttttcagaaaattcttccaggaggaaacacatcatttgatgtagtttttcttgcaagagtagtaggaaatgtagaaaa

SEQ ID NO: 687 - DNA, 99 bp, Artificial
  Hairpin sequence Mmd_374, mouse
ggcagtcgtcacctttcttccgggcactggccaagccaaagtcagtgatgatgatcttggagtcagtgcctgggtggtagtaaaggagattctcaggct

SEQ ID NO: 688 - DNA, 85 bp, Artificial
  Hairpin sequence Mmd_375, mouse
aaaagcttggcgtaattatctctgaacaggttgagcgcaaggtctctgcaaaatctgctcagggtgattattttgctggggtttt

SEQ ID NO: 689 - DNA, 96 bp, Artificial
  Hairpin sequence Mmd_376, mouse
gttatcaggaaataatcatagccctcgggtgctgggagacactgggtgtagctaagtgtctctaattccccaagggtatgattagctcacgttaac

SEQ ID NO: 690 - DNA, 98 bp, Artificial
  Hairpin sequence Mmd_377, mouse
actcgggaagcagcattcaggtctctgggtcctggatgtccttggtgcacactccaaggactcctcgtccttaagttcatagtctgtattccctgagt

SEQ ID NO: 691 - DNA, 82 bp, Artificial
  Hairpin sequence Mmd_378, mouse
cgaacacccaaaataatgtgagatcaggctcatgcgttatgcatgaggtctgatttaacatgaacattatttcttgtgtacg

SEQ ID NO: 692 - DNA, 82 bp, Artificial
  Hairpin sequence Mmd_379, mouse
cattccaagccactctctgatttgctgtgagtggcagaatcattcaccgtggtgaatcacagagagcgagccatgtggagtg

SEQ ID NO: 693 - DNA, 96 bp, Artificial
  Hairpin sequence Mmd_38, mouse
aggcagcggtcctacatgaatatggaggtctctgtctgacttagaatagctggctgagtctgatcgttcacctccatacaaccttagactctcact

SEQ ID NO: 694 - DNA, 98 bp, Artificial
  Hairpin sequence Mmd_380, mouse
gctgaacgatcgatccccatcccagcttccttccagcccttgatgttttagtacgcacataggctagagagaggttggggatggggaaagatgagagc

SEQ ID NO: 695 - DNA, 87 bp, Artificial
  Hairpin sequence Mmd_381, mouse
cagggaagcacaggtggctcatttagaagcagccatacactcagagagtggctgcctatacatgggctttcctgtgctcctctcctg

SEQ ID NO: 696 - DNA, 98 bp, Artificial
  Hairpin sequence Mmd_382, mouse
cgggagagctcagtgaaggactcaatgacgttgaagttgcacagggggctgacttcaaagaaggtcatgcagttcttctctgcataggctctggcctg

SEQ ID NO: 697 - DNA, 90 bp, Artificial
  Hairpin sequence Mmd_383, mouse
ttcacctggcgcactgggcaggaagactttattcagatccaaggaggaggctctggaatgggttttctgtggccgtggtggtggggtggg

SEQ ID NO: 698 - DNA, 87 bp, Artificial
  Hairpin sequence Mmd_384, mouse
catctggaatttctattgctcaatgggaggctgatgggggagctgatctggaaagagcctccaaaaagaaataatgagttccagatg

SEQ ID NO: 699 - DNA, 89 bp, Artificial
  Hairpin sequence Mmd_385, mouse
ttaacctcaatggtcctgatatggcaggcctccttgggctgtatggttccagattagtaggtctgtatggttcaggatttacaggttgg

SEQ ID NO: 700 - DNA, 98 bp, Artificial
  Hairpin sequence Mmd_387, mouse
caactagagagacccggttgcttgggtggatccagacaaaggtggatttccatccctggattttgtgcccacgcctctgggtcaccctgtctcccttg

SEQ ID NO: 701 - DNA, 99 bp, Artificial
  Hairpin sequence Mmd_388, mouse
agggtgcgtattacagacgaacaatgatatgccttgccagtaatggcactcagtcaaaactaccagggcatgtcattattaggtcataatacccaccct

SEQ ID NO: 702 - DNA, 98 bp, Artificial
  Hairpin sequence Mmd_389, mouse
ggcaccagctccgtcagctccagccccaggtcctgcgcgatgcggcccacgaaggtgccgtgtttggcctcctctgggacggagtagcggagctggcc

SEQ ID NO: 703 - DNA, 91 bp, Artificial
  Hairpin sequence Mmd_390, mouse
tctactaggaatgggatacattcacaggtgtggttgttggagacatgctgcgcaaagctgtgagttaaaatctgatttctctcctagggga

SEQ ID NO: 704 - DNA, 96 bp, Artificial
  Hairpin sequence Mmd_391, mouse
ccattaagcagagctggcattttagagcacgtcagaggcgctggcttcctgccctggaagtcagcgtctggcctctgagccagcattgcctgccgg

SEQ ID NO: 705 - DNA, 94 bp, Artificial
  Hairpin sequence Mmd_392, mouse
ctgggcaaaggctctcctaaggcaggcagacttcagtgttgcccatttctcagttctctgaagcttgcttacctccaaagggcctttgccatag

SEQ ID NO: 706 - DNA, 89 bp, Artificial
  Hairpin sequence Mmd_393, mouse
ctctccatatgctgtggcatctgtaactggcatgtaagggactcattttcctcacatatctcattaaagatgcaaagcaaaatggaggg

SEQ ID NO: 707 - DNA, 84 bp, Artificial
  Hairpin sequence Mmd_394, mouse
atttactattttagtgctggatccagttgcagctgaaacatttcaaccaggctgcggagttgggccagctctgtgatggtcagt

SEQ ID NO: 708 - DNA, 96 bp, Artificial
  Hairpin sequence Mmd_395, mouse
gagtgttgtgtgcaatgctttatctagttggatgtcaagacacgtgaaacttaagtgcatgacaccacactgggtagaggagggctcacagtgttc

SEQ ID NO: 709 - DNA, 79 bp, Artificial
  Hairpin sequence Mmd_396, mouse
tacaggacgtctgtgcaattcgatggagctcatcactttccggtctatgagcaattaaattgcaccaggaagccctgta

SEQ ID NO: 710 - DNA, 57 bp, Artificial
  Hairpin sequence Mmd_397, mouse
tctctgcctgaggttgtgtgagggcagactagcccaggaccaggccttggggagaga

SEQ ID NO: 711 - DNA, 91 bp, Artificial
  Hairpin sequence Mmd_398, mouse
tgcttgattagtcagattgatggaggaggccatatgtggccgtgtcaaaacttggctaccaggctcctttcctctctctgaaggacaggca

SEQ ID NO: 712 - DNA, 87 bp, Artificial
  Hairpin sequence Mmd_399, mouse
gctcaccctggaggccacagatgctgatggaagccgcacccatgctactgtggactacagcatcatcagtggcaatcggggccgagt

SEQ ID NO: 713 - DNA, 96 bp, Artificial
  Hairpin sequence Mmd_4, mouse
tgatattatgccatgacattgtgtcaatatgcgatgatgtgttgtgatggcacagcgtcatcacgtggtgacgcaacatcatgacgtaagacatca

SEQ ID NO: 714 - DNA, 96 bp, Artificial
  Hairpin sequence Mmd_40, mouse
gacggctcagaagtccatggagctgtgggccaggtagtccttgcggccaatgttgctgacctgcttggtctgcatagcttcaagtttggggcagtc

SEQ ID NO: 715 - DNA, 96 bp, Artificial
  Hairpin sequence Mmd_400, mouse
tcatgccgagcgaaaaagcccctgacgtcacctgcagccaatcagagtgagccgctcgggggcaggtgacgtcagcggagcgggcgctgggtgcgg

SEQ ID NO: 716 - DNA, 98 bp, Artificial
  Hairpin sequence Mmd_401, mouse
gagcgattcatgccgccacctcacccactgcgaggcacctgggtaatgcgtcccttgcttgtggtgcgtgagctctgtggctccgttttgttttgctc

SEQ ID NO: 717 - DNA, 92 bp, Artificial
  Hairpin sequence Mmd_402, mouse
cttcttcagaagctctgctcaccatgcttcctggtccttgcagagtcctggcctgggctgctctgggtgctgcagagtcttcttgggaggag

SEQ ID NO: 718 - DNA, 94 bp, Artificial
  Hairpin sequence Mmd_403, mouse
aagggagggtatggtgaggatggtggtcttggaggcggacatttatccaacagaatgctactattagacaggccgtttttacctagattccttt

SEQ ID NO: 719 - DNA, 68 bp, Artificial
  Hairpin sequence Mmd_404, mouse
cttgacatgtccctttaacgttagaggcatttgctgatgccactaacttatgggtcaatgtggtcaag

SEQ ID NO: 720 - DNA, 97 bp, Artificial
  Hairpin sequence Mmd_405, mouse
ctgtgcagcctgccccgcgcctgggccctcttcccacccaggtcagcaggggagagagggcaggctccccagggaggggacaggaggcagtacccag

SEQ ID NO: 721 - DNA, 99 bp, Artificial
  Hairpin sequence Mmd_406, mouse
ccttatcatagagtttactgaagtggggccagttccccgcctcttccagctttgctgccaaagttggagaggggtggagtggcctggaagactgttggg

SEQ ID NO: 722 - DNA, 97 bp, Artificial
  Hairpin sequence Mmd_407, mouse
gatgatgagctgtcctttcccgagggagcaattattcgcatcttgaacaaagaaaaccaagatgatgatggcttctgggaaggggagttcagtggtc

SEQ ID NO: 723 - DNA, 91 bp, Artificial
  Hairpin sequence Mmd_408, mouse
gagaaatcagctttaattaatttgagtgccagctctgtgtataattacccagcatgagagtttgcatgcaaatgcggaaatgctgattctt

SEQ ID NO: 724 - DNA, 88 bp, Artificial
  Hairpin sequence Mmd_409, mouse
ccatcagtcaggaaattgtatgataaataggaggtagccaggtttagtggcacttccttctgcttcagcagtttttgcctgactttgg

SEQ ID NO: 725 - DNA, 99 bp, Artificial
  Hairpin sequence Mmd_41, mouse
atcagtttgtgtgcatttgcaggaacttgtgagtctcctattgaaaatagacaggagactgacaagttcccgggaacacccacaaatcttcctactggt

SEQ ID NO: 726 - DNA, 91 bp, Artificial
  Hairpin sequence Mmd_410, mouse
gtcgttgctgacccaagcggtcaggtagatgtagaaagcgctcggattaatgatgccatctgcgtctaccagacgctgtttagtcaactac

SEQ ID NO: 727 - DNA, 88 bp, Artificial
  Hairpin sequence Mmd_411, mouse
cttcttgtataagcactgtgctaaaattgcaggaactaagattctatcttggtttttgtaataatgctagcagagtacacacaagaag

SEQ ID NO: 728 - DNA, 100 bp, Artificial
  Hairpin sequence Mmd_412, mouse
ccaccagttagcagcaggctgtggattaggagtggcagaaaggatgtctttaaataggtttccgtttctgtttgtttggtttgaggcttggtgatggagg

SEQ ID NO: 729 - DNA, 98 bp, Artificial
  Hairpin sequence Mmd_413, mouse
gcaggcgtttcctggggattaatgaccagctgggaagaaccagtggccctcggctctgcctcccagccagccattaactccaaggaaatgtcttttgc

SEQ ID NO: 730 - DNA, 86 bp, Artificial
  Hairpin sequence Mmd_414, mouse
ggtggtgcatggcagcagcacactgtgagtccaaacagcagatggcagcgctgttgagtaggctggcaagctgatgtctagccacc

SEQ ID NO: 731 - DNA, 99 bp, Artificial
  Hairpin sequence Mmd_415, mouse
ctttttcttttcttgaagcctgactccccgcgggacttccaaggcagcataatttcgggtccagactgggctgtcaggcttttccaggaaggatccagg

SEQ ID NO: 732 - DNA, 80 bp, Artificial
  Hairpin sequence Mmd_416, mouse
tgggggaggcgggctgtgctggtgcccggtgtataatcgggttaatccagtagacaaagaataaaccaatagccctccca

SEQ ID NO: 733 - DNA, 97 bp, Artificial
  Hairpin sequence Mmd_417, mouse
ctatcgaatttgtggcttttgctagctttgcagaaagggacttttaaaaatacttctcttgatagtgtaaaactagcgaaagccacaaatggaatgg

SEQ ID NO: 734 - DNA, 98 bp, Artificial
  Hairpin sequence Mmd_418, mouse
cactagttctcctcccatccccctaccccaaacttctctactctggtttaccagcaccaacaaccacgtgggggaggggtaagagagtagaaccggtg

SEQ ID NO: 735 - DNA, 95 bp, Artificial
  Hairpin sequence Mmd_419, mouse
cagccttttctttggtgtgcgaatgtgagtgtgtgtaatgatgttatctatatgccgtcggggcacctgcacatttgcatattggggaagaactg

SEQ ID NO: 736 - DNA, 80 bp, Artificial
  Hairpin sequence Mmd_42, mouse
aaccaaagggcaagatgtctgcttatgctttcttcatgcagacatgcagggaagaacataagaagaaaaacccagaggtt

SEQ ID NO: 737 - DNA, 97 bp, Artificial
  Hairpin sequence Mmd_420, mouse; Mmd_513, mouse
tgtatgtgcatgtgtgtatagttgtgtgtgcatgttcatgtctatatttgaatatacataacatacacacacacgtataaacgcaagcacacataca

SEQ ID NO: 738 - DNA, 65 bp, Artificial
  Hairpin sequence Mmd_421, mouse
tcagtcaggctgctggctatacaccaggacttatctggtcctggatataaccactgccagactga

SEQ ID NO: 739 - DNA, 97 bp, Artificial
  Hairpin sequence Mmd_422, mouse
cccatttcgagtcataatccccttttgtgacatcacaattagccacttgtggtgatgattataatgtcacaaacgcaggattaggacaaggaatggg

SEQ ID NO: 740 - DNA, 100 bp, Artificial
  Hairpin sequence Mmd_423, mouse
cagtgtaacccatgctggctgcagcaatgagggcctgctggatggcgtggctcttcttaaacactccttgctgctggccagccattgtccagtcacactg

SEQ ID NO: 741 - DNA, 99 bp, Artificial
  Hairpin sequence Mmd_424, mouse
gtcccgagagttgctgatacaagctctacaaccattgagctggagtgggaacccccagctttcaatggtggtggggaaattatgggctactttgttgat

SEQ ID NO: 742 - DNA, 93 bp, Artificial
  Hairpin sequence Mmd_425, mouse
tgctctccctggctgggggtaacctctgcccctggagccttcccttgttcctacaatgccaacctctcaggttagacccagccctggagctca

SEQ ID NO: 743 - DNA, 94 bp, Artificial
  Hairpin sequence Mmd_426, mouse
gcctgttgacatgtccagacactactccagcagaggcactgggcctggcaagaggccaggcctgagtcacagctgctggatatatcaacgtggc

SEQ ID NO: 744 - DNA, 99 bp, Artificial
  Hairpin sequence Mmd_427, mouse
ctggacgtgttagtggaggccttaaggcctgtgggcacctttaagaaaatcggaaaggtgttccgcaaagaagaggactccacggtggggatgctgcag

SEQ ID NO: 745 - DNA, 96 bp, Artificial
  Hairpin sequence Mmd_428, mouse
gctatcgatgtgaagatcctgcagcagctggtgaccttgaacgagggtattgaggcagtgcgctggctgctggaggagcggggtacactgacgagc

SEQ ID NO: 746 - DNA, 82 bp, Artificial
  Hairpin sequence Mmd_429, mouse
gttttacagctcagaggggattaggacaagagatgtgtttaaaatacgatgtgtctctctgtctctctgcagctgatggagc

SEQ ID NO: 747 - DNA, 82 bp, Artificial
  Hairpin sequence Mmd_43, mouse
tagaaaaggcaattaaaatccacaactgacttccttcccgtgtccaagatgagggacagtgcgtgggtgtgtgcccttttta

SEQ ID NO: 748 - DNA, 91 bp, Artificial
  Hairpin sequence Mmd_430, mouse
tggaagaccatcgtgcaggactacatttgttctccccatgcagagagcatgaggaagagaaaccagattgtgttcacgatggtggaggccg

SEQ ID NO: 749 - DNA, 87 bp, Artificial
  Hairpin sequence Mmd_431, mouse
gctggttttggcacttctctcactctagtgagttgcaaaaaaacaagcaggtgctagcaatctgagagaccacatgccagctccagc

SEQ ID NO: 750 - DNA, 93 bp, Artificial
  Hairpin sequence Mmd_432, mouse
ctgaggcagctgaggccatgctgtctggctcaatgaggccctcaggaacagaggagccaaagccacgggacagggcctcagatgcgatttcag

SEQ ID NO: 751 - DNA, 99 bp, Artificial
  Hairpin sequence Mmd_433, mouse
gttggtgccctgataaagccggagccctggcagataaatgctgaggtggagccaagccatttatttgaggagcagcagggctcctatcagggcgccggc

SEQ ID NO: 752 - DNA, 96 bp, Artificial
  Hairpin sequence Mmd_434, mouse
gacaggatagggtccttaaaataaactctctggtttccttcctttctttatctaaggggaggctaaagtgggtttgcacaaagggctcttcgtgtt

SEQ ID NO: 753 - DNA, 74 bp, Artificial
  Hairpin sequence Mmd_435, mouse
tttgtgtgtataaaatgggtaattgcacacacaaatgaacaattttgtatgcaattatccattttgcatacaga

SEQ ID NO: 754 - DNA, 94 bp, Artificial
  Hairpin sequence Mmd_436, mouse
caggtatccaccaaggacatgcccctggcactgatggcctgtgccctccgaaaaaaggccacagtgttccggcagcctctggtggagcagcctg

SEQ ID NO: 755 - DNA, 79 bp, Artificial
  Hairpin sequence Mmd_437, mouse
ctgggcatacagaaccccctggcagccagccttgaggtggcccctgccttccgtctggctgagagcaggactaacccag

SEQ ID NO: 756 - DNA, 97 bp, Artificial
  Hairpin sequence Mmd_438, mouse
gttgttcttaaatcaccttgaagtgtctcttgtcaaagataactccctgcattgtagaaacgtctttgacaagagaggattagggtgaaattgcaat

SEQ ID NO: 757 - DNA, 98 bp, Artificial
  Hairpin sequence Mmd_44, mouse
gtgaggagcaggttagctgggtgaaaagttcgcagcgaggggagctgtctgtttcctttgcttaatttatccactatttggctaacctcactctgaac

SEQ ID NO: 758 - DNA, 93 bp, Artificial
  Hairpin sequence Mmd_440, mouse
tgacaatccaccaagaacccttcgtgtatgtcaagcccacaatgagtgatggcacatgcaaagaggagttcacagtcaatggtgaccctgtca

SEQ ID NO: 759 - DNA, 90 bp, Artificial
  Hairpin sequence Mmd_441, mouse
gccaaaatcagagaagggattctgatgttggtcacactccaagagttttaaaatgagtggcgaacacagaatccatactctgcttatggc

SEQ ID NO: 760 - DNA, 92 bp, Artificial
  Hairpin sequence Mmd_442, mouse
cagcagaggtaagcctgtgagactaacagctttagcgcaggattcatctttccttcttgactgtggtttacagagtgctttacctccagctg

SEQ ID NO: 761 - DNA, 94 bp, Artificial
  Hairpin sequence Mmd_443, mouse
cagagatgggattcacatgaaccacagctgagttcacagagatgatttgtgagcctcagtatttgcacatatgtggtcatgcgggacctctctg

SEQ ID NO: 762 - DNA, 75 bp, Artificial
  Hairpin sequence Mmd_444, mouse
ggtatatctgcagcctctcctctcctggggaaactgaattattggagtcctcaggagtatagctgttgatatgct

SEQ ID NO: 763 - DNA, 95 bp, Artificial
  Hairpin sequence Mmd_445, mouse
gaacccagggcttcttaagggaactacaactcccagtgtacatgcaaagcagctgtgatgcacactgggagttgtagtccttgagctctcagttt

SEQ ID NO: 764 - DNA, 99 bp, Artificial
  Hairpin sequence Mmd_446, mouse
tcccacctgaagcctaatccctgctttgctacaacacaattggttgcggtggaaatgattatgttgtcacaaacagaggattagaacttcgggtggaga

SEQ ID NO: 765 - DNA, 99 bp, Artificial
  Hairpin sequence Mmd_447, mouse
tcaaaataatgctgctaattacctctgatctcctttaatgggaagctgctcccaactccacaaaggagtaaatgagaggtccacagcacagacccagga

SEQ ID NO: 766 - DNA, 63 bp, Artificial
  Hairpin sequence Mmd_448, mouse
cccagctcccgctcacccctgccacgtcaaaggaggcagaaggggagtcgggagcggagaggg

SEQ ID NO: 767 - DNA, 95 bp, Artificial
  Hairpin sequence Mmd_449, mouse
ggcttctgagtctgatgccctcataccttgagggacatgatcatcagtagcccaatgtccctcttgatggcaacttagacatagtcagaatagct

SEQ ID NO: 768 - DNA, 99 bp, Artificial
  Hairpin sequence Mmd_45, mouse
gaggtaattttggccctcctgattgttaagctcaatggagatagattttttcttcctctgtgtgtgtgtttattgtaatatctggagggctttttcttc

SEQ ID NO: 769 - DNA, 88 bp, Artificial
  Hairpin sequence Mmd_450, mouse
gcagctggaaagggcttagccctttaatgccagcagctgtatctccaatgtggctgctctgctgaggggctggatgctatccagaagc

SEQ ID NO: 770 - DNA, 99 bp, Artificial
  Hairpin sequence Mmd_451, mouse
ctgattctgcctgcgtggagcgggcacagctgtgagagccccctaggtacagcggggctgcagcgtgatcgcctgctcacgcacaggaagtgacgacag

SEQ ID NO: 771 - DNA, 98 bp, Artificial
  Hairpin sequence Mmd_452, mouse; Mmd_473, mouse
ggattataataaattaaatgcctaaactggcagagtgcaaacaattttgactcagatctaaatgtttgcactggctgtttaaacatttaatttgttcc

SEQ ID NO: 772 - DNA, 100 bp, Artificial
  Hairpin sequence Mmd_453, mouse
ggtggaggtgggtgggctgcctcccaatcctggctcaggcagggtggcctctgatctgggctccggagttgggaaagggagctgacccatggaagacacc

SEQ ID NO: 773 - DNA, 68 bp, Artificial
  Hairpin sequence Mmd_454, mouse
gatatatacattctattgtccctagaaggtgtattccttctagggacaatgtgttggctgtgtgagtc

SEQ ID NO: 774 - DNA, 99 bp, Artificial
  Hairpin sequence Mmd_455, mouse
gtctttgtcgtggtgtcagaagattaattacactcatgtgagaacaactttctcattgggtgaaaagagacattatccttctgacttgtgatagacgac

SEQ ID NO: 775 - DNA, 81 bp, Artificial
  Hairpin sequence Mmd_456, mouse
gcatgcagctttggagacgccaaggggaaaatggatttaattgattcgttacctttggtattgtaatgcctgctgcgatgt

SEQ ID NO: 776 - DNA, 87 bp, Artificial
  Hairpin sequence Mmd_457, mouse
tgccttagctagttaaaccggaagcctatggtaatcagagagaggcacttcttgccttctcctatggtttcttcattagcagaggta

SEQ ID NO: 777 - DNA, 83 bp, Artificial
  Hairpin sequence Mmd_458, mouse
acctggaaaaggcagtgatgtttccaagcacatatctgacttatatgtgtggatggtaataatgctgagtcttttccgaaggt

SEQ ID NO: 778 - DNA, 82 bp, Artificial
  Hairpin sequence Mmd_459, mouse
gaccagctcacgggaagtggccttcaatagtccgatagcttcctgtatcctgacattgtgaaggcccacttcctgctctgtc

SEQ ID NO: 779 - DNA, 99 bp, Artificial
  Hairpin sequence Mmd_46, mouse
ctggagtatcctgctttgggatttgggtcagtcaatcagcaggtatttatcctgctttctttggacccagaatactgttaactcagcaggataccacag

SEQ ID NO: 780 - DNA, 98 bp, Artificial
  Hairpin sequence Mmd_460, mouse
cccaggaggttcaaacattgtctgctgagtttggacaagagagaaattaactccaaactgaagataatgtctaagaatctccatttcaaccagttggg

SEQ ID NO: 781 - DNA, 98 bp, Artificial
  Hairpin sequence Mmd_461, mouse
gccagagggggcggcgcgcgagagtctagtggcgctggtcagcacctcggacagggactcgggcgccaacgggcaggtgcgctgcgccctctacgggc

SEQ ID NO: 782 - DNA, 79 bp, Artificial
  Hairpin sequence Mmd_462, mouse
agaggcttggagctcgaggctaattgtgaattaggagcccaattatgccctgcacaattggctccacgctaccttctct

SEQ ID NO: 783 - DNA, 100 bp, Artificial
  Hairpin sequence Mmd_464, mouse
ttaggcagctggagaaaacaatgggccagattctcactctgtgtaatcacagaggacgactccattgtatagcccattgtttctcccatgtactgcctga

SEQ ID NO: 784 - DNA, 86 bp, Artificial
  Hairpin sequence Mmd_465, mouse
ccgtgtgagtttgcgctgcaaagctccttggcatccttgcatgggttgggtgttggggagctcaaattgcagctacaactggctgg

SEQ ID NO: 785 - DNA, 93 bp, Artificial
  Hairpin sequence Mmd_466, mouse
gtgtttatttgaatgtgtgatgaggaggtcatcaaaatgaacacgctcgttgtgtttatttgaatctcacatcgctcatgagagaatacacgc

SEQ ID NO: 786 - DNA, 76 bp, Artificial
  Hairpin sequence Mmd_468, mouse
ccgcggagtctctaactggcgacagatgggccactttcttctggccacaaaggggccggaatggagcgctccgcgg

SEQ ID NO: 787 - DNA, 98 bp, Artificial
  Hairpin sequence Mmd_469, mouse
atgtctaagcttttcaagttaactcctggctcatggctctggaaggtgctgtaaacattgtcccctctgccatgagtctggattttgttcttttacat

SEQ ID NO: 788 - DNA, 99 bp, Artificial
  Hairpin sequence Mmd_47, mouse
gagtttgacagcagcctaccacagaaccagccctttgttttctcccttggcactggccaggttatcaagggctgggaccaggggctgctggggtaagtc

SEQ ID NO: 789 - DNA, 92 bp, Artificial
  Hairpin sequence Mmd_470, mouse
ttggtcctgtttcctcctgtggcccggagtttgatgtgcagggcggtgatgcccagcttcttgcacctctgggccacatcctgggcagccaa

SEQ ID NO: 790 - DNA, 94 bp, Artificial
  Hairpin sequence Mmd_471, mouse
aaaatttttgtaggtggactgggaaagcagcggattgcagatgcgtttaacagcagggtattttccgctgccatgaccagttaagggagatttt

SEQ ID NO: 791 - DNA, 89 bp, Artificial
  Hairpin sequence Mmd_472, mouse
ggcccctctgtgtggctgctgctggctcagaccccagcccaccttgggcggtgggtttgggttttggctggcacattcaccaaggggcc

SEQ ID NO: 792 - DNA, 83 bp, Artificial
  Hairpin sequence Mmd_474, mouse
gaagtcagacttgcctccgcaactgcatcacagctacatctttggagctgttgaaatccagctgacggaggaatcggtccttt

SEQ ID NO: 793 - DNA, 79 bp, Artificial
  Hairpin sequence Mmd_475, mouse
gagtttgctggctctgaatggggtatgagacttgttcttacccaaagccctgatgcactctttcagggctagcagactt

SEQ ID NO: 794 - DNA, 89 bp, Artificial
  Hairpin sequence Mmd_476, mouse
cgggtctcagaaatcaggcttccggagagtgtagtttgtttgtaataataatacgaattattctctcagcctgaggtcactagggcgtg

SEQ ID NO: 795 - DNA, 98 bp, Artificial
  Hairpin sequence Mmd_477, mouse
gtccagcaatattttaagctcccagccagacagcttttatgaagggagggatgtttggaaaacaatgggagagagagaatgggagagctgtgctgggc

SEQ ID NO: 796 - DNA, 98 bp, Artificial
  Hairpin sequence Mmd_478, mouse
ctgtccggaatatggttggttggtcctgagcatgttagaggaattcctgttcagcatgacatgctcagaggccacttggccagagatgagctgggagg

SEQ ID NO: 797 - DNA, 85 bp, Artificial
  Hairpin sequence Mmd_479, mouse
gcctccttccagcgcagcagctgccggatctcggcctcacgctcgcgctgcacctcctcctgcagctgccgcagctcccggaggc

SEQ ID NO: 798 - DNA, 93 bp, Artificial
  Hairpin sequence Mmd_48, mouse
cctttggttcacattaaaatataaattccccgtgggagagaccctattaagccttgcagggaattactaatttacattttaatgtgaatgagg

SEQ ID NO: 799 - DNA, 99 bp, Artificial
  Hairpin sequence Mmd_480, mouse
gatcacgtcaccatggccaaccgggagttggcatttaaggctggcgacgtcatcaaagtcttggatgcttccaacaaggattggtggtggggccagatc

SEQ ID NO: 800 - DNA, 91 bp, Artificial
  Hairpin sequence Mmd_481, mouse
gcggtttggagggctgcattcattttgagtgctgttgtaatcaattttagatacagtattccacaatggatcagtttgcatccaaacctgc

SEQ ID NO: 801 - DNA, 93 bp, Artificial
  Hairpin sequence Mmd_482, mouse
tccctggagccgctccactgacggcaccatcttggcacaaaaactagccgaggaggtgcccgtggacgtggcctcttacctctacactgggga

SEQ ID NO: 802 - DNA, 91 bp, Artificial
  Hairpin sequence Mmd_483, mouse
ccagctgcccgggaagcttggcggccaggctgagggaaggaagcgtcggctctctttgaaggtcggctgccagtgaatctcaggcagctgg

SEQ ID NO: 803 - DNA, 97 bp, Artificial
  Hairpin sequence Mmd_484, mouse
gtgtagatggtaacttgttagaaggttacttgttagttcaggacctcattactttctgcctgaactattgcagtagcctcctaactggttatcccac

SEQ ID NO: 804 - DNA, 85 bp, Artificial
  Hairpin sequence Mmd_485, mouse
actcttatttcctccactgcctgactttattgaatgccgtggggcatattaatcagtaaagccaagtagtgcatgaaataaaagt

SEQ ID NO: 805 - DNA, 91 bp, Artificial
  Hairpin sequence Mmd_486, mouse
gagcggttacatttggcagcattccttgttagcatttgataaacaattattgccaaatgttagcaaggaaacctgccaaatgttacagctc

SEQ ID NO: 806 - DNA, 101 bp, Artificial
  Hairpin sequence Mmd_487, mouse
tgctgttgtccttggagaaatctgggcctgagacagaggagggatgtaatttgagtgctcctttgtcacaggttaccagggtgtgcttgaggggacggtag

SEQ ID NO: 807 - DNA, 100 bp, Artificial
  Hairpin sequence Mmd_488, mouse
ggagcagccggcttcatttgtgatccgggagcctggtgccagcaagacctggaatttccggtctggttggtcttgggccccgtggagccaggttgatacc

SEQ ID NO: 808 - DNA, 100 bp, Artificial
  Hairpin sequence Mmd_489, mouse
ggtggaggaggactgttatttaaaaggcttttaaatgatgtcactatatattcttttaacatcctctaaaagcctttgaaaaaacagtctctacctccct

SEQ ID NO: 809 - DNA, 98 bp, Artificial
  Hairpin sequence Mmd_49, mouse
gcgcgagctgtccccgaacaccttgcggtaggaggaggcggagcacaaatagtgctccgatccgaagctcatggtgccggggtcggggccgggtgcgc

SEQ ID NO: 810 - DNA, 79 bp, Artificial
  Hairpin sequence Mmd_490, mouse
agtgttcgaatggaggttgcccatggtgtgttcattttatttatgatgagtattacatggccaatctcctttcggcact

SEQ ID NO: 811 - DNA, 99 bp, Artificial
  Hairpin sequence Mmd_491, mouse
tgagctgcttcttcctctctcctcttttgcacaaagagtctcatgtctgatatttagacatgatgagctttgtgcaaagtggaacctggtttcttctca

SEQ ID NO: 812 - DNA, 90 bp, Artificial
  Hairpin sequence Mmd_492, mouse
aaggatttggcatttcaatcgatgggaaatgatgtgtgatcattctaatttattatacatcattcccatcaatcgacgggttgaagcctt

SEQ ID NO: 813 - DNA, 100 bp, Artificial
  Hairpin sequence Mmd_493, mouse
cctaggtgccatcaggagggacatcaggctgtcgattgccctcgagtagcacagaacagagggacatcacccccaagtgacctccagctgacctcctagg

SEQ ID NO: 814 - DNA, 94 bp, Artificial
  Hairpin sequence Mmd_494, mouse
ctctgctgtctggccatttcgggttttccagtccgatgctcctgagggggagggtcgggccccttggaaaatccgttttcatggcaacacggag

SEQ ID NO: 815 - DNA, 98 bp, Artificial
  Hairpin sequence Mmd_495, mouse
tcattcattgtctttcttatgtttcccaacagacactgagcacattgaagagctgcagtgggtgtggagaacatacttgaacaatcagaaatgaatga

SEQ ID NO: 816 - DNA, 93 bp, Artificial
  Hairpin sequence Mmd_496, mouse
gctcatttttgccatgtggagtcagtcattctggagtggctggggacagcaggaaagccagcagagtggctgacccgggatggtttcaagggc

SEQ ID NO: 817 - DNA, 92 bp, Artificial
  Hairpin sequence Mmd_497, mouse
ctagactagcctagactgggaagcctgggctaagatgcagtaaactattttcagtttataaacaagtcctgacatcccagtttgggtgttgg

SEQ ID NO: 818 - DNA, 99 bp, Artificial
  Hairpin sequence Mmd_498, mouse
agagtgttccagccctgagccaccctcccaggagccccacagatccagtcttgactgtcccaatggggcagggaggaaaggtggccgctgggatttttt

SEQ ID NO: 819 - DNA, 98 bp, Artificial
  Hairpin sequence Mmd_499, mouse
ctcgaaaggtcacacatgtaatgaattgtgggtgcttgcctggcttcccaggcgagaaccgtaagcaattattaaatgccattgtgtgtcaggaaggg

SEQ ID NO: 820 - DNA, 99 bp, Artificial
  Hairpin sequence Mmd_5, mouse
gacgtacggtgcttaatgagaagttgcccgcgtgtttttcgctttatatgtgacgaaacattcgcggtgcacttctttttcagcatcctattctacgtt

SEQ ID NO: 821 - DNA, 91 bp, Artificial
  Hairpin sequence Mmd_50, mouse
tcgtgtggttagtgacgtgtgcttggtgtgtcttagcttggttgtgtgttttagagtggagtccatactgagtgctgtaactgccatggga

SEQ ID NO: 822 - DNA, 81 bp, Artificial
  Hairpin sequence Mmd_500, mouse
gacattcccaacattttatggcaatttgtttcatttgccgcaggcccataacgatgcaaattgccataaagtgggataatc

SEQ ID NO: 823 - DNA, 92 bp, Artificial
  Hairpin sequence Mmd_501, mouse
taatggtcagataatggatgacaataaaagttatgggtccctagctggggtgtagctcataatggttggcaagatccattattagaccatta

SEQ ID NO: 824 - DNA, 91 bp, Artificial
  Hairpin sequence Mmd_502, mouse
tggagcctgaggggctcacagctctggtccttggagctccagagaaaatgttgctccggggctgagttctgtgcaccccccttgccctcca

SEQ ID NO: 825 - DNA, 89 bp, Artificial
  Hairpin sequence Mmd_503, mouse
ggccaaaagctgccctcagctatgcatgcacagcttccattgacgtcagtaagaactgtgctcatgtaacaggaggcagaaatttggct

SEQ ID NO: 826 - DNA, 99 bp, Artificial
  Hairpin sequence Mmd_505, mouse
tcccatgtgcctttaaggaatcactcttgatctcagtcataaaattggaaagccacacaactgacaagatgatccccccaaggcagctgaaatatgggg

SEQ ID NO: 827 - DNA, 91 bp, Artificial
  Hairpin sequence Mmd_506, mouse
gtcttttaactctctattgctggtggctctgatttcatgatgcattttgcttatgaaactctagctttcagcactaaattgagttaaagat

SEQ ID NO: 828 - DNA, 94 bp, Artificial
  Hairpin sequence Mmd_507, mouse
ggtgcggggcgcgtcgcccccctcaggccaccagagcccggatacctcagaaattcggctctgggtctgtggggagcgaaatgcaacccaaacc

SEQ ID NO: 829 - DNA, 96 bp, Artificial
  Hairpin sequence Mmd_508, mouse
agcttgaagtagaaaaacaaaaatgggaaacagatgggtaatacccagaaagaaaattttctcggtattgcccatctgcttctctgcctgaaagct

SEQ ID NO: 830 - DNA, 87 bp, Artificial
  Hairpin sequence Mmd_509, mouse
ggagcctggctcactggaggctgcagagaagtgaactgaagaattacagtcggtttcacttccctgccttatgtctcttcaggtgcc

SEQ ID NO: 831 - DNA, 92 bp, Artificial
  Hairpin sequence Mmd_51, mouse
cacttttctgaagtgtgaattgcctgctgtcagcctggagttctcacctccgcaatcctggctgcaggcatatttgcatattggatgctgtg

SEQ ID NO: 832 - DNA, 98 bp, Artificial
  Hairpin sequence Mmd_510, mouse
caaggtcttggtatggtagcgccccaggatggagtaggcagggccaaggtccttgccagtcttcaggatcttggggttcacactgtagcggggccctg

SEQ ID NO: 833 - DNA, 72 bp, Artificial
  Hairpin sequence Mmd_511, mouse
ggaaggtactctgtgggagggaggagatcagagccaaggatgacattcctccttcacacttgcagtacctcc

SEQ ID NO: 834 - DNA, 86 bp, Artificial
  Hairpin sequence Mmd_512, mouse
ggctgctccttgcctttgatgtgaggcaagctggggcagtgcagcccctgttctggttgccttttgtcagataggagttgacagcc

SEQ ID NO: 835 - DNA, 100 bp, Artificial
  Hairpin sequence Mmd_514, mouse
ggcctagtcatcaccctgagccttgcactgagatgggagtggtgtaaggctcaggtatgcacagctcccatctcagaacaaggctcgggtgtgctcagct

SEQ ID NO: 836 - DNA, 73 bp, Artificial
  Hairpin sequence Mmd_516, mouse
ttacagctgattggacggggtcgatacggagcagtatataaaggttccttggatgagcgtccagttgctgtaa

SEQ ID NO: 837 - DNA, 95 bp, Artificial
  Hairpin sequence Mmd_517, mouse
gcacaccgcacagaagtcctcattctctatgggggccggggtgttcttcttggctcctggagctcgggggatgagtctgtgttcctcagatgtgc

SEQ ID NO: 838 - DNA, 96 bp, Artificial
  Hairpin sequence Mmd_518, mouse
cgcatgtcacacagccggattgtccccttgctgctgctgtatacgaaggtgttgcagtggtgggggtggaactcagctgcagtgatcacctctgtg

SEQ ID NO: 839 - DNA, 73 bp, Artificial
  Hairpin sequence Mmd_519, mouse
ggaggtgcccaggtcagtgcgctggttcatggaggcggacgtatctatgaggaacagcaggatgggcatcttc

SEQ ID NO: 840 - DNA, 90 bp, Artificial
  Hairpin sequence Mmd_520, mouse
tgcaccagggagaacagctgatggatcacaaatctctgcatttcaccaacagaaatgaatttgaatccatcaattttattcccttatgca

SEQ ID NO: 841 - DNA, 88 bp, Artificial
  Hairpin sequence Mmd_521, mouse
gggtctgcttacacattagcaactgactgtctccttcaaccaaacatgatcccacagacagtcaggagctgtggaagcaaagaaaacc

SEQ ID NO: 842 - DNA, 81 bp, Artificial
  Hairpin sequence Mmd_522, mouse
agatttcattagtctggttgctggctatattccaggacttatctggtcctggatataaccactgccagactgaatggtgct

SEQ ID NO: 843 - DNA, 85 bp, Artificial
  Hairpin sequence Mmd_523, mouse
ccgttcaaataaaagattgaaggatgctgtggcccactgccatgaggcagacaggaacacagctttacgttcaagtttagaatgg

SEQ ID NO: 844 - DNA, 88 bp, Artificial
  Hairpin sequence Mmd_526, mouse
tcctcctgcttggcgttgatgtctgctttcatctccatggcaacatgacggggaaggacagacagcaggagacgctcctgcagaggga

SEQ ID NO: 845 - DNA, 94 bp, Artificial
  Hairpin sequence Mmd_527, mouse
gtctgtgctgtggacctctcatttactcctttgtggagttgggagcagcttcccattaaaggagatcagaggtaattagcagcattattttgac

SEQ ID NO: 846 - DNA, 95 bp, Artificial
  Hairpin sequence Mmd_528, mouse
tgagcagctgttccatagaggctactatagaaacagctacaacagcataaccagcgcgagtagcgatgaggagctcctagatggagcaggtgcca

SEQ ID NO: 847 - DNA, 99 bp, Artificial
  Hairpin sequence Mmd_529, mouse
gtataaaatttctaagcagtgtgtgagaggtgtacgcagaaatcatgttggcaaaatattttgtgtaagtgcctcccacacacagctttaaaatgttac

SEQ ID NO: 848 - DNA, 87 bp, Artificial
  Hairpin sequence Mmd_53, mouse
atgacgtaaactctagttcatatttgagtgtcaaaagttggagtttgacatattccaaaatgtcactcaagatggagtttgggttat

SEQ ID NO: 849 - DNA, 99 bp, Artificial
  Hairpin sequence Mmd_530, mouse
tgggctccatgggtgtccagatgcctgatacattctccaggccaaggtatataagaacctgtggcctggggaggcgaggacagctggacaaacagctta

SEQ ID NO: 850 - DNA, 89 bp, Artificial
  Hairpin sequence Mmd_531, mouse
gccgcgtagtgaaagagctagcgtgggcagttcttttgtactccagctggagtaaaatgaacgcaggctcgcgtgttttcatcgctggt

SEQ ID NO: 851 - DNA, 93 bp, Artificial
  Hairpin sequence Mmd_532, mouse
gctcgcaggcaggggatttgaggagcggttgtgtgatttgtacagctgtcattagggtaattgcacgatattttcccaaatttctcctgcagc

SEQ ID NO: 852 - DNA, 90 bp, Artificial
  Hairpin sequence Mmd_533, mouse
ttaagctgagtgcattgtgatttccaataattgaggcagtggttctaaaagctgtctacattaatgaaaagagcaatgtggccagcttga

SEQ ID NO: 853 - DNA, 80 bp, Artificial
  Hairpin sequence Mmd_534, mouse
taccctcagagctgatctgagtgtggagattccattgacttcaaaggaagcttcatgcttggatcttctccacaggggtg

SEQ ID NO: 854 - DNA, 101 bp, Artificial
  Hairpin sequence Mmd_535, mouse
tgctgttgtccttggagaaatctgggcctgagacagaggagggatgtaatttgaatgctcctttgtcacaggttaccagggtgtgcttgaggggacggtag

SEQ ID NO: 855 - DNA, 94 bp, Artificial
  Hairpin sequence Mmd_54, mouse
agaacagggtctccttgaggggcctctgcctctatccaggattatgtttttatgaccaggaggctgaggtcccttacaggcggcctcttactct

SEQ ID NO: 856 - DNA, 84 bp, Artificial
  Hairpin sequence Mmd_55, mouse
gccagttgtggggcggagcaggctgagtttacagcgggccgcagacatctgttgagcccagcctgccctgcacccaggctgtgc

SEQ ID NO: 857 - DNA, 97 bp, Artificial
  Hairpin sequence Mmd_56, mouse
gaccaaggtctttggtcttggaggaaggtgtgctgctggaggaagccacggaggctgggctggtgggggcatctttcttcagaccacgggctttgtc

SEQ ID NO: 858 - DNA, 97 bp, Artificial
  Hairpin sequence Mmd_57, mouse
tgggaagggcattgcttattttgtatgcttctgggttgattttggaagtgatctgtgacccagaaacgcaaaggacagtgtgggctgcctttcatca

SEQ ID NO: 859 - DNA, 93 bp, Artificial
  Hairpin sequence Mmd_58, mouse
gcaccgaacgaggggttgaccaaagcagccatgatggcaatgtccgagtatgaagaccggttgtcatctggtctggtcagcccagccccgagc

SEQ ID NO: 860 - DNA, 95 bp, Artificial
  Hairpin sequence Mmd_59, mouse
ccgccgcctgggccaggcgcccgccaggtcggcgggaagaggcgcggacacgcgcaccagtcccgccgcgggggcgcgcctggctctgggcgggg

SEQ ID NO: 861 - DNA, 94 bp, Artificial
  Hairpin sequence Mmd_6, mouse
agatttgctttttctcttccatgccttgagtgtaggaccgttgacatcttaattaccctcccacacccaaggcttgcaggagagcaagccttct

SEQ ID NO: 862 - DNA, 83 bp, Artificial
  Hairpin sequence Mmd_60, mouse
gccttggcttcctgttcttcttcatactgttcccgcagcaggtcacagtcatggcgggaggactgaagagcatgagctaaggc

SEQ ID NO: 863 - DNA, 94 bp, Artificial
  Hairpin sequence Mmd_61, mouse
ggggagcaattgtactgactcagcacaagttgcttggctgtgtttacatgtggcttgaagttgcttgagtttcagtatctctgtcctgctctct

SEQ ID NO: 864 - DNA, 97 bp, Artificial
  Hairpin sequence Mmd_62, mouse
ggagtgacaggcgccgaggtgatgatggaggcttctttcctcgaaccctggaaatccattggttgtccatcatcaactccatggtgcttgtgttttt

SEQ ID NO: 865 - DNA, 92 bp, Artificial
  Hairpin sequence Mmd_63, mouse
tggtgtgctgggaaggaggccagtgtggtttctgtcctctgccttttctagcaacttccctccacactgccctctctgcccgggcaacacca

SEQ ID NO: 866 - DNA, 87 bp, Artificial
  Hairpin sequence Mmd_64, mouse
gtatttctatcttggggttctaatgactaaccacttgagctctgcttttgtggtttataatgtcaccagaactctccatggaattat

SEQ ID NO: 867 - DNA, 88 bp, Artificial
  Hairpin sequence Mmd_65, mouse
gtcagttgtttctgggttgtactgggttgtagcaggttgtgtggttttcctccctgtcatgcccagagctctgcccagtgtggcttgc

SEQ ID NO: 868 - DNA, 95 bp, Artificial
  Hairpin sequence Mmd_66, mouse
ctcactttctcagccatttcatgagagtgatgcagggaatgttccctttctgagaacatcctcctttgctcatagctggctagacgacaagtgag

SEQ ID NO: 869 - DNA, 98 bp, Artificial
  Hairpin sequence Mmd_67, mouse
cttcttccagtcatcctgaggtagatatcatacaggaatgctggggccttatggcttacagcaatccagtaatgatataaaaggtgattggaggttag

SEQ ID NO: 870 - DNA, 92 bp, Artificial
  Hairpin sequence Mmd_68, mouse
cgccctgtttaaaggaagaagtttgttctgttgtaaacagtgatgggaactgtttgctttggatgggcttggtccttatgagccaaagagcg

SEQ ID NO: 871 - DNA, 100 bp, Artificial
  Hairpin sequence Mmd_69, mouse
gaggggggagttctctcaggaagaagccaggtgaaactcaaagggtccaaagaaggcttcgctttgagtttctttctggttctgtttgaacctgacactc

SEQ ID NO: 872 - DNA, 85 bp, Artificial
  Hairpin sequence Mmd_7, mouse
gctatgtactgcacaaccctaggagggggtgccattcacatagagtataattgaatggcgccactagggttgtgcagtgtacagc

SEQ ID NO: 873 - DNA, 95 bp, Artificial
  Hairpin sequence Mmd_70, mouse
gattaattctacatgcgcctaattagacccacaattggctaattgcatccacaattagccaattgcaggcccaattggccacttgtcccataatt

SEQ ID NO: 874 - DNA, 94 bp, Artificial
  Hairpin sequence Mmd_71, mouse
cactgtaacaacgttttccttctgactgattacattacatattgctattagtgagcatcagagtgtagtgcaatcagtcaggggaaaatgagtg

SEQ ID NO: 875 - DNA, 97 bp, Artificial
  Hairpin sequence Mmd_72, mouse
ttgcagcttgtgggtcatggcgaggaagaaacaaggagggcatgttcctttcttgtcatgttcttccaagccctgggcatacaaacccactctacag

SEQ ID NO: 876 - DNA, 61 bp, Artificial
  Hairpin sequence Mmd_73, mouse
gggctatggagccgccacatcttgcagcagacactgatggatgaggggctgcggctcgccc

SEQ ID NO: 877 - DNA, 97 bp, Artificial
  Hairpin sequence Mmd_74, mouse
catgccctgatatttgtgttgggataattaccatctctcaggcctcactcggtggcaagatgagatgaaaacccataaaacaccactcagggctgtg

SEQ ID NO: 878 - DNA, 98 bp, Artificial
  Hairpin sequence Mmd_75, mouse
gggaaaaaaaaaggcagacactgtgtttgatgccaataatggttgccagtggcaccgaggttggcatcagaccctctttgtctagctgctgaatgcct

SEQ ID NO: 879 - DNA, 84 bp, Artificial
  Hairpin sequence Mmd_76, mouse
aggcagcaatttgtgtcagatttcaatctatttaagggaaaaccaatgaagtctggttgaaaaatggagagtaaattgctgtct

SEQ ID NO: 880 - DNA, 88 bp, Artificial
  Hairpin sequence Mmd_77, mouse
tctccaaactgttatttaaagcagggtttggcaaggctcagcgagcctgcaccaattttaattgtgtttttaaaactcaggatggaga

SEQ ID NO: 881 - DNA, 99 bp, Artificial
  Hairpin sequence Mmd_78, mouse
ctcttgtccttggagaaatctgggcctgagacagaggagggatgtaatgtgaatgctcctttgtcacaggttaccagggtgtgcttgaggggacggtag

SEQ ID NO: 882 - DNA, 92 bp, Artificial
  Hairpin sequence Mmd_79, mouse
gtgggtactggcctcggtgctggtggagcagtgagcacgccatacattatatctgtgacacctgccacccagcccaaggcccctaggcccac

SEQ ID NO: 883 - DNA, 81 bp, Artificial
  Hairpin sequence Mmd_8, mouse
tgggtgcgtgaggtggttgaccagagagcacacgctatatttgtgccgtttgtgacctggtccactaaccctcagtatcta

SEQ ID NO: 884 - DNA, 90 bp, Artificial
  Hairpin sequence Mmd_80, mouse
ggacagtgtgttgatgtttattggtgtctcttttctcttctagggccagcagagagaagagactccatcttccagaggttgccactgtct

SEQ ID NO: 885 - DNA, 95 bp, Artificial
  Hairpin sequence Mmd_81, mouse
ttcctttaactctcagcttctatttaaaaagctaatttgggagcctgtcgatgctcccaattcagacattttaaataggaactgaaagtcaggaa

SEQ ID NO: 886 - DNA, 82 bp, Artificial
  Hairpin sequence Mmd_82, mouse
ggtccatctttgggccaagatacgtagtcgccaacagtgagttggcgactgtatttttcgagtcctggcgcaaagcaagatc

SEQ ID NO: 887 - DNA, 91 bp, Artificial
  Hairpin sequence Mmd_83, mouse
ggccacactggcagtctggaggcttctcactgtaggagggctgacagcatgggacctcctgtctactagcctgcagtgaccttccatggcc

SEQ ID NO: 888 - DNA, 91 bp, Artificial
  Hairpin sequence Mmd_84, mouse
ggtgtgaaagggtaggagagatgctgggggtgggggtgcttgtgttttagacccccaatattatcccagtgtcccctgccttccttcttcc

SEQ ID NO: 889 - DNA, 99 bp, Artificial
  Hairpin sequence Mmd_85, mouse
caggccactccatctgttcaaacataatgctccaaagcaaggaatatgttccaaaccaaagagcatgatatttgacagatggagatgcccatacgcctg

SEQ ID NO: 890 - DNA, 98 bp, Artificial
  Hairpin sequence Mmd_86, mouse
gtcagaagccaccttctgtgcccccagcaccacgtgtctgggccacgtgagcaacgccacgtgggcctgacgtggagctggggccgcaggggtctgat

SEQ ID NO: 891 - DNA, 99 bp, Artificial
  Hairpin sequence Mmd_87, mouse
ggcagtctgggagtgatgtccctgtgtcattagtgatatgatggcaatcaacagtccaatgtccctcttgatggcactttggacatgaccctagtggct

SEQ ID NO: 892 - DNA, 93 bp, Artificial
  Hairpin sequence Mmd_88, mouse
agcctaacactccgcgggagtttccatggcgaccgaagccgggaccgcgcggcctggcagcgttgccatggagacaactccggagctggggct

SEQ ID NO: 893 - DNA, 93 bp, Artificial
  Hairpin sequence Mmd_89, mouse
gagtctagtctgccaactcctctacagccttctccacgatctctctcacctgggctggaggcccttggagttcttggcagtatctctatactt

SEQ ID NO: 894 - DNA, 95 bp, Artificial
  Hairpin sequence Mmd_9, mouse
gagctttggtacttggagagtggttatccctgtcctcttcgcttcactcatgccgaatcgtacagggtcatccactttttcagtatcaagtgcgc

SEQ ID NO: 895 - DNA, 100 bp, Artificial
  Hairpin sequence Mmd_90, mouse
gggatcaccgcctccatttaggtgtcaattgtgtaatttccagctgttgctcatctcaacagctggagttcaatatgctcagggcgtatctggaggcccc

SEQ ID NO: 896 - DNA, 81 bp, Artificial
  Hairpin sequence Mmd_91, mouse
gcagcaggtagctctgtctgctgtctctttagataaaatgtatgtctggcacggagggaacagctgagaactacctgttgt

SEQ ID NO: 897 - DNA, 92 bp, Artificial
  Hairpin sequence Mmd_92, mouse
ctttgagtgagggtaccatgctgaaggtattcatgatgcggtcaggatattcttctctgatcttgctgatgagcagggtgcccatgccagag

SEQ ID NO: 898 - DNA, 92 bp, Artificial
  Hairpin sequence Mmd_93, mouse
ttgtagttgactaaacagcgtctggtagacgcagatggcatcattaatccgagcgctttctacatctacctgaccgcttgggtcagcaacga

SEQ ID NO: 899 - DNA, 82 bp, Artificial
  Hairpin sequence Mmd_94, mouse
cttctgtagattgtggacaaagccttagaagagccaaagtatagctcgctgtatgctcagctatgtctgcgattggcagaag

SEQ ID NO: 900 - DNA, 99 bp, Artificial
  Hairpin sequence Mmd_95, mouse
gttcgaggagacactatcaaagttaaggcaggggagccagttaacatccctgcagacgtgaccggcctccccatgcctaagattgagtggtccaagaat

SEQ ID NO: 901 - DNA, 98 bp, Artificial
  Hairpin sequence Mmd_96, mouse
ttttcaagtagtcaaattctggctcatggctctggaaggtgctgtaaacattgtattctcctctgctatgagactggattttgttctacaacagaaaa

SEQ ID NO: 902 - DNA, 92 bp, Artificial
  Hairpin sequence Mmd_97, mouse
agctttctccaactgtaacaatctccacttcactgtcagttgaggtaacattaatttcttcattggcagtgacctggggagtgaaggaggct

SEQ ID NO: 903 - DNA, 99 bp, Artificial
  Hairpin sequence Mmd_98, mouse
tcatctttggcccggctccgggtctcggcccgcacggtccggccggccatgctggcggggctggggccgggacagagcccgtggcggggcggcctgtgg

SEQ ID NO: 904 - DNA, 90 bp, Artificial
  Hairpin sequence Mmd_99, mouse
ggcctctgttttgggagaacatgatgtatttattttgttctcatgagaagttcatttcatgagtgcatcatcttctttaaggcaatggcc

SEQ ID NO: 905 - DNA, 82 bp, Artificial
  Hairpin sequence Mmd_from_Hsd_20, mouse
aggtttttactcatgcaccatggttgtctgagcacataacatgcttgtctgctcataccctatggttcctgaagaggaatct

SEQ ID NO: 906 - DNA, 96 bp, Artificial
  Hairpin sequence Mmd_from_Hsd_21, mouse
tgcctctggtgaggactggggaggtggagggtagcatcattagagccagagctctgtctcagctccctctccccccacctcttctctcctcaggta

SEQ ID NO: 907 - DNA, 90 bp, Artificial
  Hairpin sequence Mmd_from_Hsd_39, mouse
cagcttgttatttggtggattgtggcagtaaatcggggcgagtgggggaacccggcgcaggaactgccgctgcggccgggagcggtgctg

SEQ ID NO: 908 - DNA, 85 bp, Artificial
  Hairpin sequence Mmd_from_Hsd_73, mouse
ggccgccgggcgcacccgtcccgttcgtccccggacgttgctctctgccccgggaacgtcgagactggagcgcccgaactgagcc

SEQ ID NO: 909 - DNA, 100 bp, Artificial
  Hairpin sequence Mmd_from_Hsd_75, mouse
agcctgatacccagcacccaggtgagtgcttggcacctggtaagcactcagtaagtatttgatgagtgcctactgtgtgccaagacattgtgctgaggct

SEQ ID NO: 910 - DNA, 97 bp, Artificial
  Hairpin sequence Mmd_from_Hsd_94, mouse
tttcctgctggctgcgggtgctctgactaggttgcactactgtgctgtgagaagcagtgcaatggtattgtcaaagcatctgggaccagcctcgaag

SEQ ID NO: 911 - DNA, 84 bp, Artificial
  Hairpin sequence Mmd_from_Hsd_97, mouse
tgcggcccagggactggtgcggaaagggcccacagtggacttggtacactgtatgccctaaccgctcagtccctgggtctggca

SEQ ID NO: 912 - DNA, 20 bp, Artificial
  Hsd_1, Mmd_1; human brain 25_K23 sequence
ctgttgccactaacctcaac

SEQ ID NO: 913 - DNA, 21 bp, Artificial
  Hsd_1, Mmd_1; human lung 1_J05, mouse brain 49_M17 sequence
tgcggggctagggctaacagc

SEQ ID NO: 914 - DNA, 20 bp, Artificial
  Hsd_1, Mmd_1; mouse brain 13_N23 sequence
tgcggggctagggctaacag

SEQ ID NO: 915 - DNA, 18 bp, Artificial
  Hsd_1, Mmd_1; mouse brain 64_E12np sequence
ggggctagggctaacagc

SEQ ID NO: 916 - DNA, 23 bp, Artificial
  Hsd_10, Mmd_10; human liver 12_H07 sequence
acaagtcttatttgagcacctgt

SEQ ID NO: 917 - DNA, 22 bp, Artificial
  Hsd_10, Mmd_10; mouse brain 104_J08 sequence
aggtcctcaataagtatttgtt

SEQ ID NO: 918 - DNA, 22 bp, Artificial
  Hsd_10, Mmd_10; mouse brain 39_C01 sequence
caaatcttatttgagcacctgt

SEQ ID NO: 919 - DNA, 21 bp, Artificial
  Hsd_100; human brain 18_N21 sequence
actccagccccacagcctcag

SEQ ID NO: 920 - DNA, 19 bp, Artificial
  Hsd_101; human skin 3_L08np sequence
tcctgtgctgtgctgcccc

SEQ ID NO: 921 - DNA, 18 bp, Artificial
  Hsd_102; human heart 5_O18 sequence
atggttcctatcctgctg

SEQ ID NO: 922 - DNA, 18 bp, Artificial
  Hsd_103, Hsd_34, Hsd_36, Hsd_64; human lung 1_N21 sequence
aaaagctgggttgagagg

SEQ ID NO: 923 - DNA, 19 bp, Artificial
  Hsd_103, Hsd_34, Hsd_36; human brain 13_G14 sequence
aaaagctgggttgagaggg

SEQ ID NO: 924 - DNA, 20 bp, Artificial
  Hsd_103, Hsd_34, Hsd_36; human brain 1_E06np sequence
gaaaagctgggttgagaggg

SEQ ID NO: 925 - DNA, 18 bp, Artificial
  Hsd_103, Hsd_34, Hsd_36; human brain 20_E16 sequence
aaagctgggttgagaggg

SEQ ID NO: 926 - DNA, 19 bp, Artificial
  Hsd_103, Hsd_34; human brain 16_E04 sequence
aaagctgggttgagagggc

SEQ ID NO: 927 - DNA, 20 bp, Artificial
  Hsd_103, Hsd_34; human brain 26_J05np sequence
aaaagctgggttgagagggc

SEQ ID NO: 928 - DNA, 21 bp, Artificial
  Hsd_103, Hsd_34; human skin 11_N18np sequence
aaaagctgggttgagagggca

SEQ ID NO: 929 - DNA, 20 bp, Artificial
  Hsd_104; human heart 13_O20np sequence
actggacttggagtcagaaa

SEQ ID NO: 930 - DNA, 25 bp, Artificial
  Hsd_105; human liver 3_A14 sequence
catatctgggctgctgtctcagaat

SEQ ID NO: 931 - DNA, 20 bp, Artificial
  Hsd_106; human lung 14_P16 sequence
taatccttgctacctgggtg

SEQ ID NO: 932 - DNA, 22 bp, Artificial
  Hsd_106; human skin 12_J23 sequence
taatccttgctacctgggtgag

SEQ ID NO: 933 - DNA, 22 bp, Artificial
  Hsd_107; human brain 10_A01np sequence
actgcccaggtgtctgctggtt

SEQ ID NO: 934 - DNA, 22 bp, Artificial
  Hsd_108, Hsd_83; human brain 7_B18np sequence
agaggataccctttgtatgttc

SEQ ID NO: 935 - DNA, 19 bp, Artificial
  Hsd_108, Hsd_83; human lung 16_G04np sequence
ggataccctttgtatgttc

SEQ ID NO: 936 - DNA, 20 bp, Artificial
  Hsd_109, Hsd_56, Hsd_68; human lung 2_D01 sequence
gtaagtgagtcaagagaagg

SEQ ID NO: 937 - DNA, 19 bp, Artificial
  Hsd_11, Mmd_11; human heart 12_F17, mouse brain 11_I07 sequence
gcgacccatacttggtttc

SEQ ID NO: 938 - DNA, 21 bp, Artificial
  Hsd_11, Mmd_11; human lung 5_D01, mouse brain 11_K11 sequence
gcgacccatacttggtttcag

SEQ ID NO: 939 - DNA, 22 bp, Artificial
  Hsd_11, Mmd_11; mouse cand254_probe17 sequence
gcgacccatacttggtttcagt

SEQ ID NO: 940 - DNA, 22 bp, Artificial
  Hsd_110; human skin 11_H21 sequence
tacccattgcatatcggagttg

SEQ ID NO: 941 - DNA, 21 bp, Artificial
  Hsd_110; human skin 13_H14 sequence
tacccattgcatatcggagtt

SEQ ID NO: 942 - DNA, 23 bp, Artificial
  Hsd_110; human skin 1_F09 sequence
tacccattgcatatcggagttgt

SEQ ID NO: 943 - DNA, 18 bp, Artificial
  Hsd_111; human lung 9_P12np sequence
gagtagtgtagtgggtga

SEQ ID NO: 944 - DNA, 20 bp, Artificial
  Hsd_12, Mmd_12; human liver 21_J09 sequence
acgcccttcccccccttctt

SEQ ID NO: 945 - DNA, 20 bp, Artificial
  Hsd_12, Mmd_12; human skin 13_J16np sequence
cgcccttcccccccttcttc

SEQ ID NO: 946 - DNA, 21 bp, Artificial
  Hsd_12, Mmd_12; mouse brain 30_L18 sequence
acgcccttcccccccttcttc

SEQ ID NO: 947 - DNA, 23 bp, Artificial
  Hsd_13, Mmd_13; human heart 13_K23np sequence
aggaagccctggaggggctggag

SEQ ID NO: 948 - DNA, 24 bp, Artificial
  Hsd_13, Mmd_13; human heart 7_H19np, mouse brain 86_F02np sequence
aggaagccctggaggggctggagg

SEQ ID NO: 949 - DNA, 22 bp, Artificial
  Hsd_13, Mmd_13; mouse cand43_probe5 sequence
aggaagccctggaggggctgga

SEQ ID NO: 950 - DNA, 21 bp, Artificial
  Hsd_14, Mmd_14; human lung 1_D04np sequence
ttgtgacagattgataactga

SEQ ID NO: 951 - DNA, 20 bp, Artificial
  Hsd_14, Mmd_14; human skin 12_D07np sequence
tgtgacagattgataactga

SEQ ID NO: 952 - DNA, 19 bp, Artificial
  Hsd_14, Mmd_14; human skin 7_A03 sequence
tgtgacagattgataactg

SEQ ID NO: 953 - DNA, 22 bp, Artificial
  Hsd_14, Mmd_14; mouse cand972_probe5 sequence
tctcggggatcatcatgtcacg

SEQ ID NO: 954 - DNA, 22 bp, Artificial
  Hsd_15, Mmd_15; human brain 11_L21, mouse brain 62_B19 sequence
aaggagcttacaatctagctgg

SEQ ID NO: 955 - DNA, 24 bp, Artificial
  Hsd_15, Mmd_15; human brain 16_I19 sequence
aaggagcttacaatctagctgggg

SEQ ID NO: 956 - DNA, 23 bp, Artificial
  Hsd_15, Mmd_15; human brain 18_J09, mouse brain 101_D04 sequence
aaggagcttacaatctagctggg

SEQ ID NO: 957 - DNA, 22 bp, Artificial
  Hsd_15, Mmd_15; human brain 26_D24np, mouse cand371_probe3 sequence
aggagcttacaatctagctggg

SEQ ID NO: 958 - DNA, 21 bp, Artificial
  Hsd_15, Mmd_15; human brain 26_N24 sequence
aactagactgtgagcttctag

SEQ ID NO: 959 - DNA, 18 bp, Artificial
  Hsd_15, Mmd_15; human lung 16_L10np, mouse brain 52_C24np sequence
aaggagcttacaatctag

SEQ ID NO: 960 - DNA, 20 bp, Artificial
  Hsd_15, Mmd_15; human skin 1_O20np, mouse brain 113_E07 sequence
aaggagcttacaatctagct

SEQ ID NO: 961 - DNA, 19 bp, Artificial
  Hsd_15, Mmd_15; mouse brain 111_A23 sequence
aaggagcttacaatctagc

SEQ ID NO: 962 - DNA, 20 bp, Artificial
  Hsd_15, Mmd_15; mouse brain 13_K21 sequence
caactagactgtgagcttct

SEQ ID NO: 963 - DNA, 22 bp, Artificial
  Hsd_15, Mmd_15; mouse brain 66_J02 sequence
caactagactgtgagcttctag

SEQ ID NO: 964 - DNA, 20 bp, Artificial
  Hsd_15, Mmd_15; mouse brain 77_J13 sequence
actagactgtgagcttctag

SEQ ID NO: 965 - DNA, 21 bp, Artificial
  Hsd_15, Mmd_15; mouse brain 80_K04 sequence
aaggagcttacaatctagctg

SEQ ID NO: 966 - DNA, 21 bp, Artificial
  Hsd_15, Mmd_15; mouse brain 88_H04np sequence
aggagcttacaatctagctgg

SEQ ID NO: 967 - DNA, 23 bp, Artificial
  Hsd_16, Mmd_16; human skin 13_I22, mouse brain 86_C23np sequence
ggagaaattatccttggtgtgtt

SEQ ID NO: 968 - DNA, 22 bp, Artificial
  Hsd_16, Mmd_16; mouse brain 21_N14 sequence
ggagaaattatccttggtgtgt

SEQ ID NO: 969 - DNA, 22 bp, Artificial
  Hsd_16, Mmd_16; mouse cand213_probe4 sequence
aggagaaattatccttggtgtg

SEQ ID NO: 970 - DNA, 21 bp, Artificial
  Hsd_17, Mmd_17; human brain 19_J01, mouse brain 39_C04np sequence
tgtcactcggctcggcccact

SEQ ID NO: 971 - DNA, 24 bp, Artificial
  Hsd_17, Mmd_17; mouse brain 103_B18 sequence
tgtcactcggctcggcccactacc

SEQ ID NO: 972 - DNA, 23 bp, Artificial
  Hsd_17, Mmd_17; mouse brain 87_H14 sequence
tgtcactcggctcggcccactac

SEQ ID NO: 973 - DNA, 22 bp, Artificial
  Hsd_17, Mmd_17; mouse cand216_probe21 sequence
gtcactcggctcggcccactac

SEQ ID NO: 974 - DNA, 22 bp, Artificial
  Hsd_18, Hsd_70, Hsd_82; human brain 14_E16np sequence
tgtcttactccctcaggcacat

SEQ ID NO: 975 - DNA, 18 bp, Artificial
  Hsd_18, Hsd_70, Hsd_82; human brain 29_E04np sequence
tgtcttactccctcaggc

SEQ ID NO: 976 - DNA, 22 bp, Artificial
  Hsd_18, Hsd_70, Hsd_82; human heart 2_F23 sequence
gtcttactccctcaggcacatc

SEQ ID NO: 977 - DNA, 20 bp, Artificial
  Hsd_19; human brain 2_N23 sequence
tatgtctgctgaccatcacc

SEQ ID NO: 978 - DNA, 19 bp, Artificial
  Hsd_19; human lung 15_E21 sequence
tatgtctgctgaccatcac

SEQ ID NO: 979 - DNA, 21 bp, Artificial
  Hsd_19; human skin 4_G20 sequence
tatgtctgctgaccatcacct

SEQ ID NO: 980 - DNA, 20 bp, Artificial
  Hsd_19; human skin 7_A06np sequence
tggtgggccgcagaacatgt

SEQ ID NO: 981 - DNA, 21 bp, Artificial
  Hsd_2, Mmd_2; human lung 2_C01, mouse brain 42_K24 sequence
atataatacaacctgctaagt

SEQ ID NO: 982 - DNA, 22 bp, Artificial
  Hsd_2, Mmd_2; human skin 10_F19, mouse brain 15_N06 sequence
atataatacaacctgctaagtg

SEQ ID NO: 983 - DNA, 22 bp, Artificial
  Hsd_2, Mmd_2; mouse cand350_probe20 sequence
ggttgtattatcattgtccgag

SEQ ID NO: 984 - DNA, 23 bp, Artificial
  Hsd_20, Mmd_from_Hsd_20; human brain 12_B10 sequence
tctgctcataccccatggtttct

SEQ ID NO: 985 - DNA, 20 bp, Artificial
  Hsd_20, Mmd_from_Hsd_20; human brain 8_O15np sequence
accatggttgtctgagcatg

SEQ ID NO: 986 - DNA, 22 bp, Artificial
  Hsd_21, Mmd_from_Hsd_21; human brain 27_B10 sequence
gtgaggactcgggaggtggagg

SEQ ID NO: 987 - DNA, 18 bp, Artificial
  Hsd_22, Hsd_53; human lung 14_O08np sequence
ccaggttcaagtcctggc

SEQ ID NO: 988 - DNA, 22 bp, Artificial
  Hsd_23; human lung 9_B23 sequence
taattttatgtataagctagtc

SEQ ID NO: 989 - DNA, 21 bp, Artificial
  Hsd_24, Hsd_43; human mix 3_E16np sequence
agtggtcggttcctggagctg

SEQ ID NO: 990 - DNA, 22 bp, Artificial
  Hsd_25; human brain 30_J18 sequence
tgtggcttgtccaagaacgttc

SEQ ID NO: 991 - DNA, 18 bp, Artificial
  Hsd_26; human liver 21_N13np sequence
tacactgacggagccagc

SEQ ID NO: 992 - DNA, 19 bp, Artificial
  Hsd_27; human skin 3_J11 sequence
tccctgtcctccaggagct

SEQ ID NO: 993 - DNA, 22 bp, Artificial
  Hsd_28; human liver 14_J22 sequence
tgtctactactggagacactgg

SEQ ID NO: 994 - DNA, 19 bp, Artificial
  Hsd_28; human skin 11_M12np sequence
ctactactggagacactgg

SEQ ID NO: 995 - DNA, 23 bp, Artificial
  Hsd_29; human skin 11_B02 sequence
cgtcccaccccccactcctgttt

SEQ ID NO: 996 - DNA, 21 bp, Artificial
  Hsd_3, Mmd_3; human brain 1_N14np sequence
aatctctgcaggcaaatgtga

SEQ ID NO: 997 - DNA, 21 bp, Artificial
  Hsd_3, Mmd_3; human brain 2_M12, mouse brain 108_B12 sequence
aaatctctgcaggcaaatgtg

SEQ ID NO: 998 - DNA, 22 bp, Artificial
  Hsd_3, Mmd_3; mouse cand234_probe26 sequence
gggaaatctctgcaggcaaatg

SEQ ID NO: 999 - DNA, 18 bp, Artificial
  Hsd_30, Hsd_61; human brain 26_G01np sequence
tcgaggagctcacagtct

SEQ ID NO: 1000 - DNA, 22 bp, Artificial
  Hsd_31; human lung 14_C06np sequence
ccctgggcttaattcccagctc

SEQ ID NO: 1001 - DNA, 24 bp, Artificial
  Hsd_32; human brain 16_L06 sequence
ttgcgggggttagagctgtgggtg

SEQ ID NO: 1002 - DNA, 20 bp, Artificial
  Hsd_33; human brain 19_I17 sequence
aaggagctcacagtctattg

SEQ ID NO: 1003 - DNA, 18 bp, Artificial
  Hsd_33; human heart 9_I07np sequence
aaggagctcacagtctat

SEQ ID NO: 1004 - DNA, 21 bp, Artificial
  Hsd_33; human lung 16_H24 sequence
caaggagctcacagtctattg

SEQ ID NO: 1005 - DNA, 19 bp, Artificial
  Hsd_35; human heart 12_F12np sequence
cgtccgcgccttgcccctg

SEQ ID NO: 1006 - DNA, 22 bp, Artificial
  Hsd_37; human heart 3_P15 sequence
gctaggagttagcttgggagcc

SEQ ID NO: 1007 - DNA, 25 bp, Artificial
  Hsd_38; human brain 30_P22np sequence
ttagggccctggctccatctccttt

SEQ ID NO: 1008 - DNA, 22 bp, Artificial
  Hsd_38; human brain 5_B05 sequence
ttagggccctggctccatctcc

SEQ ID NO: 1009 - DNA, 21 bp, Artificial
  Hsd_38; human lung 3_J17 sequence
ttagggccctggctccatctc

SEQ ID NO: 1010 - DNA, 20 bp, Artificial
  Hsd_39, Mmd_from_Hsd_39; human brain 17_K03 sequence
gaacccggcgcaggaactgc

SEQ ID NO: 1011 - DNA, 22 bp, Artificial
  Hsd_4, Mmd_4; human brain 17_D14np, mouse brain 103_B14 sequence
ttgtgtcaatatgcgatgatgt

SEQ ID NO: 1012 - DNA, 21 bp, Artificial
  Hsd_4, Mmd_4; mouse brain 112_O10 sequence
attgtgtcaatatgcgatgat

SEQ ID NO: 1013 - DNA, 22 bp, Artificial
  Hsd_4, Mmd_4; mouse brain 15_K12 sequence
tcatcacgtggtgacgcaacat

SEQ ID NO: 1014 - DNA, 21 bp, Artificial
  Hsd_40; human heart 2_E14 sequence
ctccgtttgcctgtttcgctg

SEQ ID NO: 1015 - DNA, 18 bp, Artificial
  Hsd_41, Hsd_88; human brain 20_I03np sequence
gccctgggaccctgtctg

SEQ ID NO: 1016 - DNA, 22 bp, Artificial
  Hsd_42; human lung 2_L06np sequence
ttagtggctccctctgcctgca

SEQ ID NO: 1017 - DNA, 22 bp, Artificial
  Hsd_44; human lung 2_C09 sequence
tcaaaactgaggggcattttct

SEQ ID NO: 1018 - DNA, 20 bp, Artificial
  Hsd_44; human skin 5_B02 sequence
tcaaaactgaggggcatttt

SEQ ID NO: 1019 - DNA, 22 bp, Artificial
  Hsd_45; human heart 13_O13 sequence
acgacctcgatgttggatcagg

SEQ ID NO: 1020 - DNA, 18 bp, Artificial
  Hsd_46; human lung 2_D18 sequence
aggttgggagtggaaggt

SEQ ID NO: 1021 - DNA, 19 bp, Artificial
  Hsd_47; human brain 15_G06np sequence
tctttggttatctcagctg

SEQ ID NO: 1022 - DNA, 18 bp, Artificial
  Hsd_48; human lung 9_B11np sequence
tggctcagttcagccggg

SEQ ID NO: 1023 - DNA, 22 bp, Artificial
  Hsd_49; human brain 17_F03 sequence
tccattacactaccctgcctct

SEQ ID NO: 1024 - DNA, 22 bp, Artificial
  Hsd_5, Mmd_5; human skin 4_K16, mouse cand210_ probe14 sequence
aaacattcgcggtgcacttctt

SEQ ID NO: 1025 - DNA, 23 bp, Artificial
  Hsd_5, Mmd_5; human skin 8_E07np, mouse brain 8_O13np sequence
aaacattcgcggtgcacttcttt

SEQ ID NO: 1026 - DNA, 19 bp, Artificial
  Hsd_5, Mmd_5; mouse brain 12_A15np sequence
aaacattcgcggtgcactt

SEQ ID NO: 1027 - DNA, 20 bp, Artificial
  Hsd_5, Mmd_5; mouse brain 16_C05 sequence
aagttgcccgcgtgtttttc

SEQ ID NO: 1028 - DNA, 22 bp, Artificial
  Hsd_5, Mmd_5; mouse brain 20_L19 sequence
gaagttgcccgcgtgtttttcg

SEQ ID NO: 1029 - DNA, 20 bp, Artificial
  Hsd_5, Mmd_5; mouse brain 35_F23np sequence
gaagttgcccgcgtgttttt

SEQ ID NO: 1030 - DNA, 21 bp, Artificial
  Hsd_5, Mmd_5; mouse brain 94_J19 sequence
aaacattcgcggtgcacttct

SEQ ID NO: 1031 - DNA, 21 bp, Artificial
  Hsd_5, Mmd_5; mouse brain 95_O20 sequence
aagttgcccgcgtgtttttcg

SEQ ID NO: 1032 - DNA, 19 bp, Artificial
  Hsd_50; human heart 5_I01np sequence
ccgttccttgtccgtcctt

SEQ ID NO: 1033 - DNA, 18 bp, Artificial
  Hsd_51; human lung 16_D10np sequence
gcctccttggtattccag

SEQ ID NO: 1034 - DNA, 23 bp, Artificial
  Hsd_52; human brain 20_K10 sequence
ccagttaccgcttccgctaccgc

SEQ ID NO: 1035 - DNA, 21 bp, Artificial
  Hsd_54; human heart 8_E13np sequence
aggggcgctctgtctctgttg

SEQ ID NO: 1036 - DNA, 21 bp, Artificial
  Hsd_55; human skin 8_N12 sequence
acccctggggtcctgcccacc

SEQ ID NO: 1037 - DNA, 18 bp, Artificial
  Hsd_57; human lung 7_G22np sequence
aagaagtccttgttactt

SEQ ID NO: 1038 - DNA, 22 bp, Artificial
  Hsd_58; human heart 15_I13np sequence
tctgtgcttcacccctacccag

SEQ ID NO: 1039 - DNA, 20 bp, Artificial
  Hsd_59; human brain 4_K23np sequence
cgttgcccgcatcccccacc

SEQ ID NO: 1040 - DNA, 22 bp, Artificial
  Hsd_6, Mmd_6; human brain 1_K18, mouse cand347_probe29 sequence
catgccttgagtgtaggaccgt

SEQ ID NO: 1041 - DNA, 22 bp, Artificial
  Hsd_6, Mmd_6; human heart 2_P03 sequence
atgccttgagtgtaggaccgtt

SEQ ID NO: 1042 - DNA, 21 bp, Artificial
  Hsd_6, Mmd_6; human lung 10_I24, mouse brain 106_A21 sequence
cctcccacacccaaggcttgc

SEQ ID NO: 1043 - DNA, 23 bp, Artificial
  Hsd_6, Mmd_6; mouse brain 107_C17np sequence
catgccttgagtgtaggaccgtt

SEQ ID NO: 1044 - DNA, 22 bp, Artificial
  Hsd_6, Mmd_6; mouse brain 55_L20np sequence
cctcccacacccaaggcttgca

SEQ ID NO: 1045 - DNA, 24 bp, Artificial
  Hsd_60; human brain 11_C14 sequence
gtgaacgggcgccatcccgaggct

SEQ ID NO: 1046 - DNA, 25 bp, Artificial
  Hsd_60; human heart 10_C06 sequence
gtgaacgggcgccatcccgaggctt

SEQ ID NO: 1047 - DNA, 26 bp, Artificial
  Hsd_60; human heart 13_M22 sequence
gtgaacgggcgccatcccgaggcttt

SEQ ID NO: 1048 - DNA, 22 bp, Artificial
  Hsd_60; human heart 4_B18np sequence
gtgaacgggcgccatcccgagg

SEQ ID NO: 1049 - DNA, 23 bp, Artificial
  Hsd_60; human heart 6_A01 sequence
tgaacgggcgccatcccgaggct

SEQ ID NO: 1050 - DNA, 21 bp, Artificial
  Hsd_61; human brain 14_G18 sequence
tcgaggagctcacagtctagt

SEQ ID NO: 1051 - DNA, 18 bp, Artificial
  Hsd_62; human lung 14_M21np sequence
gggttcaaaccccacctc

SEQ ID NO: 1052 - DNA, 19 bp, Artificial
  Hsd_63; human brain 26_P14np sequence
cccacgcctcctcccctcc

SEQ ID NO: 1053 - DNA, 18 bp, Artificial
  Hsd_65; human heart 13_B21np sequence
gagctgggatcactcccg

SEQ ID NO: 1054 - DNA, 20 bp, Artificial
  Hsd_66; human brain 30_N24np sequence
gcaatgttcgggggaggcag

SEQ ID NO: 1055 - DNA, 25 bp, Artificial
  Hsd_67; human lung 11_L11 sequence
aggcagtgtattgttagctggctgc

SEQ ID NO: 1056 - DNA, 27 bp, Artificial
  Hsd_67; human lung 12_A04np sequence
aggcagtgtattgttagctggctgctt

SEQ ID NO: 1057 - DNA, 24 bp, Artificial
  Hsd_67; human lung 5_M21np sequence
aggcagtgtattgttagctggctg

SEQ ID NO: 1058 - DNA, 18 bp, Artificial
  Hsd_69; human liver 19_P17np sequence
gtgaaggcccggcggaga

SEQ ID NO: 1059 - DNA, 21 bp, Artificial
  Hsd_7, Mmd_7; human heart 3_E21, mouse brain 106_F19 sequence
aatggcgccactagggttgtg

SEQ ID NO: 1060 - DNA, 20 bp, Artificial
  Hsd_7, Mmd_7; mouse brain 95_D10 sequence
aatggcgccactagggttgt

SEQ ID NO: 1061 - DNA, 22 bp, Artificial
  Hsd_7, Mmd_7; mouse cand355_probe41 sequence
gaatggcgccactagggttgtg

SEQ ID NO: 1062 - DNA, 20 bp, Artificial
  Hsd_71; human brain 20_F05np sequence
cgcccctcctgcccccacag

SEQ ID NO: 1063 - DNA, 22 bp, Artificial
  Hsd_72; human heart 3_N22np sequence
ctgctgccttgtgggtgggggg

SEQ ID NO: 1064 - DNA, 21 bp, Artificial
  Hsd_73, Mmd_from_Hsd_73; human brain 18_D19 sequence
acccgtcccgttcgtccccgg

SEQ ID NO: 1065 - DNA, 19 bp, Artificial
  Hsd_74; human heart 10_B02 sequence
attgcctctgttctaacac

SEQ ID NO: 1066 - DNA, 21 bp, Artificial
  Hsd_75, Mmd_from_Hsd_75; human brain 28_N20 sequence
cttggcacctagcaagcactc

SEQ ID NO: 1067 - DNA, 24 bp, Artificial
  Hsd_76; human brain 6_J16np sequence
ttgcagctgcctgggagtgacttc

SEQ ID NO: 1068 - DNA, 23 bp, Artificial
  Hsd_77; human skin 4_C19 sequence
ctctgctcctcctgttcgacagt

SEQ ID NO: 1069 - DNA, 18 bp, Artificial
  Hsd_78; human heart 11_J17np sequence
agaggcgcacgggggtcg

SEQ ID NO: 1070 - DNA, 20 bp, Artificial
  Hsd_79; human lung 15_E03np sequence
gcaggcgtctgcggaactgg

SEQ ID NO: 1071 - DNA, 21 bp, Artificial
  Hsd_8, Mmd_8; human brain 20_O07 sequence
tttgtgacctggtccactaac

SEQ ID NO: 1072 - DNA, 18 bp, Artificial
  Hsd_8, Mmd_8; human brain 2_A23 sequence
tttgtgacctggtccact

SEQ ID NO: 1073 - DNA, 19 bp, Artificial
  Hsd_8, Mmd_8; mouse brain 98_F03np sequence
tttgtgacctggtccacta

SEQ ID NO: 1074 - DNA, 23 bp, Artificial
  Hsd_80; human lung 2_B06np sequence
tgtcactcttgtgtgttgcagat

SEQ ID NO: 1075 - DNA, 22 bp, Artificial
  Hsd_81; human brain 25_H01 sequence
accctatcaatattgtctctgc

SEQ ID NO: 1076 - DNA, 22 bp, Artificial
  Hsd_81; human lung 12_J23np sequence
tagtgcaatattgcttataggg

SEQ ID NO: 1077 - DNA, 23 bp, Artificial
  Hsd_81; human skin 12_D12 sequence
tagtgcaatattgcttatagggt

SEQ ID NO: 1078 - DNA, 25 bp, Artificial
  Hsd_81; human skin 12_N11 sequence
tagtgcaatattgcttatagggttt

SEQ ID NO: 1079 - DNA, 22 bp, Artificial
  Hsd_84; human heart 13_E01np sequence
ttatggtttgcctgggactgag

SEQ ID NO: 1080 - DNA, 19 bp, Artificial
  Hsd_85; human mix 4_M20np sequence
ggtggtcaaaggagcccca

SEQ ID NO: 1081 - DNA, 23 bp, Artificial
  Hsd_86; human lung 10_F10np sequence
aggcagtgtattgctagcggctg

SEQ ID NO: 1082 - DNA, 25 bp, Artificial
  Hsd_86; human lung 11_O08 sequence
aggcagtgtattgctagcggctgtt

SEQ ID NO: 1083 - DNA, 24 bp, Artificial
  Hsd_86; human lung 13_P02 sequence
aggcagtgtattgctagcggctgt

SEQ ID NO: 1084 - DNA, 18 bp, Artificial
  Hsd_87; human lung 6_D19np sequence
gggccccctacagggctc

SEQ ID NO: 1085 - DNA, 22 bp, Artificial
  Hsd_89; human lung 7_O04np sequence
ttctctgttttggccatgtgtg

SEQ ID NO: 1086 - DNA, 21 bp, Artificial
  Hsd_9, Mmd_9; human brain 10_O20, mouse brain 18_B04 sequence
aatcgtacagggtcatccact

SEQ ID NO: 1087 - DNA, 21 bp, Artificial
  Hsd_9, Mmd_9; human brain 7_A24 sequence
tcgtacagggtcatccacttt

SEQ ID NO: 1088 - DNA, 20 bp, Artificial
  Hsd_9, Mmd_9; human skin 2_A11np sequence
aatcgtacagggtcatccac

SEQ ID NO: 1089 - DNA, 23 bp, Artificial
  Hsd_9, Mmd_9; mouse brain 100_A13np sequence
aatcgtacagggtcatccacttt

SEQ ID NO: 1090 - DNA, 20 bp, Artificial
  Hsd_9, Mmd_9; mouse brain 18_F01np sequence
atcgtacagggtcatccact

SEQ ID NO: 1091 - DNA, 19 bp, Artificial
  Hsd_9, Mmd_9; mouse brain 44_G16np sequence
aatcgtacagggtcatcca

SEQ ID NO: 1092 - DNA, 23 bp, Artificial
  Hsd_9, Mmd_9; mouse brain 64_L05np sequence
tcgtacagggtcatccacttttt

SEQ ID NO: 1093 - DNA, 22 bp, Artificial
  Hsd_9, Mmd_9; mouse cand212_probe16 sequence
aatcgtacagggtcatccactt

SEQ ID NO: 1094 - DNA, 20 bp, Artificial
  Hsd_90; human heart 4_P23np sequence
ccagggggagaaggtgtaag

SEQ ID NO: 1095 - DNA, 18 bp, Artificial
  Hsd_91; human heart 6_G01np sequence
agttttttgggaatgcag

SEQ ID NO: 1096 - DNA, 18 bp, Artificial
  Hsd_92; human heart 2_M05np sequence
gcgcgcgggctcttgggt

SEQ ID NO: 1097 - DNA, 20 bp, Artificial
  Hsd_93; human heart 4_B07np sequence
gtcagtgacgaagccagacc

SEQ ID NO: 1098 - DNA, 23 bp, Artificial
  Hsd_94, Mmd_from_Hsd_94; human lung 16_G20 sequence
cagtgcaatgatattgtcaaagc

SEQ ID NO: 1099 - DNA, 21 bp, Artificial
  Hsd_95; human heart 4_M23 sequence
ataagacgaacaaaaggtttg

SEQ ID NO: 1100 - DNA, 18 bp, Artificial
  Hsd_96; human skin 5_M20np sequence
gaaagcgcttcccttttg

SEQ ID NO: 1101 - DNA, 22 bp, Artificial
  Hsd_97, Mmd_from_Hsd_97; human heart 10_G17 sequence
ctgtatgccctcaccgctcagc

SEQ ID NO: 1102 - DNA, 22 bp, Artificial
  Hsd_97, Mmd_from_Hsd_97; human heart 2_F09 sequence
tggtgcggagagggcccacagt

SEQ ID NO: 1103 - DNA, 21 bp, Artificial
  Hsd_97, Mmd_from_Hsd_97; human skin 8_O20np sequence
ctgtatgccctcaccgctcag

SEQ ID NO: 1104 - DNA, 23 bp, Artificial
  Hsd_98; human brain 18_L10 sequence
ctgggatctccggggtcttggtt

SEQ ID NO: 1105 - DNA, 22 bp, Artificial
  Hsd_98; human brain 3_A01 sequence
tgagacctctgggttctgagct

SEQ ID NO: 1106 - DNA, 22 bp, Artificial
  Hsd_98; human brain 3_C09 sequence
tgggatctccggggtcttggtt

SEQ ID NO: 1107 - DNA, 21 bp, Artificial
  Hsd_98; human heart 14_G20 sequence
tgagacctctgggttctgagc

SEQ ID NO: 1108 - DNA, 21 bp, Artificial
  Hsd_98; human skin 3_M18np sequence
ctgagacctctgggttctgag

SEQ ID NO: 1109 - DNA, 22 bp, Artificial
  Hsd_99; human brain 18_M20np sequence
tttcctctctgccccatagggt

SEQ ID NO: 1110 - DNA, 22 bp, Artificial
  Hsd_from_Mmd_100, Mmd_100; mouse cand424_probe2 sequence
agggagggcaagtcaggtgacc

SEQ ID NO: 1111 - DNA, 22 bp, Artificial
  Hsd_from_Mmd_101, Mmd_101; mouse cand659_probe2 sequence
ttttgtctccaagcaaaggtca

SEQ ID NO: 1112 - DNA, 22 bp, Artificial
  Hsd_from_Mmd_105, Mmd_105; mouse cand310_probe9 sequence
aaggaaaacatctttgaagttc

SEQ ID NO: 1113 - DNA, 22 bp, Artificial
  Hsd_from_Mmd_107, Mmd_107; mouse cand906_probe13 sequence
gagttatgtgctaaattcatat

SEQ ID NO: 1114 - DNA, 22 bp, Artificial
  Hsd_from_Mmd_112, Hsd_from_Mmd_470, Mmd_112, Mmd_470; mouse cand513_probe6 sequence
gcccggagtttgatgtgcaggg

SEQ ID NO: 1115 - DNA, 22 bp, Artificial
  Hsd_from_Mmd_115, Mmd_115; mouse cand456_probe40 sequence
gacaaggcttgttgtgcccgag

SEQ ID NO: 1116 - DNA, 22 bp, Artificial
  Hsd_from_Mmd_117, Mmd_117; mouse cand100_probe35 sequence
ccggagtttggagatgccattg

SEQ ID NO: 1117 - DNA, 22 bp, Artificial
  Hsd_from_Mmd_119, Mmd_119; mouse cand469_probe10 sequence
tgccaagagacattttgcatat

SEQ ID NO: 1118 - DNA, 22 bp, Artificial
  Hsd_from_Mmd_120, Mmd_120; mouse cand578_probe13 sequence
gtagaggggcggggcatgaaga

SEQ ID NO: 1119 - DNA, 22 bp, Artificial
  Hsd_from_Mmd_123, Mmd_123; mouse cand447_probe19 sequence
ggccattaacctcagttggtca

SEQ ID NO: 1120 - DNA, 22 bp, Artificial
  Hsd_from_Mmd_126, Mmd_126; mouse cand496_probe28 sequence
tctctaaatgccccacgatgac

SEQ ID NO: 1121 - DNA, 22 bp, Artificial
  Hsd_from_Mmd_126, Mmd_126; mouse cand496_probe36 sequence
gtcctctgagggcgatttgaat

SEQ ID NO: 1122 - DNA, 22 bp, Artificial
  Hsd_from_Mmd_128, Mmd_128; mouse cand556_probe6 sequence
ggtgcccaggctccaccctgga

SEQ ID NO: 1123 - DNA, 22 bp, Artificial
  Hsd_from_Mmd_129, Mmd_129; mouse cand617_probe41 sequence
aggagatgctaggggacagtga

SEQ ID NO: 1124 - DNA, 22 bp, Artificial
  Hsd_from_Mmd_133, Mmd_133; mouse cand50_probe34 sequence
acaaggatgaatctttgttact

SEQ ID NO: 1125 - DNA, 22 bp, Artificial
  Hsd_from_Mmd_136, Mmd_136; mouse cand605_probe7 sequence
ttggtttgttagttaatggccc

SEQ ID NO: 1126 - DNA, 22 bp, Artificial
  Hsd_from_Mmd_137, Mmd_137; mouse mmHP 3254020_probe13 sequence
ggtcaacaaagactttggctga

SEQ ID NO: 1127 - DNA, 22 bp, Artificial
  Hsd_from_Mmd_138, Mmd_138; mouse cand139_probe2 sequence
ctgaggcctagtaattttcaat

SEQ ID NO: 1128 - DNA, 22 bp, Artificial
  Hsd_from_Mmd_141, Mmd_141; mouse cand620_probe22 sequence
aaaatacatctgcctcttctta

SEQ ID NO: 1129 - DNA, 22 bp, Artificial
  Hsd_from_Mmd_145, Mmd_145; mouse cand534_probe28 sequence
aagggtctaatggtcagcagag

SEQ ID NO: 1130 - DNA, 22 bp, Artificial
  Hsd_from_Mmd_147, Mmd_147; mouse cand356_probe16 sequence
ggctgcttggagggctctctct

SEQ ID NO: 1131 - DNA, 22 bp, Artificial
  Hsd_from_Mmd_148, Mmd_148; mouse cand582_probe38 sequence
aaggccattagtccaacaggtc

SEQ ID NO: 1132 - DNA, 22 bp, Artificial
  Hsd_from_Mmd_149, Mmd_149; mouse cand236_probe19 sequence
agaactctaggttcctggagtg

SEQ ID NO: 1133 - DNA, 22 bp, Artificial
  Hsd_from_Mmd_150, Mmd_150; mouse cand267_probe12 sequence
tgcaatttatgtgcaagtggtt

SEQ ID NO: 1134 - DNA, 22 bp, Artificial
  Hsd_from_Mmd_154, Mmd_154; mouse cand914_probe16 sequence
gcggccacagacacgacatcta

SEQ ID NO: 1135 - DNA, 22 bp, Artificial
  Hsd_from_Mmd_155, Mmd_155; mouse cand674_probe14 sequence
cggaggaggggctggcgaaaat

SEQ ID NO: 1136 - DNA, 22 bp, Artificial
  Hsd_from_Mmd_156, Mmd_156; mouse cand907_probe14 sequence
tcctgtgtttgctggagtaagg

SEQ ID NO: 1137 - DNA, 22 bp, Artificial
  Hsd_from_Mmd_157, Mmd_157; mouse cand670_probe9 sequence
gcaggatgagggttgacctata

SEQ ID NO: 1138 - DNA, 22 bp, Artificial
  Hsd_from_Mmd_159, Mmd_159; mouse cand625_probe19 sequence
ctgcagaagacaatttcctgtc

SEQ ID NO: 1139 - DNA, 22 bp, Artificial
  Hsd_from_Mmd_159, Mmd_159; mouse cand625_probe3 sequence
gcaggaaatggcttctaattag

SEQ ID NO: 1140 - DNA, 22 bp, Artificial
  Hsd_from_Mmd_161, Mmd_161; mouse brain 51_F13np sequence
gcggtgatgccgatggtgcgag

SEQ ID NO: 1141 - DNA, 20 bp, Artificial
  Hsd_from_Mmd_161, Mmd_161; mouse brain 83_H16 sequence
gcggtgatgccgatggtgcg

SEQ ID NO: 1142 - DNA, 22 bp, Artificial
  Hsd_from_Mmd_162, Mmd_162; mouse cand339_probe12 sequence
cagtgttcagtagccctgaaaa

SEQ ID NO: 1143 - DNA, 22 bp, Artificial
  Hsd_from_Mmd_163, Mmd_163; mouse cand378_probe17 sequence
ctaagtggtagtcagaaacgac

SEQ ID NO: 1144 - DNA, 22 bp, Artificial
  Hsd_from_Mmd_163, Mmd_163; mouse cand378_probe7 sequence
gtttccaagacattccgctagg

SEQ ID NO: 1145 - DNA, 22 bp, Artificial
  Hsd_from_Mmd_166, Mmd_166; mouse cand301_probe27 sequence
gctcatgacttatgaagtgtta

SEQ ID NO: 1146 - DNA, 22 bp, Artificial
  Hsd_from_Mmd_167, Mmd_167; mouse cand711_probe18 sequence
cgtggtcgtcgcgggcgtgggg

SEQ ID NO: 1147 - DNA, 22 bp, Artificial
  Hsd_from_Mmd_169, Mmd_169; mouse cand636_probe14 sequence
cagggtgactgacctgaagggt

SEQ ID NO: 1148 - DNA, 22 bp, Artificial
  Hsd_from_Mmd_170, Mmd_170; mouse cand51_probe4 sequence
tgcaaatgtttgcctcaagcaa

SEQ ID NO: 1149 - DNA, 22 bp, Artificial
  Hsd_from_Mmd_172, Hsd_from_Mmd_96, Mmd_172, Mmd_469, Mmd_96; mouse cand650_probe9 sequence
gctcatggctctggaaggtgct

SEQ ID NO: 1150 - DNA, 22 bp, Artificial
  Hsd_from_Mmd_173, Mmd_173; mouse cand687_probe19 sequence
ctgagggtgatttcaaaatcca

SEQ ID NO: 1151 - DNA, 22 bp, Artificial
  Hsd_from_Mmd_174, Mmd_174; mouse mmHP2021933_probe2 sequence
agctttaaagtttagtcactct

SEQ ID NO: 1152 - DNA, 22 bp, Artificial
  Hsd_from_Mmd_176, Mmd_176; mouse cand651_probe26 sequence
agacaattgtgaagggattgtg

SEQ ID NO: 1153 - DNA, 22 bp, Artificial
  Hsd_from_Mmd_178, Mmd_178; mouse cand102_probe21 sequence
aggattttgaaggaagcataat

SEQ ID NO: 1154 - DNA, 22 bp, Artificial
  Hsd_from_Mmd_180, Mmd_180; mouse cand859_probe42 sequence
ggccaggaatcagctgcgccgc

SEQ ID NO: 1155 - DNA, 22 bp, Artificial
  Hsd_from_Mmd_181, Hsd_from_Mmd_276, Mmd_181, Mmd_276; mouse cand723a_probe8 sequence
cgagaccgagatctggatgcag

SEQ ID NO: 1156 - DNA, 22 bp, Artificial
  Hsd_from_Mmd_181, Mmd_181; mouse cand723_probe14 sequence
cgagacccagacttggatttgt

SEQ ID NO: 1157 - DNA, 20 bp, Artificial
  Hsd_from_Mmd_182, Mmd_182; mouse brain 67_M09np sequence
atccctgagtgtatgtggtg

SEQ ID NO: 1158 - DNA, 22 bp, Artificial
  Hsd_from_Mmd_182, Mmd_182; mouse cand520_probe21 sequence
catatccattcaggagtgtcag

SEQ ID NO: 1159 - DNA, 22 bp, Artificial
  Hsd_from_Mmd_183, Mmd_183; mouse cand633_probe11 sequence
gaatacaggaaataagcttagg

SEQ ID NO: 1160 - DNA, 22 bp, Artificial
  Hsd_from_Mmd_184, Mmd_184; mouse cand405_probe12 sequence
gttatttctggaaatgtgtgct

SEQ ID NO: 1161 - DNA, 22 bp, Artificial
  Hsd_from_Mmd_185, Mmd_185; mouse cand439_probe12 sequence
gggactaatctgtctgtgggga

SEQ ID NO: 1162 - DNA, 22 bp, Artificial
  Hsd_from_Mmd_186, Mmd_186; mouse cand944_probe6 sequence
cgaggctacttttgattgggat

SEQ ID NO: 1163 - DNA, 21 bp, Artificial
  Hsd_from_Mmd_187, Mmd_187; mouse brain 52_F18 sequence
catctgggcaactgattgaac

SEQ ID NO: 1164 - DNA, 22 bp, Artificial
  Hsd_from_Mmd_189, Mmd_189; mouse cand579_probe3 sequence
gtttgacaggcacagggacaaa

SEQ ID NO: 1165 - DNA, 22 bp, Artificial
  Hsd_from_Mmd_19, Mmd_19; mouse cand623_probe27 sequence
gccggcagcagccgccgaacag

SEQ ID NO: 1166 - DNA, 22 bp, Artificial
  Hsd_from_Mmd_190, Mmd_190; mouse cand147_probe17 sequence
gtctgagctcagggcctggccc

SEQ ID NO: 1167 - DNA, 22 bp, Artificial
  Hsd_from_Mmd_193, Mmd_193; mouse cand372_probe7 sequence
gcagcagggtgaaactgacaca

SEQ ID NO: 1168 - DNA, 22 bp, Artificial
  Hsd_from_Mmd_195, Mmd_195; mouse cand450_probe11 sequence
ttaaggaagacctgtatagctc

SEQ ID NO: 1169 - DNA, 22 bp, Artificial
  Hsd_from_Mmd_196, Mmd_196; mouse cand743_probe27 sequence
gctgacagcattttcactatta

SEQ ID NO: 1170 - DNA, 22 bp, Artificial
  Hsd_from_Mmd_197, Mmd_197; mouse cand127_probe18 sequence
aggaggccatagtggcaactgt

SEQ ID NO: 1171 - DNA, 22 bp, Artificial
  Hsd_from_Mmd_197, Mmd_197; mouse cand127_probe3 sequence
gcaggtgctcacatgtcctcct

SEQ ID NO: 1172 - DNA, 22 bp, Artificial
  Hsd_from_Mmd_198, Mmd_198; mouse cand595_probe11 sequence
agggggagaggaggacattttg

SEQ ID NO: 1173 - DNA, 22 bp, Artificial
  Hsd_from_Mmd_20, Mmd_20; mouse cand601_probe3 sequence
caggtgctcatgtgggagagat

SEQ ID NO: 1174 - DNA, 22 bp, Artificial
  Hsd_from_Mmd_203, Mmd_203; mouse cand394_probe43 sequence
acattgtaaattcaaaataatt

SEQ ID NO: 1175 - DNA, 22 bp, Artificial
  Hsd_from_Mmd_204, Mmd_204; mouse cand480_probe13 sequence
ggccagcgtctgatgtcagcct

SEQ ID NO: 1176 - DNA, 22 bp, Artificial
  Hsd_from_Mmd_208, Mmd_208; mouse cand640_probe13 sequence
cagtgcactgatataagattat

SEQ ID NO: 1177 - DNA, 22 bp, Artificial
  Hsd_from_Mmd_211, Mmd_211; mouse cand881_probe38 sequence
taaggagcagcttcgccaaaaa

SEQ ID NO: 1178 - DNA, 20 bp, Artificial
  Hsd_from_Mmd_212, Mmd_212; mouse brain 43_A14 sequence
gtagaggagatggcgcaggg

SEQ ID NO: 1179 - DNA, 22 bp, Artificial
  Hsd_from_Mmd_215, Mmd_215; mouse cand225_probe19 sequence
gtttgggtctgctctctgaccc

SEQ ID NO: 1180 - DNA, 22 bp, Artificial
  Hsd_from_Mmd_215, Mmd_215; mouse cand225_probe2 sequence
gggttaagggggcgggaccagt

SEQ ID NO: 1181 - DNA, 22 bp, Artificial
  Hsd_from_Mmd_217, Mmd_217; mouse cand448_probe8 sequence
agggtttctagtgactccagct

SEQ ID NO: 1182 - DNA, 22 bp, Artificial
  Hsd_from_Mmd_219, Mmd_219; mouse cand478_probe21 sequence
ccacttttggagggagacttgg

SEQ ID NO: 1183 - DNA, 22 bp, Artificial
  Hsd_from_Mmd_222, Mmd_222; mouse cand156_probe4 sequence
accagccctgatggtgtgtccg

SEQ ID NO: 1184 - DNA, 22 bp, Artificial
  Hsd_from_Mmd_223, Mmd_223; mouse cand642_probe13 sequence
agggtttatgaagttatcaaag

SEQ ID NO: 1185 - DNA, 22 bp, Artificial
  Hsd_from_Mmd_225, Mmd_225; mouse cand458_probe12 sequence
gcattccctatgggaaaggtga

SEQ ID NO: 1186 - DNA, 22 bp, Artificial
  Hsd_from_Mmd_226, Mmd_226; mouse cand320_probe1 sequence
catttttctcagttagtgtcag

SEQ ID NO: 1187 - DNA, 22 bp, Artificial
  Hsd_from_Mmd_227, Mmd_227; mouse cand869_probe21 sequence
aggaatacggaaccatgcctct

SEQ ID NO: 1188 - DNA, 22 bp, Artificial
  Hsd_from_Mmd_227, Mmd_227; mouse cand869_probe3 sequence
ggcactttgtacttctgccagg

SEQ ID NO: 1189 - DNA, 22 bp, Artificial
  Hsd_from_Mmd_228, Mmd_228; mouse cand541_probe22 sequence
cgaggctcggagcagcccaggg

SEQ ID NO: 1190 - DNA, 22 bp, Artificial
  Hsd_from_Mmd_229, Mmd_229; mouse cand281_probe12 sequence
agagacctttctttaaaatctt

SEQ ID NO: 1191 - DNA, 22 bp, Artificial
  Hsd_from_Mmd_23, Mmd_23; mouse brain 69_B01np sequence
ttgaaacaatctctactgaacc

SEQ ID NO: 1192 - DNA, 22 bp, Artificial
  Hsd_from_Mmd_23, Mmd_23; mouse cand644_probe15 sequence
gttcactggagtttgtttcagt

SEQ ID NO: 1193 - DNA, 22 bp, Artificial
  Hsd_from_Mmd_231, Mmd_231; mouse cand589_probe13 sequence
aggacatcgtttacagctgcat

SEQ ID NO: 1194 - DNA, 22 bp, Artificial
  Hsd_from_Mmd_232, Mmd_232; mouse cand549_probe9 sequence
tattttcagtcactcaaactga

SEQ ID NO: 1195 - DNA, 22 bp, Artificial
  Hsd_from_Mmd_235, Mmd_235; mouse cand466_probe13 sequence
gcaggtccaggctgggcttgta

SEQ ID NO: 1196 - DNA, 22 bp, Artificial
  Hsd_from_Mmd_236, Mmd_236; mouse cand113_probe16 sequence
agccatcagcttttcccagctg

SEQ ID NO: 1197 - DNA, 22 bp, Artificial
  Hsd_from_Mmd_236, Mmd_236; mouse cand113_probe9 sequence
ccgaagctaatagcctttggct

SEQ ID NO: 1198 - DNA, 22 bp, Artificial
  Hsd_from_Mmd_237, Mmd_237; mouse cand515_probe14 sequence
agggaccgccgacgctgctacc

SEQ ID NO: 1199 - DNA, 22 bp, Artificial
  Hsd_from_Mmd_237, Mmd_237; mouse cand733_probe35 sequence
gagggaccgccgacgctgctac

SEQ ID NO: 1200 - DNA, 22 bp, Artificial
  Hsd_from_Mmd_243, Mmd_243; mouse cand129_probe40 sequence
gggtacatctggacagccgaat

SEQ ID NO: 1201 - DNA, 22 bp, Artificial
  Hsd_from_Mmd_245, Mmd_245; mouse cand87_probe1 sequence
gggattaccccctgctgctgac

SEQ ID NO: 1202 - DNA, 22 bp, Artificial
  Hsd_from_Mmd_246, Hsd_from_Mmd_266, Hsd_from_Mmd_42, Mmd_246, Mmd_266, Mmd_42; mouse cand410_probe35 sequence
agggaagaacataagaagaaaa

SEQ ID NO: 1203 - DNA, 22 bp, Artificial
  Hsd_from_Mmd_246, Hsd_from_Mmd_266, Hsd_from_Mmd_42, Mmd_246, Mmd_266, Mmd_42; mouse cand598_probe34 sequence
cagggaagaacataagaagaaa

SEQ ID NO: 1204 - DNA, 22 bp, Artificial
  Hsd_from_Mmd_247, Mmd_247; mouse cand309_probe17 sequence
aattaggctttttaattaaaag

SEQ ID NO: 1205 - DNA, 22 bp, Artificial
  Hsd_from_Mmd_249, Mmd_249; mouse cand816_probe3 sequence
tgccatggcacaaaccatgctg

SEQ ID NO: 1206 - DNA, 22 bp, Artificial
  Hsd_from_Mmd_250, Mmd_250; mouse cand82_probe8 sequence
tctattgggtaaatatgtatta

SEQ ID NO: 1207 - DNA, 22 bp, Artificial
  Hsd_from_Mmd_253, Hsd_from_Mmd_319, Mmd_253, Mmd_319; mouse cand48_probe12 sequence
tcttccaaccaaagctgggaag

SEQ ID NO: 1208 - DNA, 22 bp, Artificial
  Hsd_from_Mmd_255, Mmd_255; mouse cand538_probe14 sequence
gggtcaaaggttgctatcaccc

SEQ ID NO: 1209 - DNA, 22 bp, Artificial
  Hsd_from_Mmd_256, Mmd_256; mouse cand960_probe1 sequence
aggccccgaggagggttgtggg

SEQ ID NO: 1210 - DNA, 22 bp, Artificial
  Hsd_from_Mmd_256, Mmd_256; mouse cand960_probe22 sequence
ggaacatccctcctgggggtcc

SEQ ID NO: 1211 - DNA, 22 bp, Artificial
  Hsd_from_Mmd_258, Mmd_258; mouse cand713_probe11 sequence
aggacgaagatgacgaggagct

SEQ ID NO: 1212 - DNA, 22 bp, Artificial
  Hsd_from_Mmd_259, Mmd_259; mouse cand376_probe15 sequence
agctgctgcctccagaagtgtc

SEQ ID NO: 1213 - DNA, 22 bp, Artificial
  Hsd_from_Mmd_26, Mmd_26; mouse cand65_probe6 sequence
caaagatgaggatttctacctt

SEQ ID NO: 1214 - DNA, 22 bp, Artificial
  Hsd_from_Mmd_260, Mmd_260; mouse cand468_probe33 sequence
gcagggagcctggctcattagg

SEQ ID NO: 1215 - DNA, 22 bp, Artificial
  Hsd_from_Mmd_260, Mmd_260; mouse cand784a_probe32 sequence
ggcagggagcctggctcattag

SEQ ID NO: 1216 - DNA, 22 bp, Artificial
  Hsd_from_Mmd_261, Mmd_261; mouse cand873_probe8 sequence
aggtaggcgtacaactaggaga

SEQ ID NO: 1217 - DNA, 22 bp, Artificial
  Hsd_from_Mmd_262, Mmd_262; mouse cand360_probe7 sequence
gggtacaattagcctctggcgc

SEQ ID NO: 1218 - DNA, 22 bp, Artificial
  Hsd_from_Mmd_263, Mmd_263; mouse cand568_probe21 sequence
aggagagatctgtaggcctttc

SEQ ID NO: 1219 - DNA, 22 bp, Artificial
  Hsd_from_Mmd_264, Mmd_264; mouse cand393_probe14 sequence
tattggtcaatttcaccttgat

SEQ ID NO: 1220 - DNA, 22 bp, Artificial
  Hsd_from_Mmd_270, Mmd_270; mouse cand126_probe18 sequence
aggagcagacactgcccaggtt

SEQ ID NO: 1221 - DNA, 22 bp, Artificial
  Hsd_from_Mmd_271, Mmd_271; mouse cand377_probe20 sequence
gtcaccatggaaacaagcagag

SEQ ID NO: 1222 - DNA, 22 bp, Artificial
  Hsd_from_Mmd_272, Mmd_272; mouse cand19_probe18 sequence
gccagatgggcaggattaatta

SEQ ID NO: 1223 - DNA, 22 bp, Artificial
  Hsd_from_Mmd_274, Mmd_274; mouse cand275_probe10 sequence
ctgtgggtaaggataagcatgc

SEQ ID NO: 1224 - DNA, 22 bp, Artificial
  Hsd_from_Mmd_275, Hsd_from_Mmd_389, Mmd_275, Mmd_389; mouse cand927d_probe16 sequence
gtgtttggcctcctctgggacg

SEQ ID NO: 1225 - DNA, 22 bp, Artificial
  Hsd_from_Mmd_276, Mmd_276; mouse cand723a_probe15 sequence
cgagacccagacttggtttgtt

SEQ ID NO: 1226 - DNA, 22 bp, Artificial
  Hsd_from_Mmd_277, Mmd_277; mouse cand318_probe13 sequence
aagggtaagtggttaattgaac

SEQ ID NO: 1227 - DNA, 22 bp, Artificial
  Hsd_from_Mmd_279, Mmd_279; mouse cand666_probe18 sequence
agttcatttgattgaacttggg

SEQ ID NO: 1228 - DNA, 22 bp, Artificial
  Hsd_from_Mmd_28, Mmd_28; mouse cand386_probe17 sequence
gggtgcgggtgttggtggtgtg

SEQ ID NO: 1229 - DNA, 22 bp, Artificial
  Hsd_from_Mmd_280, Mmd_280; mouse cand860_probe10 sequence
gctgcctagtccgaggccagca

SEQ ID NO: 1230 - DNA, 22 bp, Artificial
  Hsd_from_Mmd_284, Mmd_284; mouse cand149_probe35 sequence
gggagccgccaatgacaacaaa

SEQ ID NO: 1231 - DNA, 21 bp, Artificial
  Hsd_from_Mmd_286, Mmd_286; mouse brain 15_J22 sequence
ttaagacggttgagaccagag

SEQ ID NO: 1232 - DNA, 22 bp, Artificial
  Hsd_from_Mmd_291, Mmd_291; mouse cand498_probe20 sequence
accaggagaccaggtgggagcc

SEQ ID NO: 1233 - DNA, 22 bp, Artificial
  Hsd_from_Mmd_293, Mmd_293; mouse cand138_probe15 sequence
catttttttatagcctgaggaa

SEQ ID NO: 1234 - DNA, 22 bp, Artificial
  Hsd_from_Mmd_294, Mmd_294; mouse cand214_probe3 sequence
agggatcttgttaaaaagcaga

SEQ ID NO: 1235 - DNA, 22 bp, Artificial
  Hsd_from_Mmd_295, Mmd_295; mouse mmHP 2669067_probe14 sequence
ggtcattatgagaaaagggatc

SEQ ID NO: 1236 - DNA, 22 bp, Artificial
  Hsd_from_Mmd_296, Mmd_296; mouse cand609_probe9 sequence
gcagggttacattttgccatct

SEQ ID NO: 1237 - DNA, 22 bp, Artificial
  Hsd_from_Mmd_297, Mmd_297; mouse cand461_probe9 sequence
agtgtttaaacacaatgccttt

SEQ ID NO: 1238 - DNA, 22 bp, Artificial
  Hsd_from_Mmd_298, Mmd_298; mouse cand248_probe6 sequence
cccatcgctcatttgcataatg

SEQ ID NO: 1239 - DNA, 22 bp, Artificial
  Hsd_from_Mmd_299, Mmd_299; mouse cand686_probe10 sequence
taaggagggtgaagttgaaggg

SEQ ID NO: 1240 - DNA, 22 bp, Artificial
  Hsd_from_Mmd_300, Mmd_300; mouse cand420_probe12 sequence
ctgtggttttggaattttccaa

SEQ ID NO: 1241 - DNA, 22 bp, Artificial
  Hsd_from_Mmd_301, Mmd_301; mouse cand445_probe4 sequence
gggtgctggcggtgacatgtgc

SEQ ID NO: 1242 - DNA, 22 bp, Artificial
  Hsd_from_Mmd_302, Mmd_302; mouse cand615_probe30 sequence
cagctgaggttaatgagtcccc

SEQ ID NO: 1243 - DNA, 22 bp, Artificial
  Hsd_from_Mmd_302, Mmd_302; mouse cand615_probe36 sequence
ggccatcaactctcccagctgg

SEQ ID NO: 1244 - DNA, 21 bp, Artificial
  Hsd_from_Mmd_303, Mmd_303; mouse brain 35_A09 sequence
tttcctctctgccccataggg

SEQ ID NO: 1245 - DNA, 22 bp, Artificial
  Hsd_from_Mmd_305, Mmd_305; mouse cand657_probe8 sequence
gggtgacaggcagttgttcgtg

SEQ ID NO: 1246 - DNA, 22 bp, Artificial
  Hsd_from_Mmd_306, Mmd_306; mouse mmHP1541151_probe27 sequence
cagaatgactcctctgagaaca

SEQ ID NO: 1247 - DNA, 22 bp, Artificial
  Hsd_from_Mmd_31, Mmd_31; mouse mmH P2052196_probe5 sequence
agggccttagggaactggaggg

SEQ ID NO: 1248 - DNA, 22 bp, Artificial
  Hsd_from_Mmd_311, Mmd_311; mouse cand124_probe1 sequence
aggaaagacactttttatattg

SEQ ID NO: 1249 - DNA, 22 bp, Artificial
  Hsd_from_Mmd_314, Mmd_314; mouse cand148_probe7 sequence
cagatgtgtccacagctaatta

SEQ ID NO: 1250 - DNA, 22 bp, Artificial
  Hsd_from_Mmd_316, Mmd_316; mouse cand367_probe7 sequence
atatttttgtgggaatgggccc

SEQ ID NO: 1251 - DNA, 22 bp, Artificial
  Hsd_from_Mmd_318, Mmd_318; mouse cand543_probe12 sequence
gtgacagagccctaattattaa

SEQ ID NO: 1252 - DNA, 22 bp, Artificial
  Hsd_from_Mmd_321, Mmd_321; mouse cand597_probe9 sequence
tttacagatgattaaatgctcg

SEQ ID NO: 1253 - DNA, 22 bp, Artificial
  Hsd_from_Mmd_324, Mmd_324; mouse cand224_probe10 sequence
ctgcttcgatgtgctggctgtg

SEQ ID NO: 1254 - DNA, 21 bp, Artificial
  Hsd_from_Mmd_325, Mmd_325; mouse brain 102_H06 sequence
gcagtccacgggcatatacac

SEQ ID NO: 1255 - DNA, 22 bp, Artificial
  Hsd_from_Mmd_325, Mmd_325; mouse brain 111_J08 sequence
gcagtccacgggcatatacact

SEQ ID NO: 1256 - DNA, 23 bp, Artificial
  Hsd_from_Mmd_325, Mmd_325; mouse brain 19_C16 sequence
atgcagtccacgggcatatacac

SEQ ID NO: 1257 - DNA, 20 bp, Artificial
  Hsd_from_Mmd_325, Mmd_325; mouse brain 77_O10np sequence
cagtccacgggcatatacac

SEQ ID NO: 1258 - DNA, 22 bp, Artificial
  Hsd_from_Mmd_325, Mmd_325; mouse cand101_probe16 sequence
tgcagtccacgggcatatacac

SEQ ID NO: 1259 - DNA, 22 bp, Artificial
  Hsd_from_Mmd_326, Mmd_326; mouse cand522_probe43 sequence
agggagctgaccttatccaata

SEQ ID NO: 1260 - DNA, 22 bp, Artificial
  Hsd_from_Mmd_328, Mmd_328; mouse cand697_probe25 sequence
gcagggacttcttgccagccct

SEQ ID NO: 1261 - DNA, 22 bp, Artificial
  Hsd_from_Mmd_33, Mmd_33; mouse cand162_probe18 sequence
ggcggattaaggctggagcgca

SEQ ID NO: 1262 - DNA, 22 bp, Artificial
  Hsd_from_Mmd_330, Mmd_330; mouse cand545_probe17 sequence
gcttcccgctcacccacagcag

SEQ ID NO: 1263 - DNA, 22 bp, Artificial
  Hsd_from_Mmd_333, Mmd_333; mouse cand473_probe20 sequence
ttgcaatgggtgagcagactgg

SEQ ID NO: 1264 - DNA, 22 bp, Artificial
  Hsd_from_Mmd_333, Mmd_333; mouse mmHP1301606_probe19 sequence
attgcaatgggtgagcagactg

SEQ ID NO: 1265 - DNA, 22 bp, Artificial
  Hsd_from_Mmd_334, Mmd_334; mouse cand787_probe21 sequence
tgacaagatattttgaaacaac

SEQ ID NO: 1266 - DNA, 22 bp, Artificial
  Hsd_from_Mmd_335, Mmd_335; mouse cand781_probe9 sequence
atcttcagctgattaaatgtcc

SEQ ID NO: 1267 - DNA, 22 bp, Artificial
  Hsd_from_Mmd_336, Mmd_336; mouse cand430_probe8 sequence
tggagagaccttccttctgcag

SEQ ID NO: 1268 - DNA, 22 bp, Artificial
  Hsd_from_Mmd_338, Mmd_338; mouse cand307_probe2 sequence
tcatgatgaagagtttgtcagt

SEQ ID NO: 1269 - DNA, 22 bp, Artificial
  Hsd_from_Mmd_34, Mmd_34; mouse cand553_probe18 sequence
caattgggtcaggaagatcagt

SEQ ID NO: 1270 - DNA, 22 bp, Artificial
  Hsd_from_Mmd_340, Mmd_340; mouse cand853_probe27 sequence
gacattcatcccccggtgggtc

SEQ ID NO: 1271 - DNA, 22 bp, Artificial
  Hsd_from_Mmd_343, Mmd_343; mouse cand673_probe13 sequence
gggagttagtcagctaaatccc

SEQ ID NO: 1272 - DNA, 22 bp, Artificial
  Hsd_from_Mmd_343, Mmd_343; mouse cand673_probe6 sequence
tagggatttggcagcatggccc

SEQ ID NO: 1273 - DNA, 22 bp, Artificial
  Hsd_from_Mmd_344, Mmd_344; mouse cand120_probe19 sequence
tgctagggaacacagggatttc

SEQ ID NO: 1274 - DNA, 22 bp, Artificial
  Hsd_from_Mmd_346, Mmd_346; mouse cand365_probe11 sequence
aggatgttttggatgccgctct

SEQ ID NO: 1275 - DNA, 22 bp, Artificial
  Hsd_from_Mmd_347, Mmd_347; mouse cand492_probe32 sequence
aggattctaaatgtataaattg

SEQ ID NO: 1276 - DNA, 22 bp, Artificial
  Hsd_from_Mmd_35, Mmd_35; mouse cand684_probe15 sequence
ggccagaaggctctccctgcta

SEQ ID NO: 1277 - DNA, 22 bp, Artificial
  Hsd_from_Mmd_353, Mmd_353; mouse cand395_probe7 sequence
agctagaaggagttgatggtcc

SEQ ID NO: 1278 - DNA, 22 bp, Artificial
  Hsd_from_Mmd_354, Mmd_354; mouse cand704_probe5 sequence
gccgggaagattttttggctct

SEQ ID NO: 1279 - DNA, 22 bp, Artificial
  Hsd_from_Mmd_355, Mmd_355; mouse cand323_probe1 sequence
gtattaatgaacgttacaggac

SEQ ID NO: 1280 - DNA, 22 bp, Artificial
  Hsd_from_Mmd_356, Mmd_356; mouse cand182_probe10 sequence
ccagtctggaattttcagcctt

SEQ ID NO: 1281 - DNA, 22 bp, Artificial
  Hsd_from_Mmd_358, Mmd_358; mouse cand668_probe30 sequence
gcagaggataatggcgtctgca

SEQ ID NO: 1282 - DNA, 22 bp, Artificial
  Hsd_from_Mmd_361, Mmd_361; mouse cand18_probe11 sequence
atatattgtacaaaagagcctt

SEQ ID NO: 1283 - DNA, 22 bp, Artificial
  Hsd_from_Mmd_363, Mmd_363; mouse mmHP 2216591_probe41 sequence
gcagaaatgtttccactagatt

SEQ ID NO: 1284 - DNA, 22 bp, Artificial
  Hsd_from_Mmd_367, Mmd_367; mouse cand558_probe1 sequence
ggaaattttatttgaatcaggg

SEQ ID NO: 1285 - DNA, 22 bp, Artificial
  Hsd_from_Mmd_37, Mmd_37; mouse mmH P200260_probe14 sequence
gcaatttgcagagccattaatg

SEQ ID NO: 1286 - DNA, 22 bp, Artificial
  Hsd_from_Mmd_370, Mmd_370; mouse cand546_probe16 sequence
gccgcaggctggctcattgtct

SEQ ID NO: 1287 - DNA, 22 bp, Artificial
  Hsd_from_Mmd_371, Mmd_371; mouse cand854_probe3 sequence
ccaggtgctggtggaaatagag

SEQ ID NO: 1288 - DNA, 22 bp, Artificial
  Hsd_from_Mmd_372, Mmd_372; mouse cand181_probe20 sequence
gcaggagccctaatcggattgg

SEQ ID NO: 1289 - DNA, 22 bp, Artificial
  Hsd_from_Mmd_373, Mmd_373; mouse cand767_probe3 sequence
tttcagaaaattcttccaggag

SEQ ID NO: 1290 - DNA, 22 bp, Artificial
  Hsd_from_Mmd_376, Mmd_376; mouse cand294_probe16 sequence
tgtctctaattccccaagggta

SEQ ID NO: 1291 - DNA, 22 bp, Artificial
  Hsd_from_Mmd_378, Mmd_378; mouse cand136_probe8 sequence
aaataatgtgagatcaggctca

SEQ ID NO: 1292 - DNA, 22 bp, Artificial
  Hsd_from_Mmd_382, Mmd_382; mouse mmH P1665736_probe7 sequence
aggactcaatgacgttgaagtt

SEQ ID NO: 1293 - DNA, 22 bp, Artificial
  Hsd_from_Mmd_383, Mmd_383; mouse cand675_probe1 sequence
gcactgggcaggaagactttat

SEQ ID NO: 1294 - DNA, 22 bp, Artificial
  Hsd_from_Mmd_387, Mmd_387; mouse cand265_probe18 sequence
tttgtgcccacgcctctgggtc

SEQ ID NO: 1295 - DNA, 22 bp, Artificial
  Hsd_from_Mmd_388, Mmd_388; mouse cand635_probe43 sequence
agggcatgtcattattaggtca

SEQ ID NO: 1296 - DNA, 22 bp, Artificial
  Hsd_from_Mmd_39, Mmd_39; mouse cand184_probe15 sequence
agttactaatttctgatgctct

SEQ ID NO: 1297 - DNA, 22 bp, Artificial
  Hsd_from_Mmd_39, Mmd_39; mouse cand184_probe5 sequence
aggagctatcagaacttagtga

SEQ ID NO: 1298 - DNA, 22 bp, Artificial
  Hsd_from_Mmd_391, Mmd_391; mouse cand698_probe22 sequence
cagcgtctggcctctgagccag

SEQ ID NO: 1299 - DNA, 22 bp, Artificial
  Hsd_from_Mmd_396, Mmd_396; mouse cand187_probe38 sequence
gagcaattaaattgcaccagga

SEQ ID NO: 1300 - DNA, 22 bp, Artificial
  Hsd_from_Mmd_398, Mmd_398; mouse cand361_probe11 sequence
aggaggccatatgtggccgtgt

SEQ ID NO: 1301 - DNA, 22 bp, Artificial
  Hsd_from_Mmd_399, Mmd_399; mouse cand694_probe2 sequence
gaggccacagatgctgatggaa

SEQ ID NO: 1302 - DNA, 22 bp, Artificial
  Hsd_from_Mmd_40, Mmd_40; mouse cand519_probe11 sequence
ggccaggtagtccttgcggcca

SEQ ID NO: 1303 - DNA, 21 bp, Artificial
  Hsd_from_Mmd_405, Mmd_405; mouse brain 58_M04np sequence
ctgggccctcttcccacccag

SEQ ID NO: 1304 - DNA, 22 bp, Artificial
  Hsd_from_Mmd_408, Mmd_408; mouse cand547_probe13 sequence
catgagagtttgcatgcaaatg

SEQ ID NO: 1305 - DNA, 22 bp, Artificial
  Hsd_from_Mmd_409, Mmd_409; mouse cand292_probe2 sequence
aggaaattgtatgataaatagg

SEQ ID NO: 1306 - DNA, 22 bp, Artificial
  Hsd_from_Mmd_41, Mmd_41; mouse cand98_probe29 sequence
tgcaggaacttgtgagtctcct

SEQ ID NO: 1307 - DNA, 22 bp, Artificial
  Hsd_from_Mmd_410, Mmd_410; mouse cand326_probe38 sequence
gccatctgcgtctaccagacgc

SEQ ID NO: 1308 - DNA, 22 bp, Artificial
  Hsd_from_Mmd_411, Mmd_411; mouse cand257_probe3 sequence
agcactgtgctaaaattgcagg

SEQ ID NO: 1309 - DNA, 22 bp, Artificial
  Hsd_from_Mmd_412, Mmd_412; mouse mmHP453167_probe8 sequence
attaggagtggcagaaaggatg

SEQ ID NO: 1310 - DNA, 22 bp, Artificial
  Hsd_from_Mmd_413, Mmd_413; mouse cand718_probe33 sequence
ccagctgggaagaaccagtggc

SEQ ID NO: 1311 - DNA, 22 bp, Artificial
  Hsd_from_Mmd_416, Mmd_416; mouse cand8_probe3 sequence
tgggggaggcgggctgtgctgg

SEQ ID NO: 1312 - DNA, 22 bp, Artificial
  Hsd_from_Mmd_418, Mmd_418; mouse cand425_probe43 sequence
aggggtaagagagtagaaccgg

SEQ ID NO: 1313 - DNA, 22 bp, Artificial
  Hsd_from_Mmd_422, Mmd_422; mouse cand196_probe33 sequence
ttgtgacatcacaattagccac

SEQ ID NO: 1314 - DNA, 22 bp, Artificial
  Hsd_from_Mmd_424, Mmd_424; mouse cand443_probe6 sequence
caagctctacaaccattgagct

SEQ ID NO: 1315 - DNA, 22 bp, Artificial
  Hsd_from_Mmd_426, Mmd_426; mouse cand427_probe13 sequence
caggcctgagtcacagctgctg

SEQ ID NO: 1316 - DNA, 22 bp, Artificial
  Hsd_from_Mmd_428, Mmd_428; mouse cand518_probe14 sequence
cagtgcgctggctgctggagga

SEQ ID NO: 1317 - DNA, 22 bp, Artificial
  Hsd_from_Mmd_428, Mmd_428; mouse cand518_probe8 sequence
cctgcagcagctggtgaccttg

SEQ ID NO: 1318 - DNA, 22 bp, Artificial
  Hsd_from_Mmd_429, Mmd_429; mouse cand685_probe7 sequence
aggggattaggacaagagatgt

SEQ ID NO: 1319 - DNA, 22 bp, Artificial
  Hsd_from_Mmd_430, Mmd_430; mouse cand607_probe13 sequence
aggaagagaaaccagattgtgt

SEQ ID NO: 1320 - DNA, 22 bp, Artificial
  Hsd_from_Mmd_430, Mmd_430; mouse cand607_probe6 sequence
gcaggactacatttgttctccc

SEQ ID NO: 1321 - DNA, 22 bp, Artificial
  Hsd_from_Mmd_431, Mmd_431; mouse mmHP 2175366_probe12 sequence
gtgctagcaatctgagagacca

SEQ ID NO: 1322 - DNA, 22 bp, Artificial
  Hsd_from_Mmd_433, Mmd_433; mouse cand192_probe4 sequence
gccggagccctggcagataaat

SEQ ID NO: 1323 - DNA, 22 bp, Artificial
  Hsd_from_Mmd_435, Mmd_435; mouse cand125_probe11 sequence
atgggtaattgcacacacaaat

SEQ ID NO: 1324 - DNA, 22 bp, Artificial
  Hsd_from_Mmd_436, Mmd_436; mouse cand544_probe10 sequence
ccctggcactgatggcctgtgc

SEQ ID NO: 1325 - DNA, 23 bp, Artificial
  Hsd_from_Mmd_437, Mmd_437; mouse brain 87_F08 sequence
ctggctgagagcaggactaaccc

SEQ ID NO: 1326 - DNA, 22 bp, Artificial
  Hsd_from_Mmd_438, Mmd_438; mouse cand957_probe18 sequence
ctttgacaagagaggattaggg

SEQ ID NO: 1327 - DNA, 22 bp, Artificial
  Hsd_from_Mmd_438, Mmd_438; mouse cand957_probe5 sequence
ttgaagtgtctcttgtcaaaga

SEQ ID NO: 1328 - DNA, 22 bp, Artificial
  Hsd_from_Mmd_439, Mmd_439; mouse cand165_probe14 sequence
ctgaggaggagtgtgattacca

SEQ ID NO: 1329 - DNA, 22 bp, Artificial
  Hsd_from_Mmd_44, Mmd_44; mouse cand618_probe6 sequence
gggtgaaaagttcgcagcgagg

SEQ ID NO: 1330 - DNA, 22 bp, Artificial
  Hsd_from_Mmd_443, Mmd_443; mouse cand73_probe10 sequence
tgaaccacagctgagttcacag

SEQ ID NO: 1331 - DNA, 20 bp, Artificial
  Hsd_from_Mmd_448, Mmd_448; mouse brain 72_B19 sequence
cagaaggggagtcgggagcg

SEQ ID NO: 1332 - DNA, 22 bp, Artificial
  Hsd_from_Mmd_45, Mmd_45; mouse cand540_probe12 sequence
gtgtgtgtgtttattgtaatat

SEQ ID NO: 1333 - DNA, 22 bp, Artificial
  Hsd_from_Mmd_450, Mmd_450; mouse cand536_probe8 sequence
agccctttaatgccagcagctg

SEQ ID NO: 1334 - DNA, 22 bp, Artificial
  Hsd_from_Mmd_452, Hsd_from_Mmd_473, Mmd_452, Mmd_473; mouse cand167_probe11 sequence
gcagagtgcaaacaattttgac

SEQ ID NO: 1335 - DNA, 22 bp, Artificial
  Hsd_from_Mmd_455, Mmd_455; mouse cand190_probe12 sequence
gggtgaaaagagacattatcct

SEQ ID NO: 1336 - DNA, 22 bp, Artificial
  Hsd_from_Mmd_456, Mmd_456; mouse cand239_probe7 sequence
tggagacgccaaggggaaaatg

SEQ ID NO: 1337 - DNA, 22 bp, Artificial
  Hsd_from_Mmd_459, Mmd_459; mouse cand681_probe30 sequence
aagtggccttcaatagtccgat

SEQ ID NO: 1338 - DNA, 22 bp, Artificial
  Hsd_from_Mmd_461, Mmd_461; mouse cand392_probe21 sequence
ggcgccaacgggcaggtgcgct

SEQ ID NO: 1339 - DNA, 22 bp, Artificial
  Hsd_from_Mmd_464, Mmd_464; mouse cand688_probe8 sequence
caatgggccagattctcactct

SEQ ID NO: 1340 - DNA, 22 bp, Artificial
  Hsd_from_Mmd_465, Mmd_465; mouse cand449_probe19 sequence
gagctcaaattgcagctacaac

SEQ ID NO: 1341 - DNA, 22 bp, Artificial
  Hsd_from_Mmd_466, Mmd_466; mouse cand438_probe6 sequence
gatgaggaggtcatcaaaatga

SEQ ID NO: 1342 - DNA, 22 bp, Artificial
  Hsd_from_Mmd_468, Mmd_468; mouse cand58_probe19 sequence
aggggccggaatggagcgctcc

SEQ ID NO: 1343 - DNA, 22 bp, Artificial
  Hsd_from_Mmd_47, Mmd_47; mouse cand368_probe44 sequence
tatcaagggctgggaccagggg

SEQ ID NO: 1344 - DNA, 22 bp, Artificial
  Hsd_from_Mmd_471, Mmd_471; mouse cand428_probe21 sequence
gctgccatgaccagttaaggga

SEQ ID NO: 1345 - DNA, 22 bp, Artificial
  Hsd_from_Mmd_472, Mmd_472; mouse cand141_probe5 sequence
tgctgctggctcagaccccagc

SEQ ID NO: 1346 - DNA, 22 bp, Artificial
  Hsd_from_Mmd_476, Mmd_476; mouse cand416_probe11 sequence
ccggagagtgtagtttgtttgt

SEQ ID NO: 1347 - DNA, 22 bp, Artificial
  Hsd_from_Mmd_477, Mmd_477; mouse cand415_probe21 sequence
caatgggagagagagaatggga

SEQ ID NO: 1348 - DNA, 22 bp, Artificial
  Hsd_from_Mmd_478, Mmd_478; mouse cand622_probe21 sequence
gctcagaggccacttggccaga

SEQ ID NO: 1349 - DNA, 22 bp, Artificial
  Hsd_from_Mmd_48, Mmd_48; mouse cand452_probe35 sequence
agggaattactaatttacattt

SEQ ID NO: 1350 - DNA, 22 bp, Artificial
  Hsd_from_Mmd_480, Mmd_480; mouse cand963_probe9 sequence
aggattggtggtggggccagat

SEQ ID NO: 1351 - DNA, 22 bp, Artificial
  Hsd_from_Mmd_481, Mmd_481; mouse cand269_probe17 sequence
cagtattccacaatggatcagt

SEQ ID NO: 1352 - DNA, 22 bp, Artificial
  Hsd_from_Mmd_481, Mmd_481; mouse cand269_probe2 sequence
gggctgcattcattttgagtgc

SEQ ID NO: 1353 - DNA, 22 bp, Artificial
  Hsd_from_Mmd_482, Mmd_482; mouse cand486_probe16 sequence
ccgaggaggtgcccgtggacgt

SEQ ID NO: 1354 - DNA, 22 bp, Artificial
  Hsd_from_Mmd_483, Mmd_483; mouse cand271_probe10 sequence
cggccaggctgagggaaggaag

SEQ ID NO: 1355 - DNA, 22 bp, Artificial
  Hsd_from_Mmd_485, Mmd_485; mouse cand57_probe38 sequence
cagtaaagccaagtagtgcatg

SEQ ID NO: 1356 - DNA, 22 bp, Artificial
  Hsd_from_Mmd_486, Mmd_486; mouse cand38_probe12 sequence
tgccaaatgttagcaaggaaac

SEQ ID NO: 1357 - DNA, 22 bp, Artificial
  Hsd_from_Mmd_487, Hsd_from_Mmd_535, Hsd_from_Mmd_78, Mmd_487, Mmd_535, Mmd_78; mouse cand780_probe16 sequence
ctttgtcacaggttaccagggt

SEQ ID NO: 1358 - DNA, 22 bp, Artificial
  Hsd_from_Mmd_487, Hsd_from_Mmd_535, Hsd_from_Mmd_78, Mmd_487, Mmd_535, Mmd_78; mouse cand780_probe8 sequence
ctgggcctgagacagaggaggg

SEQ ID NO: 1359 - DNA, 22 bp, Artificial
  Hsd_from_Mmd_488, Mmd_488; mouse cand255_probe13 sequence
cggtctggttggtcttgggccc

SEQ ID NO: 1360 - DNA, 22 bp, Artificial
  Hsd_from_Mmd_489, Mmd_489; mouse cand604_probe27 sequence
tttaaaaggcttttaaatgatg

SEQ ID NO: 1361 - DNA, 22 bp, Artificial
  Hsd_from_Mmd_49, Mmd_49; mouse cand732_probe22 sequence
ctcatggtgccggggtcggggc

SEQ ID NO: 1362 - DNA, 20 bp, Artificial
  Hsd_from_Mmd_490, Mmd_490; mouse brain 42_N15 sequence
agtattacatggccaatctc

SEQ ID NO: 1363 - DNA, 22 bp, Artificial
  Hsd_from_Mmd_490, Mmd_490; mouse brain 51_A22 sequence
tgagtattacatggccaatctc

SEQ ID NO: 1364 - DNA, 22 bp, Artificial
  Hsd_from_Mmd_491, Mmd_491; mouse cand247_probe17 sequence
catgatgagctttgtgcaaagt

SEQ ID NO: 1365 - DNA, 22 bp, Artificial
  Hsd_from_Mmd_492, Mmd_492; mouse cand243_probe7 sequence
atcgatgggaaatgatgtgtga

SEQ ID NO: 1366 - DNA, 22 bp, Artificial
  Hsd_from_Mmd_494, Mmd_494; mouse cand115_probe6 sequence
atttcgggttttccagtccgat

SEQ ID NO: 1367 - DNA, 22 bp, Artificial
  Hsd_from_Mmd_499, Mmd_499; mouse cand835_probe15 sequence
ccgtaagcaattattaaatgcc

SEQ ID NO: 1368 - DNA, 22 bp, Artificial
  Hsd_from_Mmd_499, Mmd_499; mouse cand835_probe2 sequence
cacatgtaatgaattgtgggtg

SEQ ID NO: 1369 - DNA, 22 bp, Artificial
  Hsd_from_Mmd_500, Mmd_500; mouse cand322_probe32 sequence
atggcaatttgtttcatttgcc

SEQ ID NO: 1370 - DNA, 22 bp, Artificial
  Hsd_from_Mmd_501, Mmd_501; mouse cand7_probe8 sequence
tgacaataaaagttatgggtcc

SEQ ID NO: 1371 - DNA, 22 bp, Artificial
  Hsd_from_Mmd_503, Mmd_503; mouse cand887_probe4 sequence
cctcagctatgcatgcacagct

SEQ ID NO: 1372 - DNA, 22 bp, Artificial
  Hsd_from_Mmd_505, Mmd_505; mouse cand949_probe25 sequence
ttaaggaatcactcttgatctc

SEQ ID NO: 1373 - DNA, 22 bp, Artificial
  Hsd_from_Mmd_506, Mmd_506; mouse cand93_probe8 sequence
gctggtggctctgatttcatga

SEQ ID NO: 1374 - DNA, 21 bp, Artificial
  Hsd_from_Mmd_507, Mmd_507; mouse brain 57_I16np sequence
cggctctgggtctgtggggag

SEQ ID NO: 1375 - DNA, 21 bp, Artificial
  Hsd_from_Mmd_507, Mmd_507; mouse brain 7_G17np sequence
tcggctctgggtctgtgggga

SEQ ID NO: 1376 - DNA, 22 bp, Artificial
  Hsd_from_Mmd_507, Mmd_507; mouse cand244_probe12 sequence
gaaattcggctctgggtctgtg

SEQ ID NO: 1377 - DNA, 22 bp, Artificial
  Hsd_from_Mmd_509, Mmd_509; mouse cand952_probe11 sequence
ctgcagagaagtgaactgaaga

SEQ ID NO: 1378 - DNA, 22 bp, Artificial
  Hsd_from_Mmd_51, Mmd_51; mouse cand99_probe20 sequence
gctgcaggcatatttgcatatt

SEQ ID NO: 1379 - DNA, 22 bp, Artificial
  Hsd_from_Mmd_510, Mmd_510; mouse cand692_probe9 sequence
gccccaggatggagtaggcagg

SEQ ID NO: 1380 - DNA, 22 bp, Artificial
  Hsd_from_Mmd_511, Mmd_511; mouse cand683_probe8 sequence
ctctgtgggagggaggagatca

SEQ ID NO: 1381 - DNA, 22 bp, Artificial
  Hsd_from_Mmd_515, Mmd_515; mouse cand389_probe14 sequence
gtgagacactgggacgcacgct

SEQ ID NO: 1382 - DNA, 22 bp, Artificial
  Hsd_from_Mmd_516, Mmd_516; mouse cand722_probe18 sequence
cttggatgagcgtccagttgct

SEQ ID NO: 1383 - DNA, 22 bp, Artificial
  Hsd_from_Mmd_517, Mmd_517; mouse cand842_probe10 sequence
tcattctctatgggggccgggg

SEQ ID NO: 1384 - DNA, 22 bp, Artificial
  Hsd_from_Mmd_518, Mmd_518; mouse cand790_probe19 sequence
tggtgggggtggaactcagctg

SEQ ID NO: 1385 - DNA, 22 bp, Artificial
  Hsd_from_Mmd_518, Mmd_518; mouse cand790_probe5 sequence
ccggattgtccccttgctgctg

SEQ ID NO: 1386 - DNA, 22 bp, Artificial
  Hsd_from_Mmd_519, Mmd_519; mouse cand676_probe12 sequence
cgtatctatgaggaacagcagg

SEQ ID NO: 1387 - DNA, 22 bp, Artificial
  Hsd_from_Mmd_52, Mmd_52; mouse cand922_probe21 sequence
taaggaggctgtagaaatgaag

SEQ ID NO: 1388 - DNA, 22 bp, Artificial
  Hsd_from_Mmd_523, Mmd_523; mouse cand433_probe8 sequence
attgaaggatgctgtggcccac

SEQ ID NO: 1389 - DNA, 22 bp, Artificial
  Hsd_from_Mmd_523, Mmd_523; mouse cand435_probe12 sequence
aggaacacagctttacgttcaa

SEQ ID NO: 1390 - DNA, 22 bp, Artificial
  Hsd_from_Mmd_524, Mmd_524; mouse cand89_probe16 sequence
ggccagtgtcatgcagaagatt

SEQ ID NO: 1391 - DNA, 21 bp, Artificial
ttcattcggctgtccagatgt

SEQ ID NO: 1392 - DNA, 22 bp, Artificial
  Hsd_from_Mmd_525, Mmd_525; mouse brain 88_L17np sequence
ttcattcggctgtccagatgta

SEQ ID NO: 1393 - DNA, 22 bp, Artificial
  Hsd_from_Mmd_526, Mmd_526; mouse cand593_probe15 sequence
aggacagacagcaggagacgct

SEQ ID NO: 1394 - DNA, 22 bp, Artificial
  Hsd_from_Mmd_526, Mmd_526; mouse cand593_probe6 sequence
ggcgttgatgtctgctttcatc

SEQ ID NO: 1395 - DNA, 22 bp, Artificial
  Hsd_from_Mmd_527, Mmd_527; mouse cand332_probe20 sequence
ttaaaggagatcagaggtaatt

SEQ ID NO: 1396 - DNA, 22 bp, Artificial
  Hsd_from_Mmd_529, Mmd_529; mouse cand741_probe25 sequence
gcagtgtgtgagaggtgtacgc

SEQ ID NO: 1397 - DNA, 22 bp, Artificial
  Hsd_from_Mmd_530, Mmd_530; mouse cand523_probe3 sequence
atgcctgatacattctccaggc

SEQ ID NO: 1398 - DNA, 22 bp, Artificial
  Hsd_from_Mmd_532, Mmd_532; mouse cand111_probe13 sequence
agggtaattgcacgatattttc

SEQ ID NO: 1399 - DNA, 22 bp, Artificial
  Hsd_from_Mmd_533, Mmd_533; mouse cand95_probe8 sequence
gatttccaataattgaggcagt

SEQ ID NO: 1400 - DNA, 22 bp, Artificial
  Hsd_from_Mmd_534, Mmd_534; mouse cand477_probe12 sequence
aaggaagcttcatgcttggatc

SEQ ID NO: 1401 - DNA, 22 bp, Artificial
  Hsd_from_Mmd_536, Mmd_536; mouse cand109_probe41 sequence
ggctgccaaacactggagcagc

SEQ ID NO: 1402 - DNA, 18 bp, Artificial
  Hsd_from_Mmd_54, Mmd_54; mouse brain 35_N12np sequence
accaggaggctgaggtcc

SEQ ID NO: 1403 - DNA, 21 bp, Artificial
  Hsd_from_Mmd_54, Mmd_54; mouse brain 74_K09 sequence
accaggaggctgaggtccctt

SEQ ID NO: 1404 - DNA, 22 bp, Artificial
  Hsd_from_Mmd_56, Mmd_56; mouse cand266_probe10 sequence
aggaaggtgtgctgctggagga

SEQ ID NO: 1405 - DNA, 22 bp, Artificial
  Hsd_from_Mmd_56, Mmd_56; mouse cand266_probe12 sequence
gggctggtgggggcatctttct

SEQ ID NO: 1406 - DNA, 22 bp, Artificial
  Hsd_from_Mmd_58, Mmd_58; mouse cand712_probe10 sequence
agccatgatggcaatgtccgag

SEQ ID NO: 1407 - DNA, 22 bp, Artificial
  Hsd_from_Mmd_59, Mmd_59; mouse cand552_probe8 sequence
cccgccaggtcggcgggaagag

SEQ ID NO: 1408 - DNA, 22 bp, Artificial
  Hsd_from_Mmd_60, Mmd_60; mouse cand838_probe12 sequence
cagtcatggcgggaggactgaa

SEQ ID NO: 1409 - DNA, 22 bp, Artificial
  Hsd_from_Mmd_67, Mmd_67; mouse cand11_probe5 sequence
gtagatatcatacaggaatgct

SEQ ID NO: 1410 - DNA, 22 bp, Artificial
  Hsd_from_Mmd_68, Mmd_68; mouse cand586_probe2 sequence
aaggaagaagtttgttctgttg

SEQ ID NO: 1411 - DNA, 22 bp, Artificial
  Hsd_from_Mmd_69, Mmd_69; mouse cand632_probe11 sequence
aggtgaaactcaaagggtccaa

SEQ ID NO: 1412 - DNA, 22 bp, Artificial
  Hsd_from_Mmd_70, Mmd_70; mouse cand200_probe39 sequence
gccaattgcaggcccaattggc

SEQ ID NO: 1413 - DNA, 22 bp, Artificial
  Hsd_from_Mmd_71, Mmd_71; mouse cand402_probe35 sequence
agagtgtagtgcaatcagtcag

SEQ ID NO: 1414 - DNA, 22 bp, Artificial
  Hsd_from_Mmd_74, Mmd_74; mouse cand734_probe31 sequence
gggataattaccatctctcagg

SEQ ID NO: 1415 - DNA, 22 bp, Artificial
  Hsd_from_Mmd_75, Mmd_75; mouse cand656_probe13 sequence
cgaggttggcatcagaccctct

SEQ ID NO: 1416 - DNA, 22 bp, Artificial
  Hsd_from_Mmd_76, Mmd_76; mouse cand645_probe3 sequence
atttgtgtcagatttcaatcta

SEQ ID NO: 1417 - DNA, 22 bp, Artificial
  Hsd_from_Mmd_80, Mmd_80; mouse cand261_probe12 sequence
gcagagagaagagactccatct

SEQ ID NO: 1418 - DNA, 22 bp, Artificial
  Hsd_from_Mmd_81, Mmd_81; mouse cand28_probe31 sequence
tttaaaaagctaatttgggagc

SEQ ID NO: 1419 - DNA, 22 bp, Artificial
  Hsd_from_Mmd_81, Mmd_81; mouse cand28_probe40 sequence
attcagacattttaaataggaa

SEQ ID NO: 1420 - DNA, 22 bp, Artificial
  Hsd_from_Mmd_82, Mmd_82; mouse cand896_probe6 sequence
ggccaagatacgtagtcgccaa

SEQ ID NO: 1421 - DNA, 22 bp, Artificial
  Hsd_from_Mmd_85, Mmd_85; mouse cand59_probe28 sequence
catctgttcaaacataatgctc

SEQ ID NO: 1422 - DNA, 22 bp, Artificial
  Hsd_from_Mmd_90, Mmd_90; mouse cand363_probe13 sequence
gctggagttcaatatgctcagg

SEQ ID NO: 1423 - DNA, 22 bp, Artificial
  Hsd_from_Mmd_90, Mmd_90; mouse cand363_probe7 sequence
tcaattgtgtaatttccagctg

SEQ ID NO: 1424 - DNA, 22 bp, Artificial
  Hsd_from_Mmd_91, Mmd_91; mouse cand629_probe16 sequence
cggagggaacagctgagaacta

SEQ ID NO: 1425 - DNA, 22 bp, Artificial
  Hsd_from_Mmd_92, Mmd_92; mouse mmHP2160056_probe22 sequence
tgatcttgctgatgagcagggt

SEQ ID NO: 1426 - DNA, 22 bp, Artificial
  Hsd_from_Mmd_93, Mmd_93; mouse cand331_probe6 sequence
gcgtctggtagacgcagatggc

SEQ ID NO: 1427 - DNA, 22 bp, Artificial
  Hsd_from_Mmd_94, Mmd_94; mouse cand696_probe8 sequence
aagccttagaagagccaaagta

SEQ ID NO: 1428 - DNA, 22 bp, Artificial
  Hsd_from_Mmd_95, Mmd_95; mouse cand584_probe24 sequence
caaagttaaggcaggggagcca

SEQ ID NO: 1429 - DNA, 22 bp, Artificial
  Hsd_from_Mmd_97, Mmd_97; mouse cand68_probe2 sequence
caatctccacttcactgtcagt

SEQ ID NO: 1430 - DNA, 22 bp, Artificial
  Hsd_from_Mmd_98, Mmd_98; mouse cand482_probe12 sequence
ggggctggggccgggacagagc

SEQ ID NO: 1431 - DNA, 22 bp, Artificial
  Mmd_102; mouse mmHP969107_probe21 sequence
tgtggaggcacggcactcgggt

SEQ ID NO: 1432 - DNA, 22 bp, Artificial
  Mmd_102; mouse mmHP969107_probe3 sequence
cgagtccaggcctccatgcagc

SEQ ID NO: 1433 - DNA, 20 bp, Artificial
  Mmd_103; mouse brain 65_C14np sequence
ttctggaggcacatggtttg

SEQ ID NO: 1434 - DNA, 22 bp, Artificial
  Mmd_104; mouse mmHP2682003_probe7 sequence
ttaaaaggggtctcaggtaagg

SEQ ID NO: 1435 - DNA, 21 bp, Artificial
  Mmd_106, Mmd_168; mouse brain 11_H05np sequence
ggtgcgcgtctctcggggtcg

SEQ ID NO: 1436 - DNA, 18 bp, Artificial
  Mmd_106, Mmd_168; mouse brain 82_M03np sequence
gcgtctctcggggtcggg

SEQ ID NO: 1437 - DNA, 22 bp, Artificial
  Mmd_108, Mmd_125, Mmd_160, Mmd_240, Mmd_251, Mmd_265, Mmd_29, Mmd_322, Mmd_323, Mmd_362, Mmd_386, Mmd_415, Mmd_463, Mmd_504; mouse brain 3_L19 sequence
tcttgaagcctgactccccgcg

SEQ ID NO: 1438 - DNA, 22 bp, Artificial
  Mmd_109; mouse mmHP121950_probe14 sequence
gctcagattgcaatgctggggt

SEQ ID NO: 1439 - DNA, 20 bp, Artificial
  Mmd_110; mouse brain 21_I02 sequence
ctctactccctgccccagcc

SEQ ID NO: 1440 - DNA, 21 bp, Artificial
  Mmd_110; mouse brain 61_E24np sequence
ggtggtgcaggcaggagagcc

SEQ ID NO: 1441 - DNA, 21 bp, Artificial
  Mmd_111, Mmd_175, Mmd_188, Mmd_421, Mmd_522; mouse brain 112_M18 sequence
gatataaccactgccagactg

SEQ ID NO: 1442 - DNA, 20 bp, Artificial
  Mmd_111, Mmd_175, Mmd_188, Mmd_421, Mmd_522; mouse brain 69_K07 sequence
gatataaccactgccagact

SEQ ID NO: 1443 - DNA, 18 bp, Artificial
  Mmd_113; mouse brain 53_K18np sequence
gtggatattcccttctat

SEQ ID NO: 1444 - DNA, 22 bp, Artificial
  Mmd_114; mouse mmHP1647321_probe8 sequence
ccccagtgtgtcagtgtagagc

SEQ ID NO: 1445 - DNA, 22 bp, Artificial
  Mmd_116; mouse mmHP1779096_probe17 sequence
ggccaggtgtgaggcatcagag

SEQ ID NO: 1446 - DNA, 24 bp, Artificial
  Mmd_118; mouse brain 63_D22 sequence
ttgagagatggggaaataggcact

SEQ ID NO: 1447 - DNA, 22 bp, Artificial
  Mmd_121; mouse cand913_probe22 sequence
acgtctgaggagcggcggcggc

SEQ ID NO: 1448 - DNA, 22 bp, Artificial
  Mmd_122; mouse mmHP2044575_probe10 sequence
agggatgtaaagtaagctggct

SEQ ID NO: 1449 - DNA, 22 bp, Artificial
  Mmd_124; mouse cand168_probe2 sequence
aggatacagagctctttaaata

SEQ ID NO: 1450 - DNA, 22 bp, Artificial
  Mmd_124; mouse cand168_probe22 sequence
ttaaagatttttgttccacctg

SEQ ID NO: 1451 - DNA, 22 bp, Artificial
  Mmd_127; mouse mmHP157519_probe4 sequence
aggggaagaggggactctgtct

SEQ ID NO: 1452 - DNA, 22 bp, Artificial
  Mmd_130; mouse mmHP115458_probe21 sequence
ccagctggaggtcatactaaat

SEQ ID NO: 1453 - DNA, 19 bp, Artificial
  Mmd_131; mouse brain 106_I09np sequence
agtcaggttgtgtggtttt

SEQ ID NO: 1454 - DNA, 22 bp, Artificial
  Mmd_132; mouse mmHP1181586_probe18 sequence
tgtcaccttacaccccgtcaag

SEQ ID NO: 1455 - DNA, 22 bp, Artificial
  Mmd_132; mouse mmHP1181586_probe7 sequence
gcagggtgtgaggggactcgtc

SEQ ID NO: 1456 - DNA, 22 bp, Artificial
  Mmd_134; mouse mmHP2549445_probe12 sequence
gcgatctaccagttcataagga

SEQ ID NO: 1457 - DNA, 22 bp, Artificial
  Mmd_135; mouse mmHP291564_probe22 sequence
ggatccagactgtcccaatgga

SEQ ID NO: 1458 - DNA, 22 bp, Artificial
  Mmd_139; mouse cand238_probe13 sequence
ggccattaactccgggtggtca

SEQ ID NO: 1459 - DNA, 22 bp, Artificial
  Mmd_140; mouse mmHP2148319_probe5 sequence
aaggagtgaaatattttgctgg

SEQ ID NO: 1460 - DNA, 19 bp, Artificial
  Mmd_142; mouse brain 108_H04np sequence
ggttggtgtactgtgtgtg

SEQ ID NO: 1461 - DNA, 21 bp, Artificial
  Mmd_142; mouse brain 52_E14 sequence
tgaggttggtgtactgtgtgt

SEQ ID NO: 1462 - DNA, 22 bp, Artificial
  Mmd_142; mouse brain 62_E07 sequence
tgaggttggtgtactgtgtgtg

SEQ ID NO: 1463 - DNA, 18 bp, Artificial
  Mmd_143; mouse brain 102_E05np sequence
tggttgtctggttgtggg

SEQ ID NO: 1464 - DNA, 19 bp, Artificial
  Mmd_144; mouse brain 7_L19np sequence
ccacagtttcctcatcccc

SEQ ID NO: 1465 - DNA, 22 bp, Artificial
  Mmd_146; mouse cand517_probe15 sequence
ctgaggacttacagattaactc

SEQ ID NO: 1466 - DNA, 22 bp, Artificial
  Mmd_146; mouse cand517_probe6 sequence
gggaaagaattaatgtgtgctc

SEQ ID NO: 1467 - DNA, 21 bp, Artificial
  Mmd_151; mouse brain 4_D20np sequence
tccctgagttcctgtcagttg

SEQ ID NO: 1468 - DNA, 22 bp, Artificial
  Mmd_152; mouse cand789_probe9 sequence
gggtatctggtgaaccaagctg

SEQ ID NO: 1469 - DNA, 22 bp, Artificial
  Mmd_153; mouse cand678_probe18 sequence
ccggaactgtggactacagctc

SEQ ID NO: 1470 - DNA, 22 bp, Artificial
  Mmd_153; mouse cand678_probe8 sequence
gtagttctagaggcacccgggt

SEQ ID NO: 1471 - DNA, 22 bp, Artificial
  Mmd_158; mouse mmHP2073048_probe4 sequence
gtgcacagggtaaattcattgg

SEQ ID NO: 1472 - DNA, 22 bp, Artificial
  Mmd_164; mouse cand658_probe20 sequence
gcagagcaaaaggcatggtggg

SEQ ID NO: 1473 - DNA, 22 bp, Artificial
  Mmd_165; mouse mmHP3434196_probe13 sequence
catgaggatagtcagtagtcca

SEQ ID NO: 1474 - DNA, 18 bp, Artificial
  Mmd_171; mouse brain 7_L13np sequence
aggtcagaggtcgatcct

SEQ ID NO: 1475 - DNA, 22 bp, Artificial
  Mmd_177, Mmd_43; mouse mmHP1442327_probe17 sequence
agggacagtgcgtgggtgtgtg

SEQ ID NO: 1476 - DNA, 18 bp, Artificial
  Mmd_179; mouse brain 22_I06np sequence
taggttatcctctgttgc

SEQ ID NO: 1477 - DNA, 22 bp, Artificial
  Mmd_18; mouse cand506_probe12 sequence
gcagctgccctggcaggcagct

SEQ ID NO: 1478 - DNA, 18 bp, Artificial
  Mmd_191; mouse brain 12_B04np sequence
tccctgtgtcccttgagc

SEQ ID NO: 1479 - DNA, 22 bp, Artificial
  Mmd_192; mouse mmHP1050998_probe13 sequence
gaattcagaatcaggaagtcat

SEQ ID NO: 1480 - DNA, 18 bp, Artificial
  Mmd_194; mouse brain 18_O06np sequence
tttcctgtccgtttcacc

SEQ ID NO: 1481 - DNA, 22 bp, Artificial
  Mmd_199; mouse mmHP909030_probe18 sequence
gggtgaagaaacctaattatac

SEQ ID NO: 1482 - DNA, 19 bp, Artificial
  Mmd_200; mouse brain 48_O10np sequence
agtgtcttagcttggttgt

SEQ ID NO: 1483 - DNA, 22 bp, Artificial
  Mmd_201; mouse mmHP2117009_probe13 sequence
ggatccaggacaagattctgcg

SEQ ID NO: 1484 - DNA, 22 bp, Artificial
  Mmd_202; mouse cand831_probe34 sequence
tcagtgctctcagtgatgggtg

SEQ ID NO: 1485 - DNA, 22 bp, Artificial
  Mmd_205; mouse mmHP1788264_probe19 sequence
cgctggccaccgagacgtgggt

SEQ ID NO: 1486 - DNA, 22 bp, Artificial
  Mmd_206; mouse cand274_probe30 sequence
ggcccagatgctaagcactgga

SEQ ID NO: 1487 - DNA, 22 bp, Artificial
  Mmd_207; mouse cand689_probe25 sequence
gctgcagcacaactatatccag

SEQ ID NO: 1488 - DNA, 22 bp, Artificial
  Mmd_209; mouse mmHP1378833_probe1 sequence
gcagaaattcttgactcagctc

SEQ ID NO: 1489 - DNA, 22 bp, Artificial
  Mmd_209; mouse mmHP1378833_probe21 sequence
aggagagttaagaaaaatctca

SEQ ID NO: 1490 - DNA, 22 bp, Artificial
  Mmd_210, Mmd_252; mouse mmHP2825016_probe18 sequence
cagtgtccctcttgatggcact

SEQ ID NO: 1491 - DNA, 22 bp, Artificial
  Mmd_213; mouse cand751_probe17 sequence
aggaagccagactgtgtcaaaa

SEQ ID NO: 1492 - DNA, 22 bp, Artificial
  Mmd_214; mouse mmHP432573_probe3 sequence
agggttatctaggagtgatgtc

SEQ ID NO: 1493 - DNA, 22 bp, Artificial
  Mmd_216; mouse mmHP855413_probe3 sequence
aactgttagactatagccaggg

SEQ ID NO: 1494 - DNA, 22 bp, Artificial
  Mmd_218; mouse cand418_probe16 sequence
ggcccttgaagggcagctgaag

SEQ ID NO: 1495 - DNA, 22 bp, Artificial
  Mmd_21; mouse mmHP533267_probe20 sequence
gggaggttaaggatgaaggcct

SEQ ID NO: 1496 - DNA, 22 bp, Artificial
  Mmd_220; mouse cand921_probe13 sequence
tgcagggcctgcagacacattc

SEQ ID NO: 1497 - DNA, 22 bp, Artificial
  Mmd_221; mouse mmHP1210358_probe10 sequence
tcagaataactgccatggaagg

SEQ ID NO: 1498 - DNA, 22 bp, Artificial
  Mmd_224; mouse cand446_probe26 sequence
agggacatcattgtgtcactag

SEQ ID NO: 1499 - DNA, 22 bp, Artificial
  Mmd_22; mouse cand472_probe7 sequence
aggaaggcgtgataatgaggct

SEQ ID NO: 1500 - DNA, 22 bp, Artificial
  Mmd_230; mouse cand330_probe21 sequence
atgcgcgtgagtccccgtgctc

SEQ ID NO: 1501 - DNA, 22 bp, Artificial
  Mmd_233; mouse brain 108_C20 sequence
aatgacaccacatatatggcag

SEQ ID NO: 1502 - DNA, 22 bp, Artificial
  Mmd_234; mouse mmHP1505538_probe23 sequence
ctttaggtcagagtctgggatg

SEQ ID NO: 1503 - DNA, 22 bp, Artificial
  Mmd_238; mouse mmHP1679825_probe38 sequence
taggcctagtctgccagcaagg

SEQ ID NO: 1504 - DNA, 21 bp, Artificial
  Mmd_239; mouse brain 107_M08 sequence
ccgtcctgaggttgttgagct

SEQ ID NO: 1505 - DNA, 20 bp, Artificial
  Mmd_239; mouse brain 116_A12 sequence
ccgtcctgaggttgttgagc

SEQ ID NO: 1506 - DNA, 22 bp, Artificial
  Mmd_239; mouse brain 18_I18 sequence
ccgtcctgaggttgttgagctg

SEQ ID NO: 1507 - DNA, 22 bp, Artificial
  Mmd_241; mouse brain 40_O20np sequence
tgagctggtggtttgggtggtt

SEQ ID NO: 1508 - DNA, 22 bp, Artificial
  Mmd_242; mouse mmHP1170304_probe1 sequence
gccaggcacacacactacaggt

SEQ ID NO: 1509 - DNA, 22 bp, Artificial
  Mmd_244; mouse cand311_probe25 sequence
gaaacatagcctcagtattttc

SEQ ID NO: 1510 - DNA, 22 bp, Artificial
  Mmd_248; mouse cand811_probe37 sequence
tgcacctgatcttttcaagtgc

SEQ ID NO: 1511 - DNA, 22 bp, Artificial
  Mmd_24; mouse cand174_probe40 sequence
catggccaagagggctccatct

SEQ ID NO: 1512 - DNA, 22 bp, Artificial
  Mmd_254; mouse mmHP2341379_probe21 sequence
gggtagttggctgatgggtgtt

SEQ ID NO: 1513 - DNA, 22 bp, Artificial
  Mmd_257; mouse cand511_probe9 sequence
aggaaaaacctgagggaagtct

SEQ ID NO: 1514 - DNA, 18 bp, Artificial
  Mmd_25; mouse brain 60_G19np sequence
tggctgtgtcttagctgg

SEQ ID NO: 1515 - DNA, 18 bp, Artificial
  Mmd_267; mouse brain 27_M18np sequence
gaaagatggtgaactatg

SEQ ID NO: 1516 - DNA, 22 bp, Artificial
  Mmd_268, Mmd_307, Mmd_467; mouse brain 69_H11 sequence
ctctcggcgccccctcgatgct

SEQ ID NO: 1517 - DNA, 22 bp, Artificial
  Mmd_269; mouse cand570_probe12 sequence
agggccctacagagcagcttgt

SEQ ID NO: 1518 - DNA, 18 bp, Artificial
  Mmd_273; mouse brain 35_C09np sequence
ctgcgtccctctcttgtt

SEQ ID NO: 1519 - DNA, 22 bp, Artificial
  Mmd_278; mouse cand384_probe17 sequence
agcagctcgggatcctgggtag

SEQ ID NO: 1520 - DNA, 22 bp, Artificial
  Mmd_27; mouse mmHP744986_probe7 sequence
atttgtaacactaaaaggctcc

SEQ ID NO: 1521 - DNA, 21 bp, Artificial
  Mmd_281; mouse brain 62_N19np sequence
cgcacccttcaagtcctggaa

SEQ ID NO: 1522 - DNA, 19 bp, Artificial
  Mmd_282; mouse brain 1_K03np sequence
tgtgcccctggcttttgtg

SEQ ID NO: 1523 - DNA, 22 bp, Artificial
  Mmd_283; mouse cand680_probe23 sequence
gctaagcaaggcatctcagcac

SEQ ID NO: 1524 - DNA, 22 bp, Artificial
  Mmd_285; mouse cand488_probe10 sequence
gccaagctcaaccttgaagatg

SEQ ID NO: 1525 - DNA, 18 bp, Artificial
  Mmd_287; mouse brain 43_E09 sequence
agggcagaggtgaggagg

SEQ ID NO: 1526 - DNA, 22 bp, Artificial
  Mmd_288; mouse mmHP1540701_probe28 sequence
catatgaggaacctaggactgt

SEQ ID NO: 1527 - DNA, 19 bp, Artificial
  Mmd_289; mouse brain 27_E23np sequence
actggacttggagtcagaa

SEQ ID NO: 1528 - DNA, 22 bp, Artificial
  Mmd_290; mouse mmHP1876696_probe21 sequence
ttcttgtgtgagaagggtaaag

SEQ ID NO: 1529 - DNA, 22 bp, Artificial
  Mmd_292; mouse cand710_probe3 sequence
gggcgagctgcctacgggtccg

SEQ ID NO: 1530 - DNA, 22 bp, Artificial
  Mmd_304; mouse cand467_probe44 sequence
tgcaaggagaatggggcctagg

SEQ ID NO: 1531 - DNA, 18 bp, Artificial
  Mmd_308; mouse brain 1_B02np sequence
ctgtgaccttgacttggt

SEQ ID NO: 1532 - DNA, 22 bp, Artificial
  Mmd_309; mouse mmHP874966_probe9 sequence
ccatgttgggtaactgctgtgc

SEQ ID NO: 1533 - DNA, 21 bp, Artificial
  Mmd_30; mouse brain 95_P09 sequence
cagggcccttgagtcttggtg

SEQ ID NO: 1534 - DNA, 22 bp, Artificial
  Mmd_310; mouse brain 64_B07 sequence
tcagccacggcttacctggaag

SEQ ID NO: 1535 - DNA, 22 bp, Artificial
  Mmd_312; mouse cand852_probe17 sequence
acaacaatttgccgctgccagc

SEQ ID NO: 1536 - DNA, 22 bp, Artificial
  Mmd_312; mouse cand852_probe3 sequence
aggaggcggaaagccgacattg

SEQ ID NO: 1537 - DNA, 22 bp, Artificial
  Mmd_313; mouse cand60_probe26 sequence
cagctgaaagggtttctctagt

SEQ ID NO: 1538 - DNA, 20 bp, Artificial
  Mmd_315; mouse brain 76_N24 sequence
tgggccctccagacctcatg

SEQ ID NO: 1539 - DNA, 22 bp, Artificial
  Mmd_317; mouse cand608_probe16 sequence
gtttttacagcagtcacacagg

SEQ ID NO: 1540 - DNA, 22 bp, Artificial
  Mmd_320; mouse mmHP325598_probe5 sequence
tcatctggtacctattgtgctc

SEQ ID NO: 1541 - DNA, 22 bp, Artificial
  Mmd_327; mouse mmHP527107_probe22 sequence
tgggtttcccctttgatgggtc

SEQ ID NO: 1542 - DNA, 23 bp, Artificial
  Mmd_329; mouse brain 1_O13 sequence
tttggggtcgccttccagagggt

SEQ ID NO: 1543 - DNA, 22 bp, Artificial
  Mmd_32; mouse cand756_probe40 sequence
gtgagccagggccctgtggaga

SEQ ID NO: 1544 - DNA, 22 bp, Artificial
  Mmd_331; mouse cand460_probe7 sequence
aaggagcaaccaggcagggaga

SEQ ID NO: 1545 - DNA, 22 bp, Artificial
  Mmd_332; mouse mmHP2109324_probe14 sequence
gaccaggagcttctccgtaact

SEQ ID NO: 1546 - DNA, 22 bp, Artificial
  Mmd_337; mouse cand246_probe3 sequence
aggagaggaatgacccataact

SEQ ID NO: 1547 - DNA, 22 bp, Artificial
  Mmd_339; mouse cand434_probe1 sequence
aggatgatgtcatctatctccc

SEQ ID NO: 1548 - DNA, 22 bp, Artificial
  Mmd_339; mouse cand434_probe18 sequence
ggaagtccatggtgacatcacc

SEQ ID NO: 1549 - DNA, 22 bp, Artificial
  Mmd_341; mouse cand86_probe20 sequence
gcagagagccaaggccaaagct

SEQ ID NO: 1550 - DNA, 22 bp, Artificial
  Mmd_342; mouse cand403_probe29 sequence
ctgaggagcacaaatgtcacaa

SEQ ID NO: 1551 - DNA, 19 bp, Artificial
  Mmd_345, Mmd_38; mouse brain 61_M12np sequence
ggaggtctctgtctgactt

SEQ ID NO: 1552 - DNA, 22 bp, Artificial
  Mmd_348; mouse cand596_probe10 sequence
aggatccccttagtccctcccg

SEQ ID NO: 1553 - DNA, 22 bp, Artificial
  Mmd_348; mouse cand596_probe12 sequence
ccggagaggacagggggctttt

SEQ ID NO: 1554 - DNA, 22 bp, Artificial
  Mmd_349; mouse cand690_probe14 sequence
ataggacaagtgagggagacaa

SEQ ID NO: 1555 - DNA, 22 bp, Artificial
  Mmd_350; mouse cand264_probe16 sequence
ataacagagggcagaatttcgc

SEQ ID NO: 1556 - DNA, 22 bp, Artificial
  Mmd_350; mouse cand264_probe4 sequence
ggccaaattctgccctcaatta

SEQ ID NO: 1557 - DNA, 22 bp, Artificial
  Mmd_351; mouse cand631_probe10 sequence
gcagacaccagccacagccttc

SEQ ID NO: 1558 - DNA, 22 bp, Artificial
  Mmd_352; mouse cand21_probe20 sequence
gcgcagggtctctggtgtttcc

SEQ ID NO: 1559 - DNA, 18 bp, Artificial
  Mmd_357; mouse brain 54_F07np sequence
ggctatctagctgtggtg

SEQ ID NO: 1560 - DNA, 22 bp, Artificial
  Mmd_359; mouse mmHP486329_probe19 sequence
ggggtcctggcttctccgctct

SEQ ID NO: 1561 - DNA, 22 bp, Artificial
  Mmd_360; mouse mmHP811508_probe6 sequence
gcagaaggccatttgaaaattt

SEQ ID NO: 1562 - DNA, 22 bp, Artificial
  Mmd_364; mouse mmHP1290061_probe10 sequence
cacccgatcccaggtctgtgct

SEQ ID NO: 1563 - DNA, 22 bp, Artificial
  Mmd_365; mouse cand682_probe29 sequence
aggactggatgtgttcatgttt

SEQ ID NO: 1564 - DNA, 22 bp, Artificial
  Mmd_365; mouse cand682_probe37 sequence
catgagaatccgcccagtcgta

SEQ ID NO: 1565 - DNA, 22 bp, Artificial
  Mmd_366; mouse cand390_probe17 sequence
gcaaggtaagtgtcctatgtga

SEQ ID NO: 1566 - DNA, 22 bp, Artificial
  Mmd_368; mouse mmHP2418251_probe5 sequence
ccagtggacactggtgcgcacc

SEQ ID NO: 1567 - DNA, 22 bp, Artificial
  Mmd_369; mouse cand399_probe17 sequence
acaaagcaatttcacagttatc

SEQ ID NO: 1568 - DNA, 22 bp, Artificial
  Mmd_369; mouse cand399_probe3 sequence
ctctcgctgtggttatgtgtct

SEQ ID NO: 1569 - DNA, 22 bp, Artificial
  Mmd_36; mouse cand566_probe27 sequence
gcaggattccttgctagcattt

SEQ ID NO: 1570 - DNA, 22 bp, Artificial
  Mmd_374; mouse cand273_probe11 sequence
ggcactggccaagccaaagtca

SEQ ID NO: 1571 - DNA, 22 bp, Artificial
  Mmd_375; mouse cand580_probe18 sequence
gctcagggtgattattttgctg

SEQ ID NO: 1572 - DNA, 22 bp, Artificial
  Mmd_377; mouse mmHP1218232_probe15 sequence
aaggactcctcgtccttaagtt

SEQ ID NO: 1573 - DNA, 22 bp, Artificial
  Mmd_379; mouse cand70_probe16 sequence
gtgaatcacagagagcgagcca

SEQ ID NO: 1574 - DNA, 21 bp, Artificial
  Mmd_380; mouse brain 77_B20 sequence
taggctagagagaggttgggg

SEQ ID NO: 1575 - DNA, 18 bp, Artificial
  Mmd_381; mouse brain 91_A17 sequence
tggctcatttagaagcag

SEQ ID NO: 1576 - DNA, 22 bp, Artificial
  Mmd_384; mouse mmHP2197503_probe16 sequence
agcctccaaaaagaaataatga

SEQ ID NO: 1577 - DNA, 18 bp, Artificial
  Mmd_385; mouse brain 105_C04np sequence
tagtaggtctgtatggtt

SEQ ID NO: 1578 - DNA, 21 bp, Artificial
  Mmd_38; mouse brain 14_I18 sequence
tctgatcgttcacctccatac

SEQ ID NO: 1579 - DNA, 19 bp, Artificial
  Mmd_38; mouse brain 75_N06 sequence
tctgatcgttcacctccat

SEQ ID NO: 1580 - DNA, 22 bp, Artificial
  Mmd_390; mouse mmHP3025219_probe1 sequence
aggaatgggatacattcacagg

SEQ ID NO: 1581 - DNA, 21 bp, Artificial
  Mmd_392; mouse brain 63_M16 sequence
ctaaggcaggcagacttcagt

SEQ ID NO: 1582 - DNA, 20 bp, Artificial
  Mmd_392; mouse brain 95_D02 sequence
tctgaagcttgcttacctcc

SEQ ID NO: 1583 - DNA, 22 bp, Artificial
  Mmd_393; mouse cand12_probe10 sequence
tctgtaactggcatgtaaggga

SEQ ID NO: 1584 - DNA, 22 bp, Artificial
  Mmd_394; mouse mmHP2549249_probe15 sequence
gctgcggagttgggccagctct

SEQ ID NO: 1585 - DNA, 22 bp, Artificial
  Mmd_394; mouse mmHP2549249_probe8 sequence
tgctggatccagttgcagctga

SEQ ID NO: 1586 - DNA, 20 bp, Artificial
  Mmd_395; mouse brain 8_M05np sequence
ctagttggatgtcaagacac

SEQ ID NO: 1587 - DNA, 18 bp, Artificial
  Mmd_397; mouse brain 72_K08np sequence
tgcctgaggttgtgtgag

SEQ ID NO: 1588 - DNA, 22 bp, Artificial
  Mmd_400; mouse cand516_probe21 sequence
gtgacgtcagcggagcgggcgc

SEQ ID NO: 1589 - DNA, 18 bp, Artificial
  Mmd_401; mouse brain 3_O07np sequence
tgcgtcccttgcttgtgg

SEQ ID NO: 1590 - DNA, 20 bp, Artificial
  Mmd_402; mouse brain 5_C21np sequence
tcctggcctgggctgctctg

SEQ ID NO: 1591 - DNA, 22 bp, Artificial
  Mmd_403; mouse cand978_probe16 sequence
tgctactattagacaggccgtt

SEQ ID NO: 1592 - DNA, 22 bp, Artificial
  Mmd_403; mouse cand978_probe3 sequence
aggatggtggtcttggaggcgg

SEQ ID NO: 1593 - DNA, 22 bp, Artificial
  Mmd_404; mouse mmHP1595839_probe41 sequence
cttatgggtcaatgtggtcaag

SEQ ID NO: 1594 - DNA, 22 bp, Artificial
  Mmd_406; mouse cand484_probe5 sequence
ggccagttccccgcctcttcca

SEQ ID NO: 1595 - DNA, 22 bp, Artificial
  Mmd_407; mouse cand703_probe4 sequence
cgagggagcaattattcgcatc

SEQ ID NO: 1596 - DNA, 18 bp, Artificial
  Mmd_414; mouse brain 10_O20np sequence
cagcagcacactgtgagt

SEQ ID NO: 1597 - DNA, 22 bp, Artificial
  Mmd_417; mouse cand193_probe30 sequence
gctagctttgcagaaagggact

SEQ ID NO: 1598 - DNA, 22 bp, Artificial
  Mmd_419; mouse mmHP683_probe18 sequence
tcggggcacctgcacatttgca

SEQ ID NO: 1599 - DNA, 22 bp, Artificial
  Mmd_420, Mmd_513; mouse brain 116_B14 sequence
agttgtgtgtgcatgttcatgt

SEQ ID NO: 1600 - DNA, 22 bp, Artificial
  Mmd_423; mouse cand408_probe2 sequence
ctggctgcagcaatgagggcct

SEQ ID NO: 1601 - DNA, 22 bp, Artificial
  Mmd_423; mouse cand408_probe22 sequence
acactccttgctgctggccagc

SEQ ID NO: 1602 - DNA, 20 bp, Artificial
  Mmd_425; mouse brain 67_J02 sequence
tgctctccctggctgggggt

SEQ ID NO: 1603 - DNA, 22 bp, Artificial
  Mmd_427; mouse mmHP1499491_probe4 sequence
aggccttaaggcctgtgggcac

SEQ ID NO: 1604 - DNA, 22 bp, Artificial
  Mmd_432; mouse cand134_probe27 sequence
catgctgtctggctcaatgagg

SEQ ID NO: 1605 - DNA, 22 bp, Artificial
  Mmd_432; mouse cand134_probe34 sequence
aggagccaaagccacgggacag

SEQ ID NO: 1606 - DNA, 22 bp, Artificial
  Mmd_434; mouse mmHP2810075_probe14 sequence
aggggaggctaaagtgggtttg

SEQ ID NO: 1607 - DNA, 18 bp, Artificial
  Mmd_440; mouse brain 51_A03 sequence
gaacccttcgtgtatgtc

SEQ ID NO: 1608 - DNA, 20 bp, Artificial
  Mmd_441; mouse brain 100_M10 sequence
aagggattctgatgttggtc

SEQ ID NO: 1609 - DNA, 22 bp, Artificial
  Mmd_441; mouse brain 103_I17 sequence
aagggattctgatgttggtcac

SEQ ID NO: 1610 - DNA, 26 bp, Artificial
  Mmd_441; mouse brain 109_H12 sequence
aagggattctgatgttggtcacactc

SEQ ID NO: 1611 - DNA, 25 bp, Artificial
  Mmd_441; mouse brain 109_J22 sequence
aagggattctgatgttggtcacact

SEQ ID NO: 1612 - DNA, 24 bp, Artificial
  Mmd_441; mouse brain 48_C24 sequence
aagggattctgatgttggtcacac

SEQ ID NO: 1613 - DNA, 19 bp, Artificial
  Mmd_441; mouse brain 52_L05 sequence
aagggattctgatgttggt

SEQ ID NO: 1614 - DNA, 23 bp, Artificial
  Mmd_441; mouse brain 66_P22np sequence
gggattctgatgttggtcacact

SEQ ID NO: 1615 - DNA, 24 bp, Artificial
  Mmd_441; mouse brain 86_C01 sequence
agggattctgatgttggtcacact

SEQ ID NO: 1616 - DNA, 22 bp, Artificial
  Mmd_442; mouse mmHP1626774_probe9 sequence
gactaacagctttagcgcagga

SEQ ID NO: 1617 - DNA, 22 bp, Artificial
  Mmd_444; mouse mmHP565280_probe15 sequence
gagtcctcaggagtatagctgt

SEQ ID NO: 1618 - DNA, 22 bp, Artificial
  Mmd_445; mouse cand567_probe2 sequence
agggaactacaactcccagtgt

SEQ ID NO: 1619 - DNA, 22 bp, Artificial
  Mmd_445; mouse cand761_probe23 sequence
aagggaactacaactcccagtg

SEQ ID NO: 1620 - DNA, 22 bp, Artificial
  Mmd_446; mouse cand560_probe44 sequence
ttgtcacaaacagaggattaga

SEQ ID NO: 1621 - DNA, 22 bp, Artificial
  Mmd_446; mouse cand843a_probe19 sequence
atgttgtcacaaacagaggatt

SEQ ID NO: 1622 - DNA, 22 bp, Artificial
  Mmd_447; mouse cand327_probe37 sequence
caaaggagtaaatgagaggtcc

SEQ ID NO: 1623 - DNA, 22 bp, Artificial
  Mmd_449; mouse mmHP693133_probe7 sequence
ccctcataccttgagggacatg

SEQ ID NO: 1624 - DNA, 18 bp, Artificial
  Mmd_451; mouse brain 72_M07 sequence
agcgggcacagctgtgag

SEQ ID NO: 1625 - DNA, 22 bp, Artificial
  Mmd_453; mouse mmHP1778980_probe15 sequence
ggctccggagttgggaaaggga

SEQ ID NO: 1626 - DNA, 22 bp, Artificial
  Mmd_453; mouse mmHP1778980_probe5 sequence
ctgcctcccaatcctggctcag

SEQ ID NO: 1627 - DNA, 22 bp, Artificial
  Mmd_454; mouse mmHP3397717_probe17 sequence
agggacaatgtgttggctgtgt

SEQ ID NO: 1628 - DNA, 22 bp, Artificial
  Mmd_457; mouse mmHP2347_probe4 sequence
ccggaagcctatggtaatcaga

SEQ ID NO: 1629 - DNA, 21 bp, Artificial
  Mmd_458; mouse brain 31_P17 sequence
tggatggtaataatgctgagt

SEQ ID NO: 1630 - DNA, 18 bp, Artificial
  Mmd_460; mouse brain 76_E12np sequence
acattgtctgctgagttt

SEQ ID NO: 1631 - DNA, 22 bp, Artificial
  Mmd_462; mouse mmHP1121223_probe5 sequence
gctcgaggctaattgtgaatta

SEQ ID NO: 1632 - DNA, 22 bp, Artificial
  Mmd_46; mouse mmHP3140720_probe6 sequence
gggatttgggtcagtcaatcag

SEQ ID NO: 1633 - DNA, 22 bp, Artificial
  Mmd_474; mouse cand55_probe43 sequence
ccagctgacggaggaatcggtc

SEQ ID NO: 1634 - DNA, 22 bp, Artificial
  Mmd_475; mouse cand411_probe13 sequence
gccctgatgcactctttcaggg

SEQ ID NO: 1635 - DNA, 22 bp, Artificial
  Mmd_479; mouse cand655_probe29 sequence
gcagcagctgccggatctcggc

SEQ ID NO: 1636 - DNA, 21 bp, Artificial
  Mmd_484; mouse brain 101_B22 sequence
aaggttacttgttagttcagg

SEQ ID NO: 1637 - DNA, 20 bp, Artificial
  Mmd_484; mouse brain 15_J05 sequence
aggttacttgttagttcagg

SEQ ID NO: 1638 - DNA, 18 bp, Artificial
  Mmd_484; mouse brain 30_J21 sequence
aaggttacttgttagttc

SEQ ID NO: 1639 - DNA, 20 bp, Artificial
  Mmd_484; mouse brain 88_K04 sequence
aaggttacttgttagttcag

SEQ ID NO: 1640 - DNA, 22 bp, Artificial
  Mmd_493; mouse mmHP3423784_probe36 sequence
cagagggacatcacccccaagt

SEQ ID NO: 1641 - DNA, 22 bp, Artificial
  Mmd_495; mouse mmHP200292_probe14 sequence
ctgcagtgggtgtggagaacat

SEQ ID NO: 1642 - DNA, 22 bp, Artificial
  Mmd_496; mouse mmHP2299700_probe14 sequence
gcagagtggctgacccgggatg

SEQ ID NO: 1643 - DNA, 22 bp, Artificial
  Mmd_497; mouse cand451_probe7 sequence
agcctgggctaagatgcagtaa

SEQ ID NO: 1644 - DNA, 22 bp, Artificial
  Mmd_498; mouse cand145_probe42 sequence
gcagggaggaaaggtggccgct

SEQ ID NO: 1645 - DNA, 22 bp, Artificial
  Mmd_502; mouse brain 13_F04 sequence
ctcacagctctggtccttggag

SEQ ID NO: 1646 - DNA, 20 bp, Artificial
  Mmd_502; mouse brain 69_K13 sequence
ctcacagctctggtccttgg

SEQ ID NO: 1647 - DNA, 22 bp, Artificial
  Mmd_508; mouse cand801_probe10 sequence
acagatgggtaatacccagaaa

SEQ ID NO: 1648 - DNA, 18 bp, Artificial
  Mmd_50; mouse brain 58_N11np sequence
tgtgtcttagcttggttg

SEQ ID NO: 1649 - DNA, 22 bp, Artificial
  Mmd_512; mouse mmHP668435_probe3 sequence
cttgcctttgatgtgaggcaag

SEQ ID NO: 1650 - DNA, 20 bp, Artificial
  Mmd_514; mouse brain 11_B21 sequence
gcactgagatgggagtggtg

SEQ ID NO: 1651 - DNA, 20 bp, Artificial
  Mmd_514; mouse brain 34_M13 sequence
cacagctcccatctcagaac

SEQ ID NO: 1652 - DNA, 21 bp, Artificial
  Mmd_514; mouse brain 78_H11np sequence
gcactgagatgggagtggtgt

SEQ ID NO: 1653 - DNA, 22 bp, Artificial
  Mmd_520; mouse cand627_probe2 sequence
cagctgatggatcacaaatctc

SEQ ID NO: 1654 - DNA, 22 bp, Artificial
  Mmd_521; mouse mmHP1528040_probe19 sequence
gacagtcaggagctgtggaagc

SEQ ID NO: 1655 - DNA, 22 bp, Artificial
  Mmd_528; mouse mmHP166179_probe15 sequence
cgcgagtagcgatgaggagctc

SEQ ID NO: 1656 - DNA, 22 bp, Artificial
  Mmd_531; mouse mmHP141642_probe4 sequence
agctagcgtgggcagttctttt

SEQ ID NO: 1657 - DNA, 22 bp, Artificial
  Mmd_53; mouse mmHP3172562_probe11 sequence
ttgagtgtcaaaagttggagtt

SEQ ID NO: 1658 - DNA, 22 bp, Artificial
  Mmd_55; mouse brain 105_K22 sequence
tgttgagcccagcctgccctgc

SEQ ID NO: 1659 - DNA, 22 bp, Artificial
  Mmd_57; mouse mmHP1485966_probe13 sequence
cccagaaacgcaaaggacagtg

SEQ ID NO: 1660 - DNA, 22 bp, Artificial
  Mmd_61; mouse mmHP2157333_probe14 sequence
tgaagttgcttgagtttcagta

SEQ ID NO: 1661 - DNA, 22 bp, Artificial
  Mmd_62; mouse cand398_probe5 sequence
cgaggtgatgatggaggcttct

SEQ ID NO: 1662 - DNA, 22 bp, Artificial
  Mmd_63; mouse cand457_probe32 sequence
ggccagtgtggtttctgtcctc

SEQ ID NO: 1663 - DNA, 22 bp, Artificial
  Mmd_64; mouse cand789a_probe20 sequence
atgtcaccagaactctccatgg

SEQ ID NO: 1664 - DNA, 20 bp, Artificial
  Mmd_65; mouse brain 13_L04np sequence
gtagcaggttgtgtggtttt

SEQ ID NO: 1665 - DNA, 22 bp, Artificial
  Mmd_66; mouse cand938_probe28 sequence
catgagagtgatgcagggaatg

SEQ ID NO: 1666 - DNA, 18 bp, Artificial
  Mmd_72; mouse brain 79_E14np sequence
ttcttgtcatgttcttcc

SEQ ID NO: 1667 - DNA, 22 bp, Artificial
  Mmd_73; mouse cand715_probe23 sequence
atggatgaggggctgcggctcg

SEQ ID NO: 1668 - DNA, 22 bp, Artificial
  Mmd_77; mouse cand483_probe2 sequence
aaactgttatttaaagcagggt

SEQ ID NO: 1669 - DNA, 22 bp, Artificial
  Mmd_77; mouse cand483_probe21 sequence
gtgtttttaaaactcaggatgg

SEQ ID NO: 1670 - DNA, 23 bp, Artificial
  Mmd_79; mouse brain 100_J16 sequence
tgacacctgccacccagcccaag

SEQ ID NO: 1671 - DNA, 21 bp, Artificial
  Mmd_79; mouse brain 105_A12np sequence
tgacacctgccacccagccca

SEQ ID NO: 1672 - DNA, 20 bp, Artificial
  Mmd_79; mouse brain 114_E09 sequence
tgacacctgccacccagccc

SEQ ID NO: 1673 - DNA, 22 bp, Artificial
  Mmd_79; mouse brain 70_D05 sequence
gacacctgccacccagcccaag

SEQ ID NO: 1674 - DNA, 22 bp, Artificial
  Mmd_83; mouse mmHP2052148_probe11 sequence
actgtaggagggctgacagcat

SEQ ID NO: 1675 - DNA, 22 bp, Artificial
  Mmd_84; mouse cand6_probe6 sequence
ggagagatgctgggggtggggg

SEQ ID NO: 1676 - DNA, 21 bp, Artificial
  Mmd_86; mouse brain 103_B05np sequence
gtgggcctgacgtggagctgg

SEQ ID NO: 1677 - DNA, 22 bp, Artificial
  Mmd_86; mouse brain 117_K04 sequence
cgtgggcctgacgtggagctgg

SEQ ID NO: 1678 - DNA, 20 bp, Artificial
  Mmd_86; mouse brain 30_D11 sequence
tgggcctgacgtggagctgg

SEQ ID NO: 1679 - DNA, 22 bp, Artificial
  Mmd_86; mouse brain 61_L02np sequence
agcaccacgtgtctgggccacg

SEQ ID NO: 1680 - DNA, 20 bp, Artificial
  Mmd_86; mouse brain 82_N13np sequence
gtgggcctgacgtggagctg

SEQ ID NO: 1681 - DNA, 23 bp, Artificial
  Mmd_86; mouse brain 86_J05np sequence
agcaccacgtgtctgggccacgt

SEQ ID NO: 1682 - DNA, 20 bp, Artificial
  Mmd_86; mouse brain 86_O04 sequence
cgtgggcctgacgtggagct

SEQ ID NO: 1683 - DNA, 22 bp, Artificial
  Mmd_87; mouse mmHP1815876_probe21 sequence
tgatggcactttggacatgacc

SEQ ID NO: 1684 - DNA, 22 bp, Artificial
  Mmd_88; mouse cand279_probe13 sequence
cctggcagcgttgccatggaga

SEQ ID NO: 1685 - DNA, 25 bp, Artificial
  Mmd_89; mouse brain 51_M21 sequence
gtctagtctgccaactcctctacag

SEQ ID NO: 1686 - DNA, 22 bp, Artificial
  Mmd_99; mouse mmHP1974380_probe11 sequence
atttattttgttctcatgagaa

SEQ ID NO: 1687 - DNA, 100 bp, Artificial
  Hairpin sequence Mmd_1, dog
ggttgggcagggtgcggggctagggctaacagcagtcttactgagggtttcttggaaaccacacacatgctgttgccactaacctcaaccttactcggtc

SEQ ID NO: 1688 - DNA, 100 bp, Artificial
  Hairpin sequence Mmd_1, cow
ggttgggcggggtgcggggctagggctaacagcaggctcactgacggtttcccggaaaccacacacatgctgttgccactaacctcaaccttactcggtc

(19) United States
(12) Patent Application Publication
(10) Pub. No.: US 2009/0215865 A1
(43) Pub. Date: Aug. 27, 2009

(54) Nucleic Acid Molecules and Collections Thereof, Their Application and Identification

(76) Inventors: Ronald H.A. Plasterk, Bussum (NL); Eugene Berezikov, Vleuten (NL); Edwin P.J.G. Cuppen, Utrecht (NL)

Correspondence Address: TRASKBRITT, P.C., P.O. BOX 2550, SALT LAKE CITY, UT, 84110, US

(21) Appl. No.: 12/087,566

(22) PCT Filed: Jan. 10, 2006
(86) PCT No.: PCT/IL2006/000010

§ 371 Date: Jan. 26, 2009

(51) Int. Cl. A61K 31/7105; G01N 33/00; C12Q 1/68; C07H 21/02; C12N 15/00; C40B 40/08

(52) U.S. Cl. 514/44 A; 436/63; 435/6; 536/23.1; 435/320.1; 536/24.31; 506/17

(57) ABSTRACT

The invention provides a method for characterising a sample comprising nucleic acid derived from a cell. The method comprises determining whether a sample comprises at least a minimal sequence of at least one new microRNA (miRNA) according to the invention or a mammalian ortholog thereof and characterizing the sample on the basis of the presence or absence of the miRNA. The invention further provides new nucleic acid molecules and collections thereof and their use in therapeutic and diagnostic applications. The invention furthermore provides a method for identifying a miRNA molecule or a precursor molecule thereof.

The invention relates to nucleic acid molecules and collections thereof. The invention further relates to the use of nucleic acid molecules in therapeutic and diagnostic applications. The invention furthermore relates to a method for identifying a miRNA molecule or a precursor molecule thereof.

MicroRNAs (miRNAs) are non-coding RNAs that regulate the expression of genes at the post-transcriptional level (reviewed in Bartel, 2004). Although only recently discovered, they have been found to play key roles in a wide variety of biological processes, including cell fate specification, cell death, proliferation, and fat storage (Brennecke, 2003, Poy et al., 2004, reviewed in Ambros, 2004). About 200 different miRNAs have now been described for mouse and human (Griffiths-Jones, 2004). The molecular requirements and mechanism by which miRNAs regulate gene expression are currently being clarified (Bartel, 2004), but individual biological functions remain largely unknown. Temporal and spatial expression of miRNAs may be key features driving cellular specificity.

MiRNAs, like siRNAs, are known in the context of RNA interference (RNAi). RNAi is the silencing of gene expression by the administration of double-stranded RNA (dsRNA). Endogenous RNAi seems to be a primitive sort of immune system, aimed at the defense of genomes against molecular parasites like viruses and transposons. During the process of RNAi, the dsRNA is converted into a shorter form: the siRNAs. siRNA is shorthand for “short interfering RNA”, and synthetic versions of these 21 nucleotide long molecules are widely used to induce RNAi in mammalian cell systems because they circumvent the aspecific interferon response of these cells to dsRNA. The miRNAs are another species of small RNA molecules. MiRNAs, however, are always encoded by the genome itself, as hairpin structures, whereas siRNAs can both be artificial as well as endogenous (Hamilton & Baulcombe 1999; Aravin et al, 2001; Reinhart & Bartel 2002; Ambros et al, 2003). Both molecules feed largely into one and the same process that can either lead to mRNA degradation or to the inhibition of protein synthesis. As a rule, siRNAs cause mRNA destruction, whereas miRNAs can do both: in plants the majority of miRNAs direct cleavage, whereas miRNAs in animals most often induce translation inhibition; however, examples of translation inhibition in plants and cleavage in animals have been found (Chen 2004; Yekta et al, 2004).

MiRNA genes are transcribed by RNA polymerase II and transcripts are subsequently capped and poly-adenylated (Cai et al., 2004). Therefore, expression patterns of miRNAs in C. elegans can be easily determined by fusing green fluorescent protein (GFP) to upstream sequences (Johnson et al, 2003; Johnston & Hobert 2003). The nascent transcript of the miRNA is named pri-miRNA (primary mRNA) and can contain more than one miRNA. The individual miRNA-containing hairpin precursor (or pre-miRNA) is excised from this pri-miRNA by the enzyme Drosha (Lee et al, 2003) in the nucleus, and is assisted by a dsRNA-binding protein, gripper (G. Hannon, Cold Spring Harbor, N.Y., USA). Drosha is an animal-specific RNaseIII enzyme, and is essential for the production of miRNA precursor structures that can be exported from the nucleus. In plants, this role appears to be taken by one of the Dicer homologues (DCL1; Park et al, 2002; Reinhart et al, 2002; Xie et al, 2004).

The pre-miRNA is then exported to the cytosol (Yi et al, 2003; Bohnsack et al, 2004; Lund et al, 2004), where it is further processed by Dicer (Grishok et al, 2001; Hutvagner et al, 2001; Ketting et al, 2001). This enzyme basically can take any dsRNA and convert it to si/miRNAs (Bernstein et al, 2001) and there have been many models for how this is achieved. However, now it seems clear that the human Dicer enzyme does so by binding, as a monomer, to one end of the dsRNA through the PAZ (=Piwi-Argonaute-ZwiLle) domain (Lingel et al, 2003; Song et al, 2003; Yan et al, 2003), which seems to specifically recognize dsRNA ends produced by RNaseIII enzymes (Ma et al, 2004). This positions the two RNaseIII domains of the Dicer monomer such that they form one active site approximately 21 basepairs away (Zhang et al, 2004). In the case of miRNAs, this mode of action usually leads to the production of only one miRNA of specific sequence, as only the paired end of the pre-miRNA hairpin can be recognized. The mode of action of production of miRNAs from pre-miRNAs is unpredictable in that specific miRNAs cannot be predicted on the basis of the nucleic acid sequence of the pre-miRNA.

The complex that is ultimately responsible for silencing has been named the RNA-induced silencing complex (RISC), which incorporates both si- and miRNAs. Only single-stranded RNA is incorporated, however, and which of the two strands makes it into RISC is determined by the thermodynamically asymmetric nature of the siRNA: the strand with the most loosely basepaired 5′ end is in most cases incorporated (Khvorova et al, 2003; Schwarz et al, 2003). P. Zamore (Worcester, Mass., USA) reported that this asymmetry is sensed by Dicer in complex with the dsRNA-binding protein R2D2, which literally takes this strand to the RISC complex (Lee et al, 2004; Pham et al, 2004; Tomari et al, 2004). What happens next is determined by a combination of factors: the origin of the small RNA (that is, whether it has been processed by Drosha and/or Dicer), associated proteins and the extent of basepairing between the target mRNA and the si/miRNA.

One of the outcomes is cleavage of the mRNA. The protein that executes this cleavage (“Slicer”) remains elusive, but it is known what chemistry this enzyme should use: a 3′ hydroxyl and a 5′ phosphate group characterize the cleavage product (Martinez & Tuschl 2004; Schwarz et al, 2004). Also, RISC behaves like a true enzyme, so it catalyses many rounds of cleavage. The other outcome, translation inhibition, is not completely elucidated either. The step of translation that is actually inhibited could be initiation and/or elongation. Alternatively the process of translation could not be inhibited at all. One way of translational silencing might involve nascent chain degradation.

Currently, about 200 different mammalian miRNAs are known. A published estimate of the total number of miRNA genes in the human genome has been that the human genome contains at most 255 miRNA genes (Lim et al., 2003). The invention surprisingly found that there are many more different miRNA expressed in mammalian cells. At least ˜1000 putative miRNAs in the human genome are conserved in at least some other vertebrates, and there are also a substantial number of species-specific miRNAs.

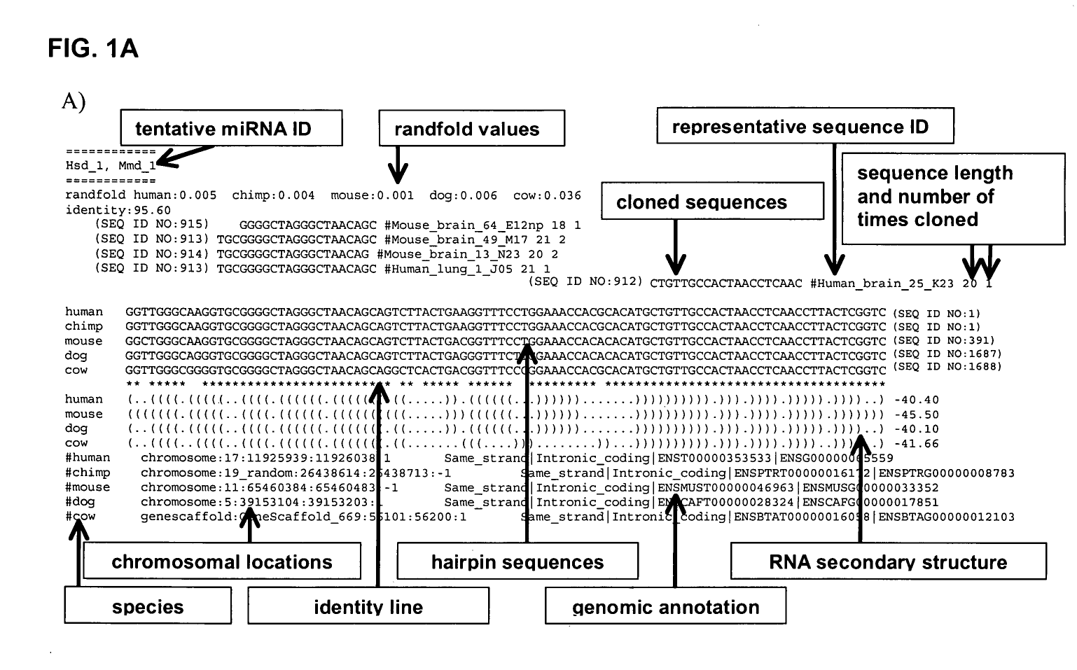

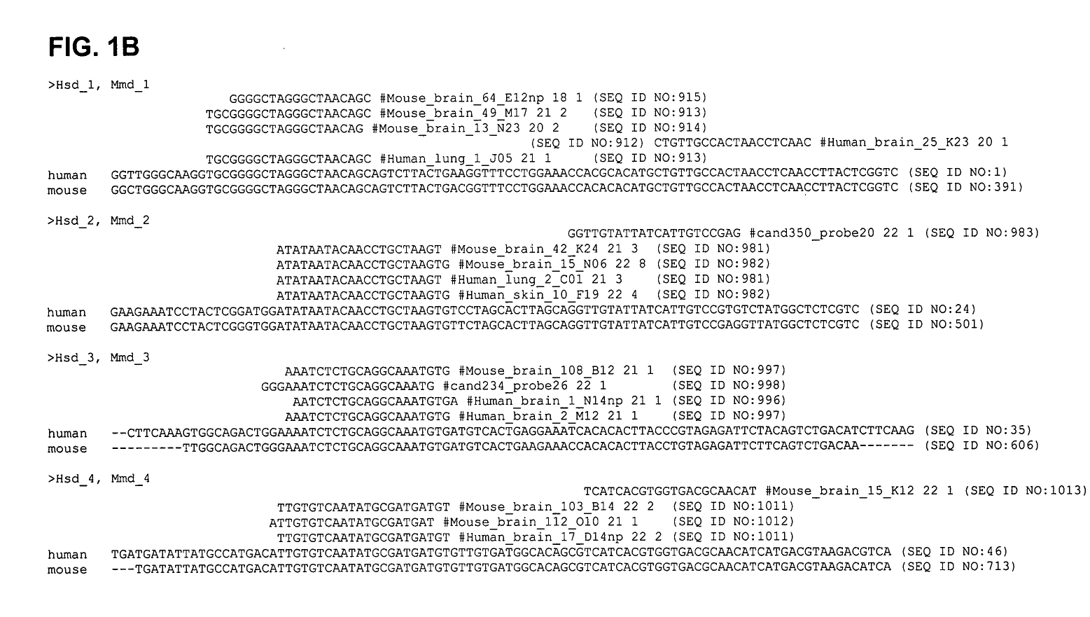

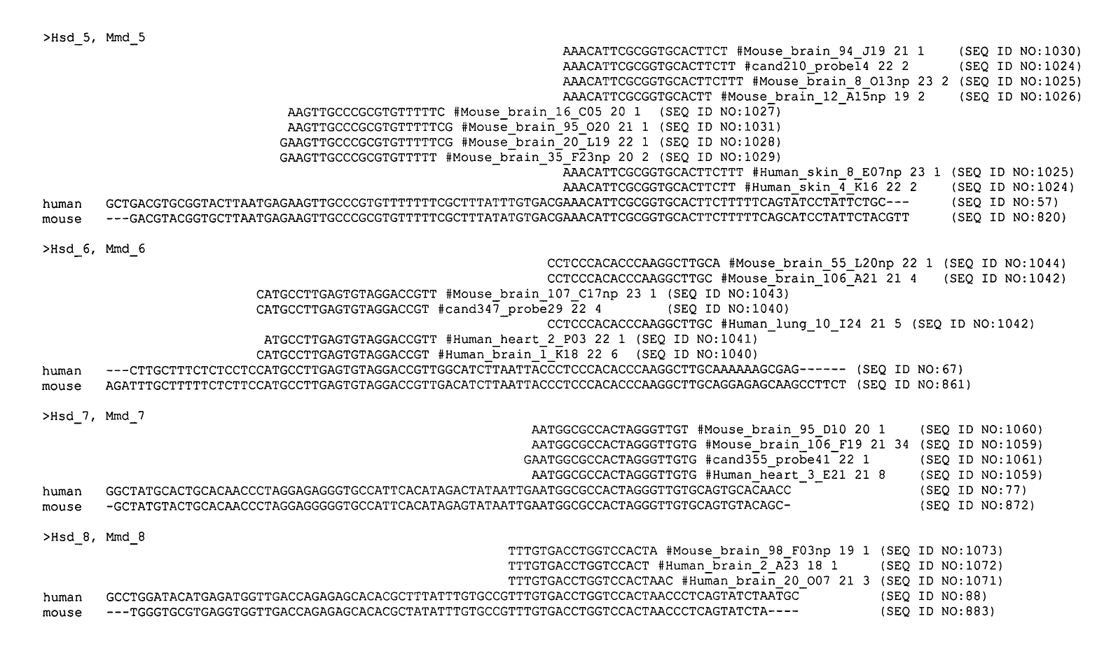

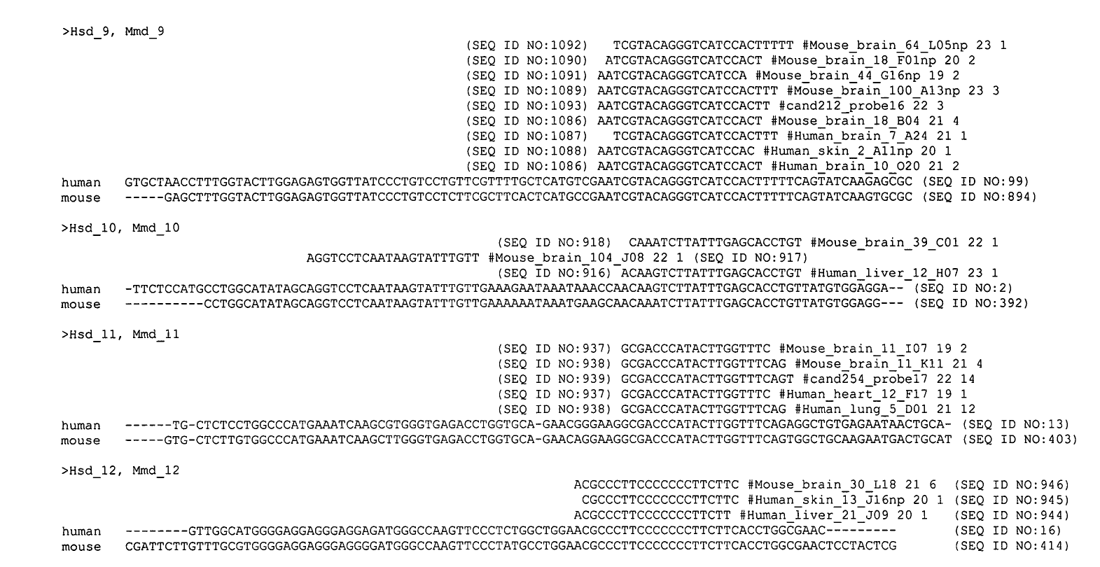

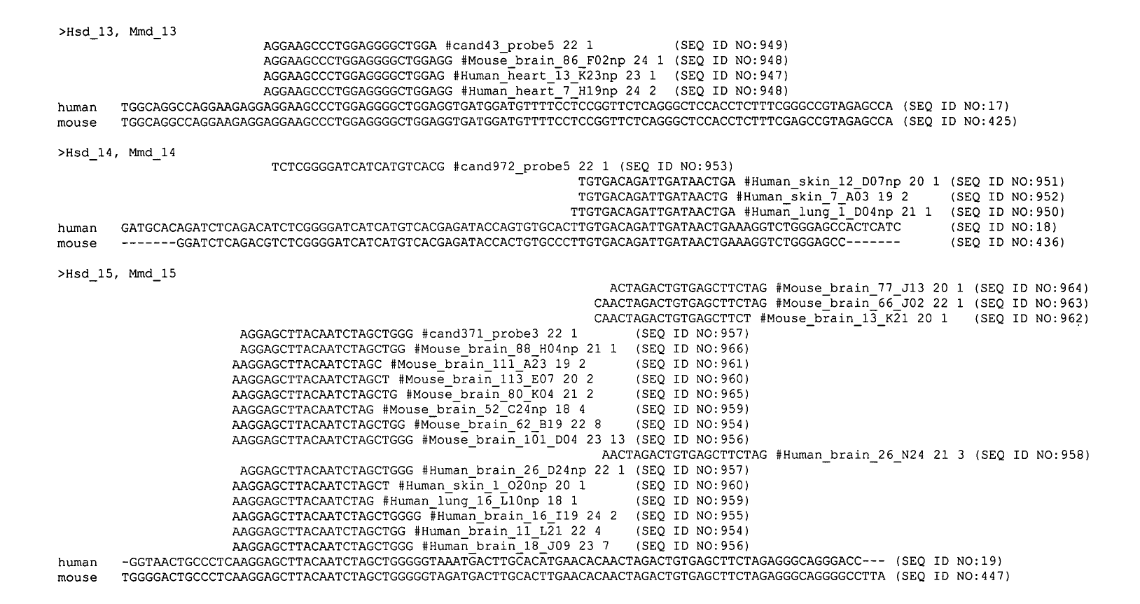

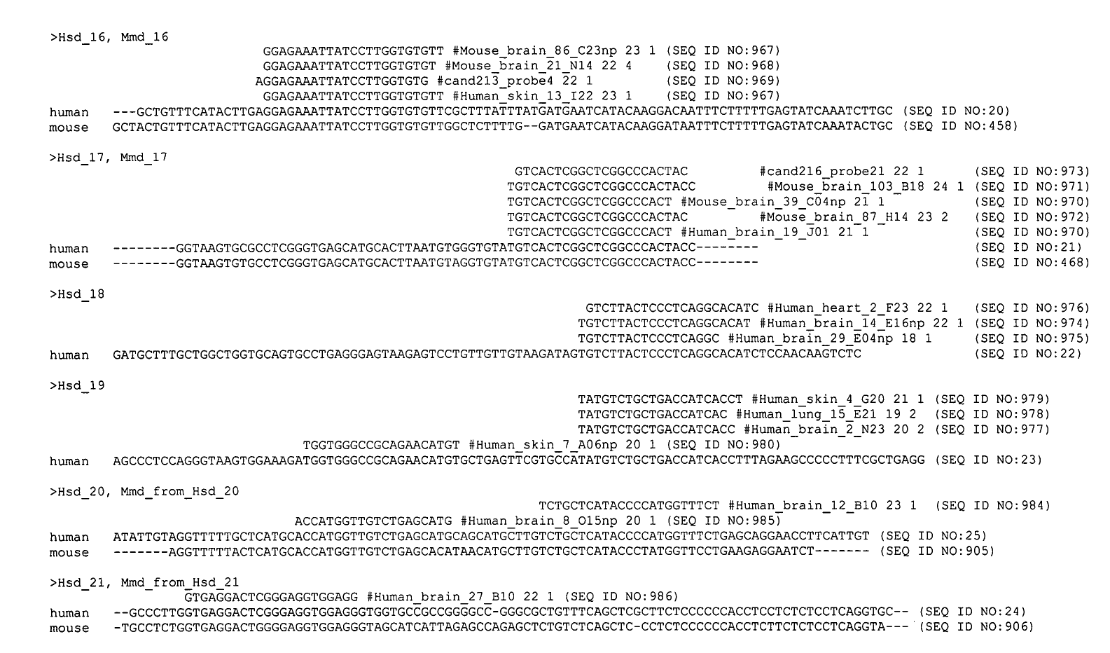

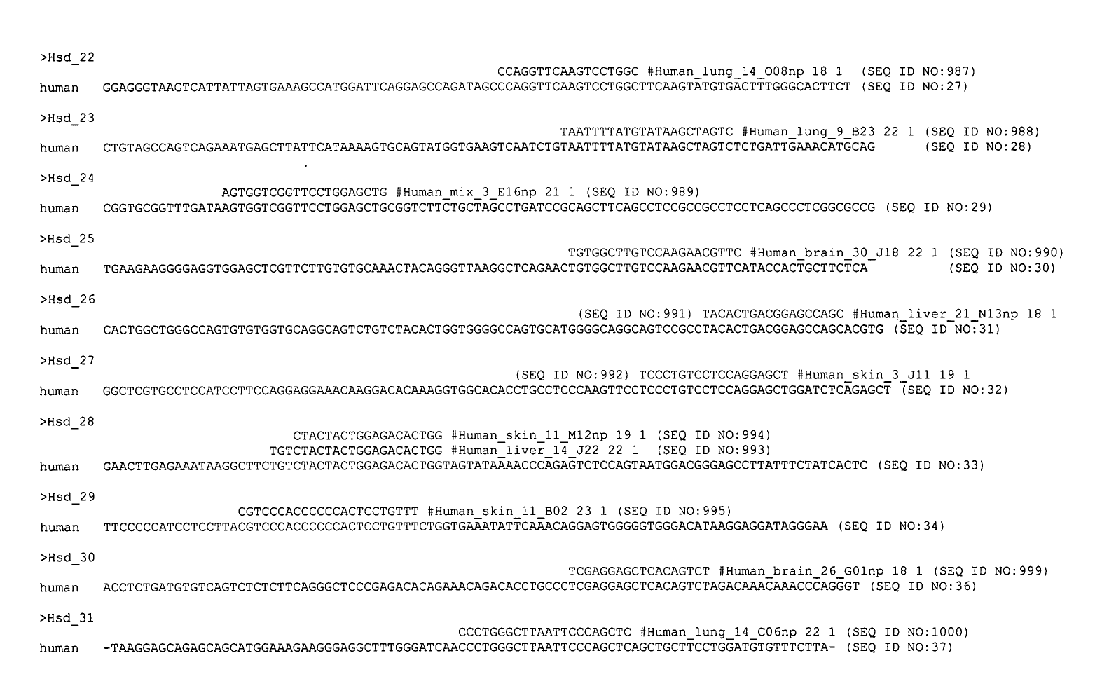

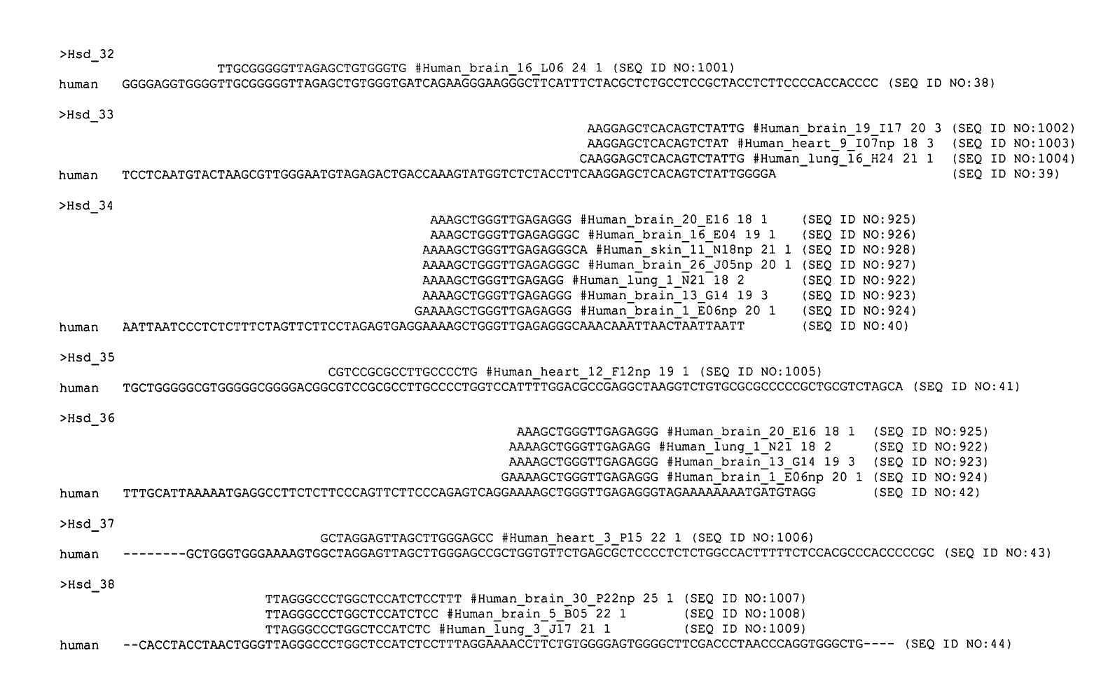

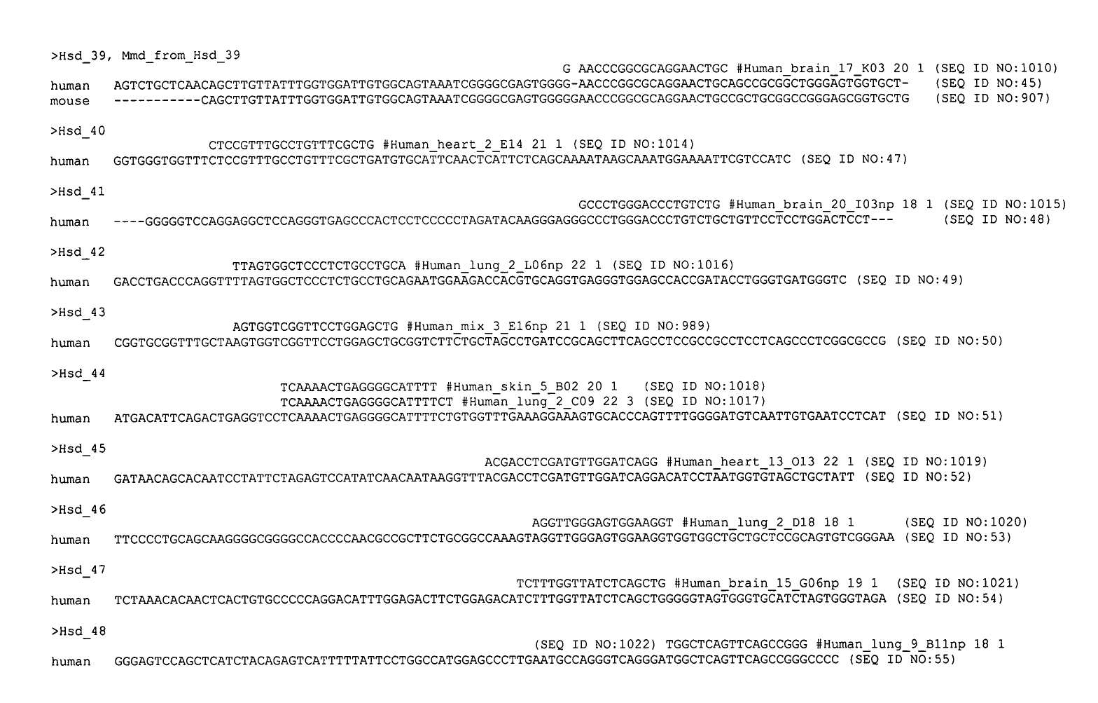

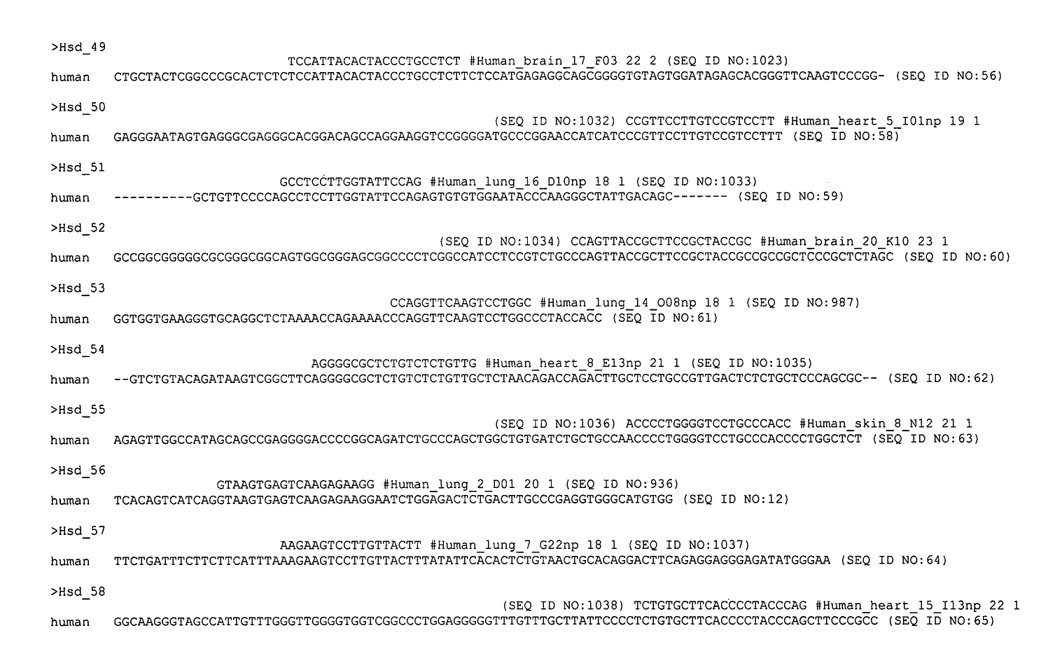

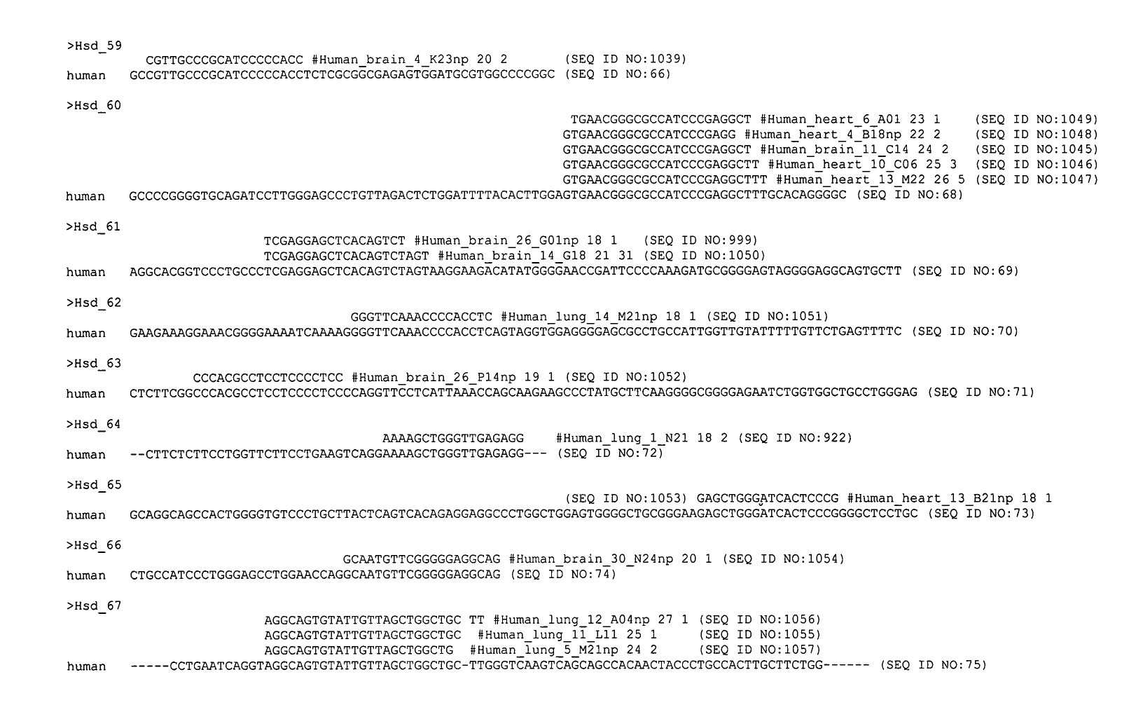

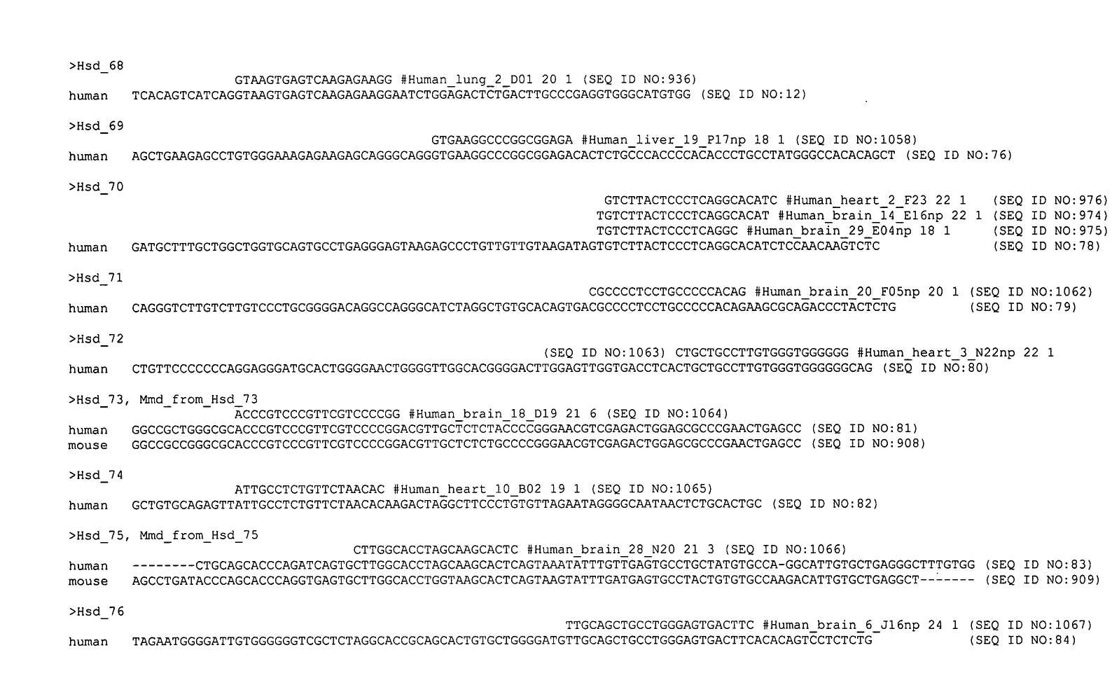

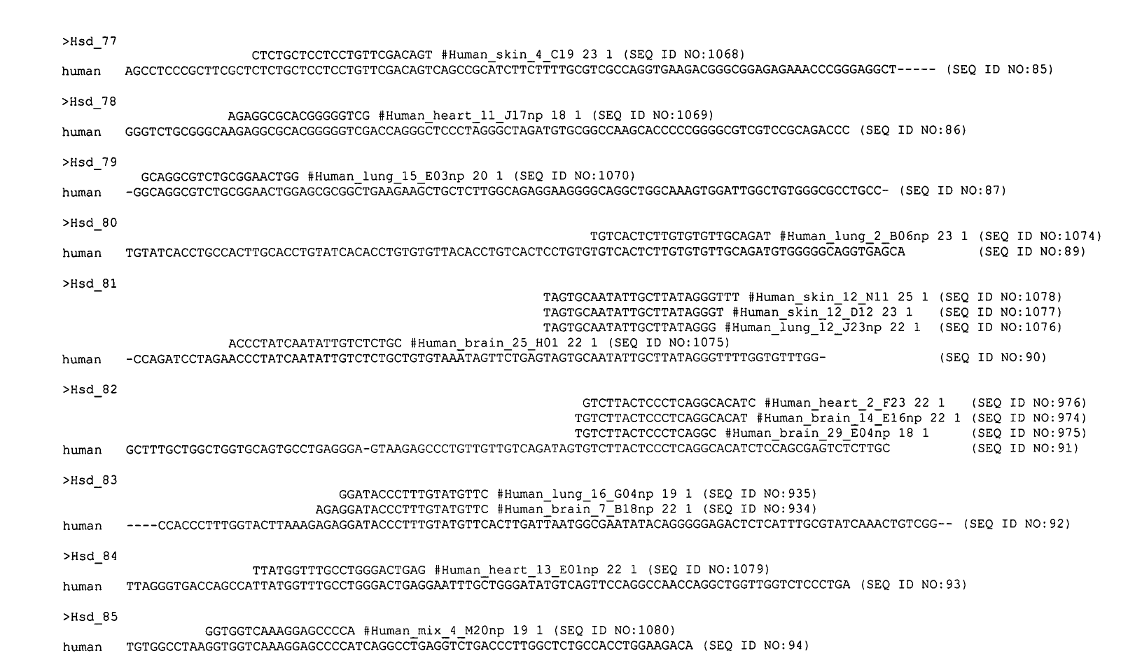

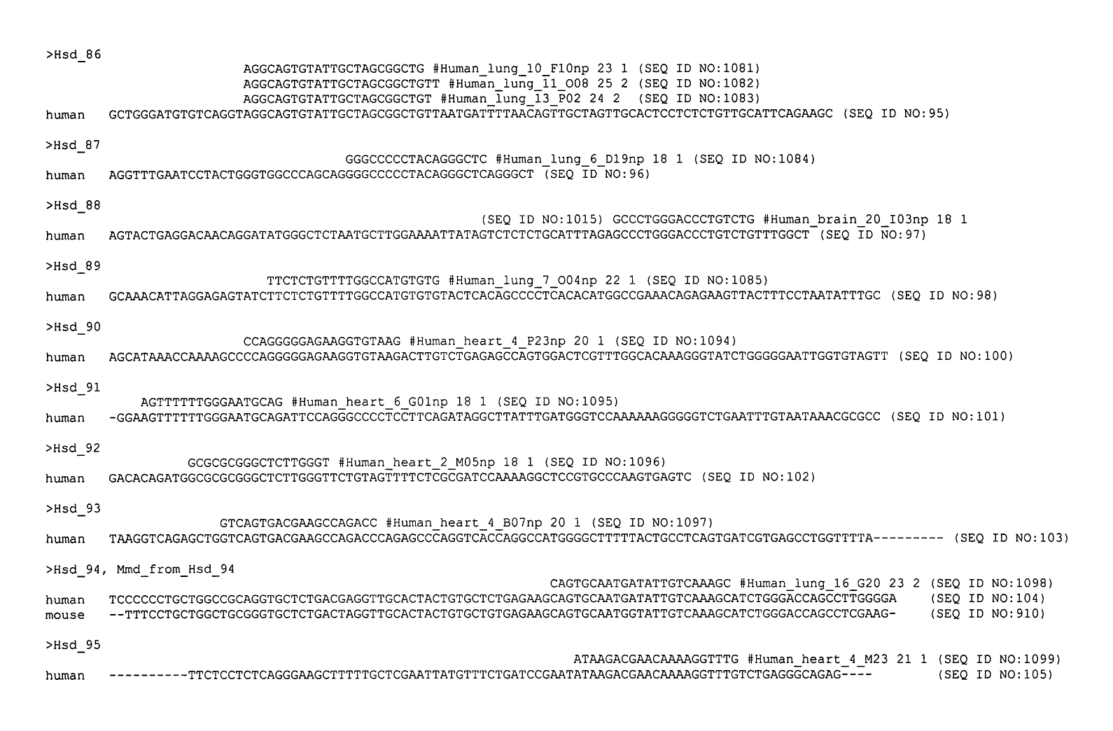

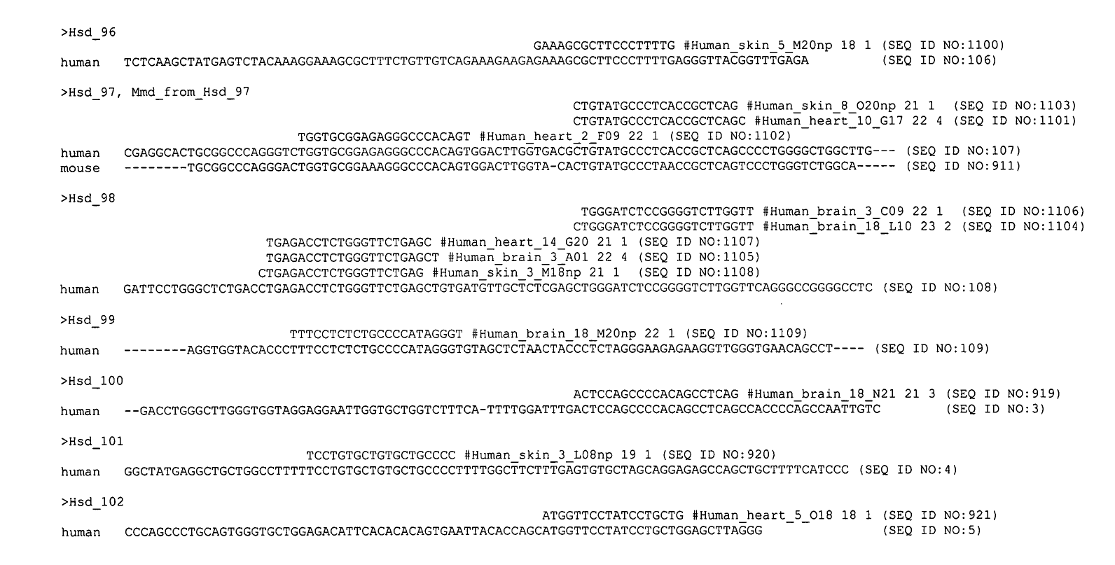

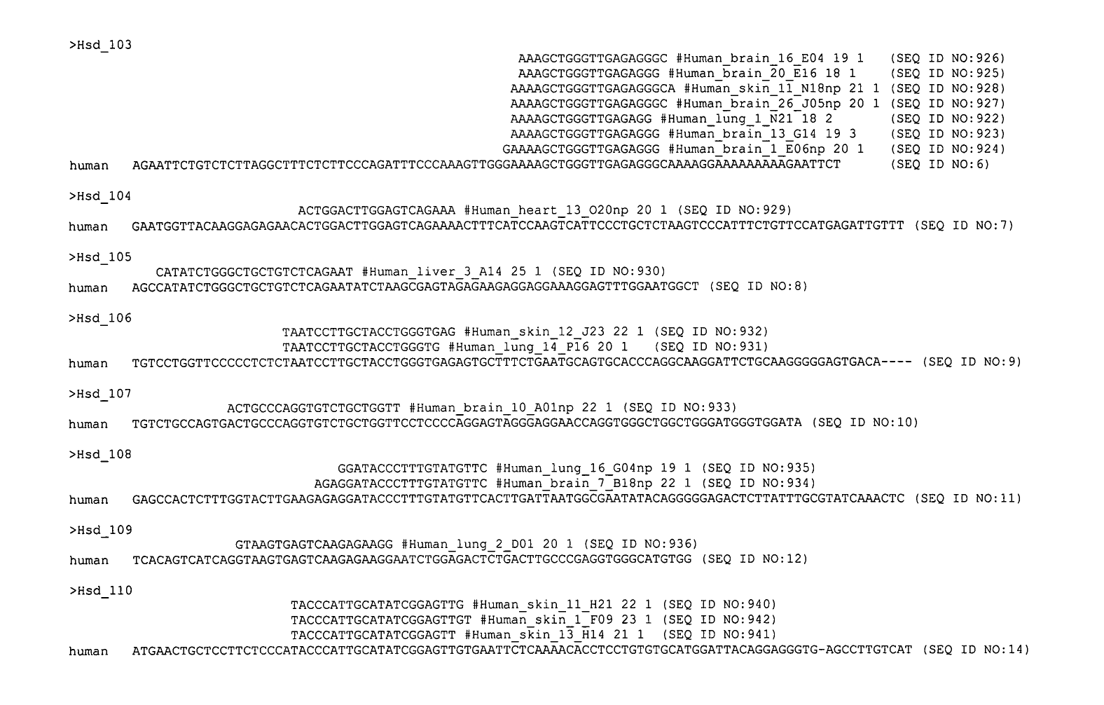

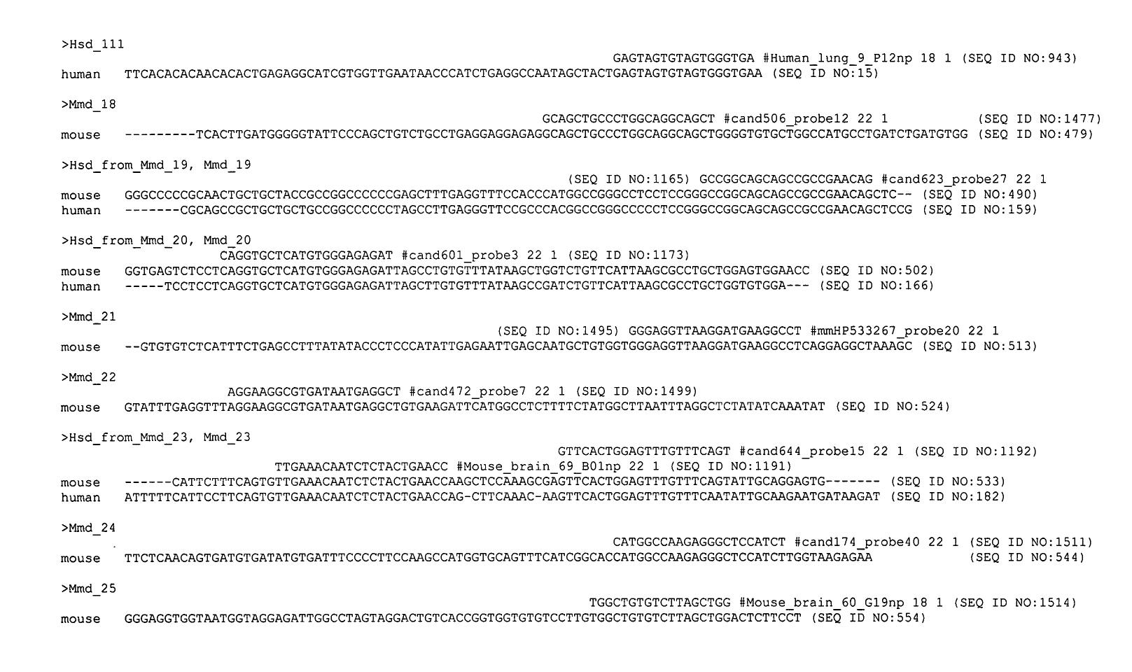

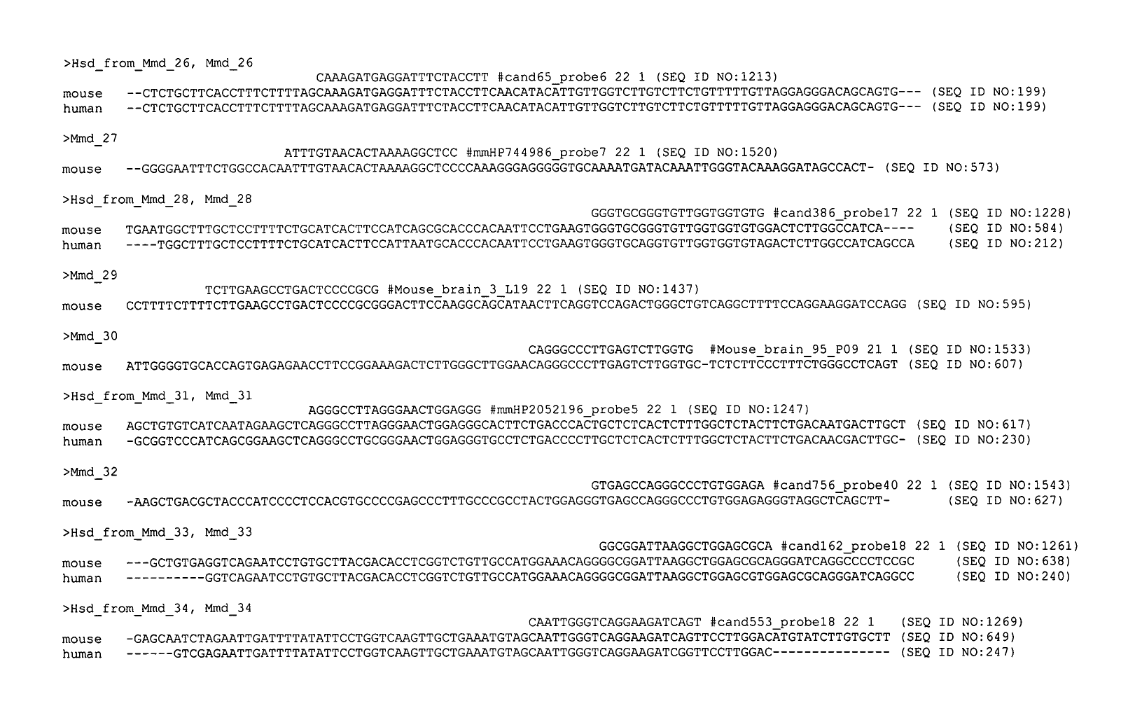

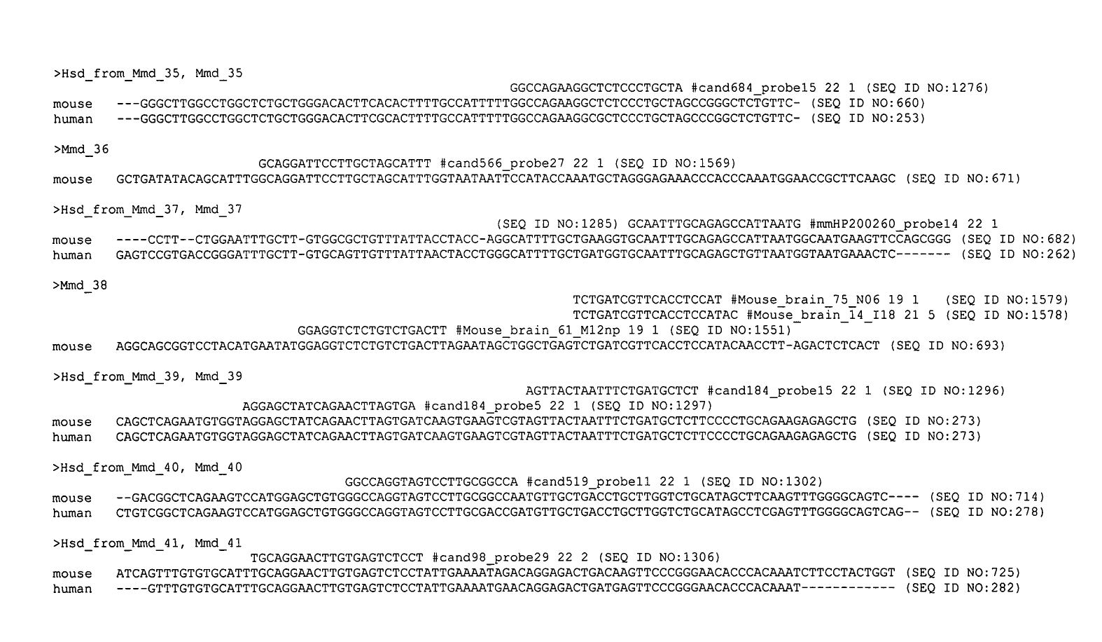

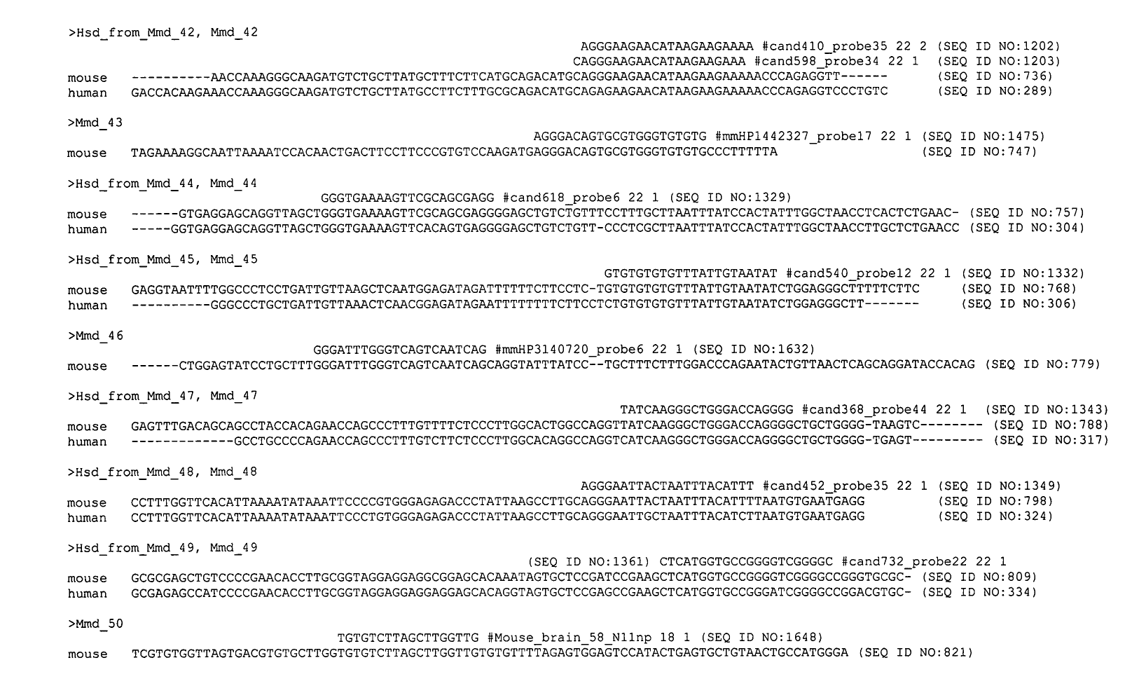

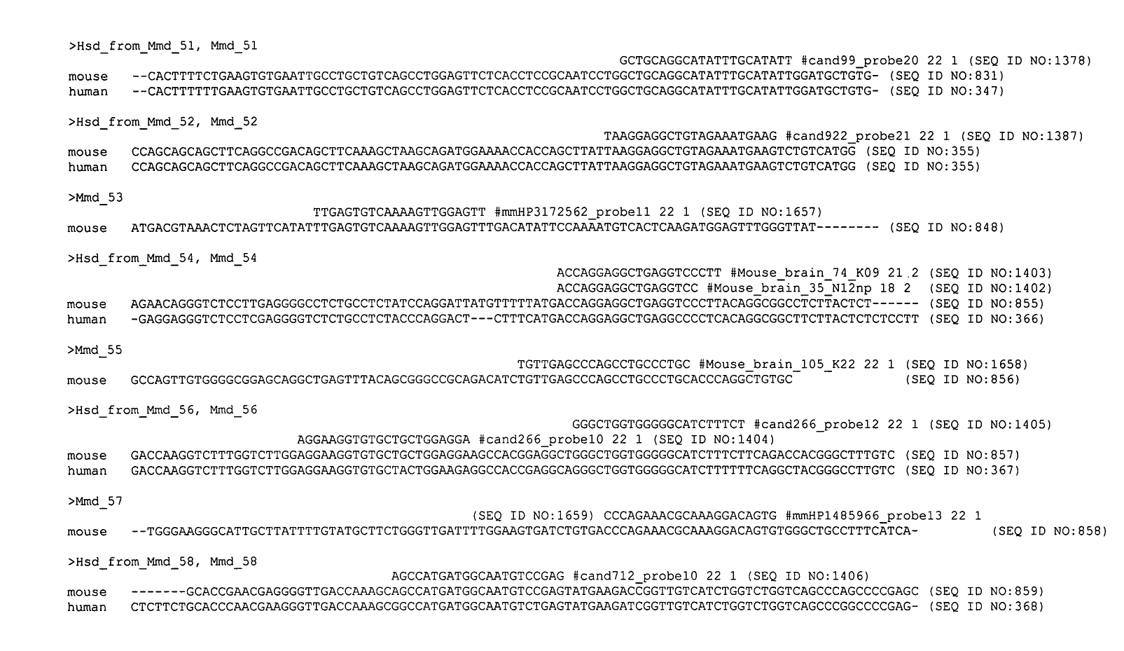

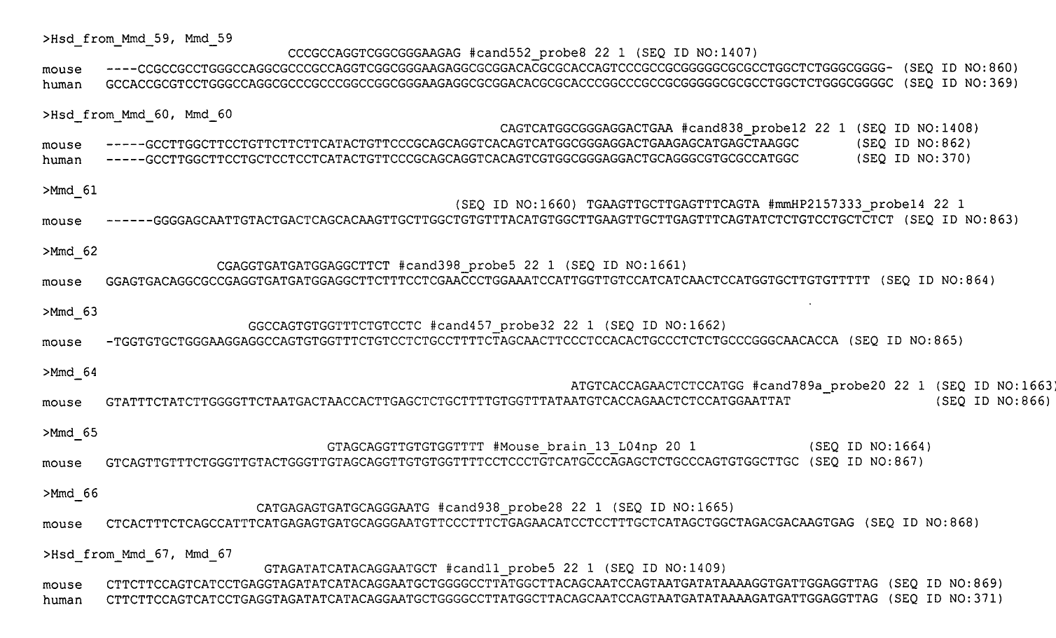

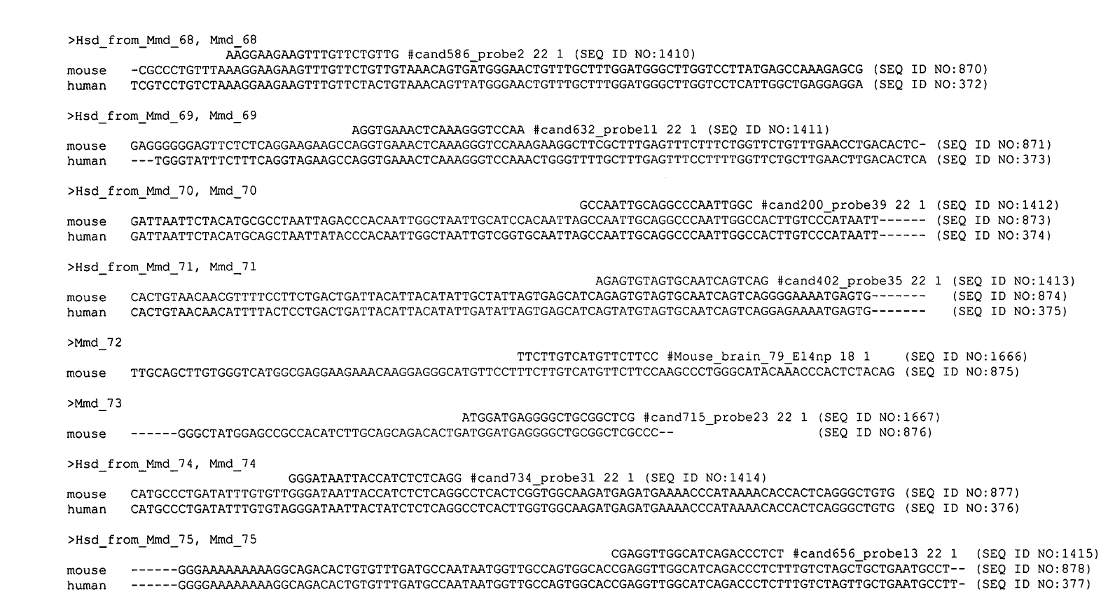

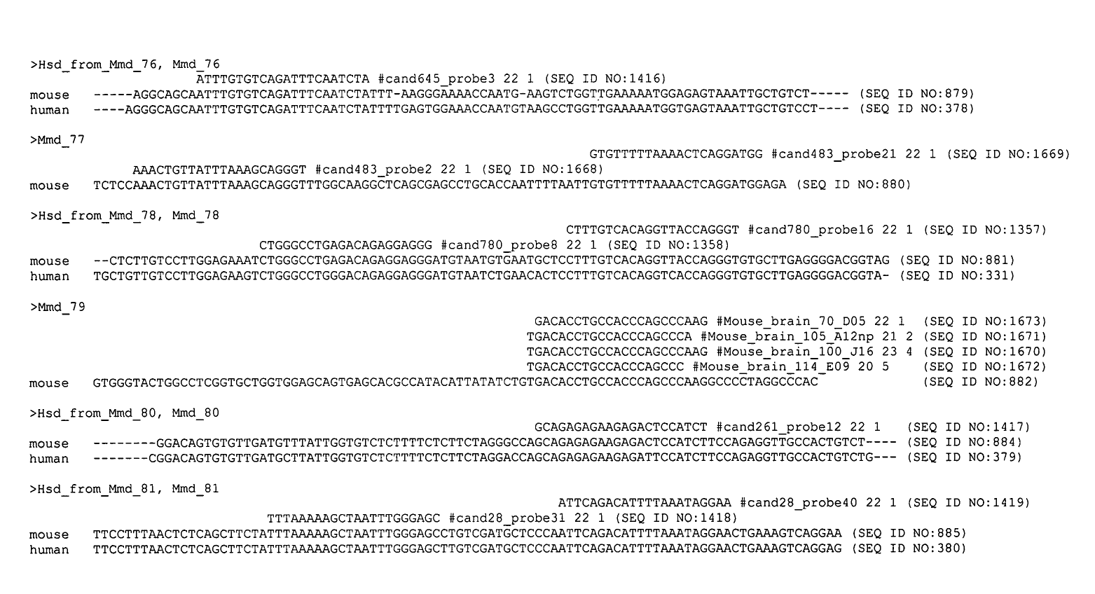

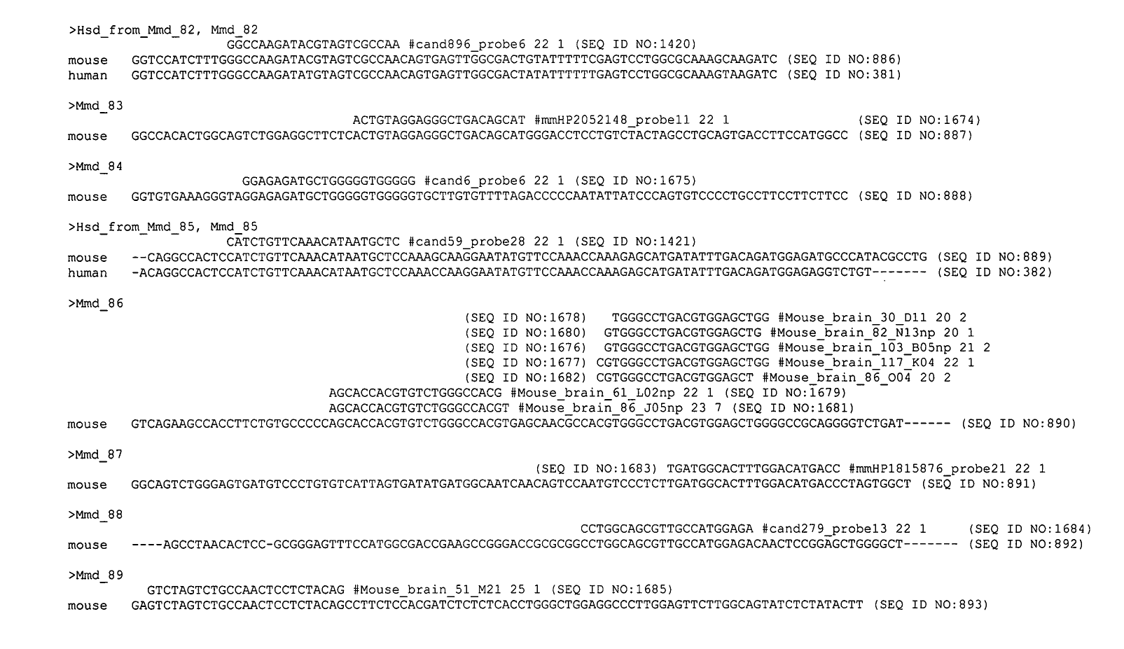

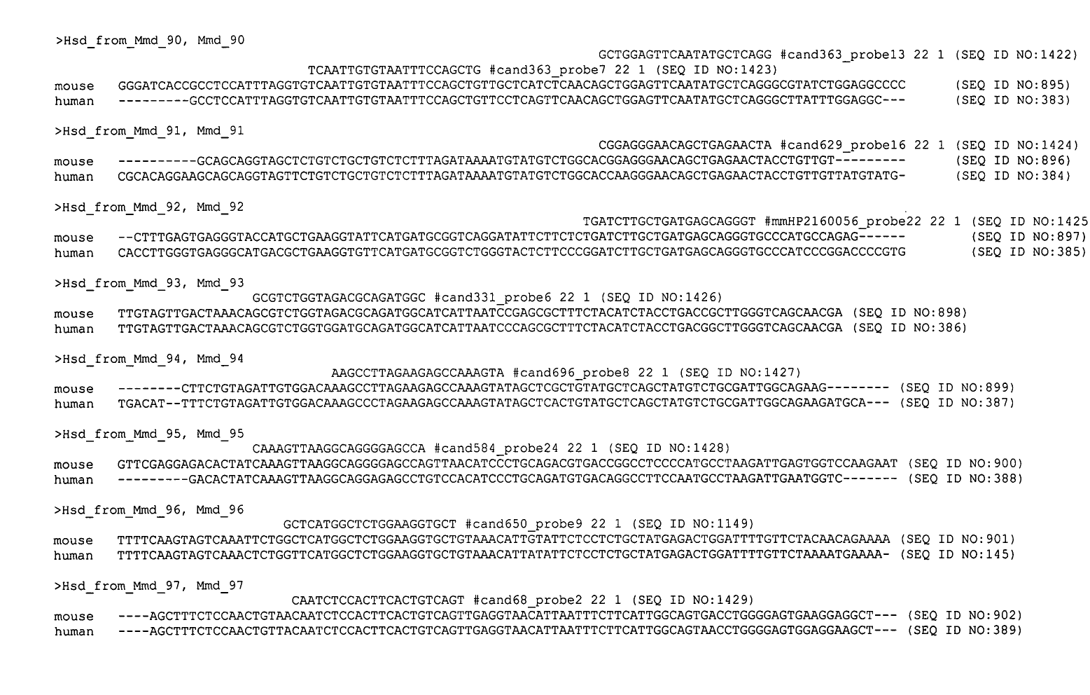

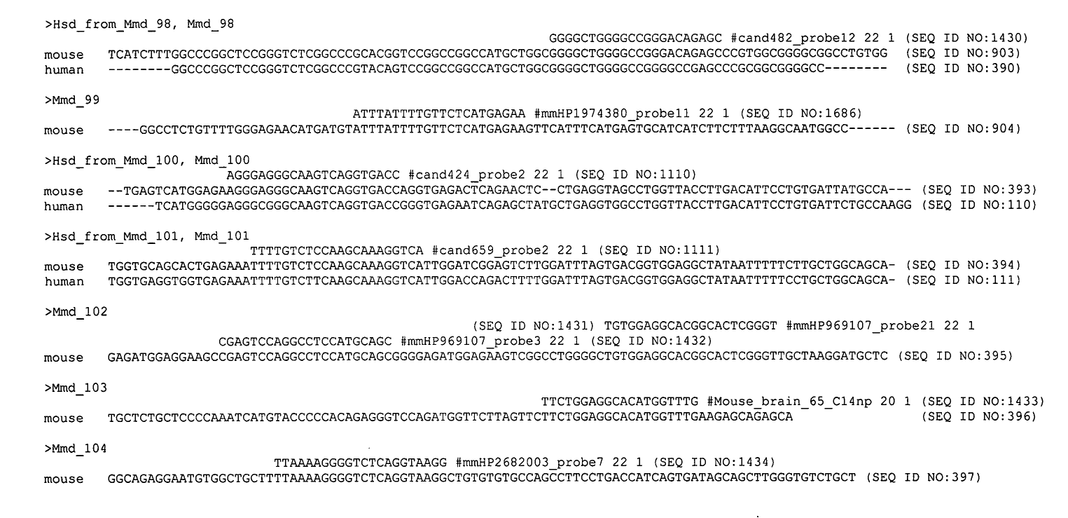

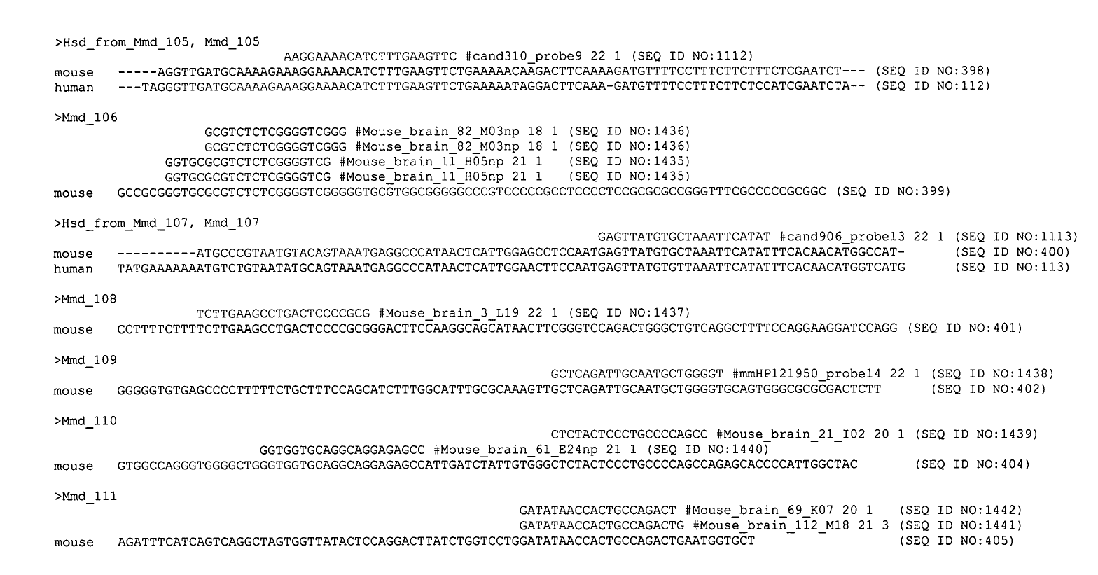

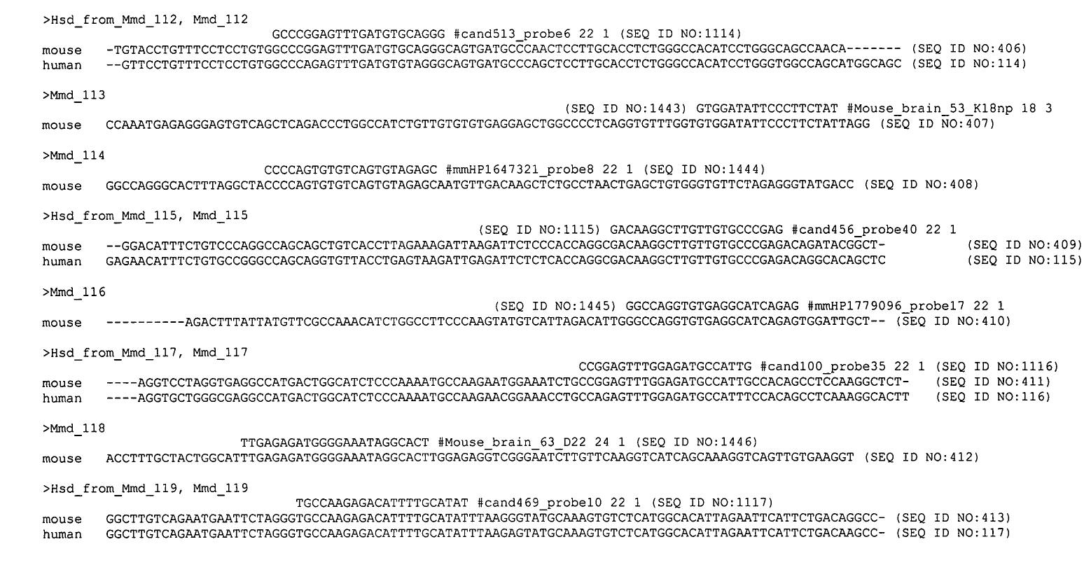

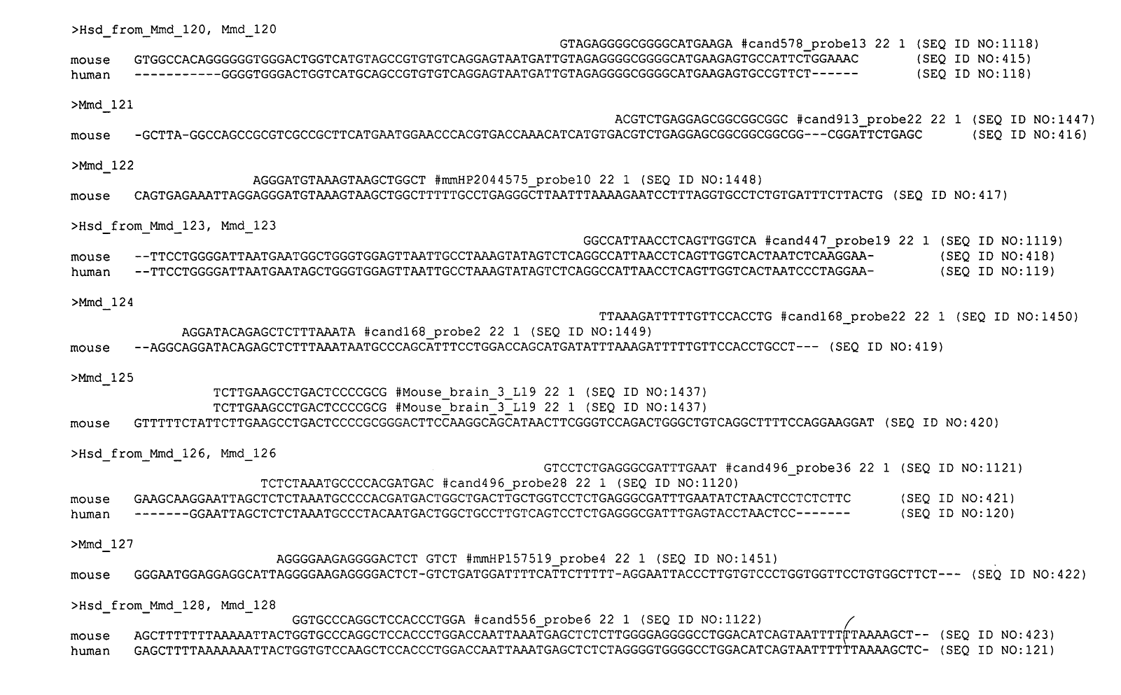

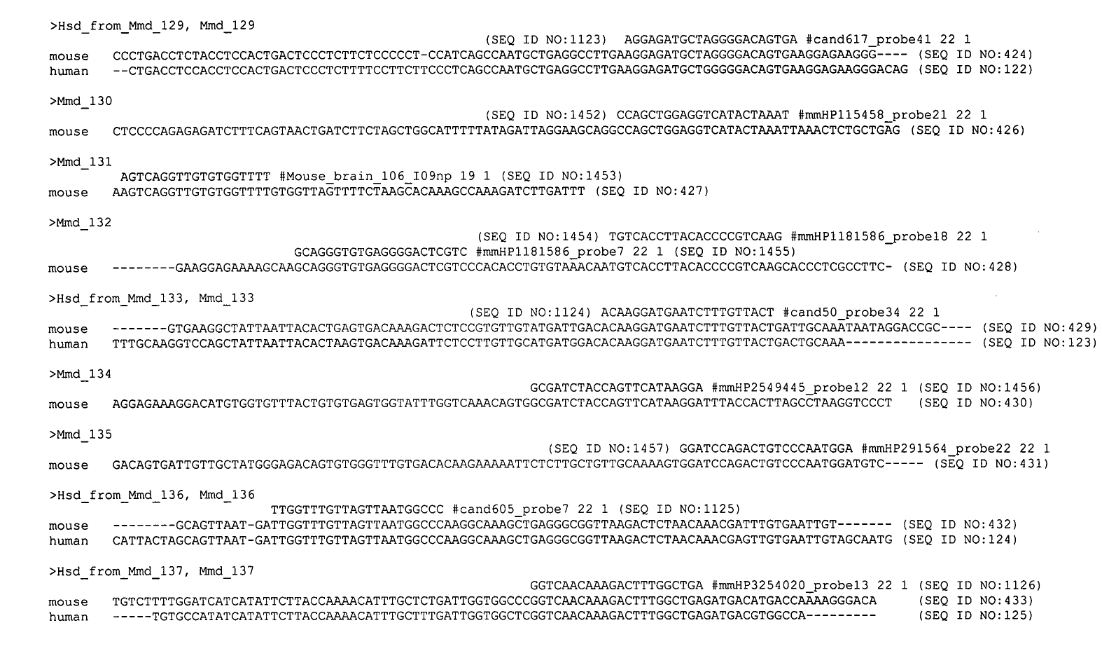

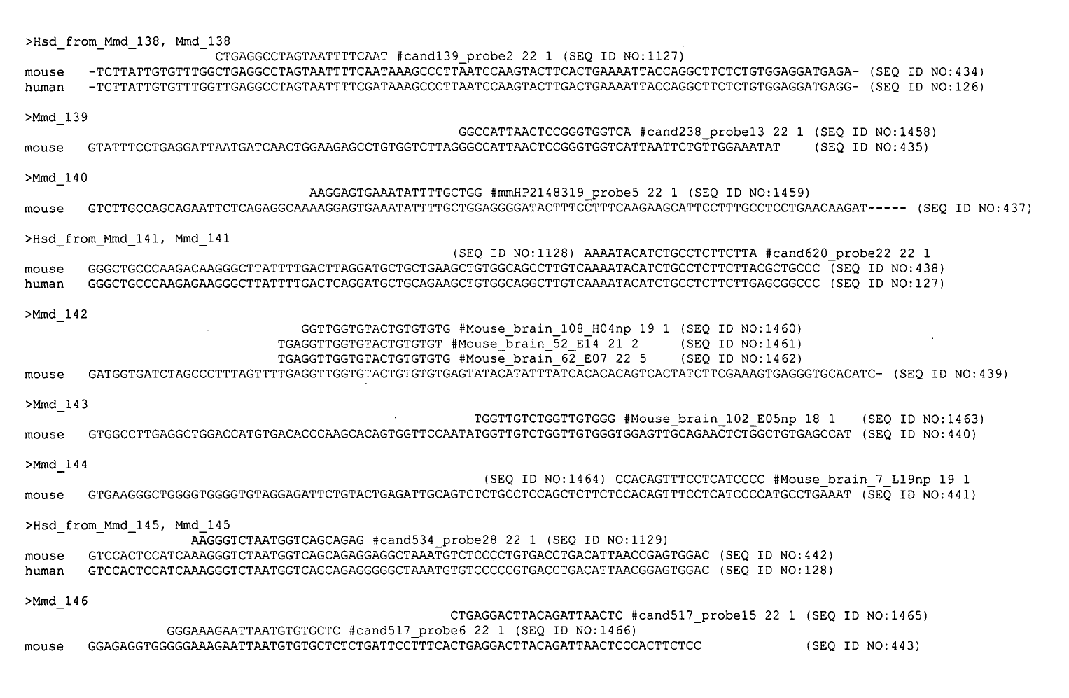

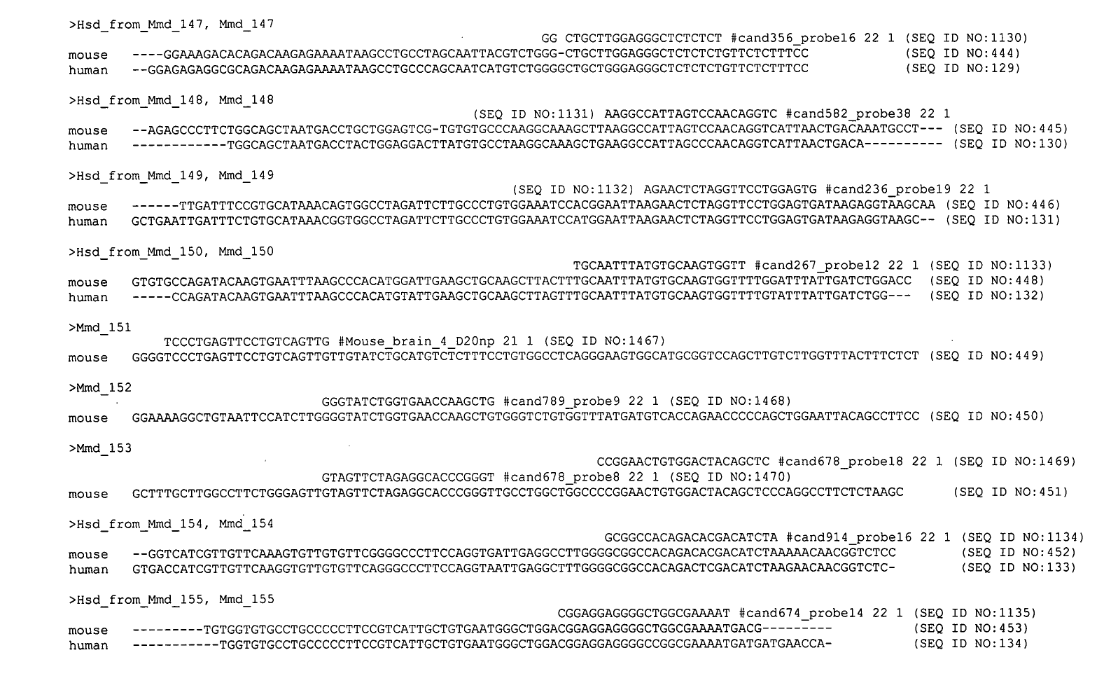

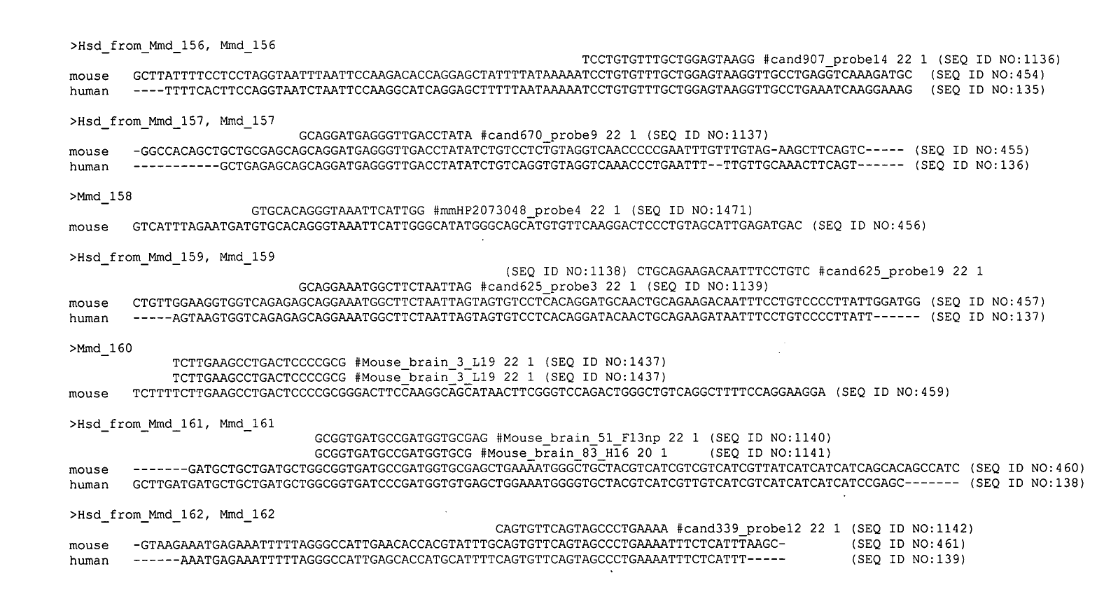

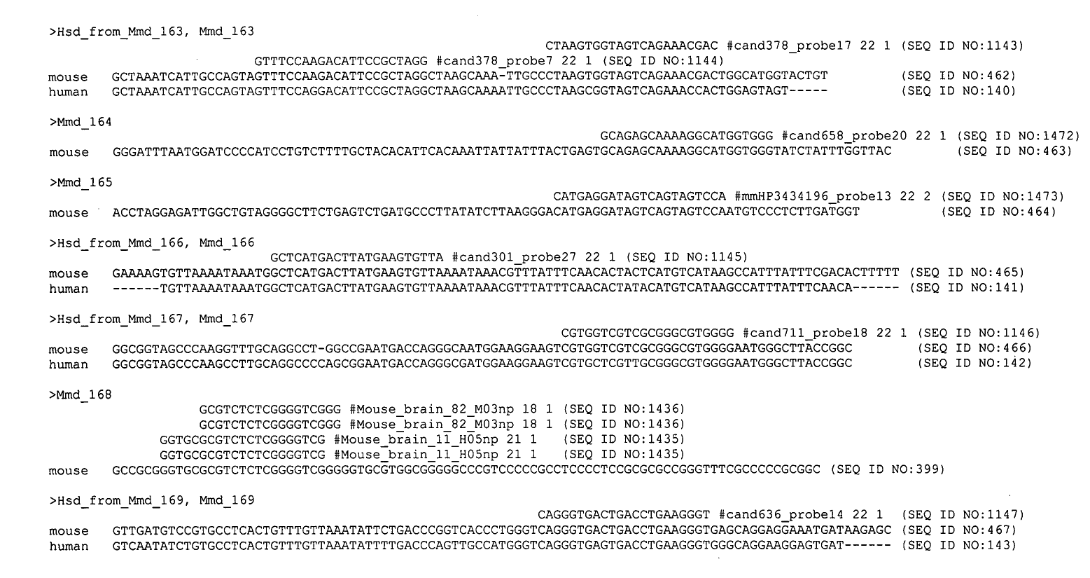

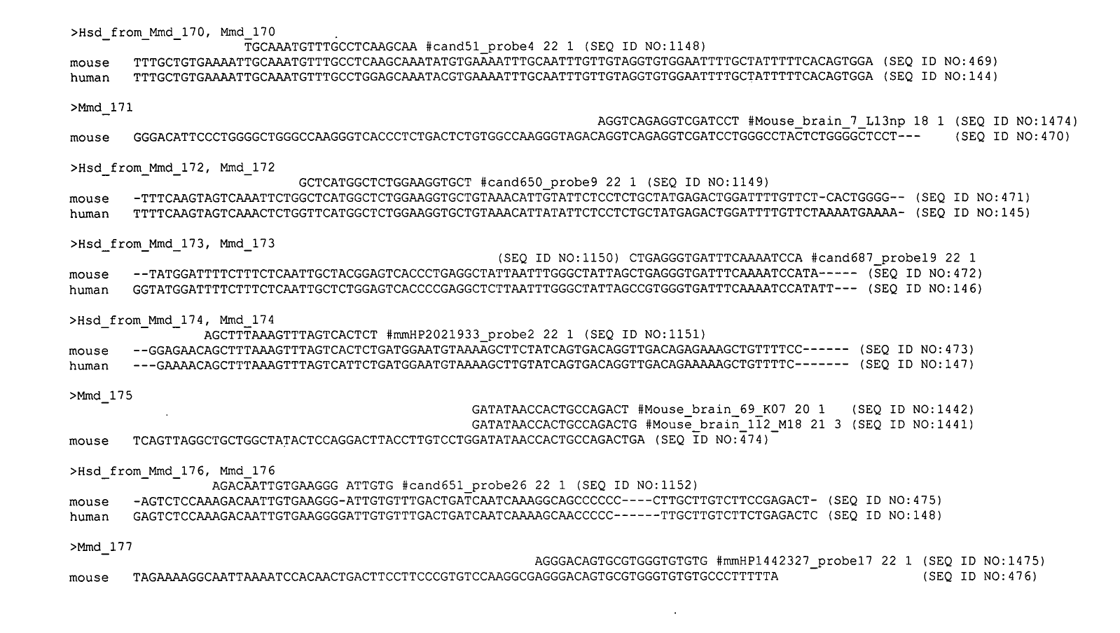

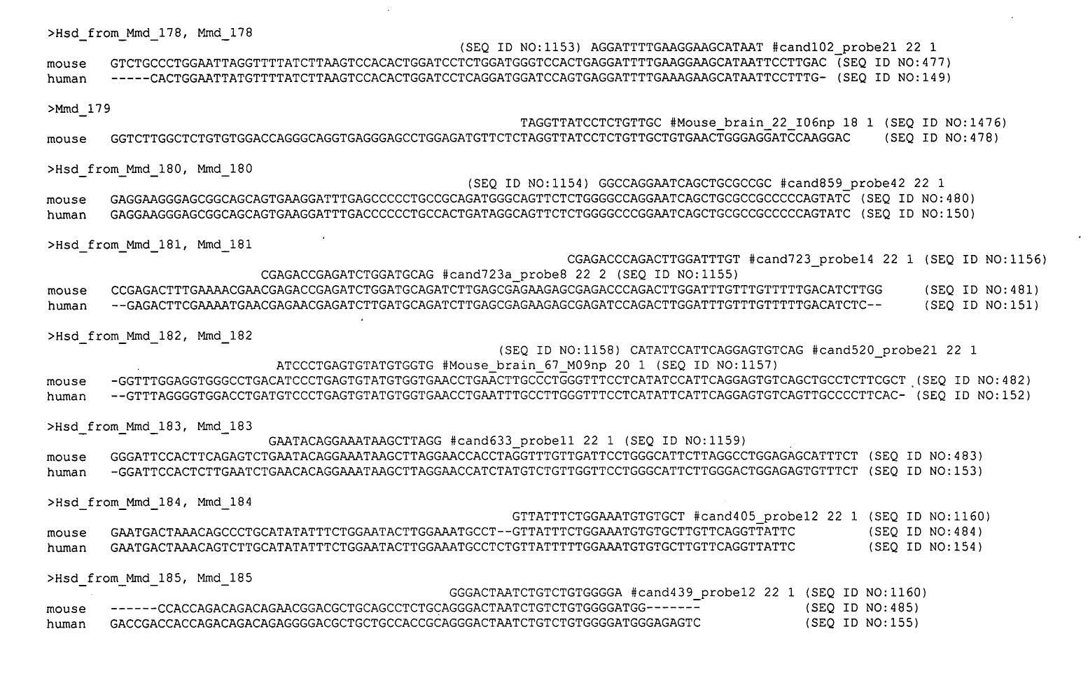

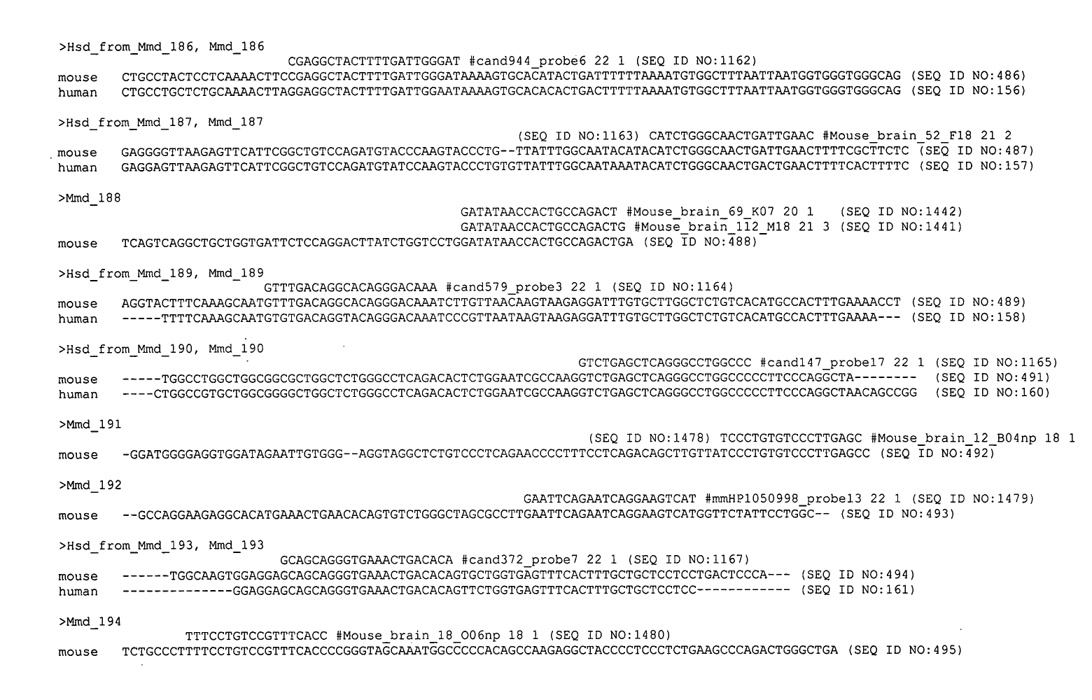

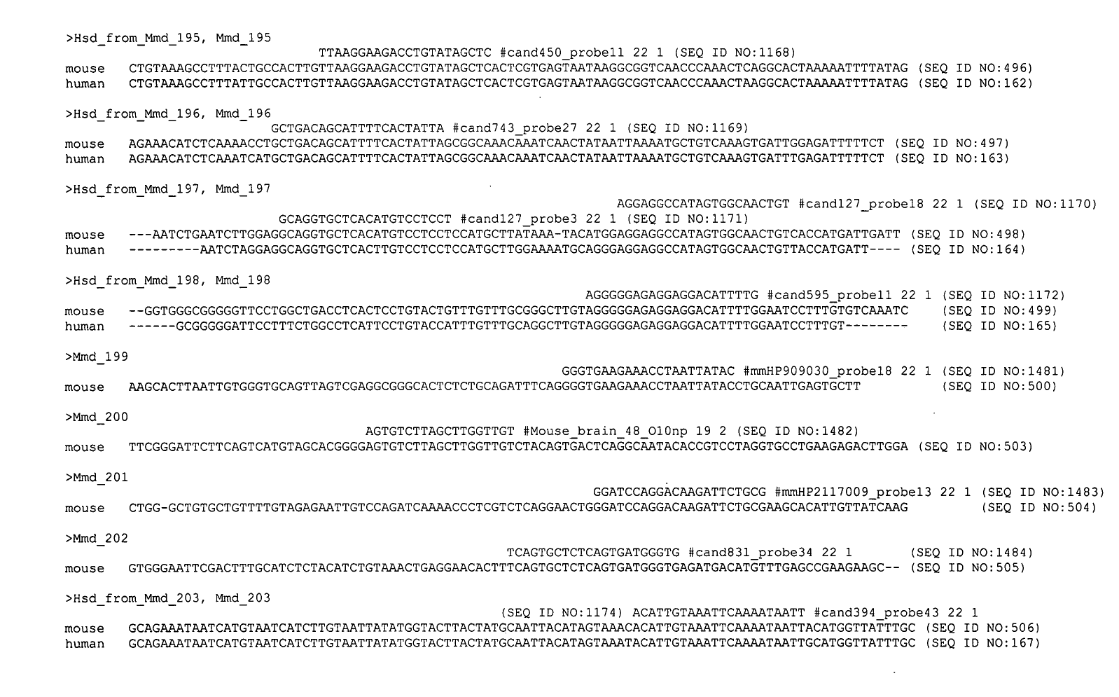

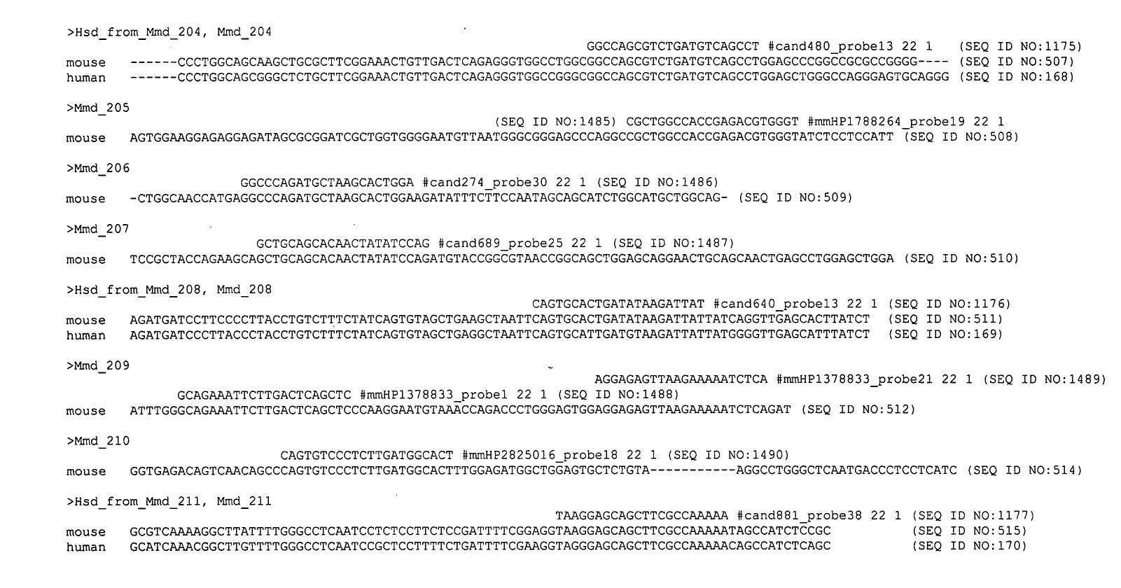

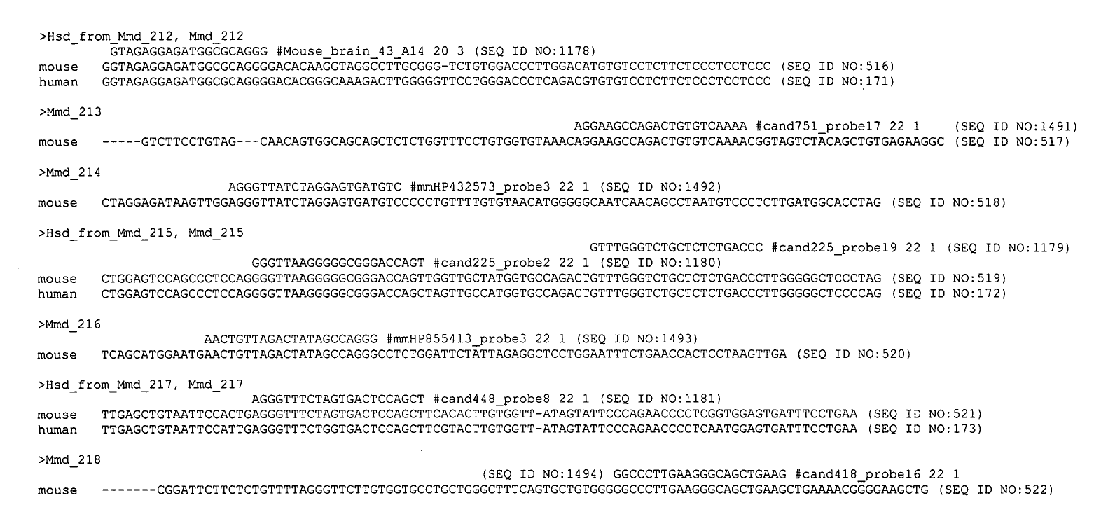

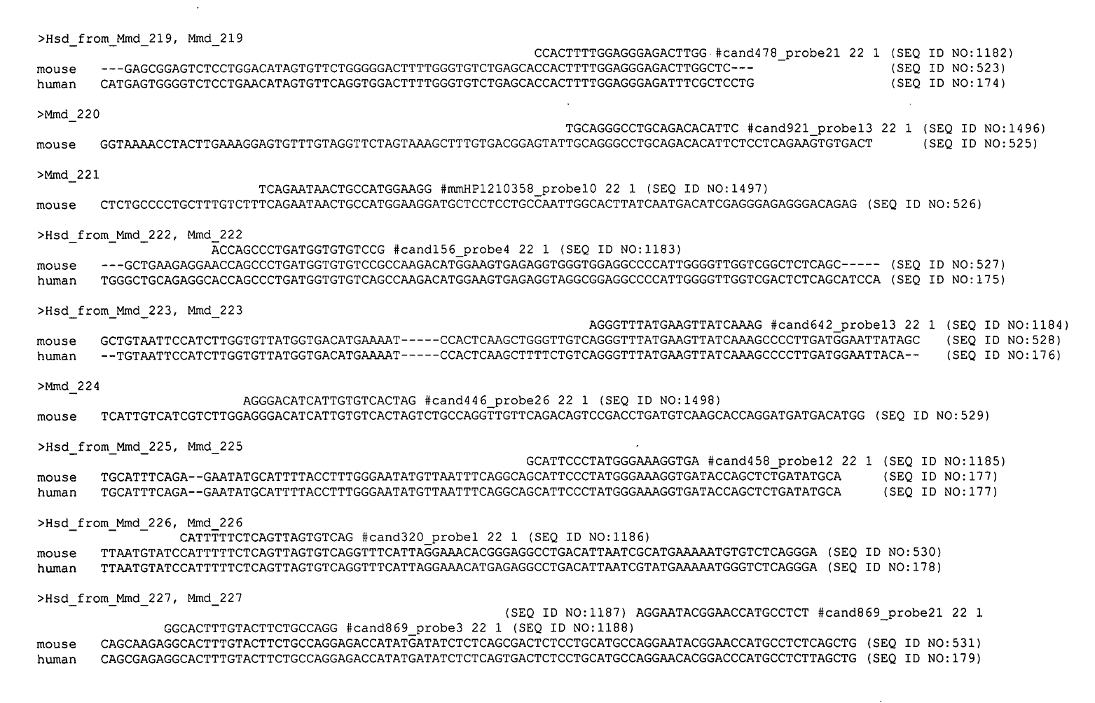

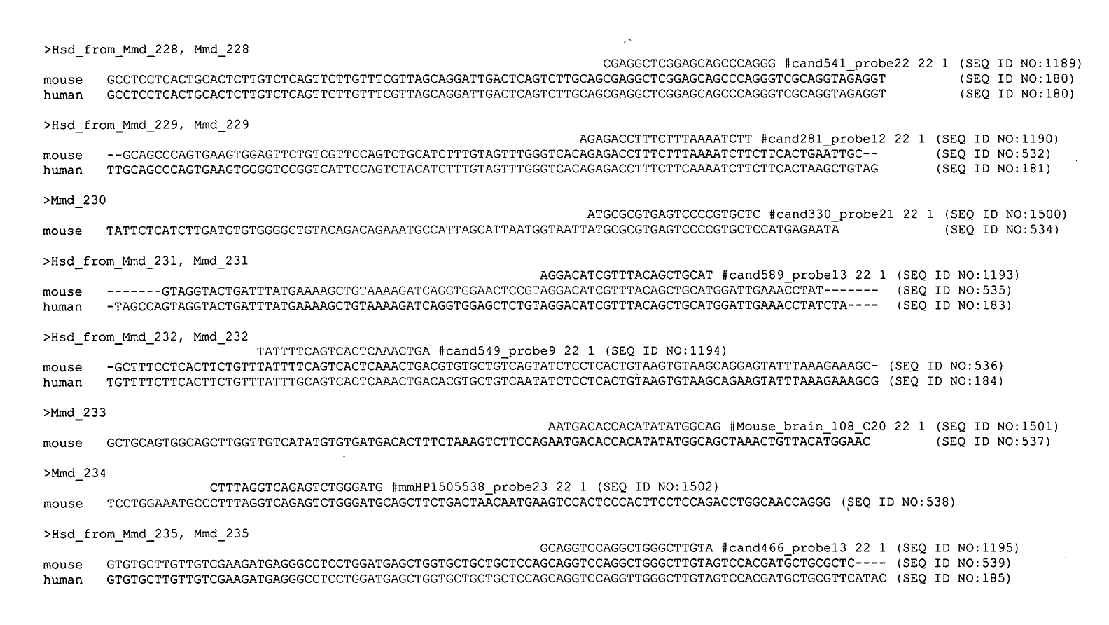

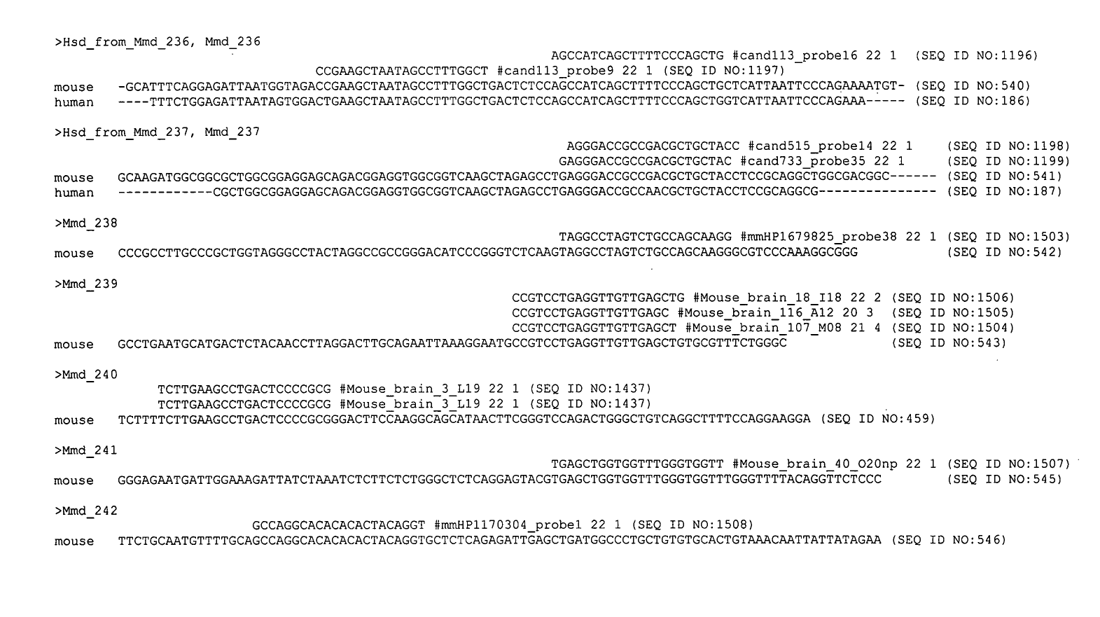

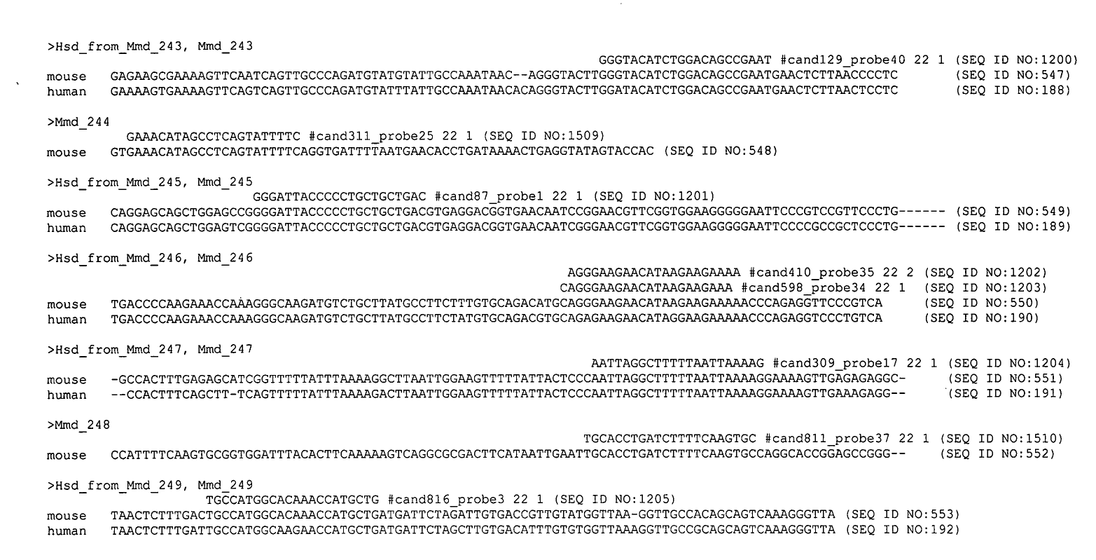

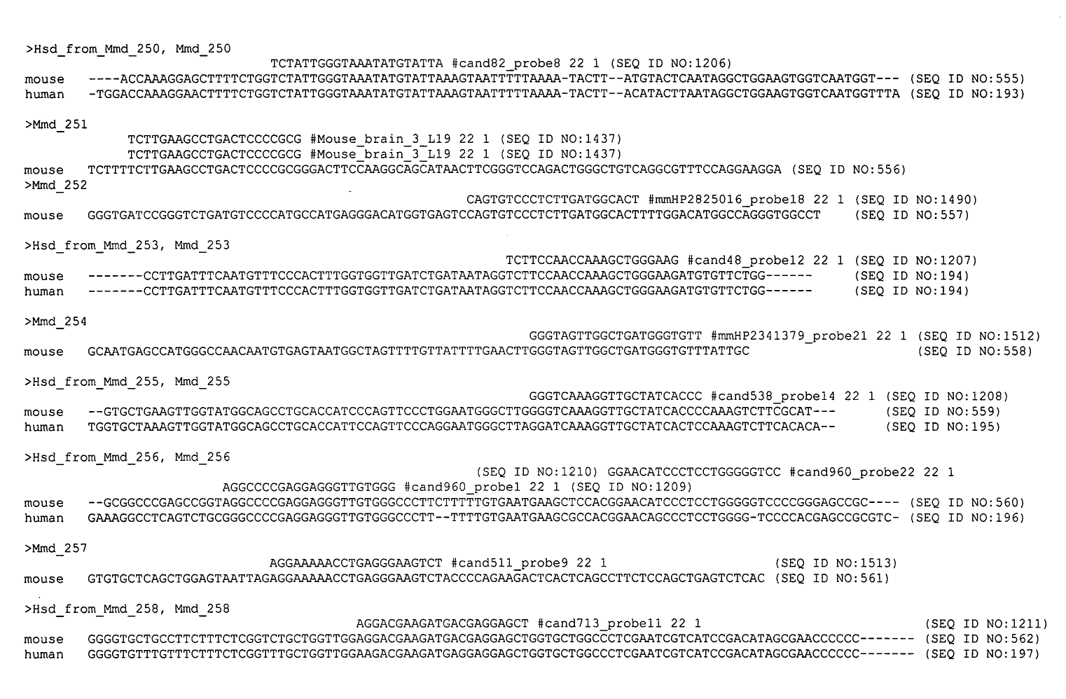

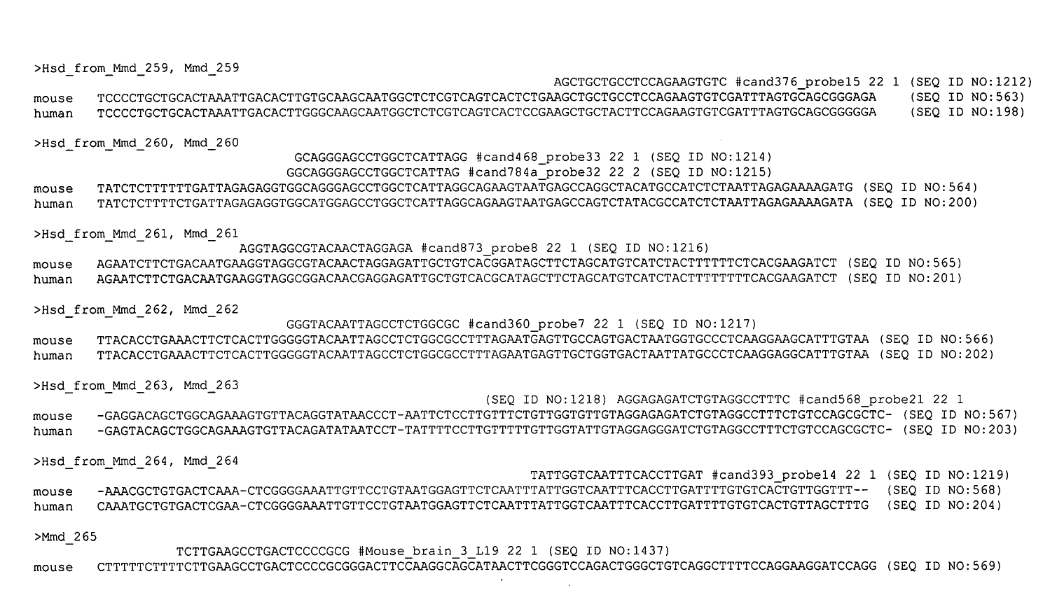

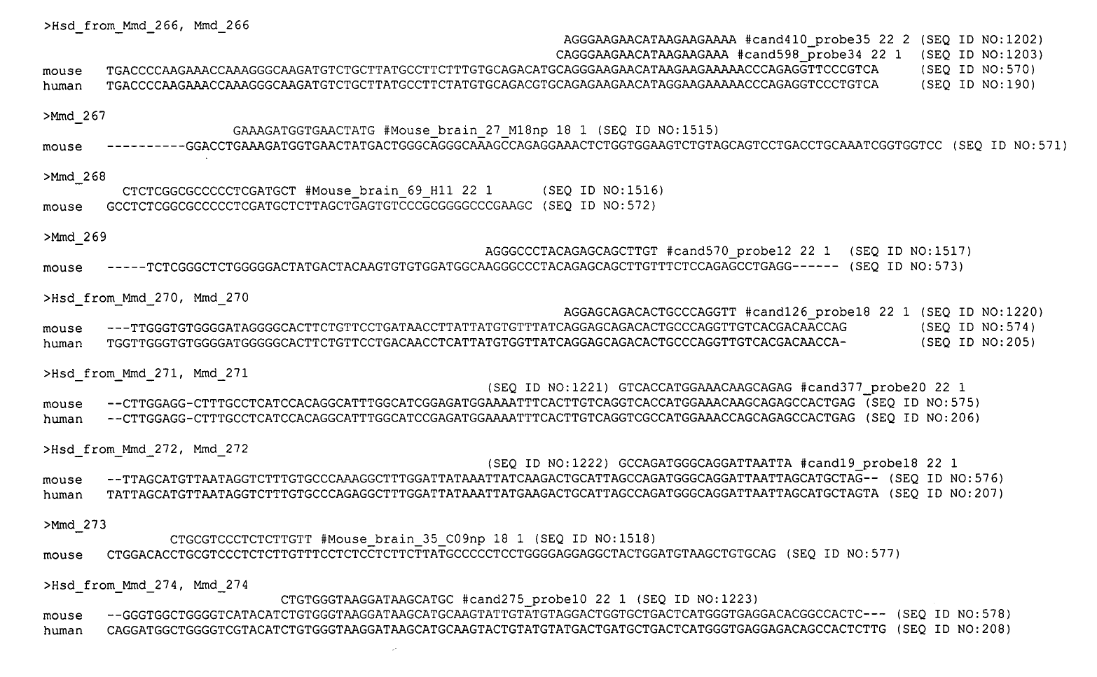

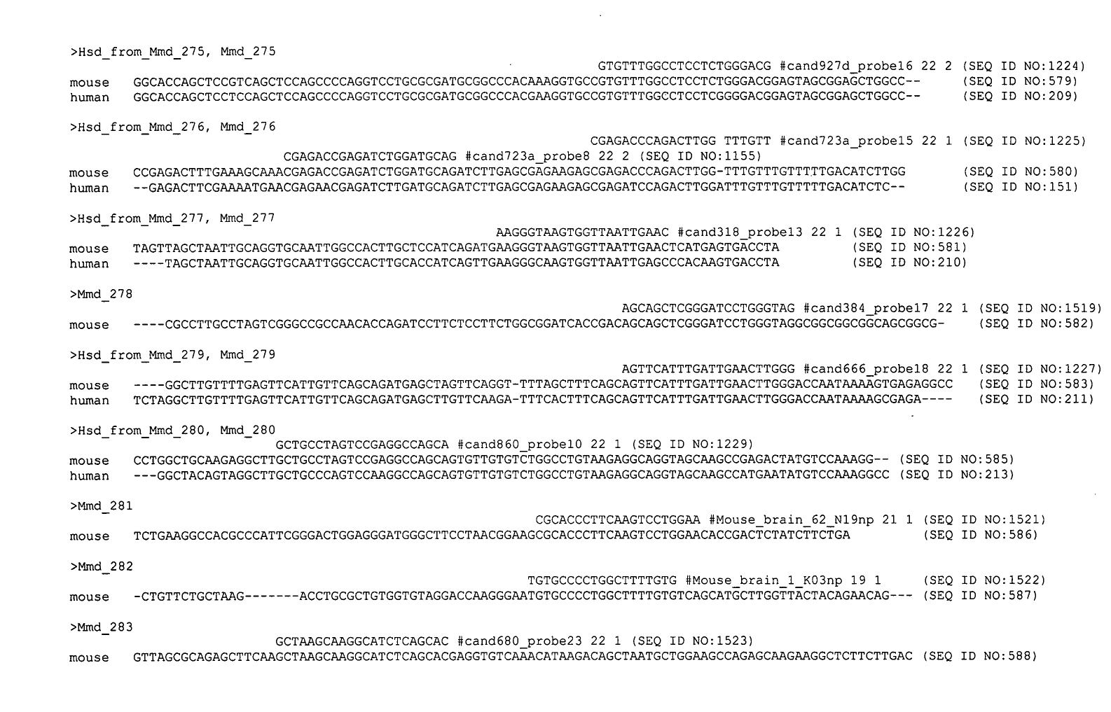

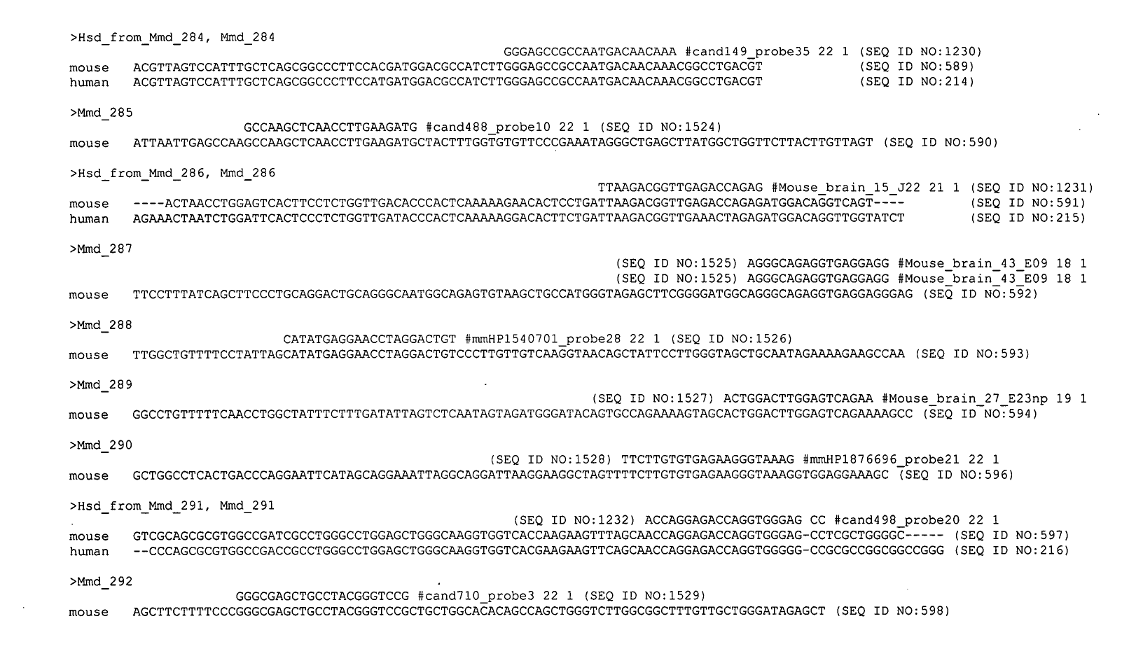

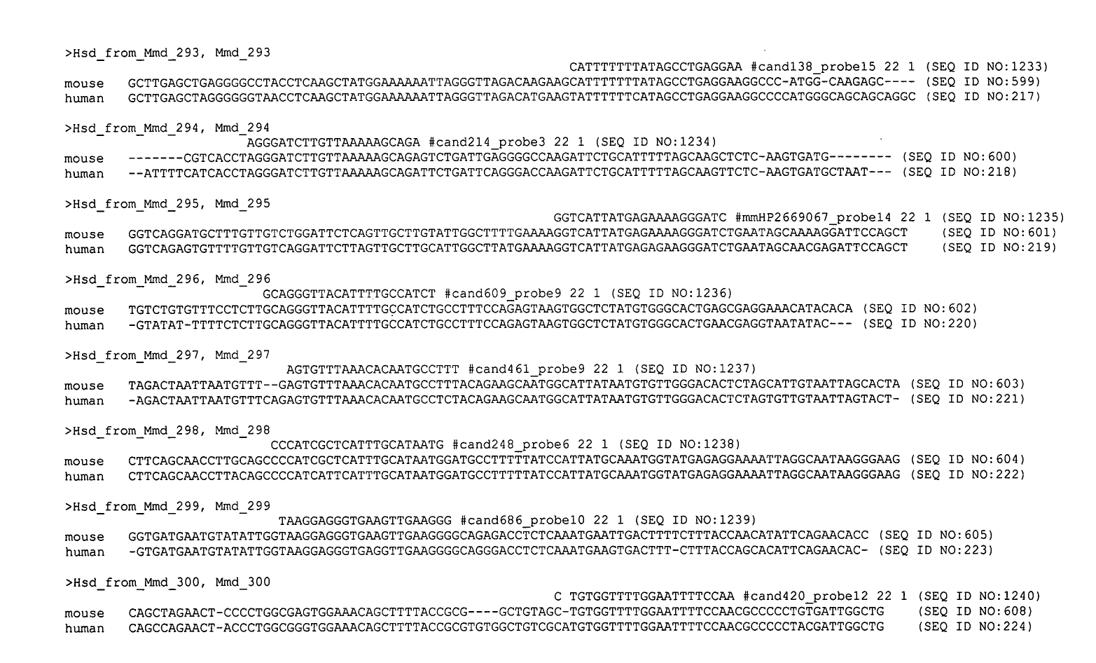

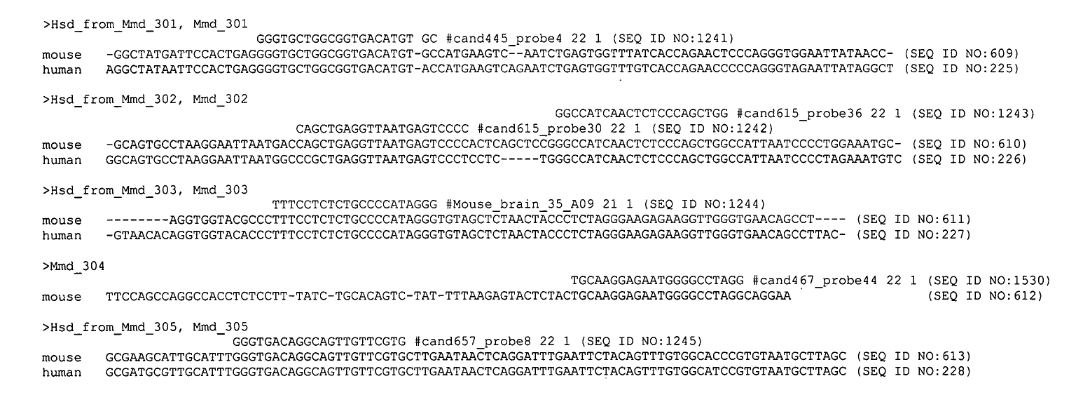

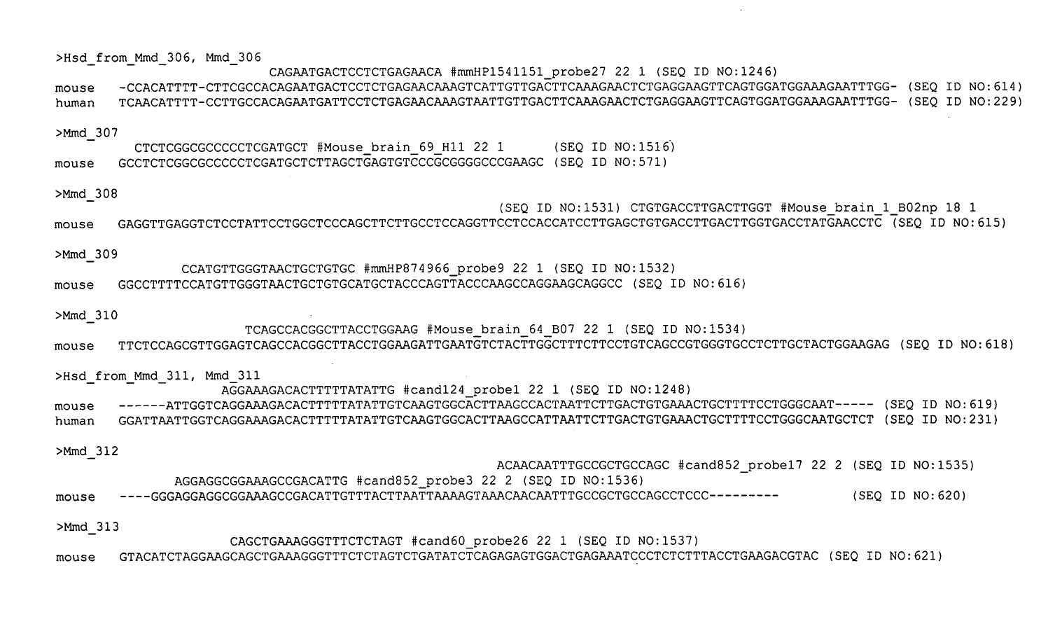

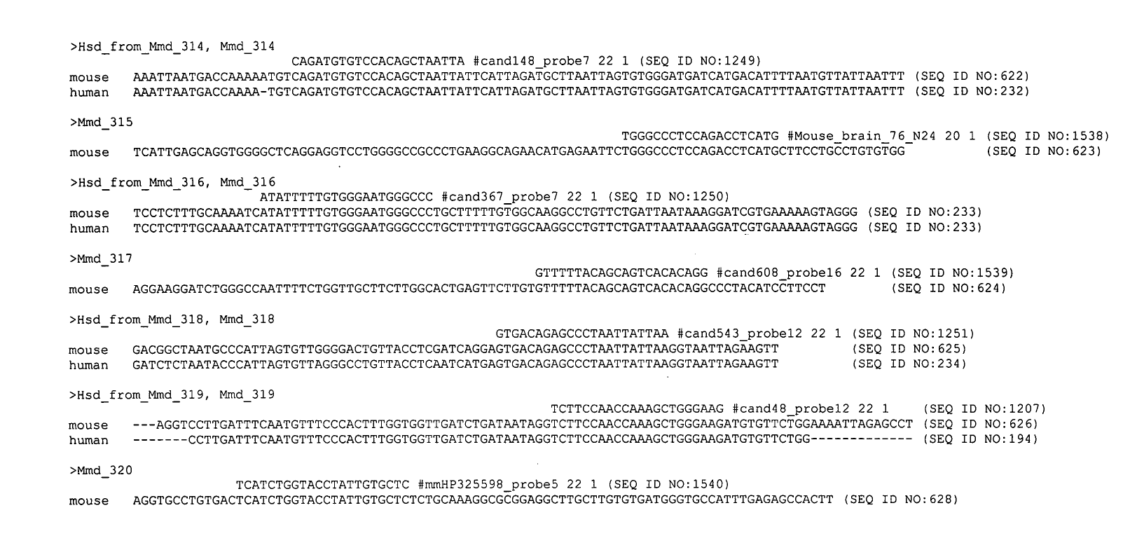

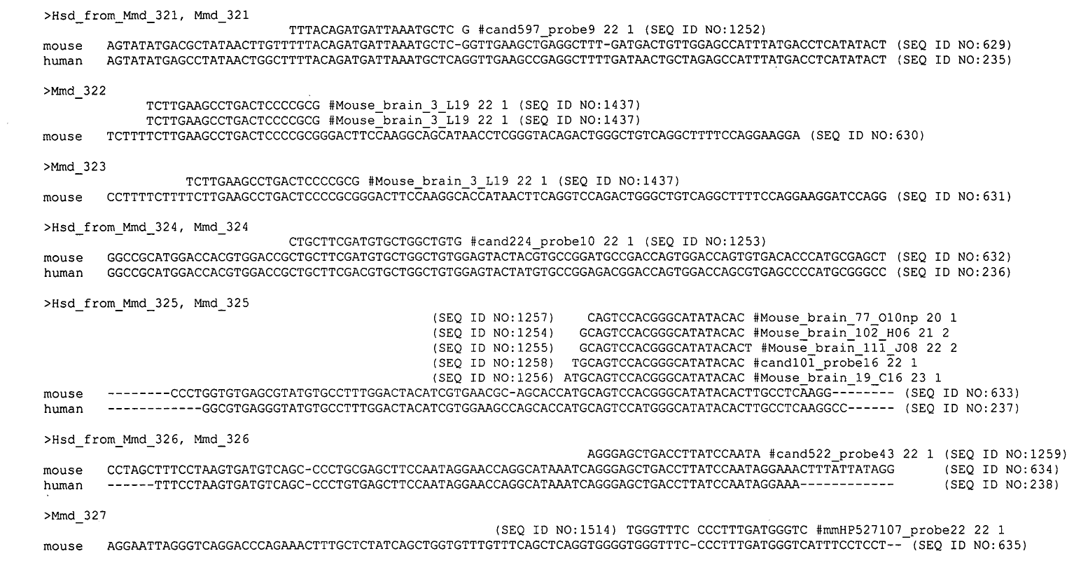

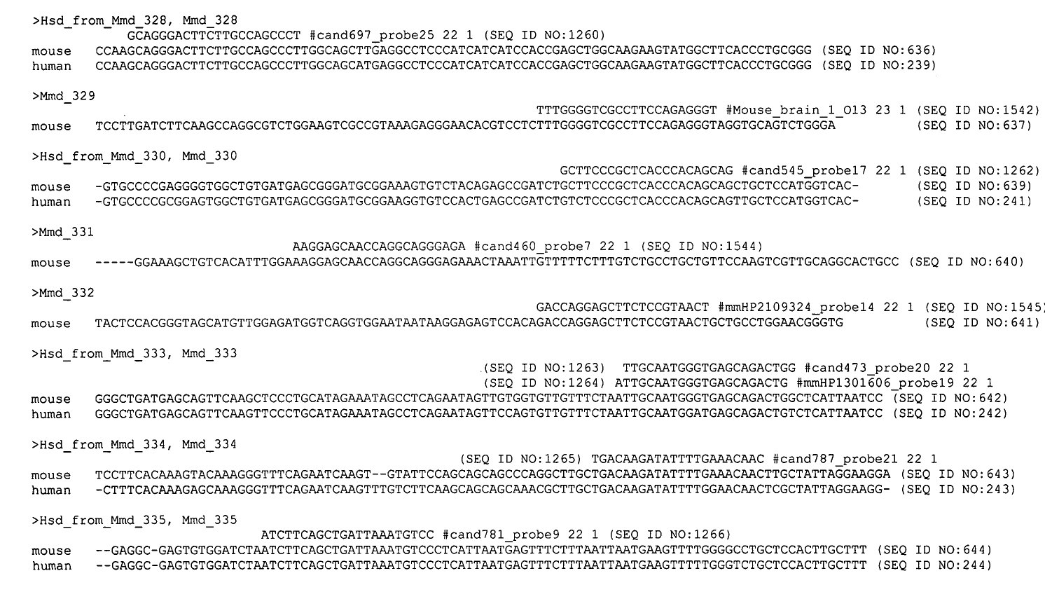

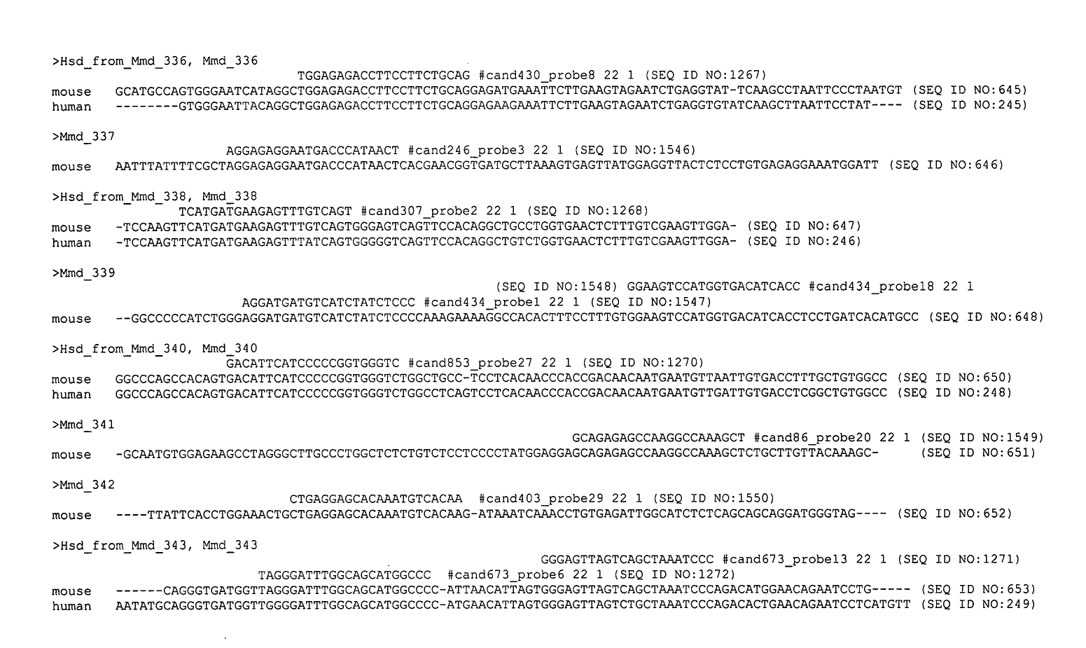

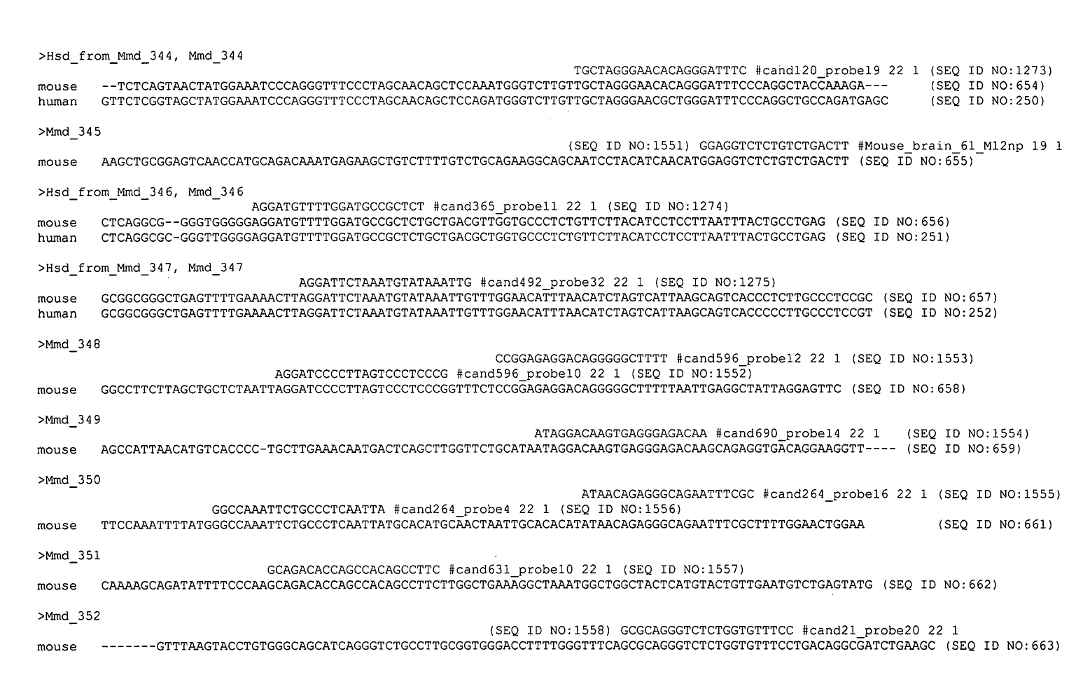

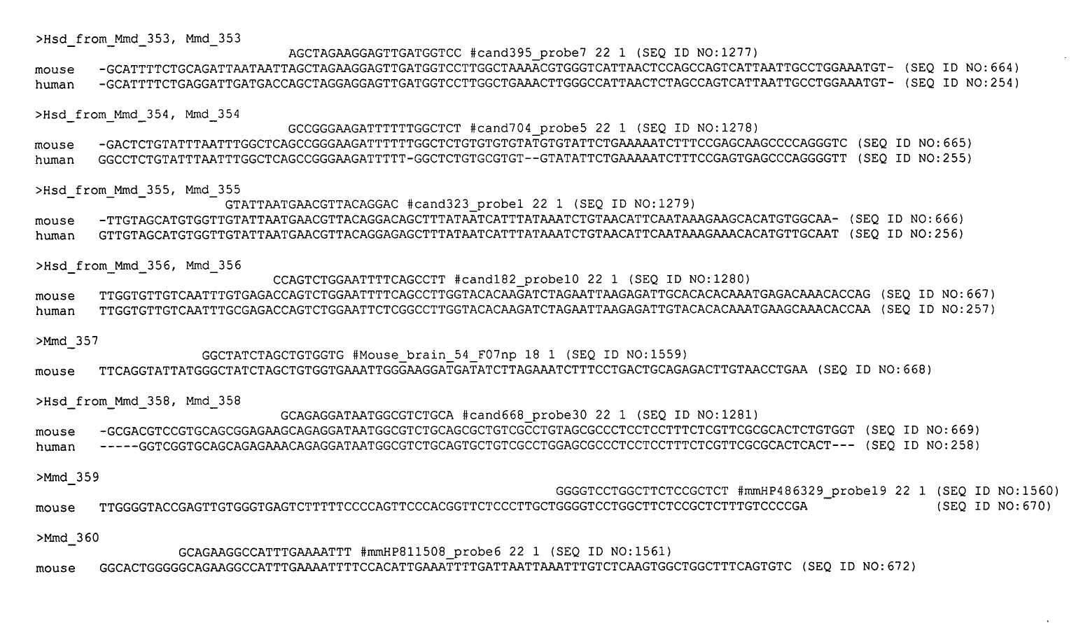

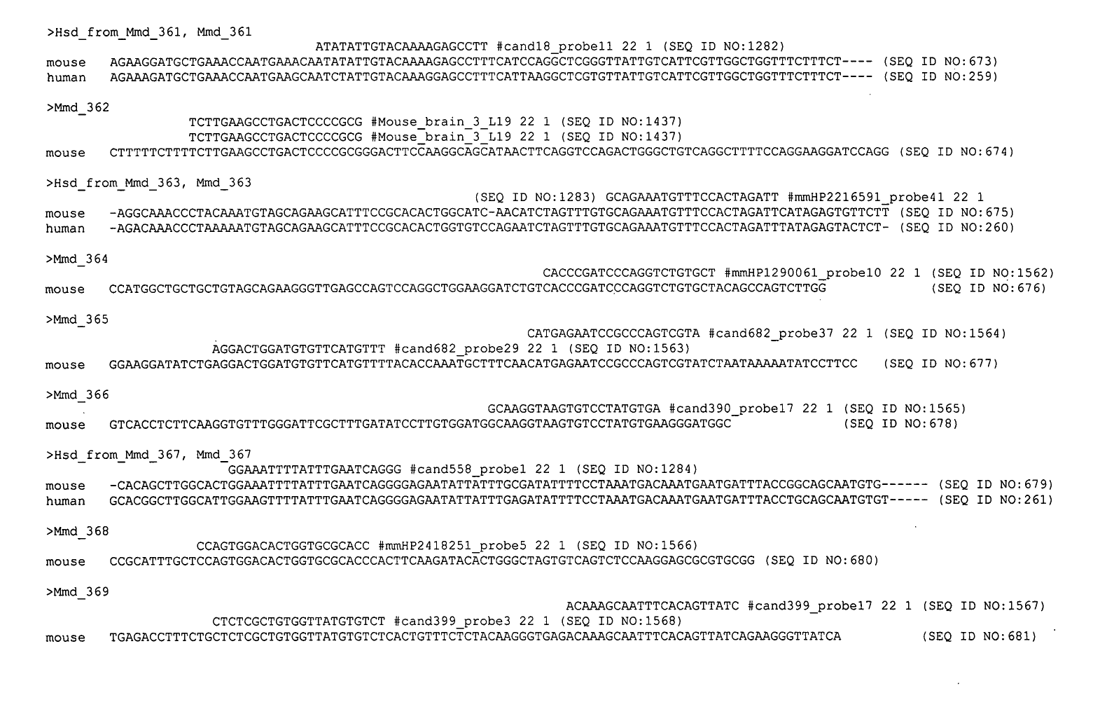

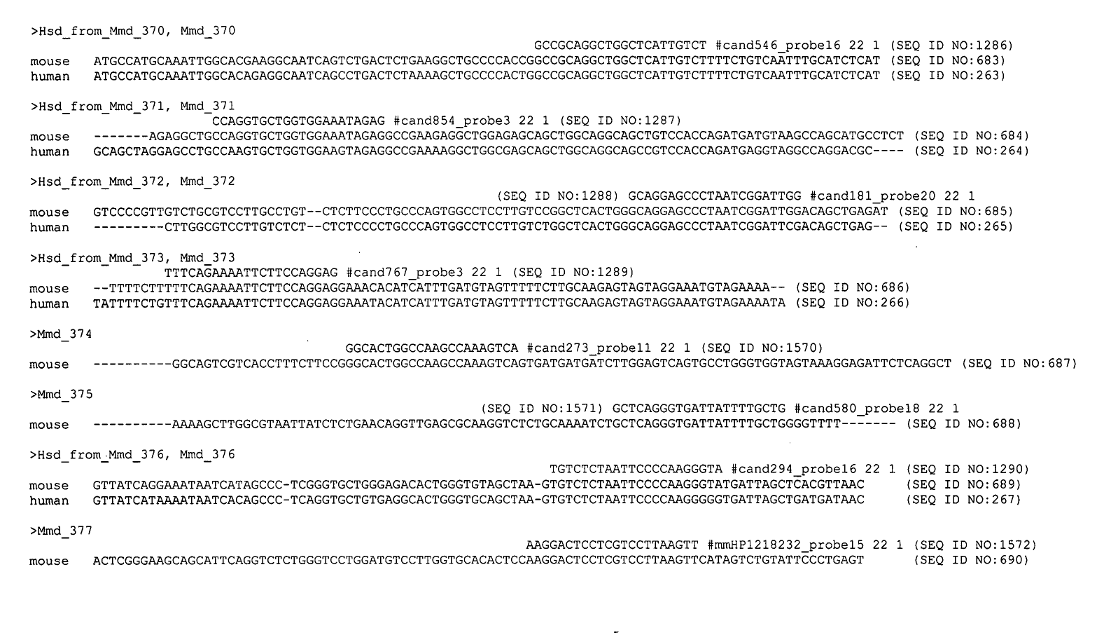

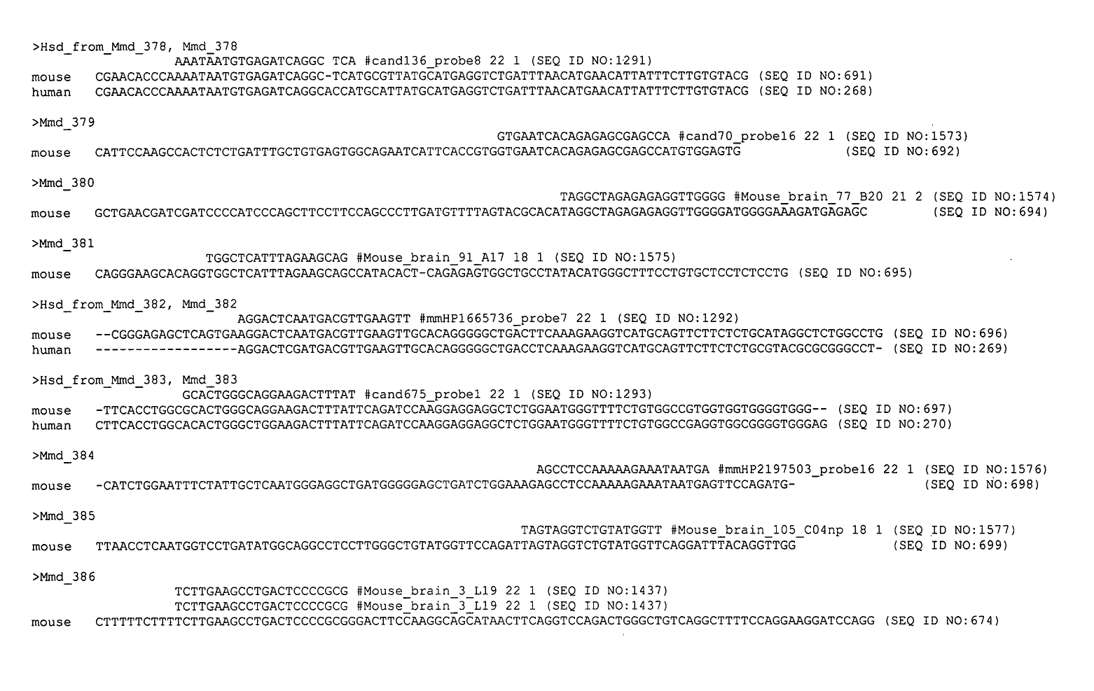

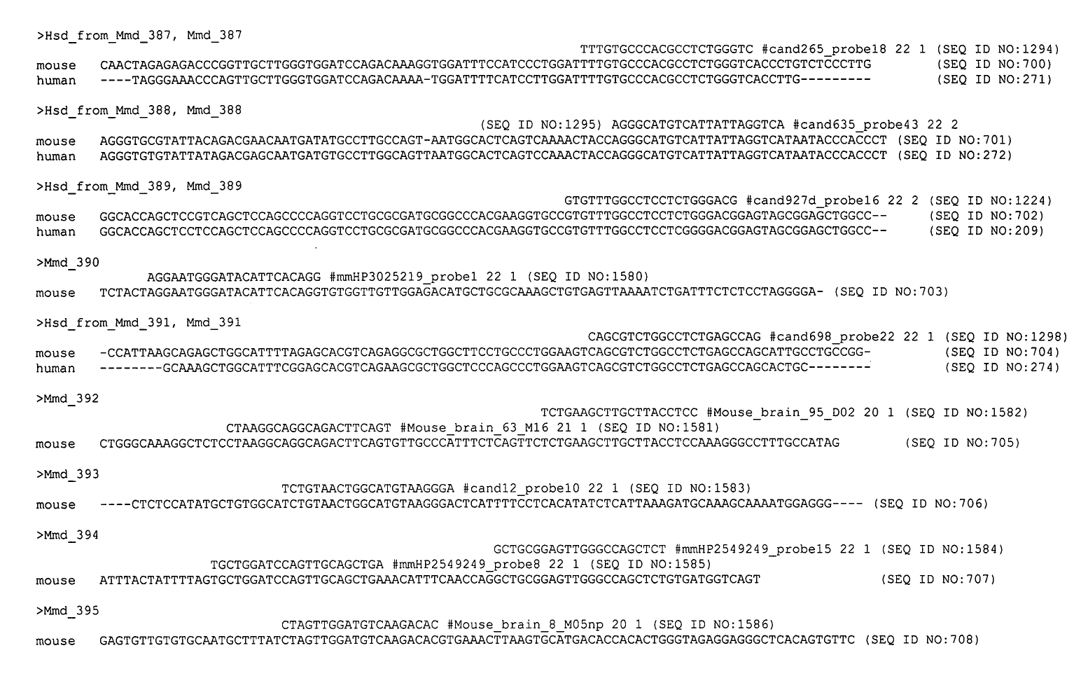

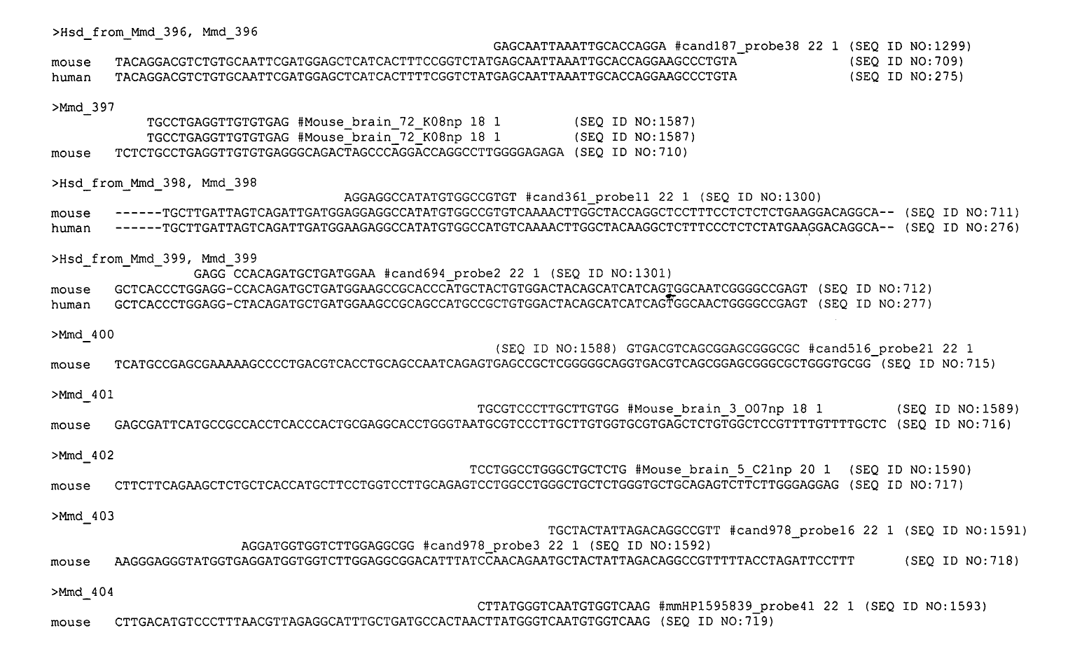

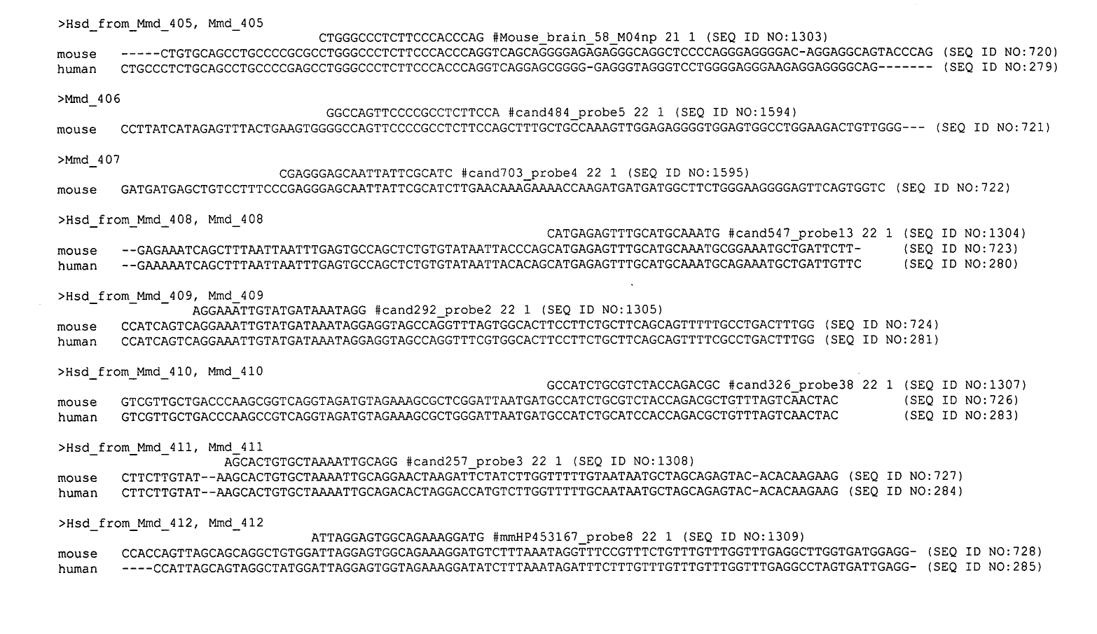

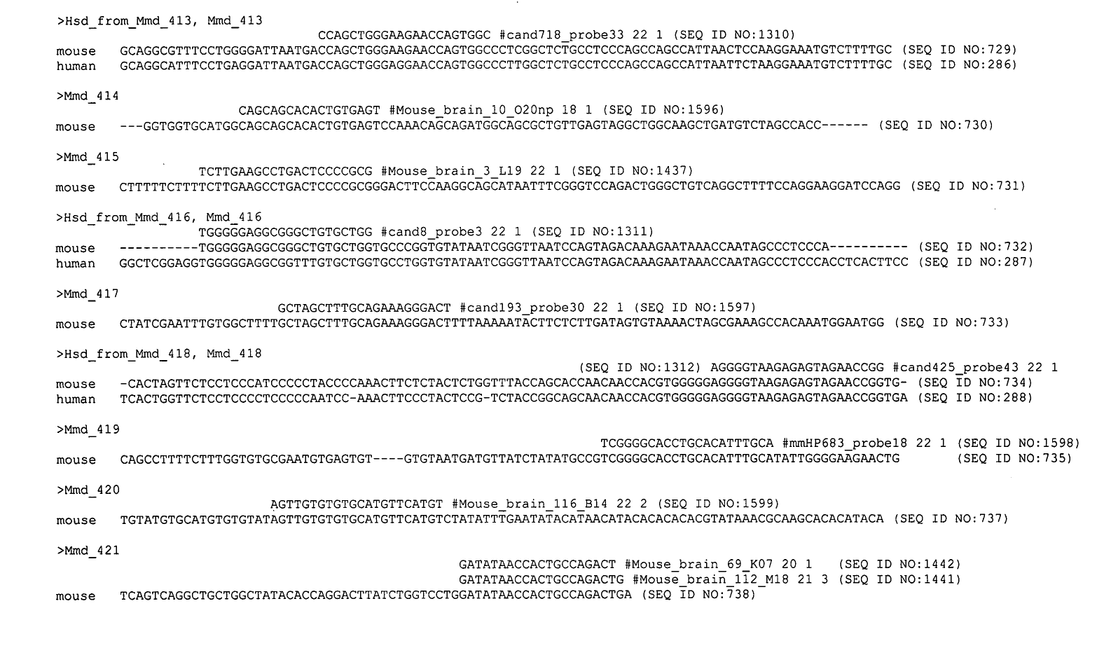

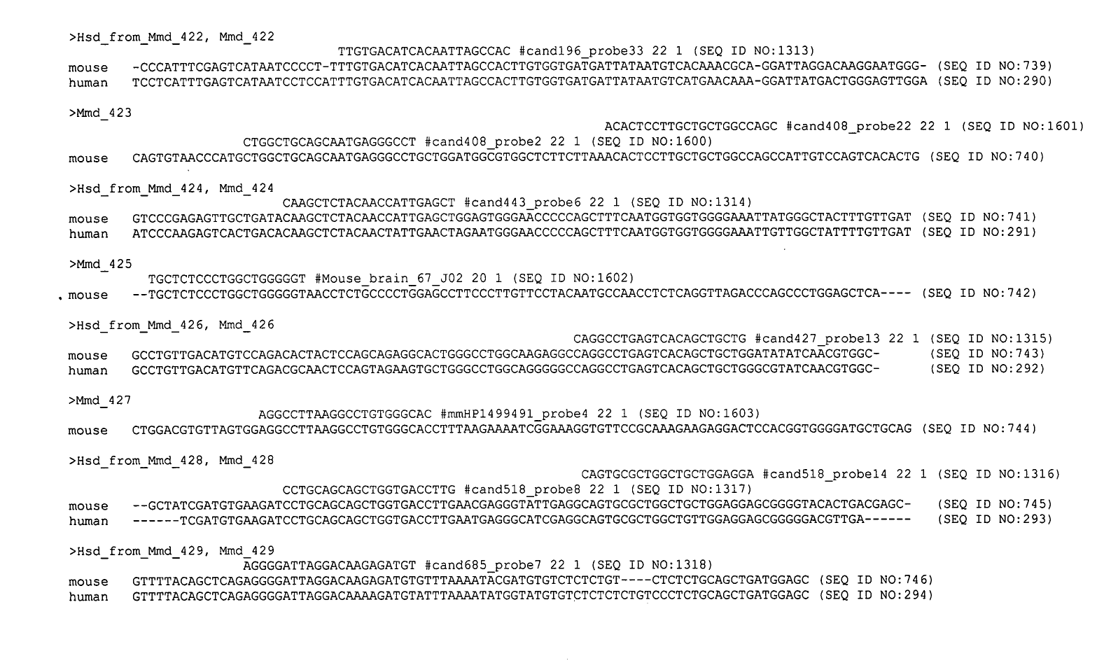

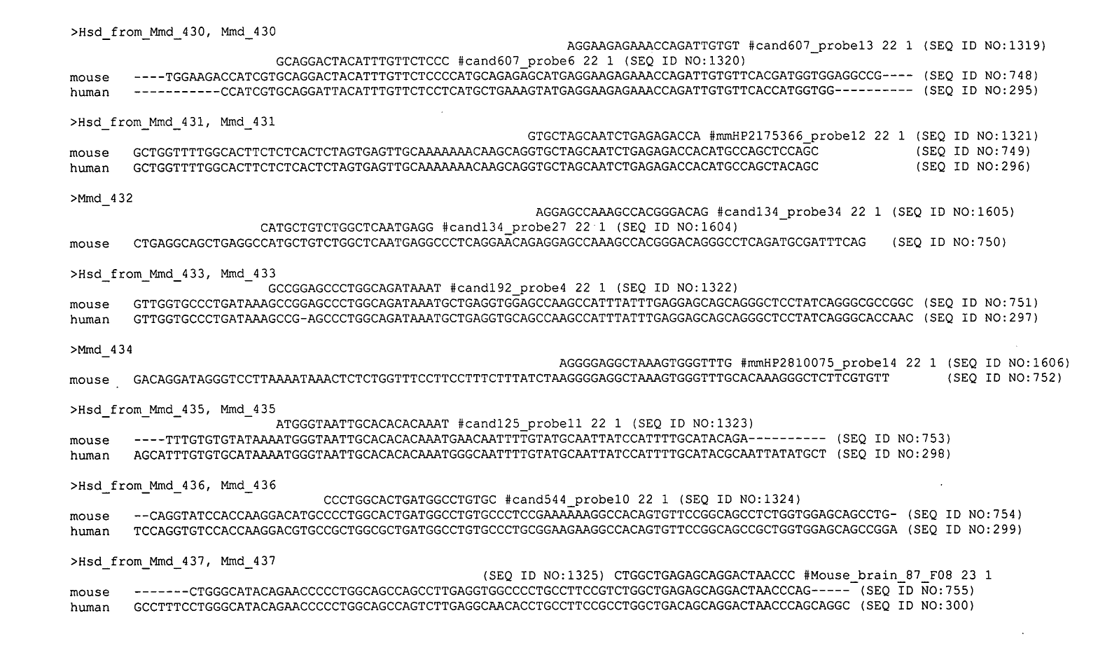

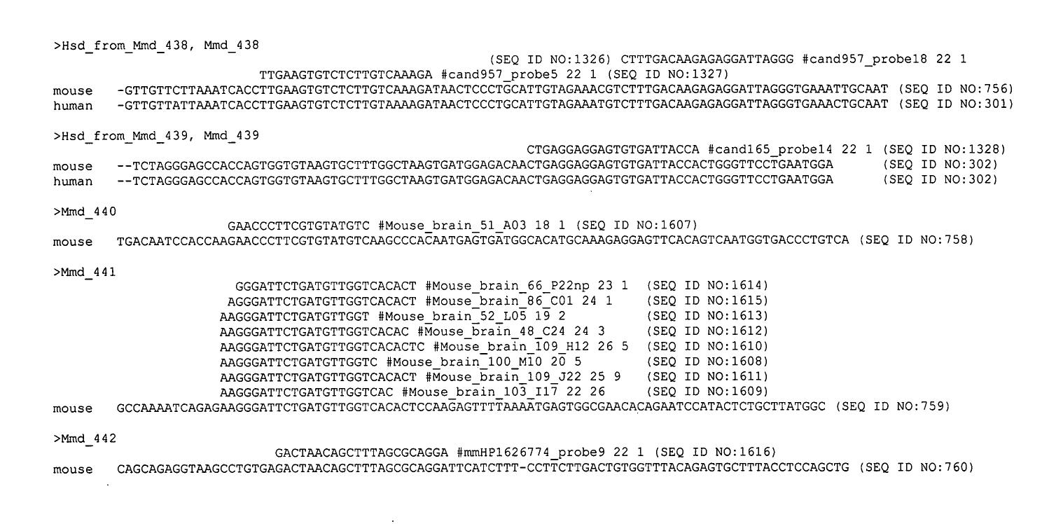

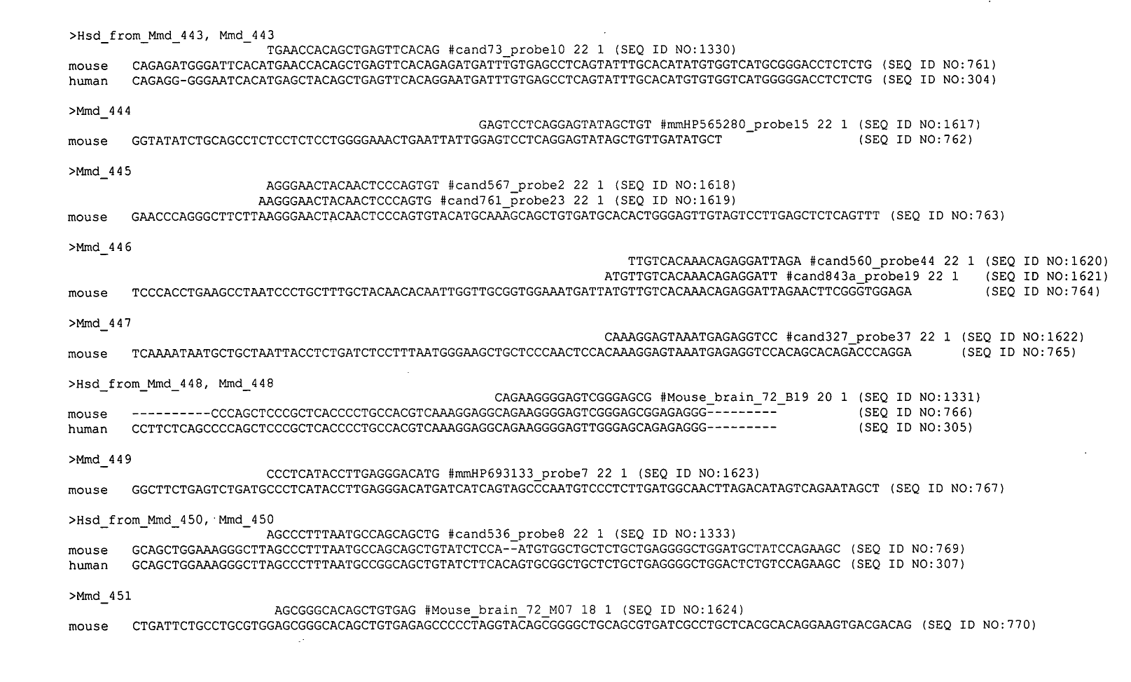

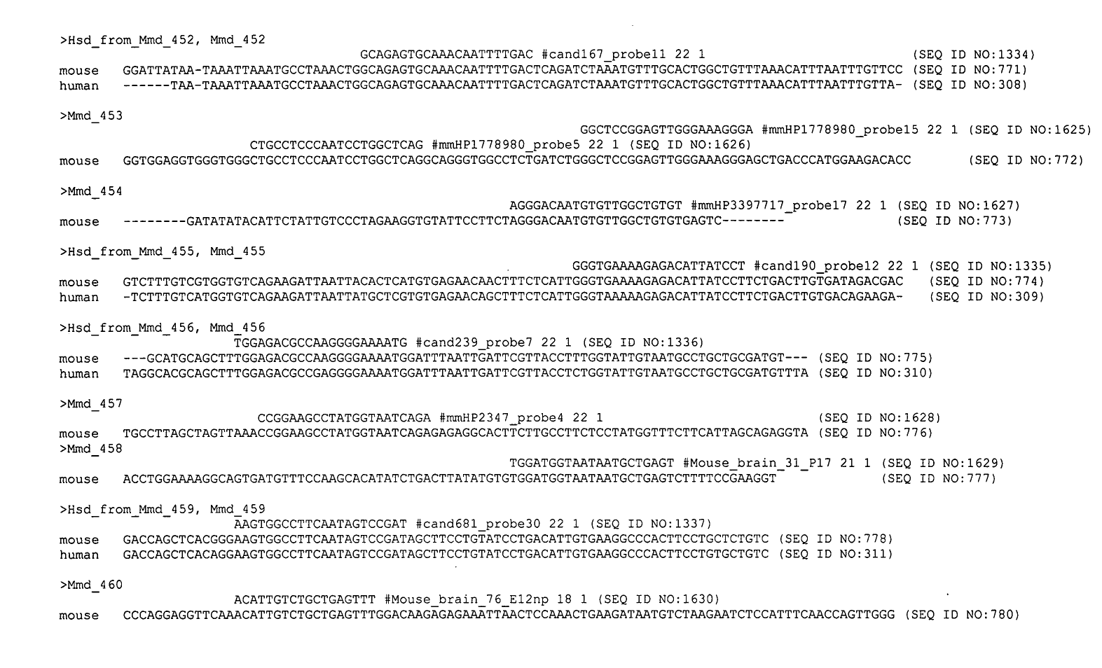

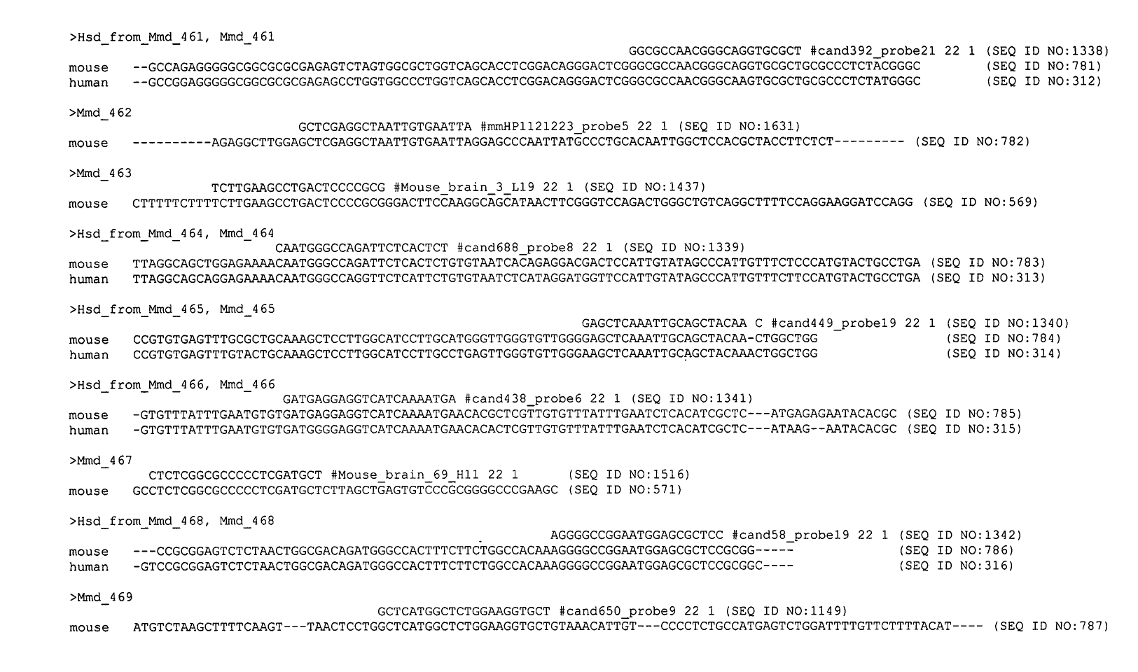

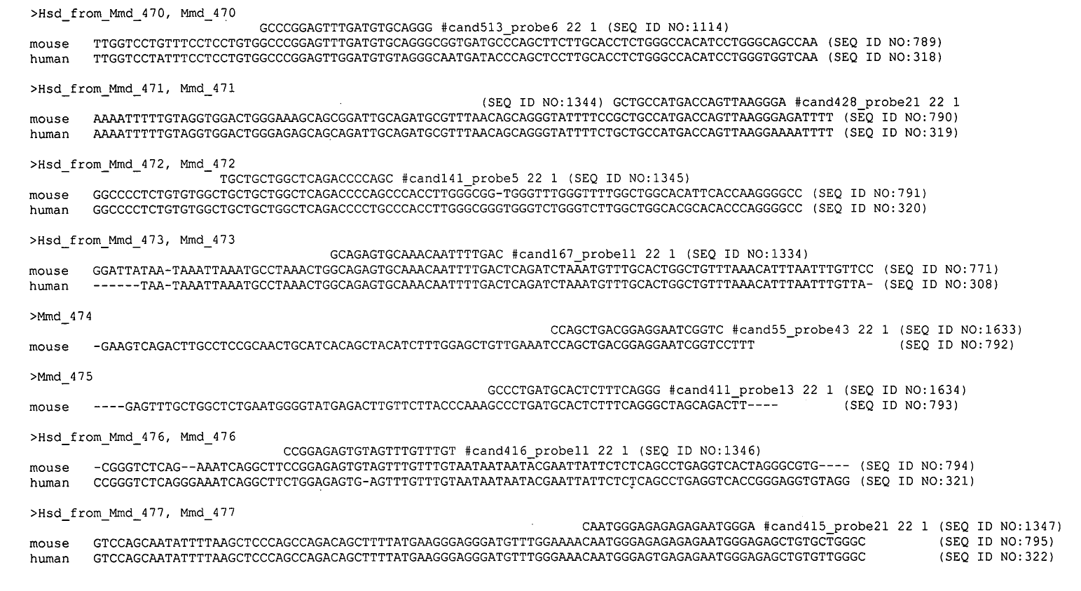

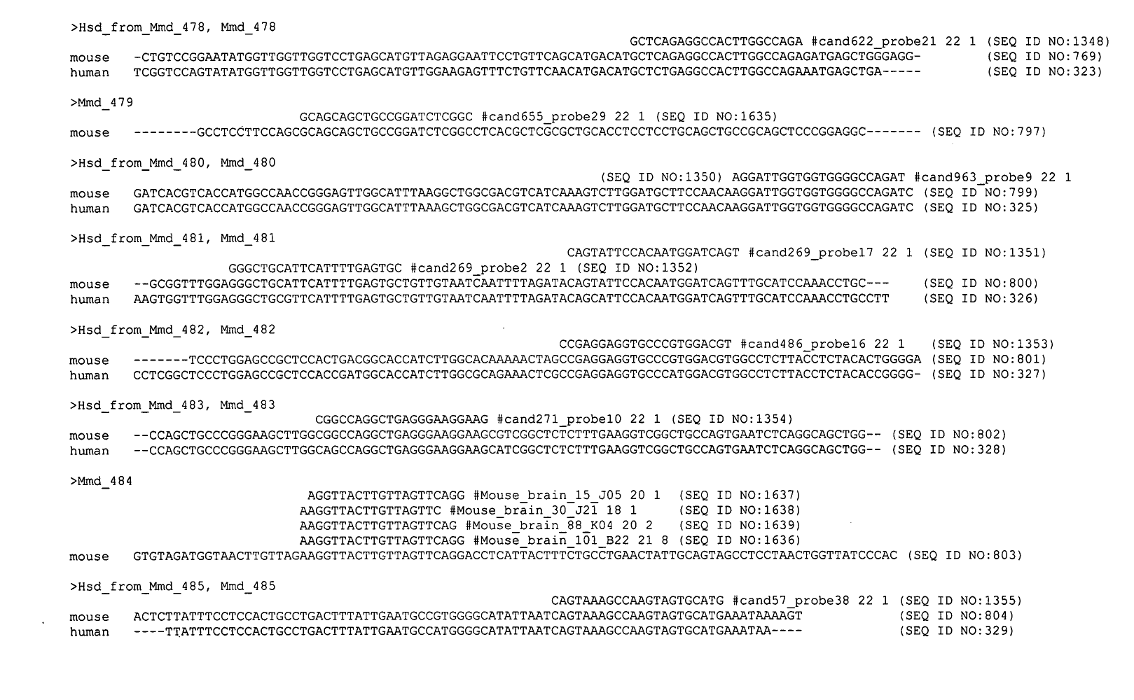

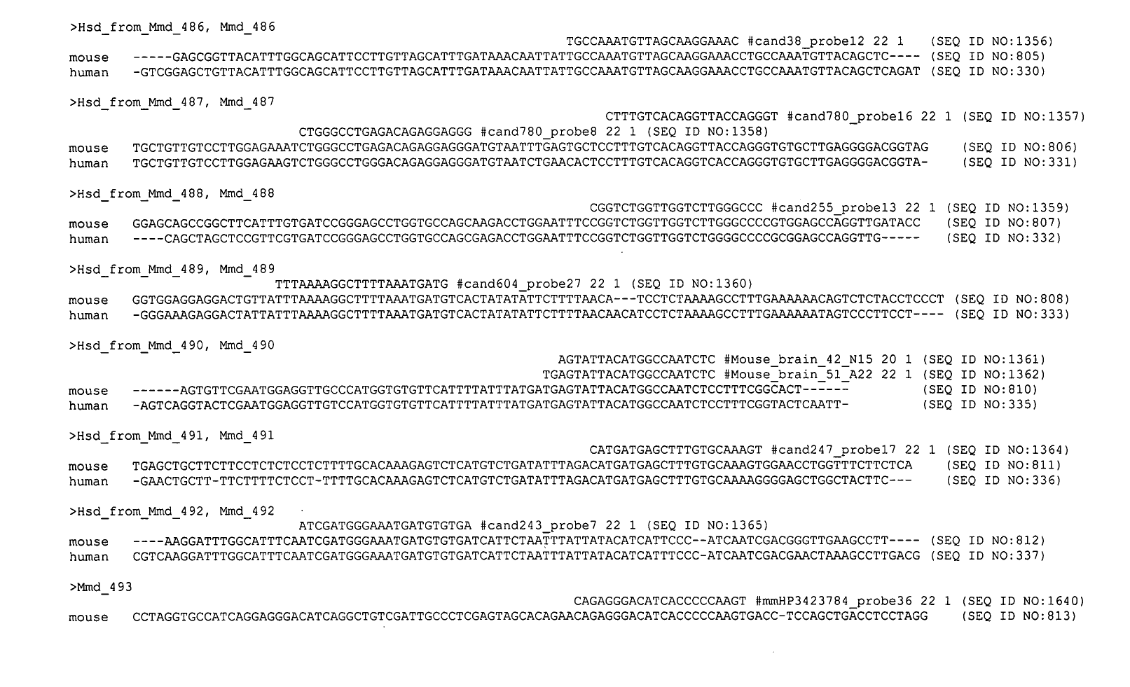

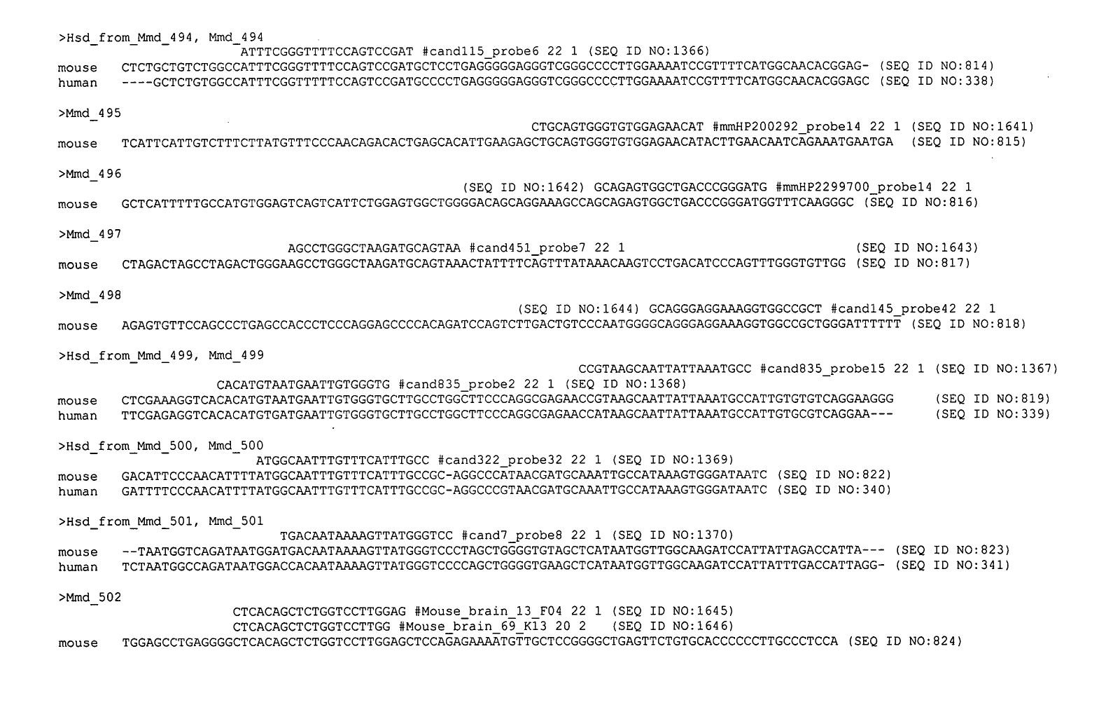

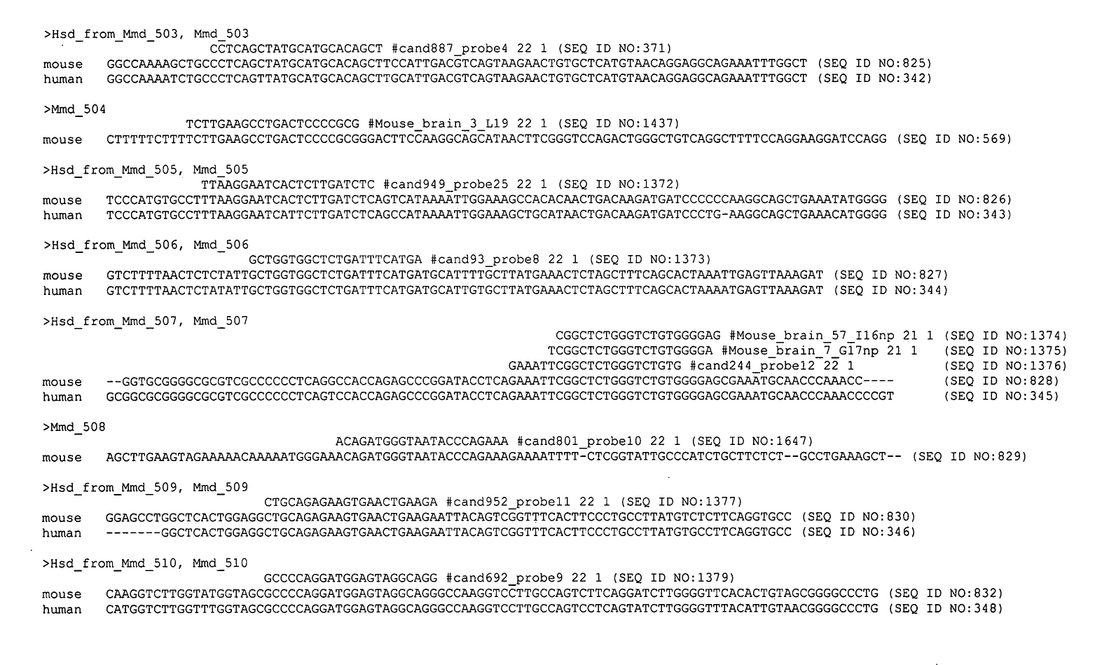

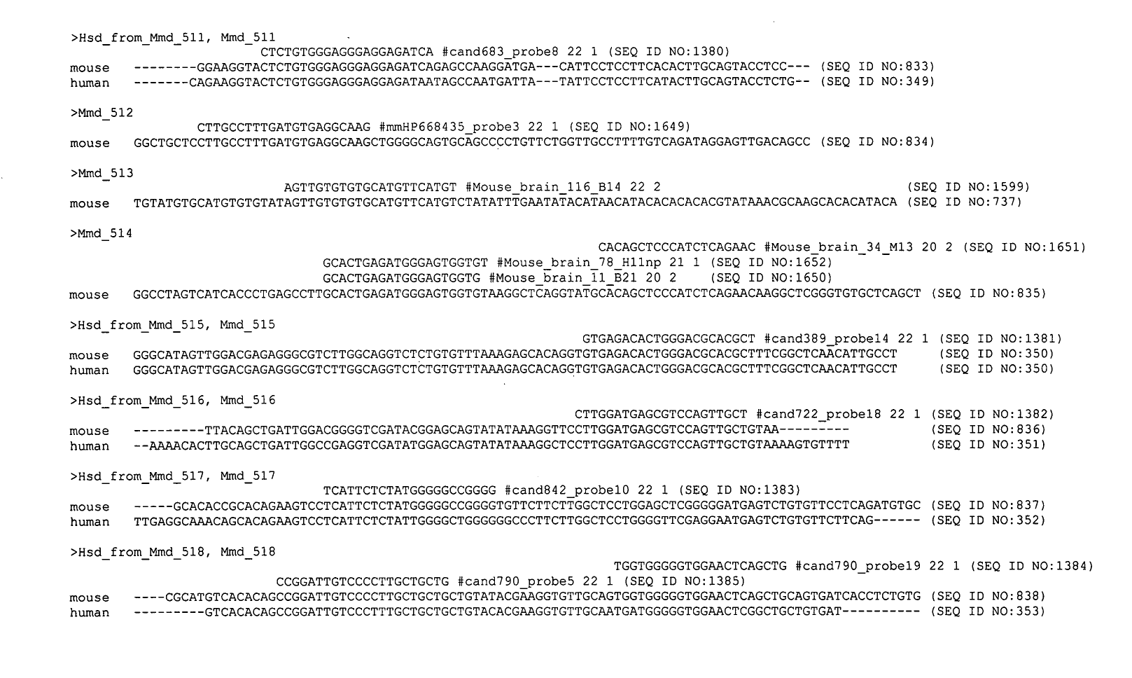

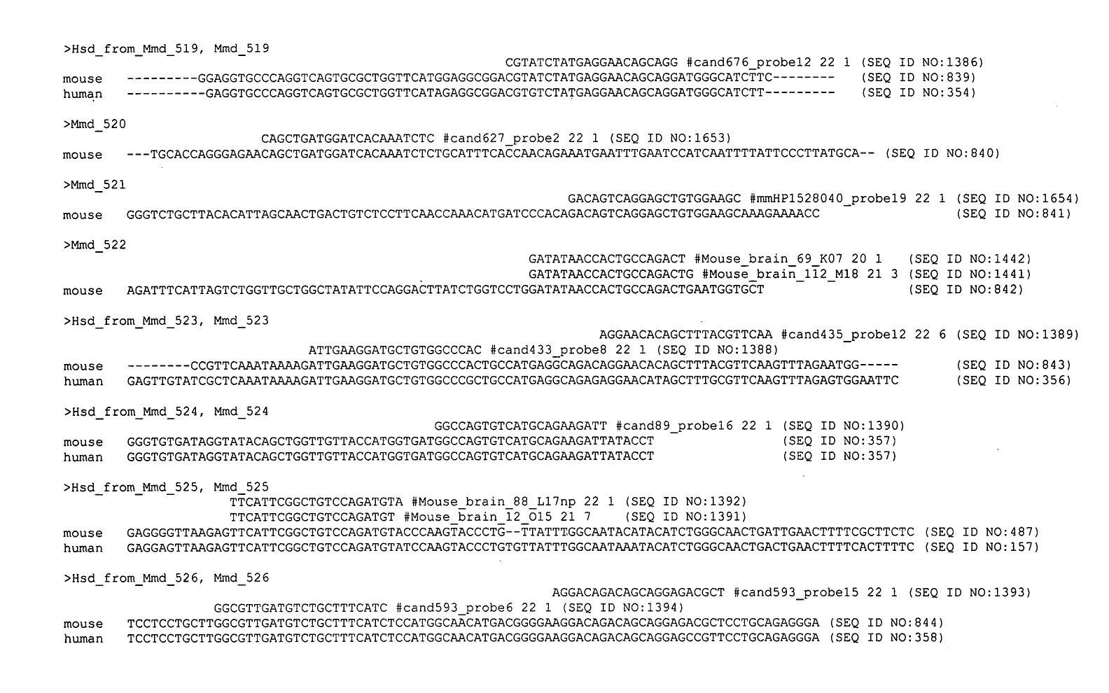

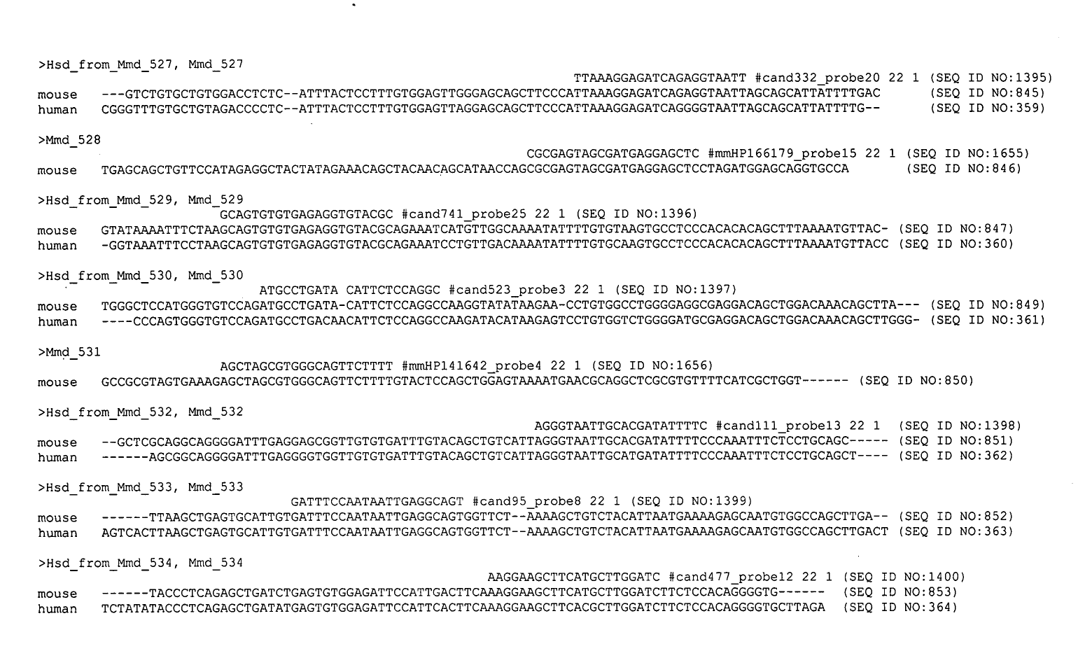

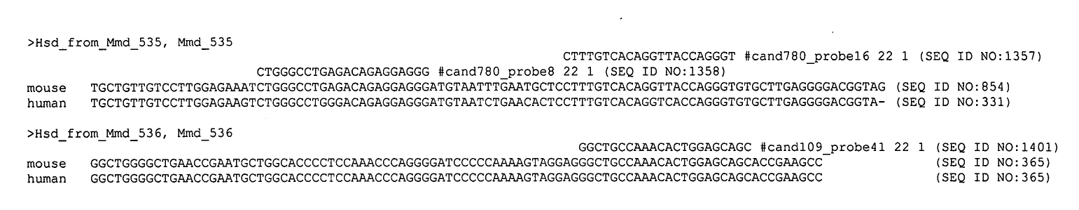

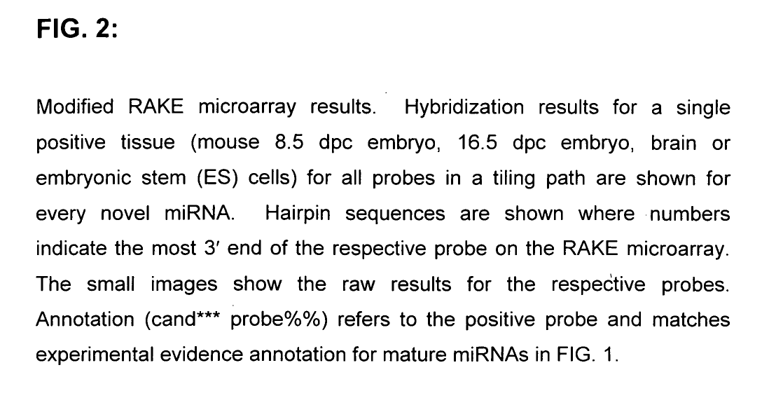

The invention provides novel miRNA sequences and precursors and complements thereof. The larger RNA species from which miRNA are excised have various names such as pre-miRNA, pri-miRNA and as used in the invention hairpin RNA. The invention provides many different miRNA and at least some of the larger RNA species from which they are derived. The miRNA and hairpin RNA provided by the invention are listed in FIG. 1. This figure contains a substantial amount of information on the miRNA, the cloning source, the hairpin RNA structure, mammalian homologues thereof, and extracted data from experimental results of FIG. 2, etc. The various elements of FIG. 1 are detailed in FIG. 1A. Different cell types were analysed for the presence of the respective miRNAs. In cases where a miRNA was produced by a cell, the structure and nucleotide sequence of the miRNA was determined. The invention thus further provides a method for analysing a sample comprising nucleic acid from a cell by determining the presence therein of a particular miRNA or hairpin RNA of FIG. 1. Correlation of the detected miRNAs with the pre-miRNAs revealed that accurate prediction of miRNA directly on the basis of the nucleic acid sequence of a pre-miRNA is not possible. The results found by the modified RAKE-approach, as detailed in FIG. 2A, for example in, one instance, showed a resulting miRNA from one strand of a predicted miRNA precursor, in another instance from two strands of a precursor. Moreover there was a significant variability of the position of the miRNA in the predicted precursor, the amount and sequence of nucleotides at either end of a strand.

It was found that miRNAs and hairpin RNAs of the invention are differentially expressed in cells of various origins. A probe specific for an individual miRNA or hairpin RNA can thus be used to differentiated samples on the basis of the expression of the respective miRNA or hairpin RNA. The invention therefore provides a method for characterising a sample comprising nucleic acid derived from a cell, said method comprising determining whether said sample comprises at least a minimal sequence of at least one miRNA (miRNA) of the invention or a mammalian homologue thereof and/or whether said sample comprises a precursor of said miRNA (hairpin RNA) of the invention or mammalian homologue thereof and characterizing said sample on the basis of the presence, relative abundance, or absence of said miRNA or hairpin RNA.

FIG. 1 depicts miRNA and precursors thereof (further referred to herein as hairpin RNA) of the invention. The hairpin RNA provided in FIG. 1 is typically shorter than the actual precursor RNA found in the cell. It contains the sequences that form the stem-loop structure from which miRNA are excised.

MiRNA were detected in various biological sources, depending on the miRNA and the biological source. Analysis of the structure of the miRNA revealed that miRNA produced from hairpin RNA are a heterologous group wherein the individual miRNA share a typically central, sequence. The individual miRNA produced from a pre-miRNA differ from each other at the 5′ end, the 3′ end, or both ends. A minimal sequence of a miRNA of the invention is a sequence that is shared by all identified miRNA variants from one half of the pre-miRNA or hairpin RNA. The half may be the half having the 5′ of the pre-miRNA or hairpin RNA or the half having the 3′ end of the pre-miRNA or hairpin RNA. A minimal sequence of a miRNA containing an uneven number of nucleotides is typically a sequence of at least 10 nucleotides comprising the central nucleotide of the miRNA and at least the 4 nucleotides next to the central nucleotide at either the 5′ or the 3′ side of the central nucleotide. For a miRNA containing an even number of nucleotides, a minimal sequence is typically a sequence of 10 nucleotides comprising the two central nucleotides of the miRNA and at least the 4 nucleotides next to the central nucleotides at either the 5′ or the 3′ side of the two central nucleotides. In another embodiment a minimal sequence of a miRNA of FIG. 1, comprises at least the “seed” sequence of said miRNA, i.e. nucleotides 2-8 of a miRNA of FIG. 1.

As different miRNA are differently expressed in various cell types or tissues, a method of the invention can be used to characterized the source of the sample. For instance, a probe specific for a miRNA that is expressed in heart tissue but not in embryonic cells can be used to classify a sample as either not containing RNA from the heart or vice versa, not containing nucleic acid derived from embryonic cells. For miRNA expressed in other tissues or cells similar characterisations are possible.

Nucleic acid obtained from a natural source can be either DNA or RNA. In the present invention it is preferred that said nucleic acid comprises RNA. The nucleic acid is preferably directly derived from a cell. However, the nucleic acid can also have undergone one or more processing steps such as but not limited to chemical modification. A miRNA or pre-miRNA of the invention, or complement thereof can also be used to analyse DNA samples, for instance, by analysing a sequence of an obtained (pre-) miRNA it is possible to determine the species that the cell belonged to that provided the nucleic acid for the analysis.

Characterisation of a sample on the basis of the presence, relative abundance, or absence of a particular miRNA and/or hairpin RNA can be used as an indicator for the presence or absence of disease, such as cancer. For instance, when a sample from a tissue comprises a different expression pattern of miRNA and/or hairpin RNA when compared to a comparable tissue from a normal individual, or when compared to a comparable tissue from an unsuspected part of said tissue from the same individual. A difference in the presence of one miRNA and/or hairpin RNA provides an indication in this type of analysis. However, the accuracy (i.e. predictive value) of the analysis typically increases with increasing numbers of different miRNA and/or hairpin RNA that are analysed. Thus a method for the characterisation of a sample of the invention preferably comprises determining whether said sample comprises at the least minimal sequence of 5 different miRNA or hairpin RNA of FIG. 1 or a mammalian homologue thereof. Preferably, at the least minimal sequence of 10, preferably at least 20 more preferably at least 60 different miRNA and/or hairpin RNA of FIG. 1 or a mammalian homologue thereof. A method of the invention may of course further include detection of miRNA and/or hairpin RNA of the art. It is preferred that the presence or absence of at least a minimal sequence of a miRNA of FIG. 1 is determined in a method of the invention. It is typically the miRNA that exerts an expression regulating function in a cell. The presence of pre-miRNA and/or hairpin RNA in a sample is of course indicative for the presence of at least the minimal sequence of the corresponding miRNA in said sample, although this does not always have to be true. Preferably, a method of the invention, further comprises determining whether said sample comprises at least a minimal sequence of at least five miRNA (miRNA) of FIG. 1, or a mammalian homologue thereof wherein said at least five miRNA are derived from at least five different hairpin RNA and characterizing said sample on the basis of the presence or absence of said miRNA.

A sample can comprise cells. Typically, however, a sample has undergone some type of manipulation prior to analysing the presence or absence therein of a miRNA and/or hairpin RNA according to the invention. Such manipulation, typically, though not necessarily comprises isolation of at least (part of) the nucleic acid of the cells. The nucleic acid in a sample may also have undergone some type of amplification and/or conversion prior to analysis with a method of the invention. miRNA can be detected directly via complementary probe specific for said miRNA or indirectly. Indirect forms include, but are not limited to conversion into DNA or protein and subsequent specific detection of the product of the conversion. Conversion can also involve several conversions. For instance, RNA can be converted into DNA and subsequently into RNA which in turn can be translated into protein. Of course such conversions may involve adding the appropriate signal sequences such as promoters, translation initiation sites and the like. Other non-limiting examples include amplification, with or without conversion of said miRNA in said sample for instance by means of PCR or NASBA or other nucleic acid amplification method. All these indirect methods have in common that the converted product retains at least some of the specificity information of the original miRNA and/or hairpin RNA, for instance in the nucleic acid sequence or in the amino acid sequence or other sequence. Indirect methods can further comprise that nucleotides or amino acids other than occurring in nature are incorporated into the converted and/or amplified product. Such products are of course also within the scope of the invention as long as they comprise at least some of the specificity information of the original miRNA and/or hairpin RNA. By at least some of the specificity information of the original miRNA and/or hairpin RNA is meant that the converted product (or an essential part thereof) is characteristic for the miRNA and/or hairpin RNA of which the presence or absence is to be determined.

The cell comprising said nucleic acid can be any type of cell. As mentioned above, it can be an embryonic cell, a foetal cell or other pre-birth cell, or it can be a cell of an individual after birth, for instance a juvenile or an adult. It can also be a cell from a particular part of a body or tissue of a mammal. Preferably, said cell is an aberrant cell, preferably a cell with an aberrant proliferation phenotype such as a tumour cell or a tissue culture cell. Preferably a cancer cell, or a cell suspected of being a cancer cell. In another preferred embodiment said cell is a cell that is infected with a pathogen. Preferably said pathogen is a virus or a (myco)bacterium.

A method of the invention is particularly suited for determining the stage of said aberrant cell. For instance, tumorigenic cells can have varying degrees of malignancy. While progressing through the various degrees of malignancy the pattern of expression of (pre-) miRNA changes and can be detected. Such a pattern can thus be correlated with the degree of malignancy. A method of the invention can thus be used for determining a prognosis for the individual suffering from said cancer.

The cell is preferably a lung cell, a skin cell, a brain cell, a liver cell, an embryonic cell, a heart cell, an embryonic cell line or an aberrant cell derived there from.

Changes in expression are better detected when a test sample is compared with a reference. Thus in one aspect the invention provides a method for determining whether a cell in a sample is different from a reference cell, comprising determining whether expression of at least one at least one miRNA of FIG. 1 or a mammalian homologue thereof or at least one hairpin RNA of FIG. 1 or a mammalian homologue thereof, in said cell is different when compared to said reference cell. Preferably it is determined whether the expression of at least 5 miRNA or hairpin RNA is different in said cell in said sample when compared to a reference cell. Expression is different when there is at least a factor of two difference in the level of expression. Preferably, the difference is a difference between detectable miRNA expression and not detectable. Preferably said at least 5 miRNA or pre-miRNA are of FIG. 1. Expression levels can be compared by comparing steady state levels or by comparing synthesis rates.

A cell as used herein is a cell of a mammal, preferably a mouse, a rat, a primate or a human. A sample is for example characterized for the presence or absence of a disease, for belonging or not belonging to a certain species, or for being in a specific stage of development. In many instances however, a sample is best characterized by determining the presence, relative abundance, or absence therein of a collection of miRNAs and/or hairpin RNAs of the invention, as a sample of an organism usually displays a natural and/or pathological variation in diverse parameters.

Another reason why a sample is preferably characterized on the basis of a collection of miRNAs and/or hairpin RNAs, is that a disorder manifests itself in variable manners in different individuals. These two causes of variability can however, be calculated in through providing detection information of a collection of miRNAs and/or hairpin RNAs. For example, a characteristic expression profile of a disease is composed of a collection of miRNAs and/or hairpin RNAs. By comparing an expression profile of said collection in a sample to a reference expression profile of said collection that is characteristic of said disease, an individual from whom this sample is taken, is thus tested for presence or absence of said disease. The process of determining whether a sample matches an expression profile of a disease or a species depends on multiple factors. A miRNA itself has more or less distinctive power within, for example, a disorder or a species. Further a miRNA as part of a collection represents a percentage of a total collection. Characterizing a sample thus preferably comprises, apart from determining the absence or presence of one miRNA, determining the absence or presence of more miRNAs. Absence or presence of a miRNA is for example a positive or a negative indicator for a disease or a species. A collection or an expression profile preferably comprises one or more positive and/or negative indicators. Said positive and/or negative indicators are for example expressed as a percentage of a total number of miRNAs or as an absolute number of miRNAs. When expressing indicators in percentages, a weight is optionally attributed to an indicator. An indicator with a higher distinctive power is herein preferably given a higher weight than an indicator with a low distinctive power.

In one embodiment the invention provides a method according to the invention, comprising determining whether said sample comprises at least a minimal sequence of at least two, preferably at least three, more preferably at least four, most preferably at least five miRNAs of FIG. 1 or a mammalian homologue thereof wherein said miRNA are preferably derived from different precursor miRNA (pre-miRNA) and characterizing said sample on the basis of the presence or absence of said miRNA. The presence on a different hairpin RNA as depicted in FIG. 1, or on different mammalian homologs thereof is indicative for the presence on different precursor miRNA. In a preferred embodiment said characterization of said sample is a test for a disease. In many instances a test comprising more miRNAs has a higher diagnostic value, however, this need not always be the case. In another preferred embodiment of the invention one or more miRNAs according to the invention are determined in a sample, in combination with one or more other miRNAs. In a further preferred embodiment at least one miRNA according to the invention is determined in a sample in combination with one or more other miRNAs, resulting in determining a total of at least 10, preferably at least 15, more preferably at least 20 or most preferably at least 25 miRNAs. In a preferred embodiment said other miRNAs determined in a sample are involved in the same type of disorder as said miRNA according to the invention that is determined in said sample. Alternatively, a test is composed of miRNAs with indicative values of two or more diseases or two or more species.

Said sample preferably comprises nucleic acid of a differentiated cell. Differentiated as used herein is either cellular differentiated or evolutionary differentiated. Preferably differentiated is cellular differentiated. A differentiated cell is derived from any part of an organism. Said cell is preferably derived from a part of an organism that is associated with a disease. For example, when characterizing a sample for cancer, said cell is preferably derived from a tumour. In another preferred embodiment said sample comprises nucleic acid of an embryonic cell. An embryonic cell can be derived from any organism but is preferably derived from a mammal. A sample comprising nucleic acid derived from an embryonic cell, is for example taken for early diagnosis of a disease in an organism. A embryonic cell is in one embodiment an embryonic stem cell. In a further preferred embodiment said sample comprises nucleic acid of a cell with an aberrant proliferation phenotype. An aberrant proliferation phenotype indicates that a proliferation process has somehow been disturbed. The disturbance is either caused by internal factors or by external factors or by a combination thereof. An aberrant proliferation phenotype is for example found in hepatitis, a bowel disease or a cancer. Preferably a cell with an aberrant proliferation phenotype is a tumour cell and/or cell line cell. A tumour cell is for example a leukemic cell, such as a leukemic B-cell. Said tumour cell line cell is for example obtained from a cell line that is cultured from a cell derived from a tumour of an organism, preferably a mammal. Alternatively said tumour cell line cell is obtained from a cell line that is cultured from a cell wherein tumour characteristics have been induced artificially, for example with a chemical substance. In a preferred embodiment the invention provides a method for characterizing a sample comprising nucleic acid derived from a cell according to the invention, wherein said cell is a lung cell, a skin cell, a brain cell, a liver cell, an embryonic cell, a heart cell, or an embryonic cell line.

In one embodiment the invention provides a method for determining whether a cell in a sample is modified when compared to a reference cell, comprising determining whether expression of at least one at least one miRNA of FIG. 1 or a mammalian homologue thereof and/or a hairpin RNA of FIG. 1 or a mammalian homologue thereof in said cell is altered when compared to said reference cell. A reference cell as used herein is for example a healthy or pathological counterpart of respectively a pathological or healthy cell. A reference cell is for example another cell of the same cell type of the same organism wherefrom said sample is taken but preferably from another organism. The other organism is preferably comparable in species and/or constitution and/or development and/or age. In a preferred embodiment said cell is a differentiated cell. In another preferred embodiment is an embryonic cell. In a further embodiment said cell is a cell with an aberrant proliferation phenotype. Preferably said cell with an aberrant proliferation phenotype is a tumour cell and/or cell line cell. In one embodiment the invention provides a method for determining whether a cell in a sample is modified when compared to a reference cell according to the invention, wherein said cell is a lung cell, a skin cell, a brain cell, a liver cell, an embryonic cell, a heart cell, or an embryonic cell line.

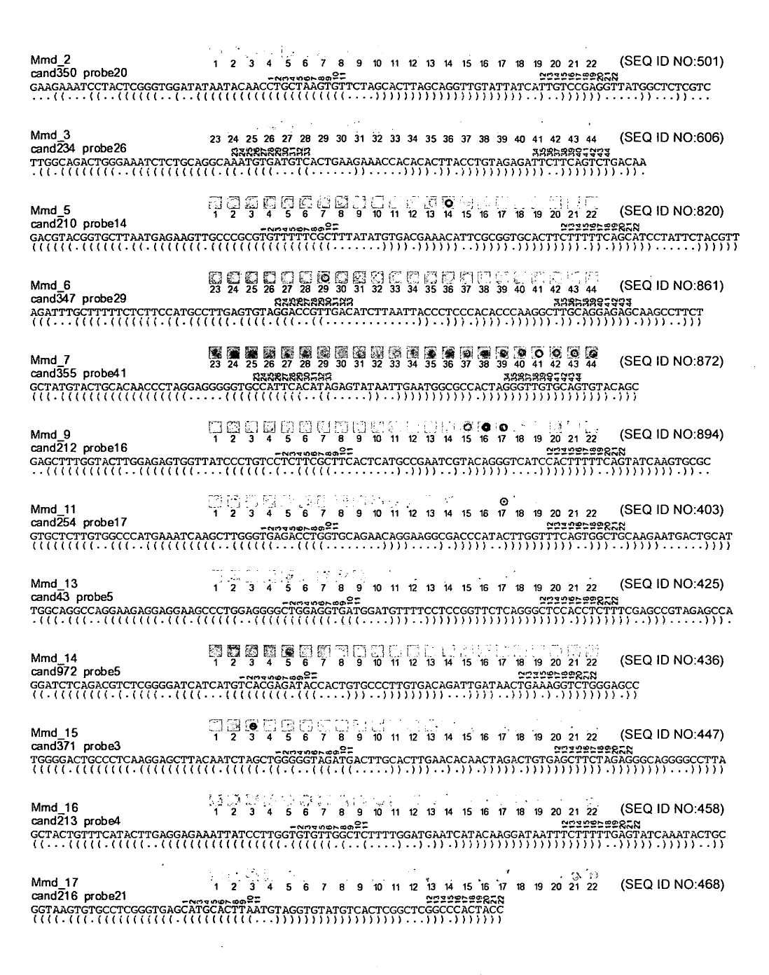

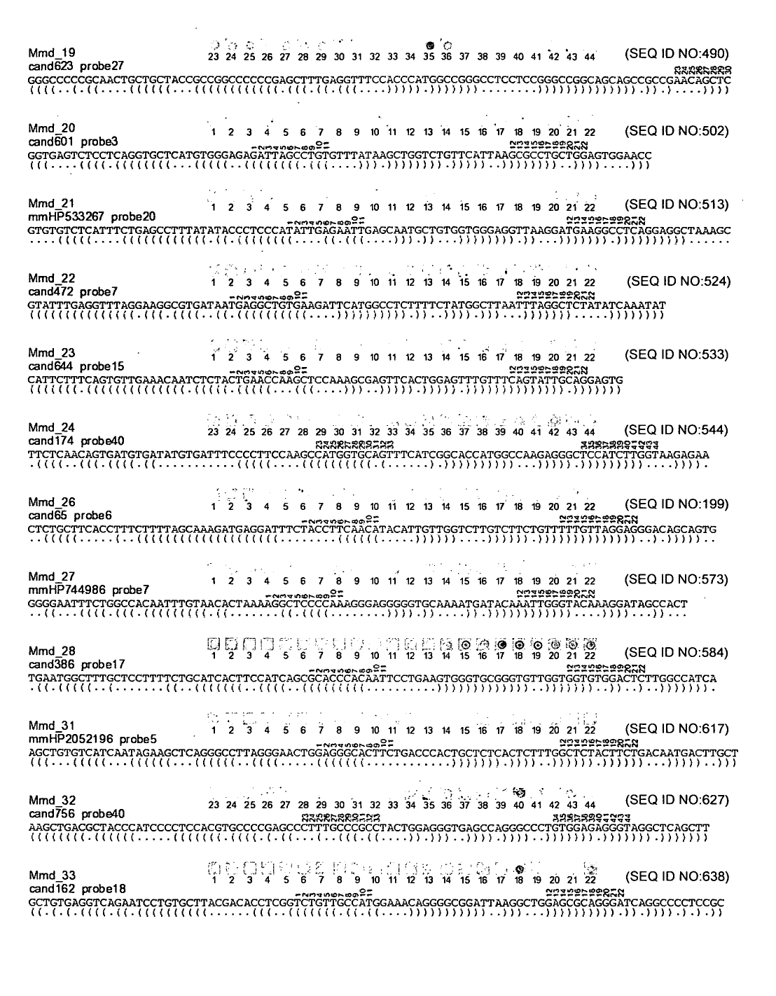

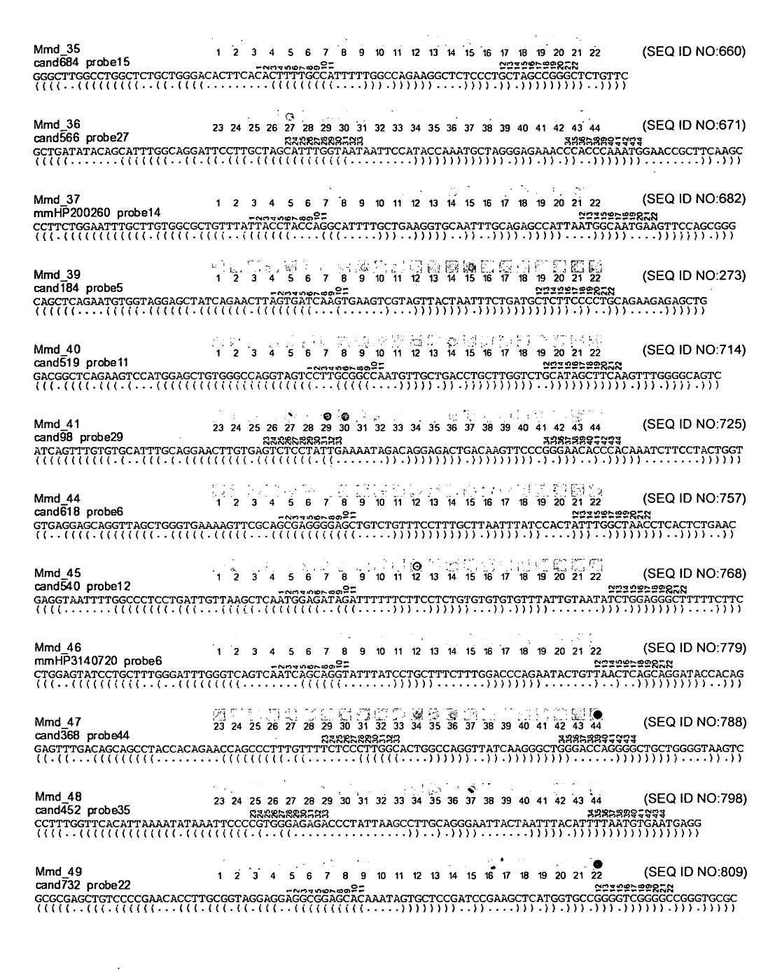

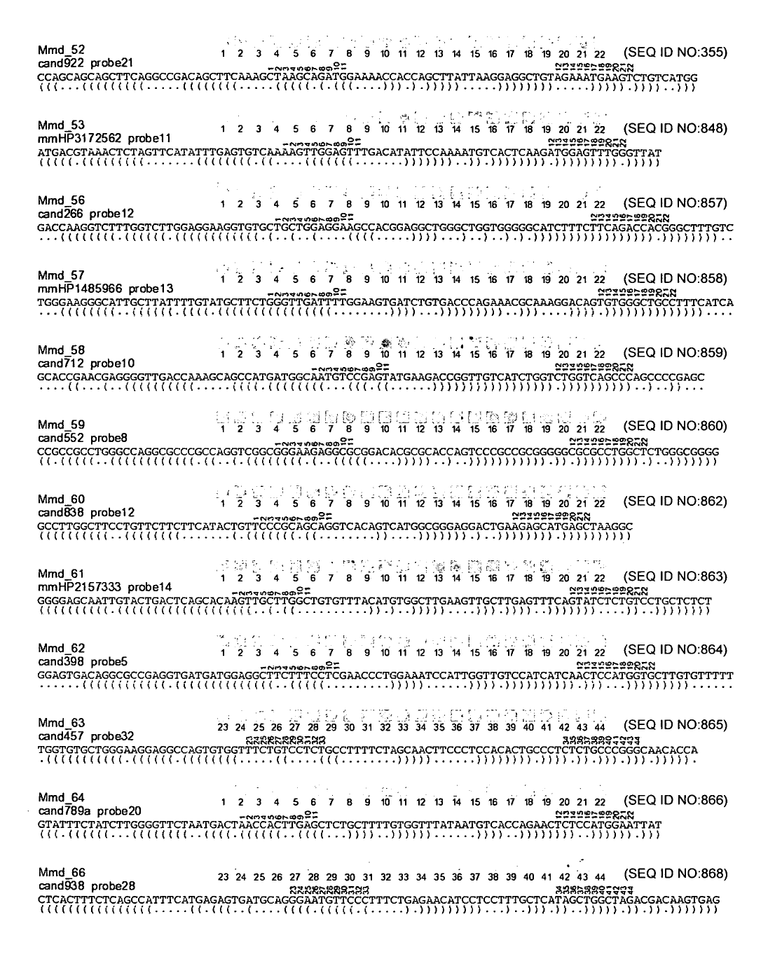

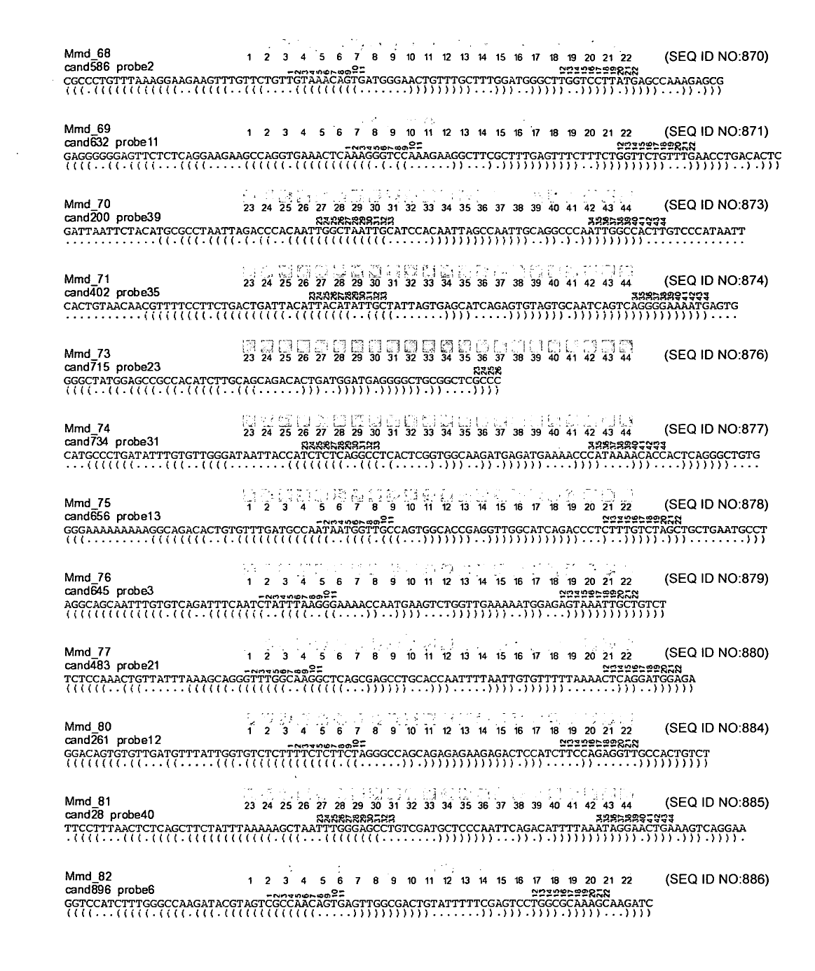

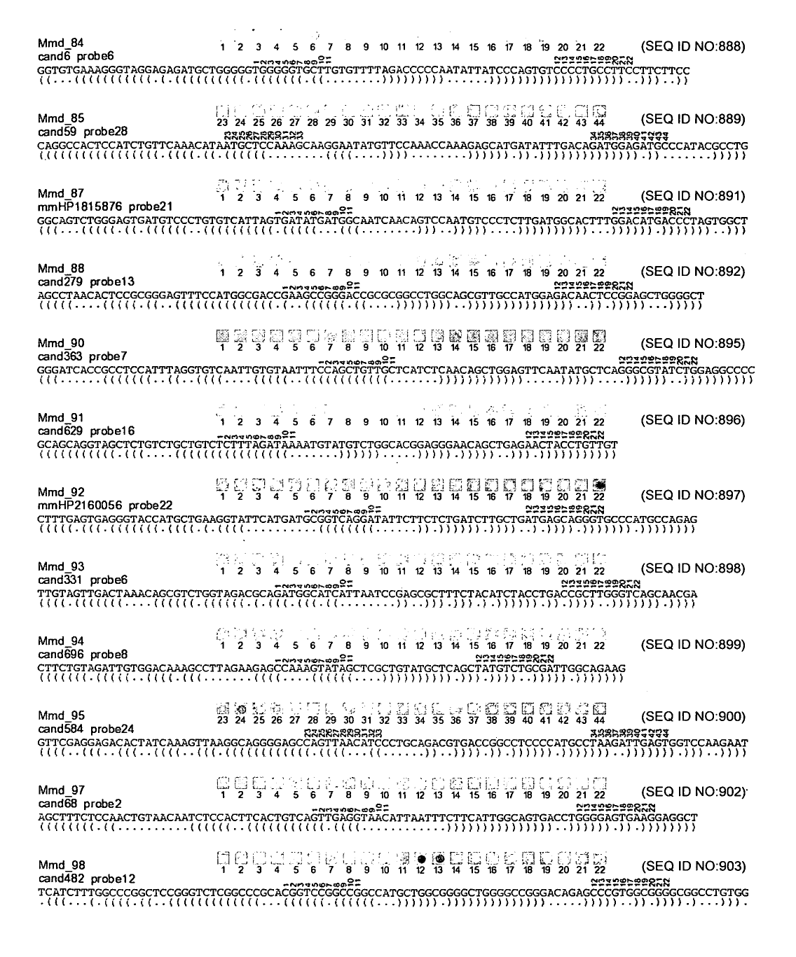

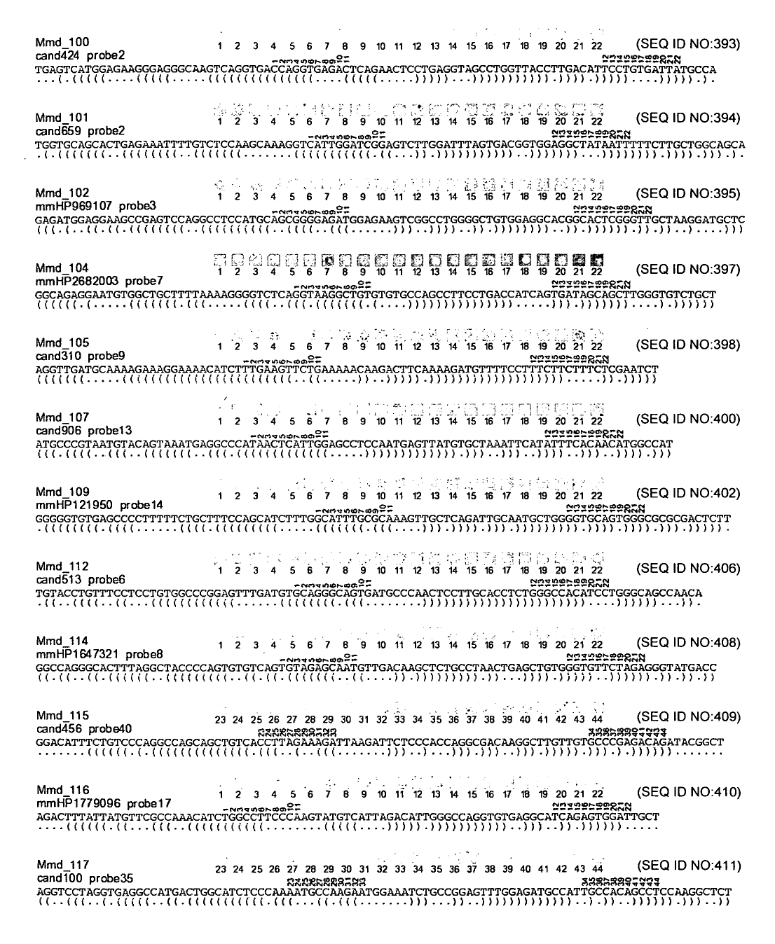

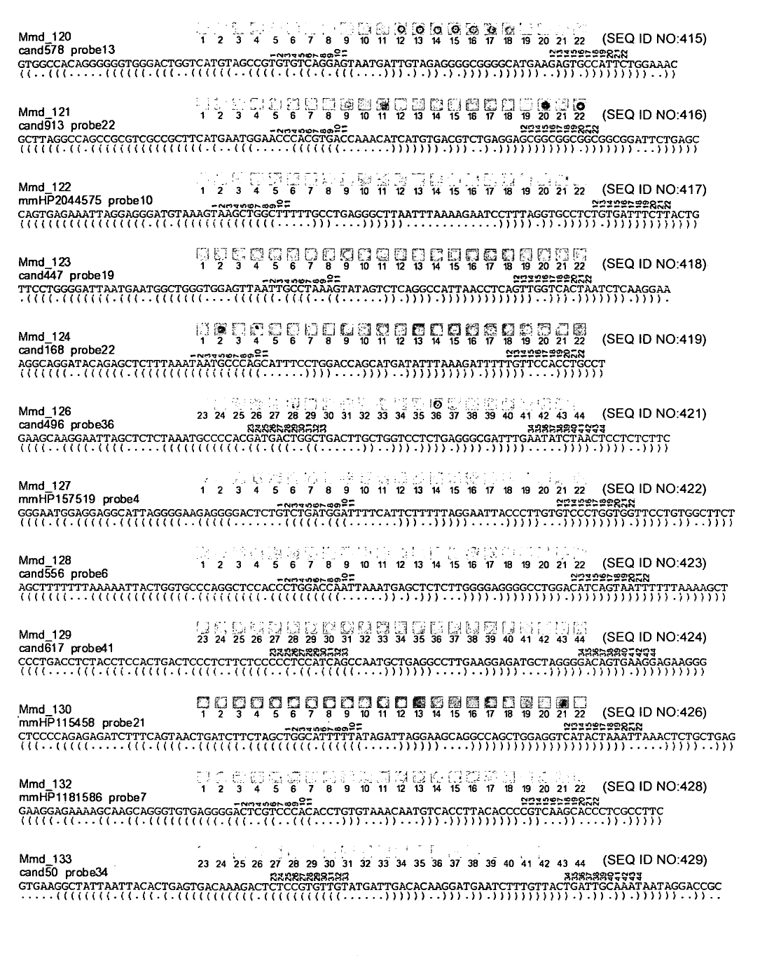

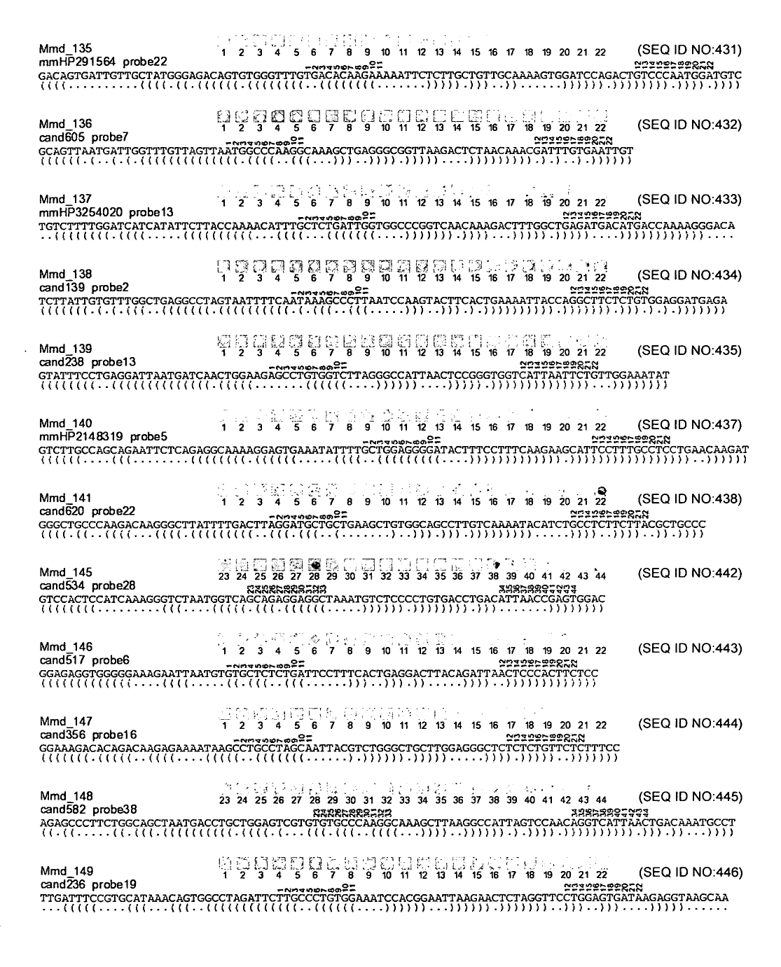

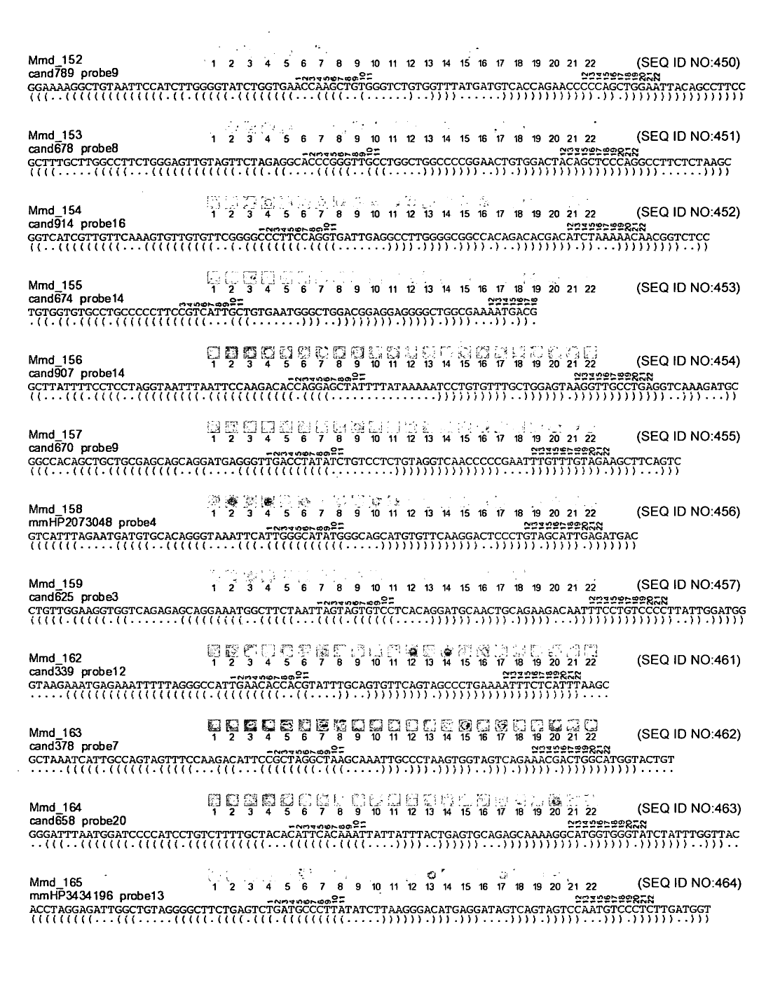

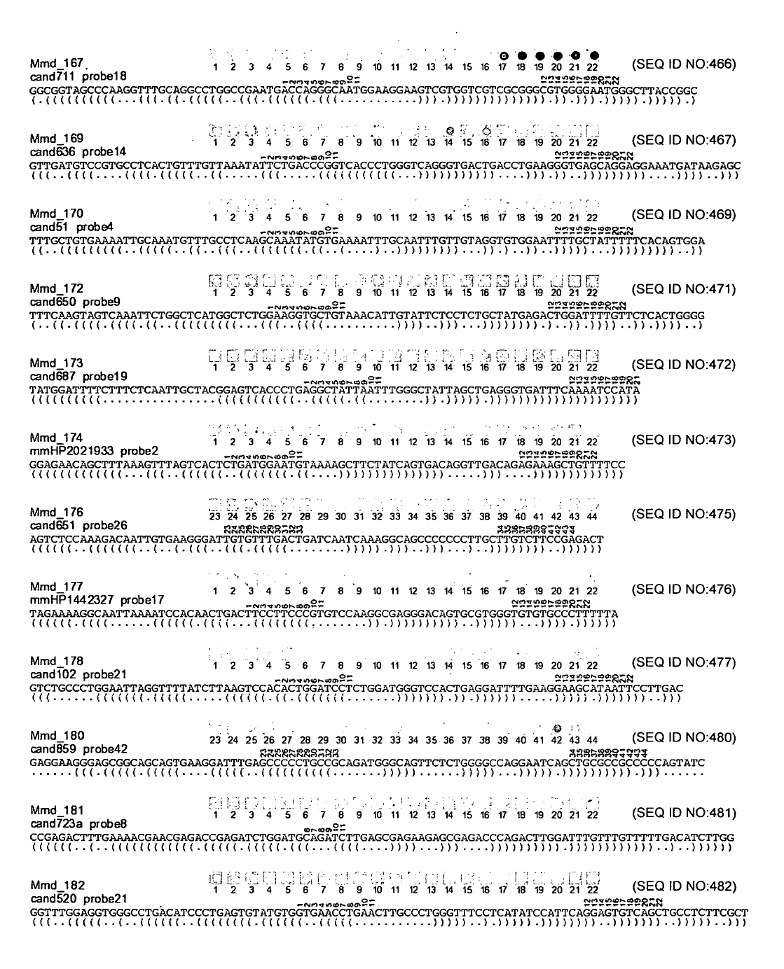

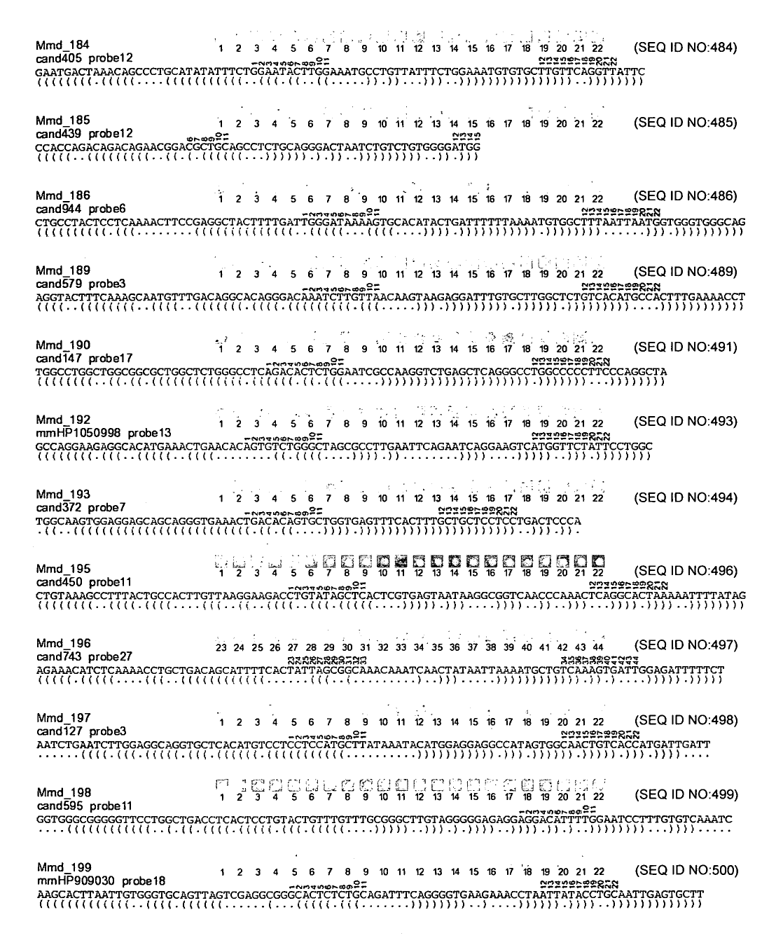

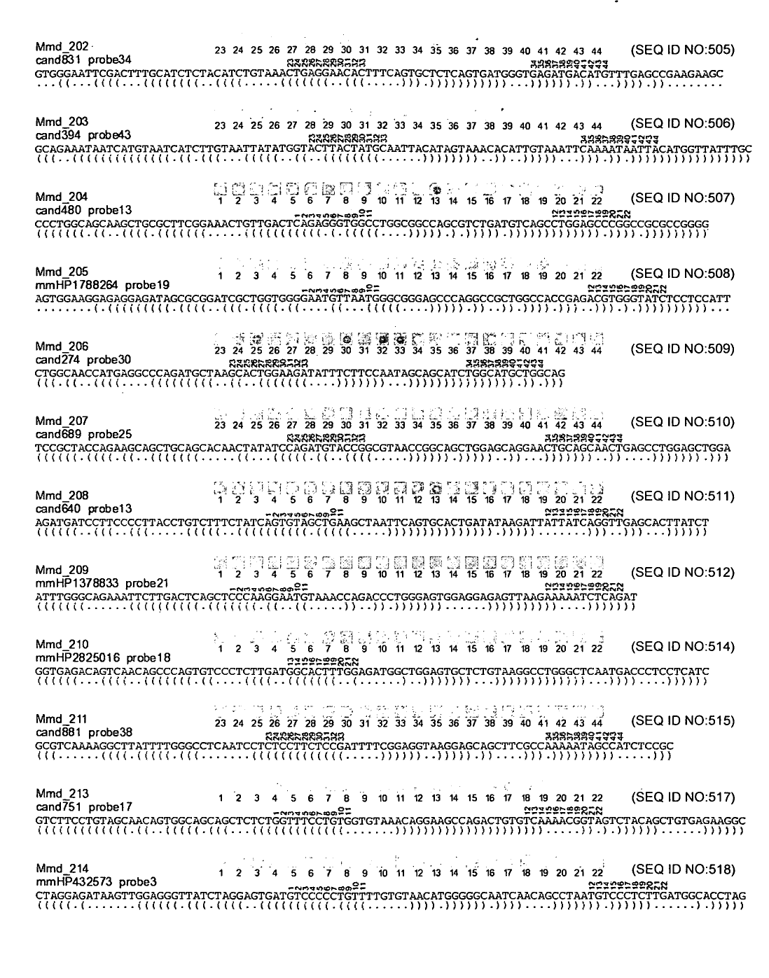

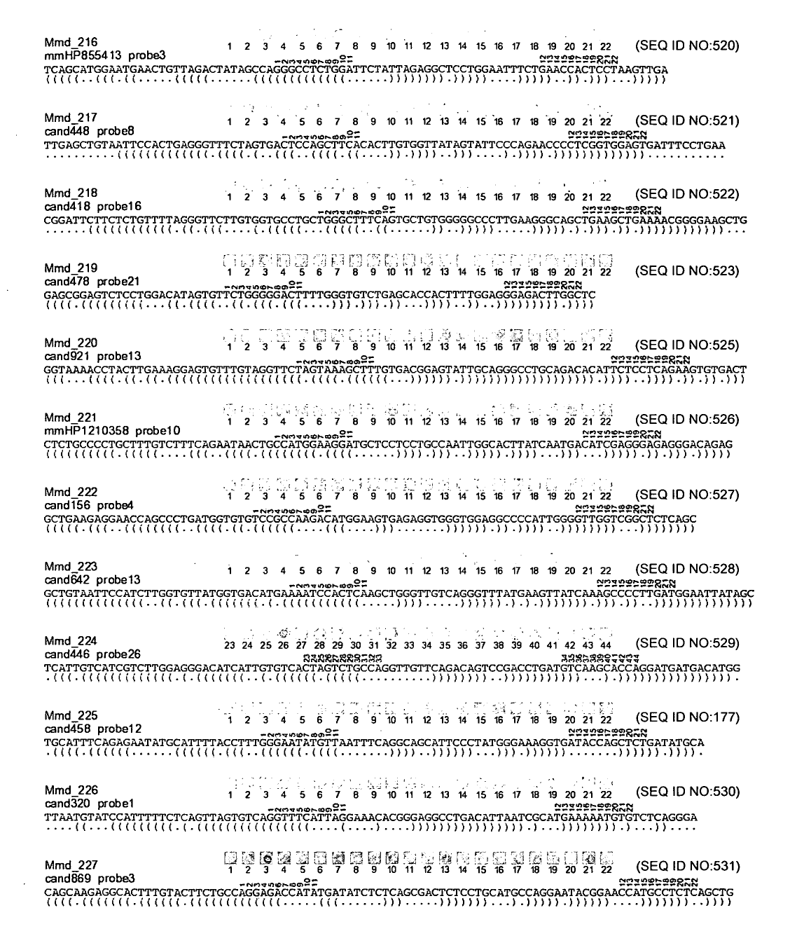

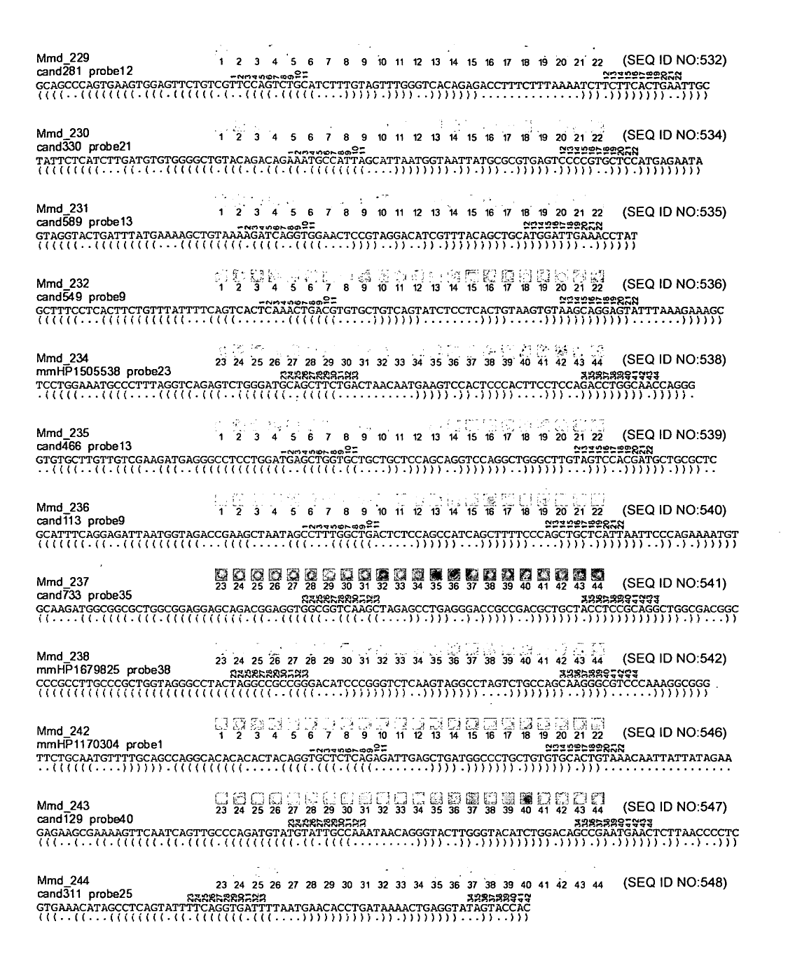

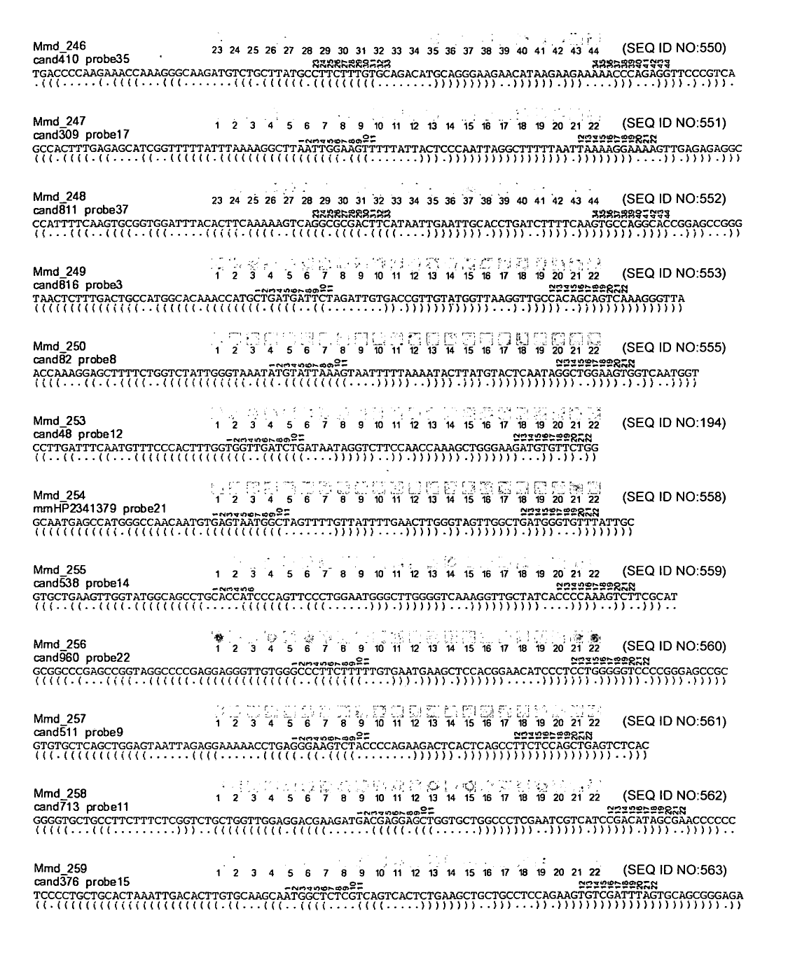

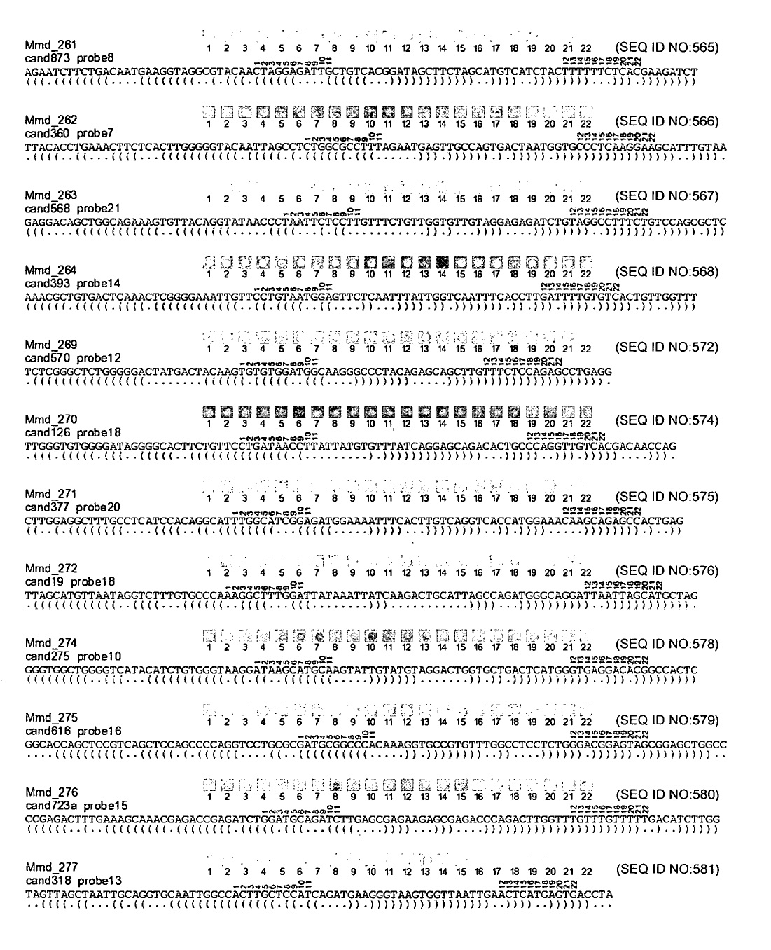

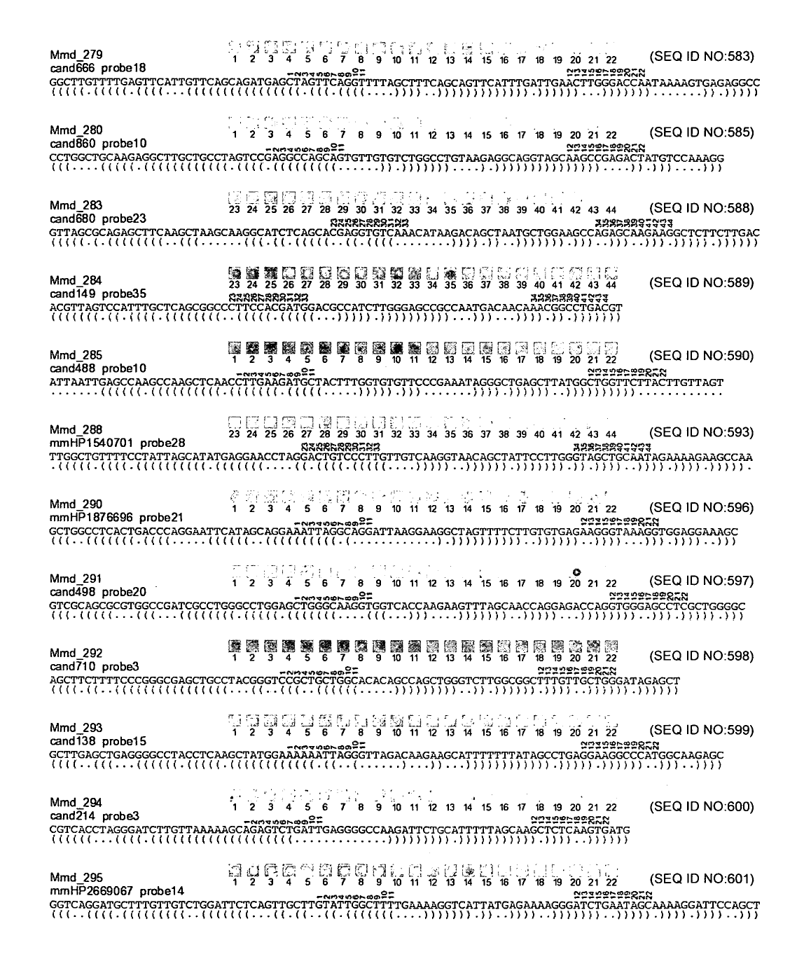

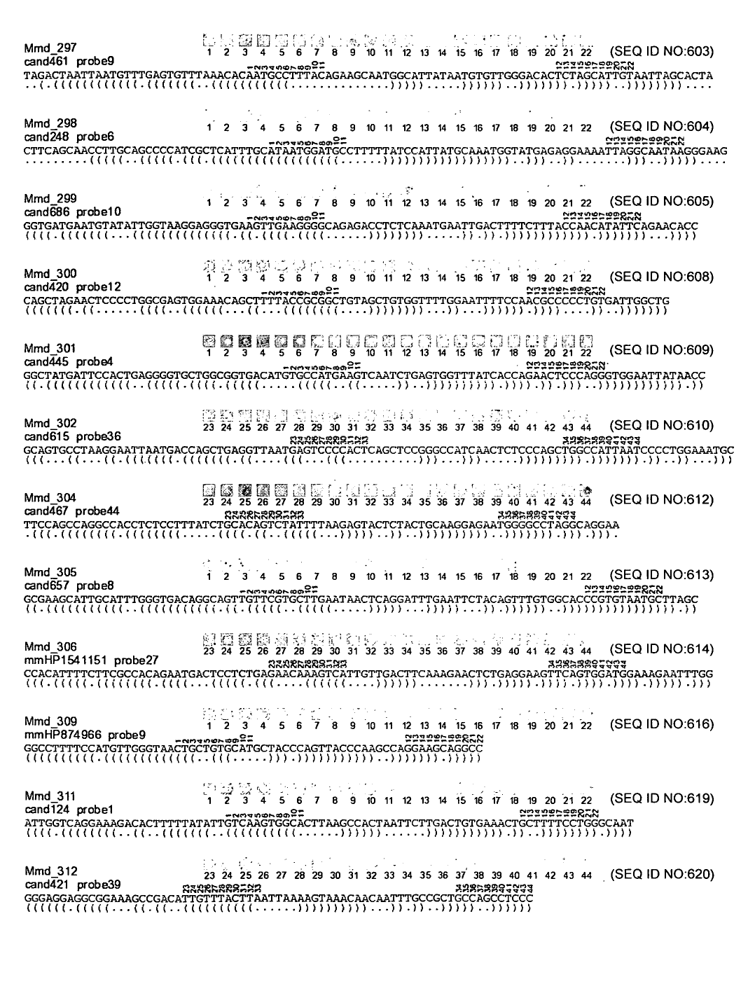

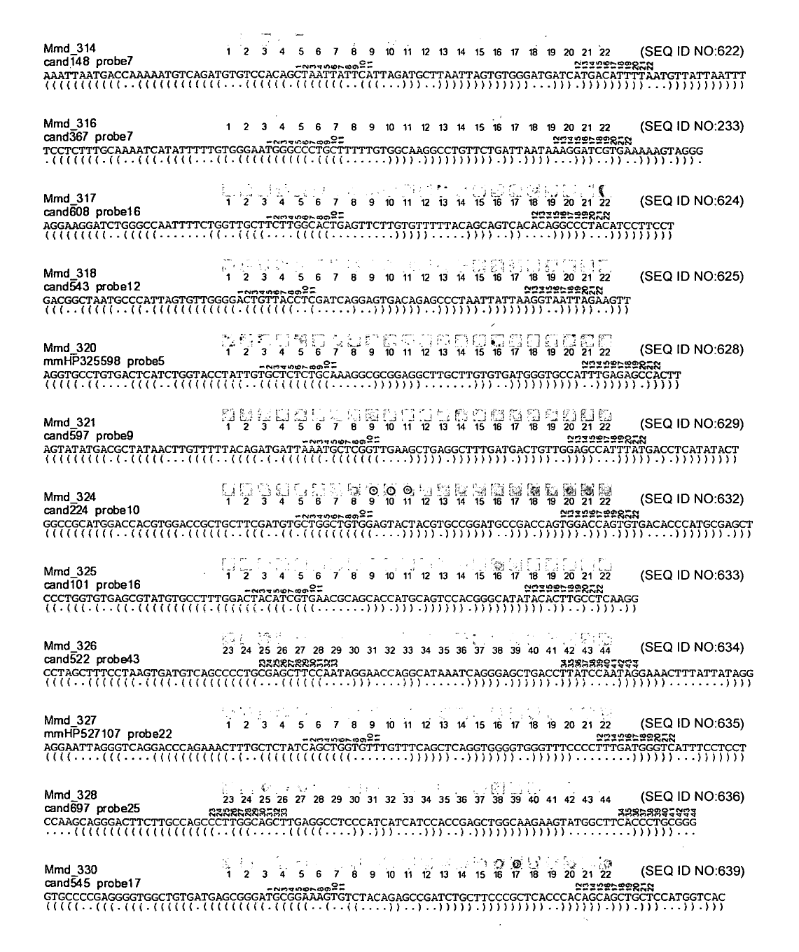

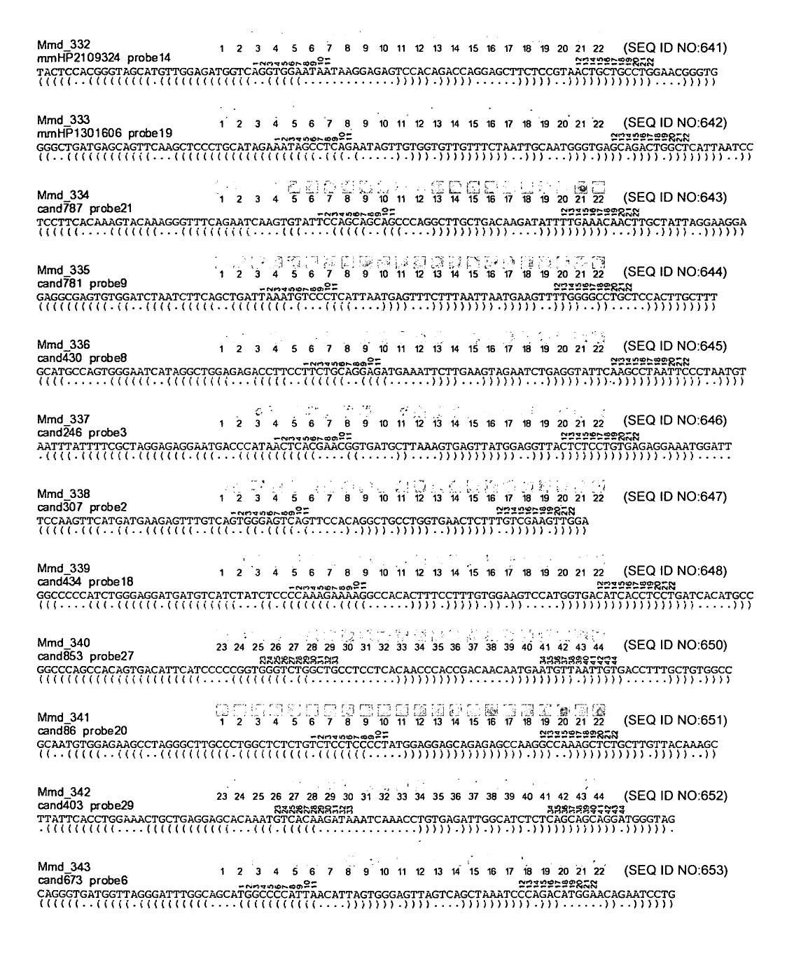

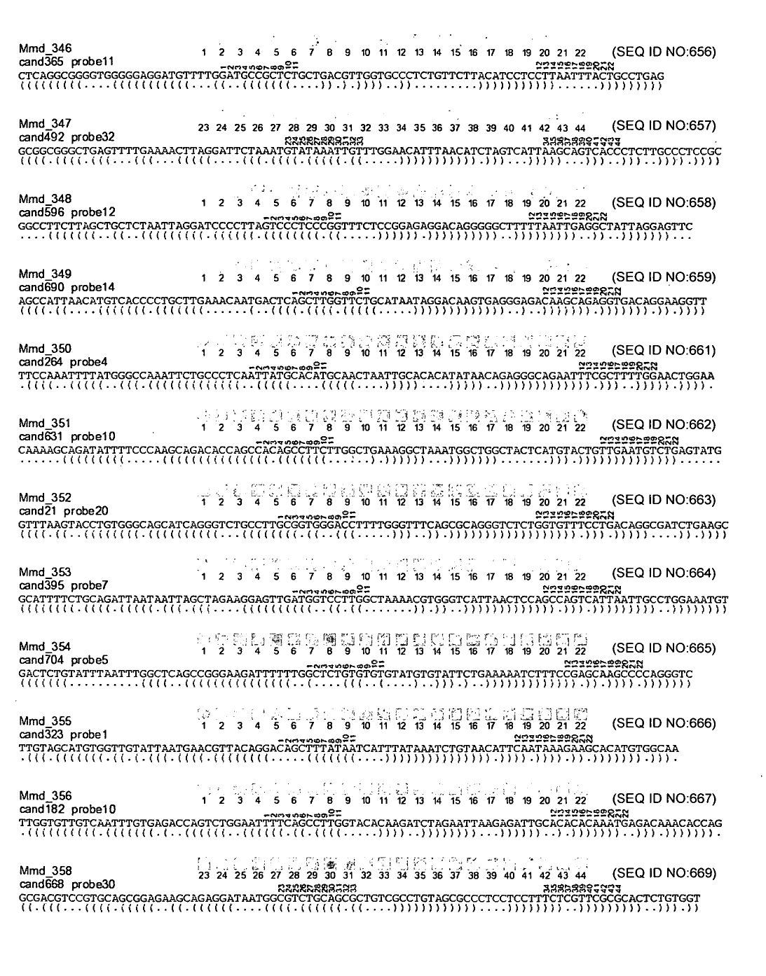

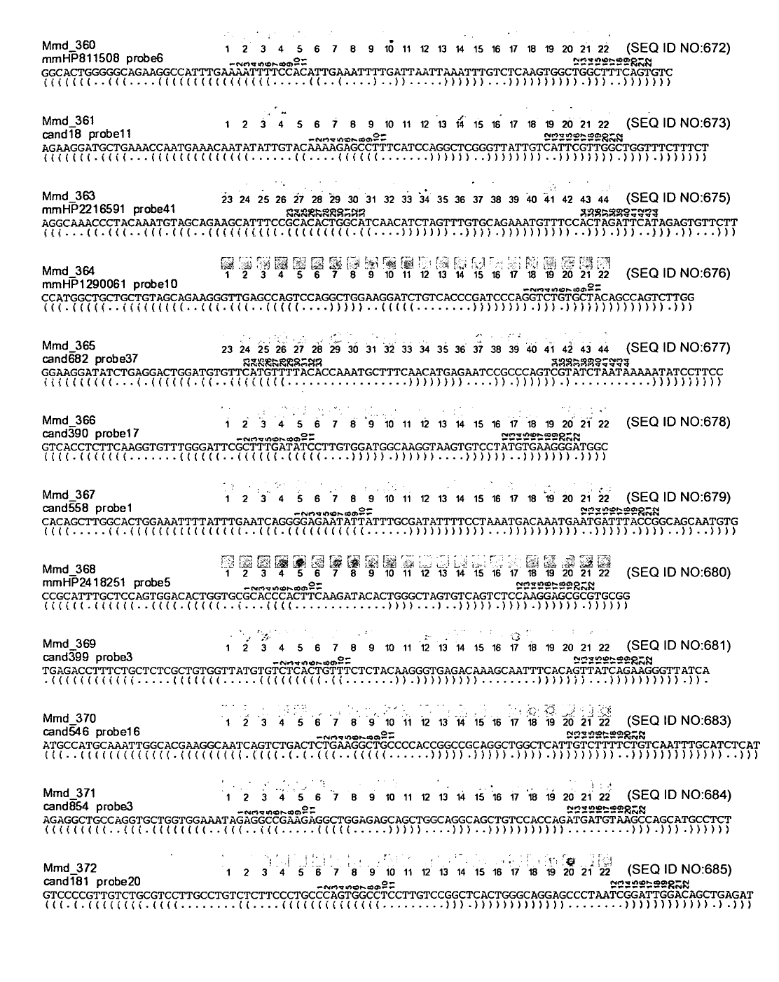

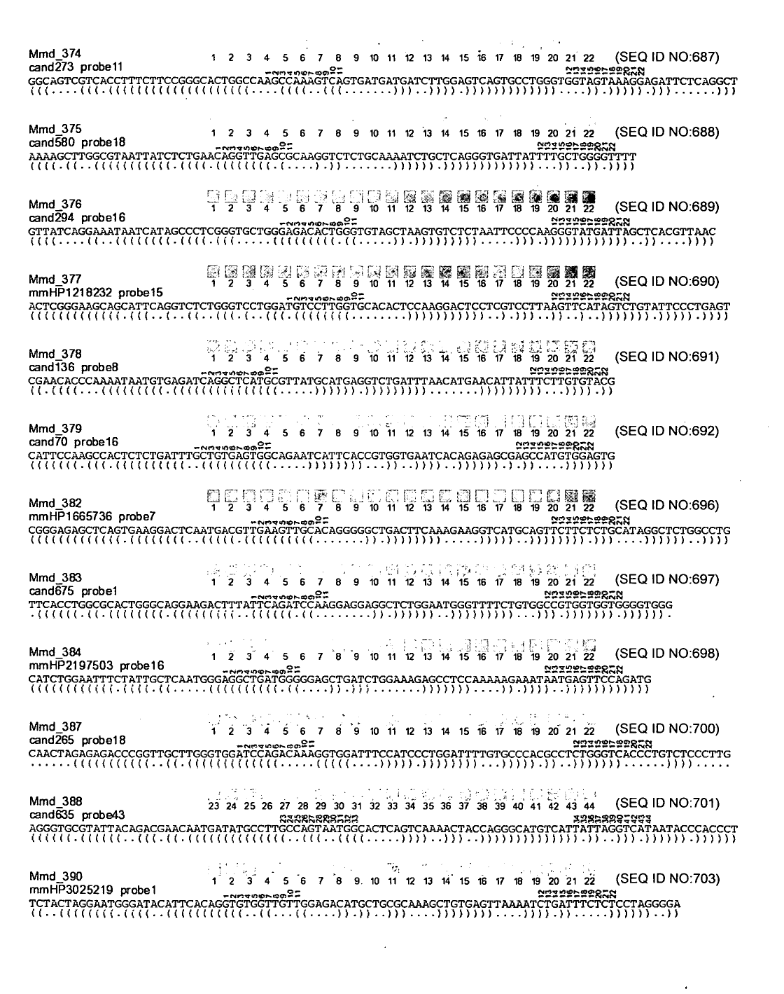

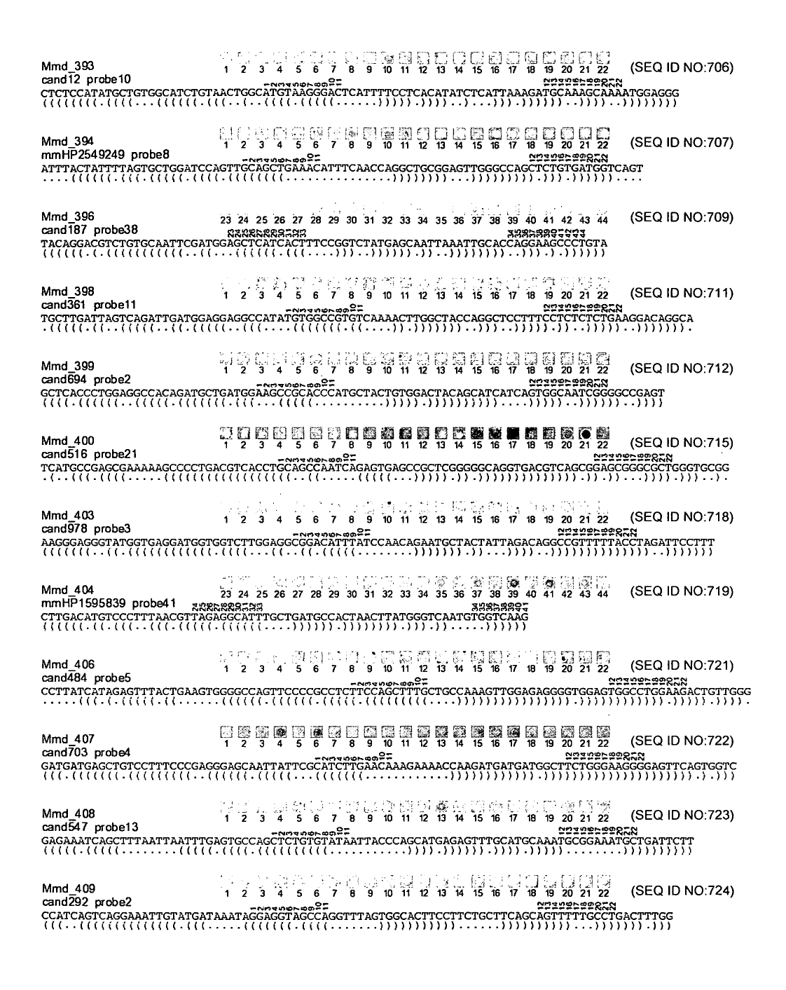

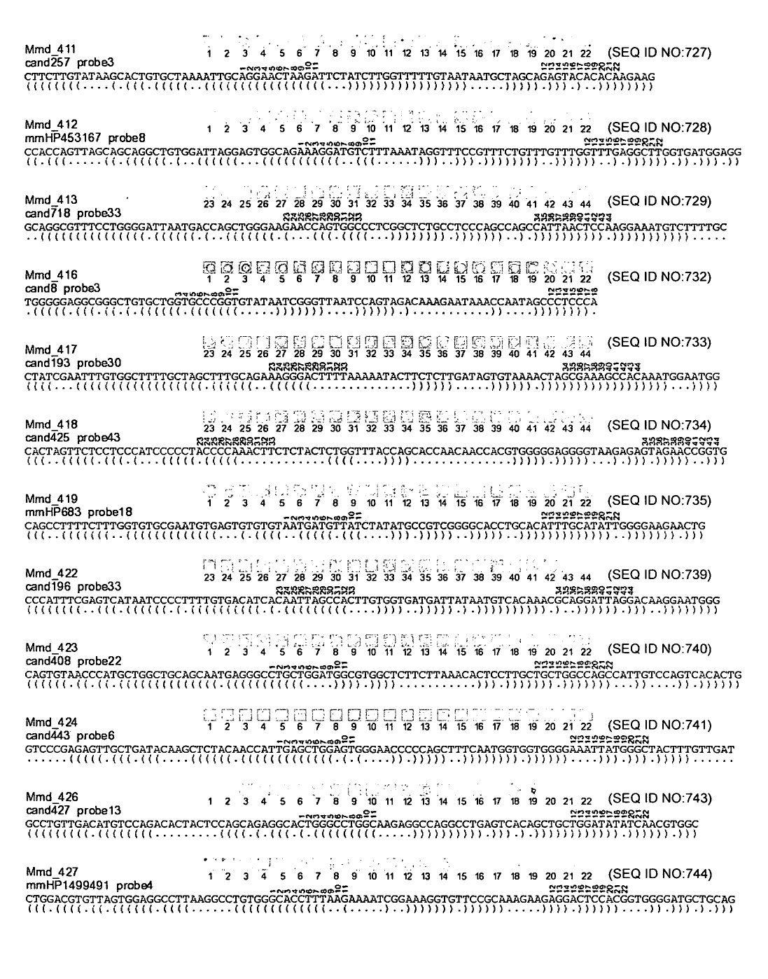

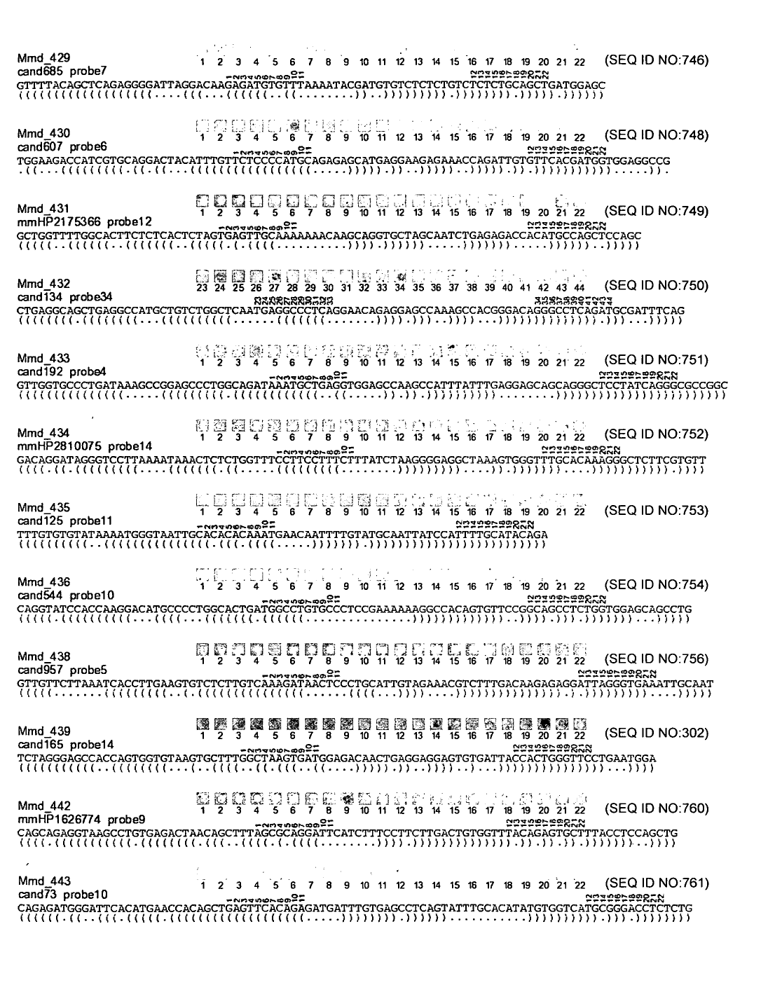

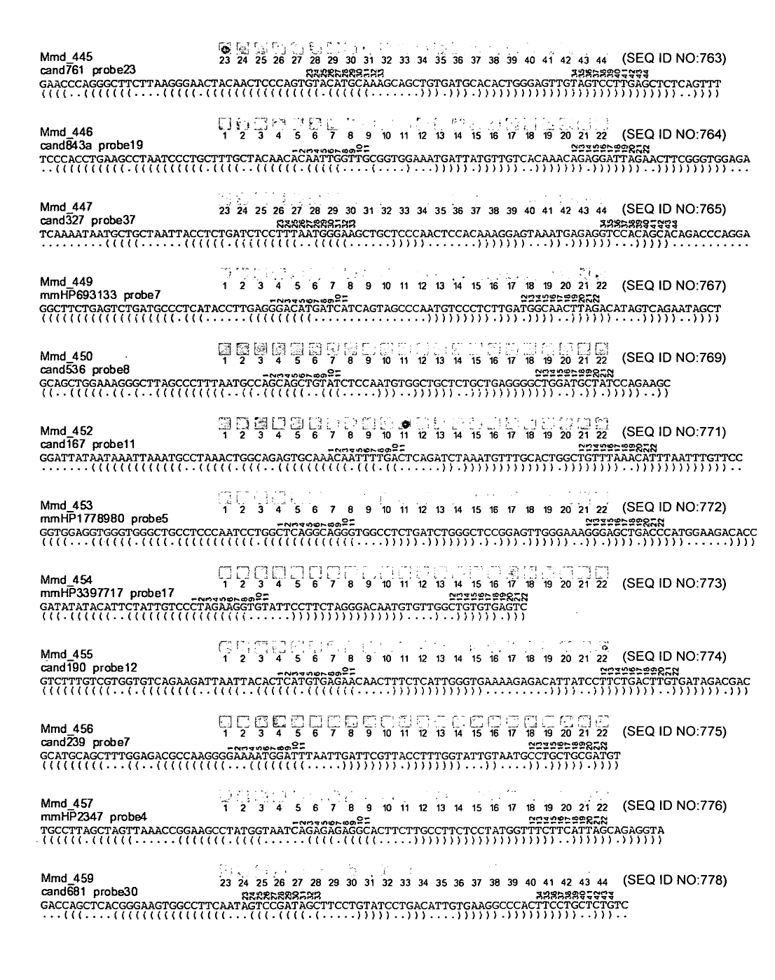

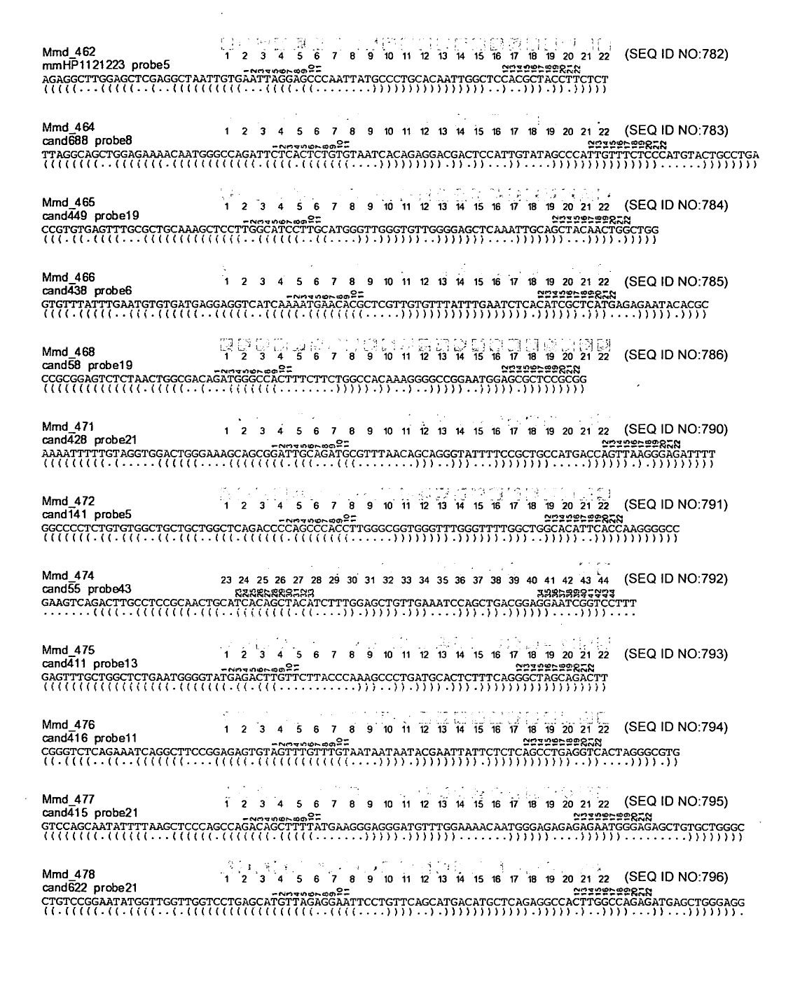

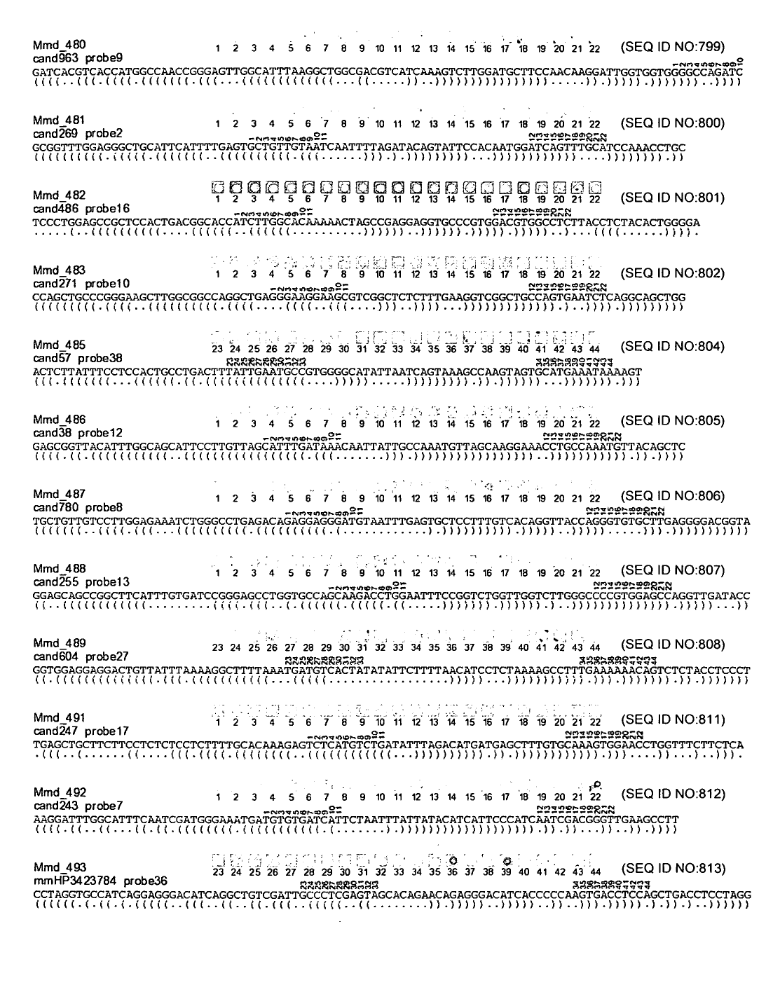

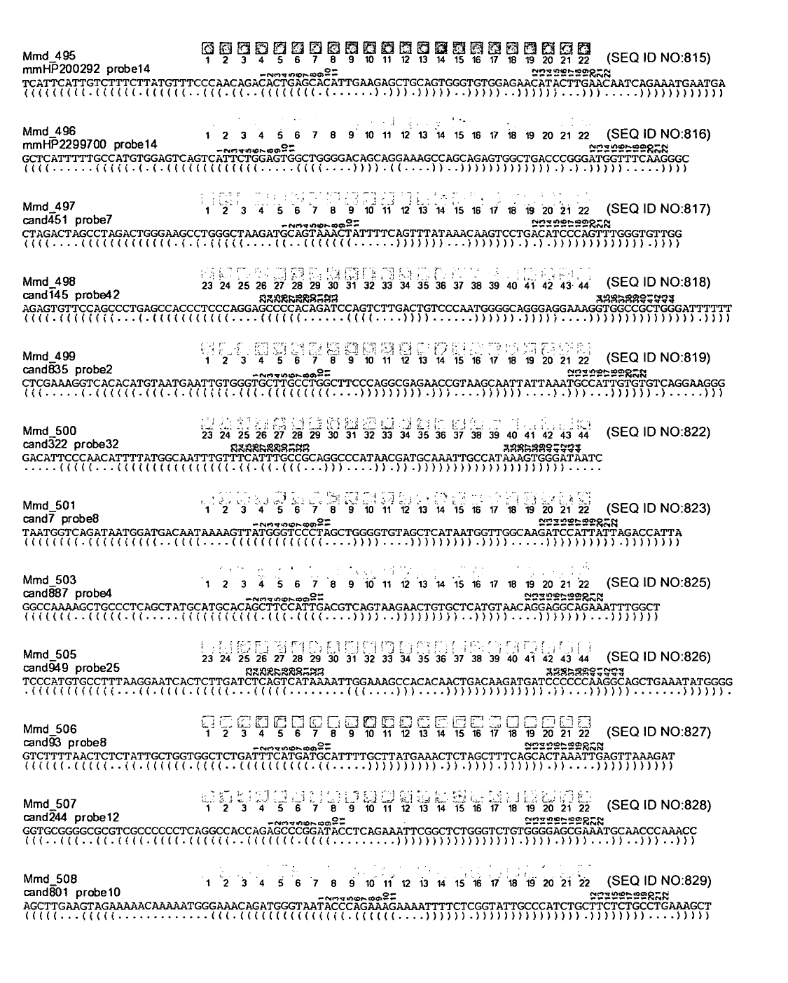

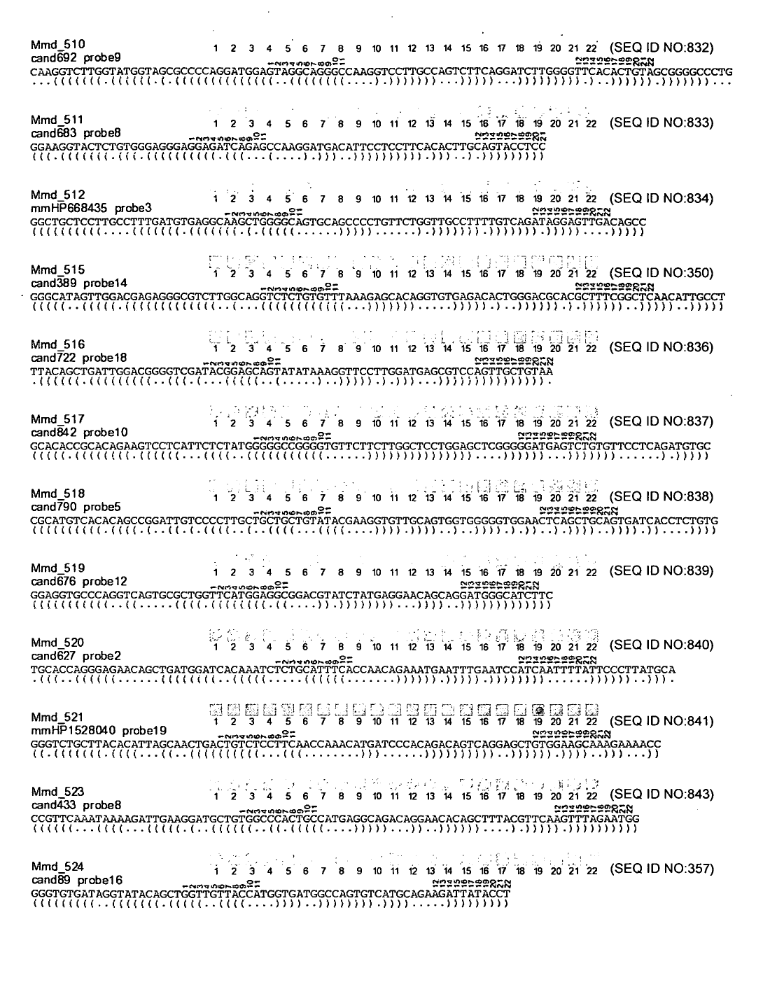

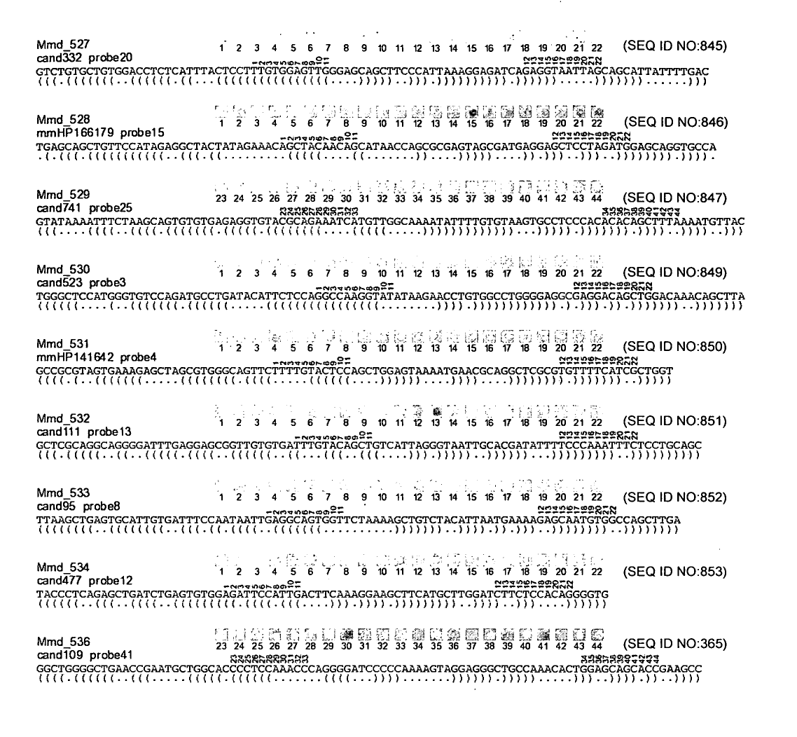

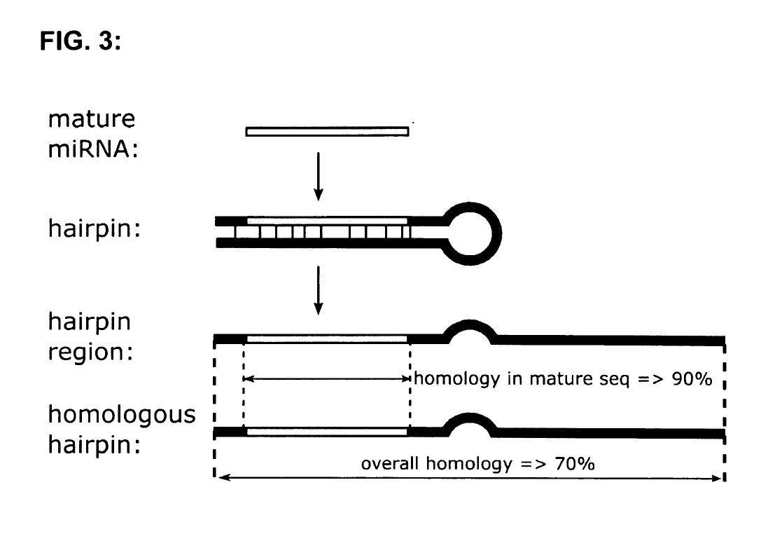

A mammalian homologue of a hairpin RNA as depicted in FIG. 1 is a sequence that comprises at least 70% sequence identity with a hairpin RNA of FIG. 1 that can fold in a similar stem loop (hairpin) structure as the corresponding hairpin RNA of FIG. 1 (graphically depicted in FIG. 3). A mammalian homologue of a miRNA as depicted in FIG. 1 is a sequence that exhibits 90% sequence identity with at least 20, preferably consecutive, nucleotides of the corresponding miRNA of FIG. 1 (graphically depicted in FIG. 3). Preferably, said mammalian homologue of a miRNA of FIG. 1 is present in a mammalian homologue of the corresponding hairpin RNA. Preferably, said miRNA homologue is present in a part of said hairpin homologue that can form a stem structure.

The presence, relative abundance or absence of a miRNA of FIG. 1 or a mammalian homologue thereof and/or a hairpin RNA of FIG. 1 or a mammalian homologue thereof in a sample, can be determined by using a detection method. Typically a method for the specific detection of nucleic acid is used. Currently there are many methods for the specific detection of nucleic acids. Typically, though not necessarily these use a probe that specifically recognizes at least part of the nucleic acid to be tested. Such probe is often nucleic acid, but can also be an analogue thereof. For instance, various nucleotide analogues are presently available that mimic at least some of the base pairing characteristics of the “standard” nucleotides A, C, G, T and U. Alternatively, nucleotide analogues such as inosine can be incorporated into such probes. Other types for analogues include LNA, PNA, morpholino and the like. Further methods for the specific detection of nucleic acid include but are not limited to specific nucleic acid amplification methods such as polymerase chain reaction (PCR) and NASBA. Such amplification methods typically use one or more specific primers. A primer or probe preferably comprises at least 12 nucleotides having at least 90% sequence identity to a sequence as depicted in FIG. 1, or the complement thereof.

The present invention provides an isolated nucleic acid molecule comprising:
a) a nucleotide sequence as shown in FIG. 1, and/or
b) a nucleotide sequence which is a complement of a), and/or
c) a nucleotide sequence which has an identity of at least 80% to a sequence of a) or b) and/or
d) a nucleotide sequence which hybridizes under stringent conditions to a sequence of a), b) or c).

A complement of a nucleic acid sequence as used herein is a sequence wherein most, but not necessarily all bases are replaced by their complementary base: adenine (A) by thymidine (T) or uracil (U), cytosine (C) by guanine (G), and vice versa. Identity of sequence in percentage is preferably determined by dividing the number of identical nucleotides between a given and a comparative sequence by the length of the comparative sequence. In a preferred embodiment the invention provides a nucleic acid molecule according to the invention, wherein the identity of sequence c) to a sequence of a) or b) is at least 90%. In a more preferred embodiment said identity of sequence c) to a sequence of a) or b) is at least 95%. Preferably, said sequence identity to a miRNA of FIG. 1 or its complement is 90% in a stretch of preferably 20 nucleotides of said miRNA. Nucleotides A, C, G and U as used in the invention, are either ribonucleotides, deoxyribonucleotides and/or other nucleotide analogues, such as synthetic nucleotide analogues. A nucleotide analogue as used in the invention is, for example, a peptide nucleic acid (PNA), a locked nucleic acid (LNA), or alternatively a backbone- or sugar-modified ribonucleotide or deoxyribonucleotide. Furthermore the nucleotides are optionally substituted by corresponding nucleotides that are capable of forming analogous H-bonds to a complementary nucleic acid sequence. An example of such a substitution is the substitution of U by T. Stringent conditions under which a nucleotide sequence hybridizes to a sequence according to the invention are highly controlled conditions. Stringent laboratorial hybridization conditions are known to a person skilled in the art.

In a preferred embodiment the invention provides a nucleic acid molecule according to the invention, which is a miRNA molecule or an analogue thereof. A further preferred embodiment of the invention provides a hairpin RNA molecule and a DNA molecule encoding miRNA or hairpin molecule. In another embodiment the invention provides an miRNA homologue of FIG. 1 or a mammalian homologue of a miRNA of FIG. 1. A homologue as used herein is a sequence, preferably a gene or a product of this gene that has evolved from a common ancestor in two or more species.

An isolated nucleic acid according to the invention preferably has a length of from 18 to 100 nucleotides, more preferably from 18 to 80 nucleotides. Mature miRNA usually has a length of from 18 to 26 nucleotides, mostly approximately 22 nucleotides. In a preferred embodiment the invention thus provides a nucleic acid molecule according to the invention having a length of from 18 to 26 nucleotides, preferably of from 19-24 nucleotides, most preferably 20, 21, 22 or 23 nucleotides. MiRNAs are also provided by the invention as precursor molecules. The invention thus further provides a nucleic acid molecule according to the invention which is a pre-miRNA, a hairpin RNA as depicted in FIG. 1 or a DNA molecule coding therefore. Precursor or hairpin molecules usually have a length of from 50-90 nucleotides. The invention provides a nucleic acid molecule according to the invention, having a length of 50-90 nucleotides of a hairpin RNA of FIG. 1. In a preferred embodiment the invention thus provides a nucleic acid molecule according to the invention, which is a pre-miRNA or a DNA molecule coding therefore, having a length of 60-110 nucleotides. The invention further provides a nucleic acid molecule according to the invention which has a length of more than 110 nucleotides, as a precursor miRNA is for example produced by processing a primary transcript. In a preferred embodiment the invention provides a nucleic acid molecule according to the invention, wherein said pre-miRNA is a pre-miRNA of FIG. 1 or a mammalian homologue or ortholog thereof.

As mentioned above, single-stranded miRNA is incorporated in a RISC. A miRNA precursor molecule is often partially double-stranded. Usually a miRNA precursor molecule is at least partially self-complementary and forms double-stranded parts such as loop- and stem-structures. The invention in one embodiment provides a nucleic acid molecule according to the invention, which is single-stranded. In another embodiment the invention provides a nucleic acid molecule according to the invention, which is at least partially double-stranded. In one embodiment of the invention a nucleic acid molecule according to the invention is selected from RNA, DNA, or nucleic acid analogue molecules or a combination thereof. In another embodiment of the invention aforementioned nucleic acid molecule is a molecule containing at least one modified nucleotide analogue. In a further embodiment the invention provides use of said nucleic acid molecule according to the invention in a therapeutic and/or diagnostic application.

A nucleic acid molecule according to the invention is in one embodiment part of a collection of nucleic acid molecules. Such a collection is preferably, but not exclusively, used in a test. A collection of nucleic acid molecules is for example used in a test as described above, for instance to determine absence or presence of a disease in an individual by testing a sample taken from this individual. A collection of nucleic acid molecules usually has a higher predictive value in any experimental setting when the number of nucleic acid molecules provided herein is larger. Thus, in one embodiment, the invention provides a collection of nucleic acid molecules, comprising at least 5, preferably at least 10, more preferably at least 20 nucleic acid molecules comprising a nucleotide sequence as shown in FIG. 1. A collection of nucleic acid molecules according to the invention, is in one embodiment used for the diagnosis of diseases such as cancer, heart disease, viral infections or disease susceptibility.

Further provided is a collection of nucleic acid molecules, comprising at least 5, preferably at least 10, more preferably at least 20 nucleic acid molecules that are complementary to miRNAs shown in FIG. 1, or that have nucleotide sequences which hybridize under stringent conditions to miRNAs shown in FIG. 1. A collection of nucleic acid molecules are preferably used in the diagnosis of cancer, heart disease, viral infections and other diseases.

A nucleic acid molecule according to the invention can be obtained by any method. Non-limiting examples are chemical synthesis methods or recombinant methods. A nucleic acid molecule according to the invention is in one embodiment modified. Said modification is for example a nucleotide replacement. Said modification is for example performed in order to modify a target specificity for a target in a cell, for instance a specificity for an oncogene. Said modified nucleic acid molecule preferably has an identity of at least 80% to the original miRNA, more preferably of at least 85%, most preferred of at least 90%. In another embodiment a nucleic acid molecule according to the invention is modified to form a siRNA molecule. For example, a miRNA molecule is processed in a symmetrical form and subsequently generated as a double-stranded siRNA. In a preferred embodiment the invention provides a nucleic acid molecule according to the invention, which is selected from RNA, DNA or nucleic acid analogue molecules which preferably further comprises at least one nucleotide analogue. In one embodiment a nucleic acid molecule of the invention is present in a recombinant expression vector. A recombinant expression vector according to the invention for example comprises a recombinant nucleic acid operatively linked to an expression control sequence. Said vector is any vector capable of establishing nucleic acid expression in an organism, preferably a mammal. Said vector is preferably a viral vector or a plasmid. In a preferred embodiment introduction of said vector in an organism establishes transcription of said nucleic acid. In a preferred embodiment after said transcription the transcript is processed to result in a pre-miRNA molecule and/or a hairpin molecule and subsequently in a miRNA molecule.

Nucleic acids according to the invention are in one embodiment provided as a probe. Many different kinds of probes are presently known in the art. Probes are often nucleic acids, however, alternatives having the same binding specificity in kind, not necessarily in amount are available to the person skilled in the art, such alternatives include but are not limited to nucleotide analogues. In one embodiment the invention provides a set of probes comprising at least one nucleic acid molecule according to the invention. In a preferred embodiment the invention provides a set of probes according to the invention, wherein said nucleic acid molecule is a miRNA molecule of FIG. 1 or a functional part, derivative and/or analogue thereof. In a further preferred embodiment the invention provides a set of probes according to the invention, wherein said nucleic acid molecule is a complement of a miRNA molecule or a functional part, derivative and/or analogue thereof. In a further preferred embodiment the invention provides a set of probes comprising a collection of nucleic acid molecules according to the invention. A collection in this embodiment preferably is a collection of nucleic acid molecules, comprising at least 5, preferably at least 10, more preferably at least 20 nucleic acid molecules comprising a nucleotide sequence as shown in FIG. 1 or a mammalian homologue thereof, or is a collection of nucleic acid molecules, comprising at least 5, preferably at least 10, more preferably at least 20 nucleic acid molecules with a nucleotide sequence which is a complement of a nucleotide sequence as shown in FIG. 1, or with a nucleotide sequence which hybridizes under stringent conditions to a nucleotide sequence as shown in FIG. 1, or is a combination thereof.

Further provided is an array comprising one or more nucleic acids of the invention. An array is used to analyze one or more samples at the same time. Preferably said array comprises at least two probes, wherein at least one probe comprises a nucleic acid molecule according to the invention. In a preferred embodiment said array comprises a set of probes comprising a collection of nucleic acid molecules according to the invention, or a combination of collections of nucleic acid molecules according to the invention. In one embodiment an array of the invention is a microarray. Said microarray preferably comprises oligonucleotides. A set of probes or an array or microarray according to the invention is in a preferred embodiment used in a diagnostic test.

A diagnostic test as used in the invention, is a test wherein a nucleic acid molecule according to the invention is used to subject a sample of an organism to a diagnostic procedure. Said organism preferably is a mammal, more preferably a human being. A sample as used in the invention preferably is a biological sample. A biological sample is for example a bodily fluid. A preferred biological sample is a tissue sample. A tissue sample is, for instance, used to determine a stage of differentiation or development of a cell. Alternatively a cell type or tissue type is classified as corresponding with a disorder. Said disorder is, for example, characterized by a typical expression level of a miRNA molecule or a typical expression pattern of miRNA molecules. The invention provides a nucleic acid molecule according to the invention for diagnostic applications as well for therapeutic applications. A diagnostic of therapeutic application according to the invention relates to a disorder, for example a viral infection or cancer. Recently miRNAs have been described to be an important causal factor in cancer (Lu et al., 2005; He et al., 2005; O'Donnell et al., 2005; Alvarez-Garcia and Miska, 2005) or a powerful indicator for prognosis and progression of cancer (Calin et al., 2005). A cancer is for example leukemia.

In one embodiment the invention provides a pharmaceutical composition, comprising as an active agent at least one nucleic acid molecule according to the invention, and optionally a pharmaceutically acceptable carrier. A pharmaceutical composition according to the invention further optionally comprises another additive. Such another additive can for example be a preservative or a colorant. Alternatively an additive is a known pharmaceutically active compound. A carrier is any suitable pharmaceutical carrier. A preferred carrier is a compound that is capable of increasing the efficacy of a nucleic acid molecule to enter a target-cell. Examples of such compounds are liposomes, particularly cationic liposomes. A composition is for example a tablet, an ointment or a cream. Preferably a composition is an injectable solution or an injectable suspension. In one embodiment the invention provides a pharmaceutical composition according to the invention for diagnostic applications. In another embodiment the invention provides a pharmaceutical composition according to the invention for therapeutic applications. In a preferred embodiment the invention provides a pharmaceutical composition according to the invention, as a modulator for a developmental or pathogenic disorder. In a preferred embodiment said developmental or pathogenic disorder is cancer. A miRNA molecule for example functions as a suppressor gene or as a regulator of translation of a gene.

A nucleic acid molecule according to the invention is administered by any suitable known method. The mode of administration of a pharmaceutical composition of course depends on its form. In a preferred embodiment a solution is injected in a tissue. A nucleic acid molecule according to the invention is introduced in a target cell by any known method in vitro or in vivo. Said introduction is for example established by a gene transfer technique known to the person skilled in the art, such as electroporation, microinjection, DEAE-dextran, calcium phosphate, cationic liposomes or viral methods.

A nucleic acid molecule according to the invention is in one embodiment used as a marker of a gene. A marker identifies a gene, for example a gene involved in cancer or another developmental disorder. A marker is, for instance, a miRNA that is typically differentially expressed in a disorder or is a set of two or more miRNAs that display a typical expression pattern in a disorder. A nucleic acid molecule is alternatively for example labelled with a fluorescent or a radioactive label. A nucleic acid molecule according to the invention is, in another embodiment, a target for a diagnostic or therapeutic application. For example, a miRNA molecule according to the invention is inhibited or activated and the effect of the inhibition or activation is determined by measuring differentiation of a cell type. In another embodiment, a nucleic acid according to the invention is not a target itself, but alternatively used to address a target in a cell. A target in a cell is preferably a gene. Preferably said gene is at least partially complementary to said nucleic acid molecule. For example, a miRNA according to the invention is used to find a gene in a cell that has a sequence that is at least partially complementary to the sequence of said miRNA. In a preferred embodiment the invention provides a pharmaceutical composition as a marker or modulator of expression of a gene. In another preferred embodiment the invention provides a pharmaceutical composition according to the invention, wherein said gene is at least partially complementary to said nucleic acid molecule. A modulator of expression of a gene is for example a miRNA. A miRNA that functions as a tumour-suppressor is for instance provided and expressed in and/or delivered to a tumour cell thus suppressing the development of a tumour. In a preferred embodiment the invention provides a use of a nucleic acid molecule according to the invention, for down regulating expression of a gene. Down regulating expression of a gene is for example important in cancer. In an alternative embodiment a miRNA is introduced and/or expressed in a cell of a tissue that does not express said miRNA. As a result said cell of said tissue for example shows a different differentiation type. Such a procedure is for example used as a tissue reprogramming procedure.

At present, there are essentially two approaches for identification of novel miRNA genes: cloning of size-fractionated (18-25 nt) RNAs and computational prediction based on different structural features of miRNAs followed by experimental verification. Cloning of size-fractioned RNAs is a laborious procedure and has resulted in a restricted amount of identified miRNAs. Established methods for validation of predicted miRNA genes rely on construction of size-fractionated cDNA libraries. This is a technically challenging procedure that does not scale well. Moreover it requires testing many tissues and developmental time points. Established methods of experimental validation of predicted miRNAs thus do not scale for the analysis of thousands of candidates regions. The invention surprisingly found a high-throughput approach for testing candidate miRNA regions. The invention provides a modified RAKE assay for high-throughput expression studies of candidate miRNA regions. The provided assay allows exact mapping of 3′ ends of mature miRNAs, thus providing information on both structure and expression profiles of novel miRNA genes. Different microarray technologies, including RAKE assay, have been applied for expression profiling of known miRNAs. However, microarrays were not previously used for detection of novel, computationally predicted miRNAs. The unique method of combining a computational method with a modified RAKE assay, provided by the invention, has led to the discovery of numerous new miRNAs. Furthermore the provided method offers an opportunity to discover further miRNAs.

Cross-species sequence comparison is a powerful approach to identify functional genomic elements, but its sensitivity decreases with increasing phylogenetic distance, especially for short sequences. In addition, taxon-specific elements may be missed. To overcome the limitations of classical phylogenetic footprinting methods, the invention applied the phylogenetic shadowing approach (Boffelli et al., 2003), allowing unambiguous sequence alignments and accurate conservation determination at single nucleotide resolution level. This approach is based on the alignment of phylogenetically closely related species; since these show only few sequence differences, many different (but related) genomes need to be aligned to identify invariant (conserved) positions. In the invention 700 bp regions surrounding 122 miRNAs in 10 different primate species were sequenced, including orangutan, gorilla, 2 chimpanzee and 2 macaque species, tamarin, spider monkey, wooly monkey and lemur. Besides the region spanning the pre-miRNA, no additional conserved regions common to different miRNAs could be found, suggesting that, in contrast to C. elegans (Ohler et al., 2004), no common cis-acting elements can be immediately recognized in mammalian miRNAs. In the invention it was surprisingly found that there is a prominent drop of conservation immediately flanking pre-miRNA regions. This characteristic conservation pattern can also be recognized in pairwise alignments between more diverged species like human and mouse and was used to identify novel miRNA genes by screening mouse-human and rat-human whole-genome sequence alignments for this typical conservation profile. Additional stringent filtering for the ability of candidate regions to fold into a thermodynamically favorable stable hairpin, as calculated by Randfold software (Bonnet et al., 2004), resulted in the identification of 976 candidate miRNAs, containing 83% of all known human miRNAs (158 out of 189, based on miRNA registry v.3.1).

Screening for homologues in additional vertebrate genomes (zebrafish, chicken, opossum, cow and dog) revealed that 678 candidates are conserved in at least one other species besides rodents. A substantial part of the predictions consists of miRNAs unique to mammals. Both the genomic distribution and the extent of supportive data for expression are comparable for the mammalian-specific subset and the set of candidates that are also conserved in at least one non-mammalian species. Even though the degree of genome coverage varies for the species used in the comparisons, this data suggests that there are a significant number of lineage-specific miRNAs and indicates that both rapidly and slowly evolving miRNAs exist (let-7 being a typical example of a slow evolver).

Fourteen novel candidates share homology with known miRNAs and an additional 60 share homology with at least one other candidate, making up novel subfamilies. In addition to the established clustering behavior of miRNAs (Bartel 2004, Rodriguez et al., 2004), the ratio between the number of miRNA genes in inter- and intragenic regions is similar for both known and novel miRNAs. Although a fair proportion of candidates are predicted on the strand opposite to annotated transcripts, the disproportionate presence of miRNA genes in introns is intriguing and may reflect expression mechanisms by co-transcription with the host gene and processing of spliced introns. 171 of the predicted novel miRNAs reside in genomic regions that are annotated as exons. In experimental approaches, such candidates are often discarded as potential cloning artifacts, but these regions can be processed into mature miRNAs. Work by Cullen and co-workers (Cai et al., 2004) demonstrated that a transcript harbouring simultaneously a miRNA and an ORF is efficiently used for both miRNA and protein production. About 25% (44) of the exonic candidates reside in non-coding parts and although 127 candidates overlap with annotated protein coding sequences, 75 are predicted on the opposite strand.

Support for the expression of candidate miRNAs is provided through various sources. Three candidates are present in the FANTOM2 database of expressed sequences and 11 candidates reside in gene clusters containing one or more known miRNAs. These miRNAs are likely to be co-expressed from the same primary transcript (Bartel, 2004, Rodriguez et al., 2004). Systematic human transcriptome analysis using high-density oligonucleotide tiling arrays (Kapranov et al., 2002) is in progress and in the invention it was found that the genomic regions encoding 64 known and 214 novel miRNAs has now been covered. From this set, 13 known (20%) and 72 novel (34%) miRNAs are expressed in the SK-N-AS cell line, for which data is publicly available. Although poly (A)+ RNA was used for these experiments and properties of miRNA-containing transcripts remain largely to be elucidated, both intergenic and intronic miRNAs were detected. Various lines of research support the finding that at least some miRNAs are processed from poly-adenylated RNA (Cai et al., 2004, Lee et al., 2004).

To provide experimental support for the predicted miRNAs, in the invention Northern blotting experiments were performed for 69 candidates, confirming the expression of 16 mature miRNAs (23%). Although these verification rates are lower than previously published rates using cloning- and PCR-based approaches (38 out of 93; Lim et al., 2003), they may be an under-representation as a result of a bias in the set of already known miRNAs for highly expressed and thus most easily detectable miRNAs, the sensitivity of the detection method, and spatio-temporal limitations of the RNA samples used. Therefore, we developed another potentially more sensitive strategy for candidate miRNA validation based on the RAKE (RNA-primed Array-based Klenow Extension, Nelson et al., 2004) assay.

This assay is based on the ability of an RNA molecule to function as a primer for Klenow polymerase extension when fully base-paired with a single stranded DNA molecule. As the exact 3′-end of the miRNA should be known for successful extension and computational predictions are not optimal for predicting the correct start and end of the mature miRNA, we designed a tiling path of probes complementary to both known and predicted miRNA precursors. Such a tiling path RAKE assay is less prone to false positives than standard hybridization assays, as it depends on the presence of a fully matching 3′-end of the miRNA and hence distinguishes between miRNA family members that differ in their 3′ sequences. Flanking tiling path probes function as negative controls. Although some rules have been put forward to determine which strand of the stem is preferentially loaded as mature miRNA in the RISC complex (Khvorova et al., 2003; Schwarz et al., 2003), such computational predictions can only be done when the precise ends of the processed miRNA duplex are known. In addition, due to the nature of the hairpin sequence it is often difficult to predict which strand of the genomic DNA encodes a precursor. To take a fully unbiased approach, we designed tiling paths of 11 probes covering each arm of the stem-loop structure, for the sense as well as the anti-sense genomic sequence, resulting in sets of 44 probes per candidate miRNA gene. Due to G-U pairing allowed in RNA folding and different nucleotide composition of the complementary DNA strand, anti-sense transcripts do not necessarily fold into stable stem-loop structures and for such candidates only 22 probes were included. The central position in the tiling path was determined by predicting the most likely Dicer/Drosha processing sites from secondary structure hairpin information. We designed a custom validation microarray with 44,000 features, covering 271 known mouse miRNAs and 676 of the predicted miRNAs that are conserved between mouse and human, and filled up the array with 199 additional candidates based on stringent randfold criteria (Bonnet et al., 2004) and mouse and rat genome conservation. These arrays were probed with 4 different sources of small RNAs: mouse embryos at embryonic days 8.5 and 16.5, adult mouse brain and embryonic stem (ES) cells (FIG. 2). Mature miRNAs were semi-manually annotated after pre-processing the raw microarray output data using custom scripts. A redundant set of 221 of the known miRNAs (82%), 429 of the candidate conserved miRNAs (63%), and 126 of the extra set (63%) were found positive (FIG. 2). As different genomic loci can produce an identical mature miRNA from a different hairpin (e.g. miR-1-1 and miR-1-2), the total number of non-redundant mature miRNAs is lower. Interestingly, for more than half of the known miRNAs, the most prominent 3′ end observed in the RAKE assay differed from the annotated form, including 8 mature miRNAs residing in the other arm of the hairpin, suggesting that originally the star-sequence was annotated. In addition, for various candidate and known miRNAS, multiple subsequent probes (2 or 3) resulted in a positive signal, indicating that 3′ end processing of miRNAs is not a completely accurate process at the single nucleotide level. These findings are in line with the observed variation in ends of cloned miRNAs (Aravin and Tuschl, 2005).

The second approach we pursued to experimentally confirm novel miRNAs is deep sequencing of size-fractionated small RNA libraries of isolated human and mouse tissues. Although it was suggested previously that such efforts had reached near saturation (Lim et al., 2003), only limited numbers of library clones from a selected set of vertebrate tissues have been sequenced (Lagos-Quintana et al., 2001, Lim et al., 2003, Bentwich et al., 2005). Moreover, our computational predictions and microarray(RAKE)-based confirmations suggested many novel miRNAs to be discovered. Therefore, we generated seven high-titer non-concatomerized libraries of size-fractioned small RNA's from mouse brain and various human fetal tissues (brain, skin, heart, lung, and mixed tissues) and sequenced 83,040 clones. After vector and quality trimming 51,044 inserts longer than 17 bases were recovered that represent 8,768 and 7,306 non-redundant mouse and human sequences, respectively. We established a computational pipeline for automated annotation of the cloned sequences, taking into account unique chromosomal position, location in repetitive elements or rRNA, tRNA, snoRNA genes, conservation data from 9 vertebrate genomes (human, mouse, rat, dog, cow, chicken, opossum, zebrafish, and fugu), and secondary structure information using randfold (Bonnet et al., 2004). This analysis was applied to the mouse and human cloned fragments, as well as to all known human and mouse miRNAs and the positive candidates identified using the RAKE assay. 214 out of 238 mouse (90%) and 306 out of 319 human (96%) miRNAs, as deposited in miRBase (Griffiths-Jones, 2004), passed the automated filtering and annotation, showing that the false negative rate is low for the known miRNAs. For the sequenced small RNAs, 21,537 mouse (69%) and 13,120 human (66%) clones passed this filtering. Known abundant miRNA sequences dominate this set, but interestingly about 2% of the reads represent 115 novel mouse and 111 novel human miRNA genes (FIG. 1).

Taken together, we identified 535 novel mouse (RAKE and cloning) and 111 novel human (cloning only) miRNA genes. Although only 17 miRNAs were cloned from both human and mouse samples, the majority of the novel mouse miRNAs has a clear human homologue (over 90% identity for the mature miRNA and 70% for the pre-miRNA), adding up to 401 and 542 of newly discovered miRNA genes in the human and mouse genomes, respectively.

In one embodiment the invention provides a method of identifying a human miRNA or a mouse miRNA. In a further embodiment a method according to the invention comprises an additional step. Said step comprises determining an ortholog or a homologue of a gene. An ortholog or a homologue is determined by comparison of sequences. A human homologue or ortholog is for example determined of a mouse sequence or vice versa, a mouse ortholog is determined of a human sequence. A homologue of a miRNA of FIG. 1 is preferably a mammalian homologue. Mammalian homologues of a miRNA of FIG. 1, comprise at least 90% sequence identity in a stretch of at least 20 consecutive nucleotides of a miRNA of FIG. 1, and are preferably situated in a larger RNA that comprises 70% sequence identity with the corresponding hairpin RNA of said miRNA, wherein said larger RNA is preferably capable of forming a stem loop structure as predicted by an appropriate computer model, and wherein said homologue is preferably situated in a predicted stem region in said larger RNA.

MiRNAs are single strand products derived of longer stem-loop precursors; they can base-pair to messenger RNAs, and thus prevent their expression. Animal genomes contain hundreds of miRNA genes and thousands of genes that are targeted by them. miRNAs often have striking organ-specific expression and can thus be used to discriminate between different cell types.

Historically miRNAs were discovered as freak regulators in weird worms: mutants defective in the timing of cell division in the larvae of the nematode C. elegans were found to be defective in a gene lin-4, which encoded a small RNA that was shown to bind to and silence translation of the lin-14 mRNA (Lee at al., 1993). The general relevance of this landmark discovery became clearer when a second small RNA, let-7, was found to be strongly conserved from worms to flies and human (Reinhart et al., 2000), and when subsequently additional miRNAs were discovered. The current picture is that the human genome contains probably at least 500 miRNA genes (Bartel 2004, Berezikov et al., 2005), which are likely to regulate thousands of target genes (Lim et al., 2005, Lewis et al., 2005). Only the 7 base seed sequence (position 2-8 from the 5′ end) seems required for miRNA action in animal cells; why then is the entire miRNA so strongly conserved? Surely other positions contribute small but nevertheless significant effects to miRNA action, but additional explanations may be that the other sequences within the miRNA are required for processing of the precursors, so before the miRNA is mature, and one can not rule out that miRNAs serve other unknown functions in the cell, for which these other sequences are required.

Independent of the discovery of miRNAs, gene silencing by siRNAs was discovered: RNA interference (Fire et al., 1997). The similarity was not immediately recognized, but the central agents in RNAi were RNA molecules of the same size as miRNAs, and since the RNase that makes siRNAs out of longer double stranded RNA had been discovered (Bernstein et al., 2001), it did not—as the phrase is since the 1953 double helix paper—escape anybody's notice that perhaps Dicer might also be responsible for making miRNAs (which was indeed confirmed by a series of parallel papers that showed Dicer mutants are defective in miRNA synthesis). Since then an impressive body of genetic and biochemical analysis has lead to the conclusion that the complexes that silence a mRNA and are guided by a small RNA (RISC, for RNA induced silencing complex) may differ from organism to organism, from tissue to tissue, and there may even be parallel pathways within one cell but in essence miRNAs and siRNAs act via a fairly similar complex, which always contains at least one member of the family of Argonaut proteins.

The precise mechanism by which miRNAs silence mRNAs is unclear, with several issues that need to be resolved. The original discovery of the first miRNA lin-4 indicated that the target mRNA was left intact and not changed in stead-state levels (Lee et al., 1993); the miRNA was thought to silence but not degrade its target. Since then it has been found that miRNA silencing is actually accompanied by a drop of the levels of the target mRNAs; the drop is often modest, a factor of 2-3 is common, which seems insufficient to fully explain the drop in protein levels, suggesting that also intact mRNAs are silenced (Bagga et al., 2005). The discrepancy with earlier data may be explained because the original study measured RNA levels by RNase protection rather than Northern blots, a technique that is not so sensitive to partial degradation of RNA. A second point that needs to be clarified is whether the translation-suppressing effect of miRNAs is on initiation or elongation of translation, with a recent study showing that introduction of an IRES (Internal Ribosome Entry Site) overrules miRNA repression, suggesting the action is on initiation (Pillai et al., 2005).

What is the function of miRNAs? The virtual lack of miRNA mutants discovered in forward mutant hunts in genetic systems such as Drosophila or C. elegans may partly be attributed to the small size of the miRNAs as targets of mutagenesis; in addition the miRNAs seem fairly tolerant of a single base change as long as it does not affect the “seed sequence” of 7 nucleotides. Furthermore researchers trying to map a mutation to a protein coding region may have chosen to ignore mutations in non-coding miRNA sequences. However, probably the most important explanation that the miRNAs have been missed in mutant screens is that their knock-out has often no phenotype. In a recent study miRNAs in the nematode genome were knocked out, and the result was that single mutants did not while multiple mutants did have a phenotype (Abbott et al., 2005). We also see this with knock-down of miRNAs in zebrafish embryos using morpholinos. The conclusion is that there is much redundancy; possibly the very high levels of miRNAs in a cell (often more than 50,000 copies) is best achieved by a set of related miRNA encoding genes, and the loss of one of them leads to a modest reduction of levels that is not immediately resulting in a strong visible phenotype. As so often in biology, this raises the question why so many miRNA genes have been strongly conserved if there seems so little selective pressure, and as so often the answer needs to lay in subtle effects that are not recognized under laboratory conditions.

As the seed sequence seems to determine the target specificity of the miRNA the present invention further provides a nucleic acid sequence comprising at least nucleotides 2-8 of a miRNA as depicted in FIG. 1, or the seed sequence of a mammalian homologue of a miRNA as depicted in FIG. 1. In a preferred embodiment said nucleic acid sequence comprising at least nucleotides 2-8 of a miRNA of FIG. 1 comprises between about 18 and 26 nucleotides. Preferably, between about 20-24 nucleotides, more preferably about 22 nucleotides.

As described, knock out mutants of single miRNAs give few hints about the function of miRNAs. One indication of function comes from the study of the expression pattern of miRNAs: our laboratory showed recently that many miRNAs have striking organ specific expression, or even expression restricted to single tissue layers within one organ. This indicates that they play no general housekeeping role in cell metabolism, but most likely a role in an aspect of the difference between differentiated cells (Wienholds et al., 2005). An example of such expression patterns is miR-206 in muscle and miR-34A in the cerebellum. A second hint comes from the crudest miRNA knock out experiment possible: the knock out of all miRNAs (plus siRNAs), by disruption of the Dicer gene, which encodes the nuclease that make miRNAs. (Wienholds et al., 2003). As perhaps expected, this mutation is lethal. In mouse Dicer function is even required for stem cell formation. In zebrafish, however, it is not. Thus one can cross two Dicer heterozygous fish, and analyze the homozygous progeny: it develops normally until approximately a week of age, at which time growth stops and the animals eventually die. The fish embryos have formed most of their organs by 24-48 hour, and after a week swim around, eat and behave as real little beasts, all of this without Dicer. Analysis of miRNA levels show part of the explanation: maternal rescue. In the first days of development even Dicer mutant embryos form new zygotic miRNAs, and this must be done by maternal Dicer function (Dicer mRNA and/or protein in the oocyte). Still it is noteworthy that—with the exception of a few miRNAs—in the first 24-48 hours of development only low levels are seen, also in the wildtype (Wienholds et al., 2005). Thus the temporal pattern of miRNA expression is that they appear long after most cells have differentiated and tissues have been formed. The slow rise of levels must be the result of accumulation over time: many miRNA genes are embedded in introns of protein coding genes, and are initially transcribed together with their “host” mRNA, and therefore presumably equimolar to the mRNAs; while the mRNA levels remain modest, the miRNA levels build up over time, because the miRNAs are much less turned over than their host mRNAs. An elegant experiment (Giraldez et al., 2005) further drove down the point that miRNAs play no great role in early development: the maternal expression of Dicer can be removed by transplanting germ cells from Dicer mutant embryos into wildtype embryos of the same age: when the fish grow up they are fertile, but their germ line is genetically Dicer mutant. In this situation the fish do not have maternal Dicer, and indeed the animals now arrest earlier in development, but they still form several tissues. The conclusion of these experiments is that miRNAs are required for full development, have an expression patterns suggestive of developmental roles, but are not required for initial tissue differentiation. The abovementioned studies can be further refined with the discovery of the miRNAs of FIG. 1 as new targets for expression of miRNAs in development have now become available.

Some recent studies describe how miRNAs can tune gene expression in development. One study describes the role of mir-61 in determining the fate of one cell in vulval development of the nematode via a feedback loop: cell fate is determined by mutually exclusive expression of one gene or another, and one protein turns on the expression of a miRNA, which tunes down the expression of the second protein (Yoo and Greenwald, 2005). Another recent study describes how miR-196 acts upstream of Hox genes (Hornstein et al., 2005). Genes in the Notch signaling cascade are regulated by a set of miRNAs (Lai et al., 2005). All of these cases can be referred to as programmed miRNA action: the action of miRNAs is an integral part of a developmental event. The logical consequence is that the action is under positive evolutionary pressure, and indeed the Notch-pathway study could exploit the evolutionary conservation of the target sites among insect species to recognize them in 3′ UTRs of genes.

A prerequisite for such developmental switches is that at some moment in time the miRNA and its target mRNA are expressed in the same tissue, so that the miRNA can exert its action and silence the expression of its target. Intuitively this is what one might expect to be the rule: if a mRNA is a “genuine target” of a miRNA, the two need to be co-expressed. In other words: a naïve approach to discover biologically relevant miRNA/target pairs would be the following: screen the sequence of the crucial seven base pair “seed” sequence of each miRNA against the 3′UTR of all known genes; take the sets of miRNA/mRNA pairs that result, then filter the entire set by only accepting the pairs where miRNA and target mRNA are expressed in the same tissue. This would seem logical, since how could the two interact if they are not expressed in the same cells? Interestingly two recent studies show the situation is more complex than that. One study was done in Drosophila (Stark et al., 2005), the other in mammals (Farh et al., 2005), and in essence the conclusion are largely the same. The first striking result is this. If one takes miRNAs known to be expressed in a certain type of tissue (say muscle), and looks at the expression levels of genes whose 3′UTR contain a (potential) target-site of such a miRNA (defined operationally as a perfect match to the 7 base seed sequence), then genes with a target site are expressed at higher levels in tissues that do not express the miRNA than in tissues that do! So real partners (miRNAs plus targets) are not necessarily co-expressed. Is this effect cause or consequence? Both of these studies compare miRNA levels to mRNA transcript levels, and since miRNAs can reduce transcript levels (see above) the cause/consequence relation is not entirely clear in all cases. Thus saying that mRNAs and miRNAs avoid co-expression may be an overstatement, since the reduction of a mRNA may also be the consequence of the action of the miRNA, not a consequence of avoidance at the transcriptional level. Bartel and coworkers addressed this point in an elegant fashion: they looked at genes in mouse that do not have a miRNA target, while the human ortholog does. These mouse genes are nevertheless still significantly avoiding expression in the tissues that express the miRNA. This suggests the avoidance is really at the transcriptional level, and is not absence as a result of miRNA action (because the mouse version of the gene sees no miRNA action in that tissue).

Then there is a second effect. Both papers find evidence for “anti-targets”: there is selective avoidance of target sites in genes that are expressed at high levels in tissues where the miRNAs are expressed. Since gene expression is reduced by miRNAs, the acquisition of new miRNA target sites for miRNAs expressed in that tissue (probably not an infrequent event in evolution, since the crucial seed sequence is only 7 nucleotides long) is bad news and will be selected against if it results in an undesired knock down of that gene.

So how do the examples of programmed miRNA action, serving as developmental switches, relate to the notion of avoidance of co-expression? If the miRNA relates to its target as vacuum cleaner to dust, how can the two be seen as fine-tuned partners in a subtle developmental switch? The answer is provided by a beautiful distinction made in the study by Bartel and colleagues (Farh et al., 2005): targets of miRNAs fall into two classes: conserved and non-conserved. This is here operationally defined as those targets which are or are not conserved in the 3′UTRs of human versus orthologous mouse genes. The majority is not conserved, a minority is. Now here is the discovery: the conserved targets are in genes that do not avoid co-expression with their miRNAs, the non-conserved do avoid it.

The class of conserved targets is explained by the essential role the miRNA plays in developmental switches, such as those discussed above in the vulva and the Notch pathways, and we can refer to those cases as programmed miRNA silencing. A second type of conserved targets are those where gene expression is required in one phase of development, but after cell fate determination the miRNAs survey cells to wipe out the remaining traces of expression of these mRNAs that are not meant to be expressed in that tissue. The miRNA system is a vacuum cleaner removing the last speckle of undesired transcripts. Alternatively the system may serve to tune down but deliberately not shut off their targets. Together with the late onset and perseverance of expression of many miRNAs and the differentiation of tissues in embryos of fish devoid of all miRNAs, this indicates that the primary function of many miRNAs may not be to switch cell fate, but rather to dampen the expression of undesired genes, to remind a cell of the fate it has chosen previously: remember you are a muscle cell, do not have the nerve to highly express other genes!

The non-conserved majority has a completely different explanation: apparently 3′UTRs of genes are full of sequences to which miRNAs can bind; this is not surprising if the only truly essential feature is homology to the 7 nucleotide seed sequence: with a 3′UTR of one or two thousand base pairs, and with hundreds of different miRNAs, there will often be matches. In evolution such new “miRNA” recognition sequences pop up all of the time, and there is nothing wrong with them per se. The problem appears only if the target is in a gene that needs to be expressed at a significant level precisely in the tissue in which the corresponding miRNA is present at high levels, ready to silence any miRNA that matches its seed sequence. For these genes the match to this miRNA may be a nuisance, with negative fitness as result, and thus these matches are counter selected. Newly appearing miRNA target sequences (of no function, and thus under no evolutionary pressure to remain conserved) will not be selected against, and have essentially neutral fitness effects if the miRNA that could bind to them is not expressed in the same tissue. These target sequences have no physiological relevance, and thus are therefore ignored by evolution, neither selected for nor against, as long as they are not expressed in the tissues that express the miRNAs. These 7 base pair sequences are to the organism like EcoRI restriction sites in DNA (GAATTC): of no concern or interest (as long as there is no EcoRI around in the cell).

The combinations between miRNAs and their target can thus be classified in at least three groups: positively selected, neutral and negatively selected.

1. The positively selected or programmed interactions can be genuine cell fate switches, such as the switch of the 2nd vulval cell fate by miR-61 in worms, where at a crucial phase in time a cell needs to make a choice. A second type of programmed targets are those where after cell fate determination all traces of mRNAs that were required in a previous developmental stage need to be removed, or levels of genes need to remain tuned down significantly. Such interactions may be expected to be conserved, since they contribute positively to stable establishment or maintenance of cell fate.
2. The second class of combinations is neutral. There are two possibilities. The first one is trivial: miRNAs and targets are not expressed in the same tissue. If a gene is expressed uniquely in gut epithelium, the presence of a target for a muscle miRNA is irrelevant. A second class of pairs is real, meaning the miRNA and its target do interact in real life, but the effect is evolutionarily neutral. A gene may be tuned down a bit, or it may not, and the organism does not care. Note that these interactions are neutral in an evolutionary sense, no selective effect, but not in a biochemical sense, since the miRNAs do down-regulate (and experimental knock out of the miRNA would therefore result in an upward effect on target gene expression). The class of neutral but active miRNA-target interactions may turn out to be very large. While the first class (programmed interactions) will be conserved among species, the second class is not.
3. The third class of miRNA-target interactions are those where the miRNA is expressed in the same tissue as the mRNA, shutting off genes that need to be expressed. The avoidance data suggest that there is selective pressure against such co-expression, and they have been referred to as anti-targets. There is inevitably a steady state level of recently appeared target sites in anti-target genes, but these will be filtered out eventually by selective pressure.

Given these distinctions, there are several ways that mutation of miRNA-target interactions may cause disease. A miRNA may mutate and lose function; there is in many cases some level of redundancy, but this is at a gross level (visible in the laboratory), while loss of even one miRNA gene may have subtle negative disease-causing effects. Also a programmed miRNA target site may mutate, releasing the gene from miRNA-control. Finally a gene may acquire a novel and undesired miRNA target sequence: there are numerous sequences that are only one mutation away from becoming a target for one of the miRNAs expressed in the same tissue. Some of these mutations will result in undesired reduction of gene activity, and may cause disease. So the three possible causes of disease are: 1. mutation of a miRNA gene 2. mutation of a programmed miRNA target site 3. mutation that creates a new target-site in an anti-target gene.

On a more positive note: given complex combinatorial effects of regulation of genes by often more than one miRNA, each of which has a subtle effect on gene expression, polymorphisms in miRNA targets may be the ideal substrate for the type of small variations in development that natural selection can act upon in evolution. Protein coding changes may often either fully disrupt protein function altogether, which rarely contributes positively to fitness, they may leave the protein unaltered, or reduce the activity of the encoded protein. On the other hand miRNA target changes may sculpture expression patterns with great finesse. The many gradual differences that add up to make a mouse embryo out of a mouse zygote and a fish out of a fish zygote are certainly mostly differences in timing and levels of expression of factors that perform essentially identical biochemical actions, rather than differences in protein action. Therefore fine tuning of gene expression by gain or loss of miRNA target sequences may be expected to be a major mechanism in evolution and disease processes.

### EXAMPLES

### Material and Methods

Sequencing and Analysis of miRNA Regions in Primates.

Nested primer sets for PCR amplification of ˜700 bp regions for 144 known miRNA genes were designed using custom interface to primer3 software (http://primers.niob.knaw.nl). Primer selection was based solely on human sequences. Genomic DNAs of 10 primate species (NIA Aging Cell Repository DNA Panel PRP00001) were purchased from Coriell Cell Repositories (Camden, N.J.). All PCR reactions were carried out in a total volume of 10 μl with 0.5 Units Taq Polymerase (Invitrogen, Carlsbad Calif.) according to the manufacturer's conditions and universal cycling conditions (60 seconds 94° C., followed by 30 cycles of 94° C. for 20 seconds, 58° C. for 20 seconds and 72° C. for 60 seconds). PCR products were sequenced from both ends using an ABI3700 capillary sequencer (Applied Biosystems, Foster City Calif.). Sequences were quality trimmed and assembled using phred/phrap software (Ewing et al., 1998, Gordon et al., 1998) and aligned using POA (Lee et al., 2002).

Computational Prediction of miRNA Genes

All the analyses were performed using in-house developed software (Perl) when not stated otherwise. Whole-genome alignments (WGA) for human (July 2003 assembly), mouse (October 2003 assembly) and rat (June 2003 assembly) were downloaded from the UCSC Genome Bioinformatics site (http://genome.uscsc.edu). We first screened WGAs for blocks that fit miRNA-like conservation profile, i.e. have a conserved stem-loop region of ˜100 nt and non-conserved flanks of ˜50 nt. Technically, for every position we first calculated the percentage of conservation over a sliding window of 15 nt and assigned a value from 0 to 9 and ‘o’, where ‘o’ represents 100% identity, 9 between 90 and 100%, etcetera. Next, the resulting conservation string was searched by the following regular expression to define the conservation profile: /([0-8]{50,60})([o98]{53,260})([0-8]{50,60})/. At the next step we used RNAfold software (Hofacker, 2003) to evaluate the potential of conserved regions to form fold-back structures. The secondary structures matching the following regular expression were accepted: /((\((?:\.*\( ){24,})(\.{2,17}|\.*\({1,8}\.*\){1,8}\.*\({1,8}\.*\){1,8}\.*)(\)(?:\.*\)){50,}))/x (detailed scripts are available from the authors upon request). This step resulted in 12,958 candidate regions from human/mouse alignments and 12,530 candidate regions from human/rat alignments, which included 167 and 154 known human miRNAs, respectively. The original human/mouse and human/rat WGAs contained 187 and 172 annotated human miRNAs (miRNA registry v.3.1), respectively. Thus, the combined sensitivity of conservation profiling and fold-back structure selection steps is almost 90%. We did not calculate directly the contribution of the first, conservation-profiling, step to the filtering of candidate miRNA regions. It was reported previously, however, that about 800,000 stem-loops could be identified in conserved human/mouse non-coding regions (Lim et al., 2003). Therefore, we can estimate that conservation profiling is a very efficient filter that removes more than 98% of all potential fold-back structures while retaining 90% of real miRNAs. In cases where overlapping candidate regions were predicted on different DNA strands, the candidate with lower free folding energy was selected. This ‘naïve’ approach correctly identified the orientation of 144 known miRNAs out of 165 tested (87%).

As the third filtering step we used a recently discovered property of miRNAs to have lower folding free energies than random sequences with the same nucleotide content (Bonnet et al., 2004). Application of the Randfold program (filtering for regions with p<=0.005) further reduced the number of candidates 18-fold, to 716 for human/mouse and 639 for human/rat datasets. The sensitivity of this filtering step, when using p<=0.005 cutoff for randfold value, is about 85% (143 of 167 known miRNAs retained in human/mouse-, and 134 of 154—in human/rat dataset). The cutoff value of 0.005 is very stringent but provides an optimal sensitivity/specificity ratio for filtering.

Next, we intersected human/mouse and human/rat predictions using human genomic coordinates and orientation. It appeared that only 379 candidate regions that included 119 known miRNAs, were predicted in both datasets, and a substantial fraction of the predictions was set-specific, i.e. 337 candidates that include 24 known miRNAs, were found in human/mouse but not in human/rat WGA, whereas 260 candidates (including 15 known miRNAs) were found in human/rat but not human/mouse datasets. The detailed analysis of non-overlapping predictions revealed that about two thirds of them actually could be mapped to the corresponding genomic regions in the second rodent species (mouse predictions to the rat genome and vice versa) but failed to satisfy either conservation profiling or randfold criteria (for rodent sequences) or were simply not present in the initial WGA and hence were not picked up by our computational pipeline in a particular dataset. This analysis illustrates the value of combining data from two rodent species rather than concentrating on one, e.g. human/mouse, dataset.

In total, we have identified 976 candidate miRNA regions that satisfy the following criteria: (1) have characteristic miRNA-like conservation profiles in human/mouse or human/rat alignments; (2) form fold-back structures, and (3) have randfold value p<=0.005 for both human and rodent sequences. These 976 candidate regions included 158 known miRNAs (based on data from miRNA registry v.3.1). The initial whole-genome human/murine alignments, then combined, covered 189 known miRNAs. Therefore, the sensitivity of our analysis, based on this dataset, is 83% (158/189). At the same time, the specificity of the predictions ideally should be inferred from experimental verifications of all predictions. It is possible, however, to use conservation of candidate regions in additional genomes as an indirect measure of robustness of predictions. We have used zebrafish, chicken, opossum, cow and dog genomes to search for orthologs of our predicted candidates. Since opossum and cow genomes were not assembled at the time of analysis, we utilized Genotrace software (Berezikov et al., 2002) to make partial assemblies of regions of interest from trace data. The region from a genome was considered as orthologous to the candidate region if it (1) had at least 16 identical matches to the candidate sequence in at least 18 bp long hit, (2) was folded into a hairpin and (3) passed the randfold free energy criterion. It appeared that 678 out of 976 candidates (˜70%) are conserved in at least one more species besides rodents.

To produce additional candidate microRNA genes, the mouse genome was scanned for potential hairpins with a sliding window of 100 nt, and randfold values were calculated for resulting hairpins (mononucleotide shuffling, 1000 iterations). From a large set of hairpins that have low randfold values but are not necessarily conserved in other species, a subset of 199 was randomly selected.

Characterization of Candidate miRNA Regions

To put the predicted miRNA candidates into genomic context, we used the Ensembl (version 24) annotation of the human genome. We have searched our candidates against the ncRNA subset of the FANTOM database (Okazaki et al., 2002) and found that 3 regions (cand428, cand523 and cand420) overlap with or reside next to non-coding RNAs. Data for Affymetrix high-resolution tiling arrays (Kapranov et al., 2002) were downloaded from the UCSC Genome web site (http://hgdownload.cse.ucsc.edu/goldenPath/10april2003/database/affyTranscription.txt.gz and affyTransfrags.txt.gz), remapped to the July 2003 human genome assembly and intersected with candidate region predictions. Candidate regions that overlapped or resided within 50 bp from an annotated Transfrag region were associated with a given Transfrag fragment.

Northern Blot Analysis of Predicted miRNA Regions

We performed Northern blot analysis of 69 candidates representing different subgroups of candidates, such as broadly (zebrafish) or narrowly (rodents only) conserved, clustered or in families, located in introns, exons or intergenic. We limited our analysis to testing the expression of miRNAs in 3 mouse embryonic stages (8.5, 12.5 and 16.5 dpc), mouse ES cells, and mouse brain. Since we cannot predict the exact position of the mature miRNA in a stem, we used 35 nt-long probes that cover most of the hairpin arm. The arm containing a mature miRNA sequence was predicted on the basis of conservation level. For some candidate regions both arms of the hairpin were tested. For the candidates conserved in zebrafish, we also performed Northern blot analysis on RNA from zebrafish embryos (7, 14, 21 and 28 days) and a Dicer mutant (Wienholds et al., 2003).

RNA was isolated using mirVana miRNA isolation kit (Ambion, Austin Tex.), separated on 12% denaturing polyacrylamide gels alongside RNA Decadeä marker (Ambion, Austin Tex.), transferred by electroblotting to positively charged nylone membranes (Roche, Basel). Blots were hybridized overnight at 37° C. with radioactively (32P) labeled DNA oligo probes in modified Church and Gilbert buffer, washed three times with 2×SSC, 0.1% SDS at 37° C., and visualized using phosphoimaging (Typhoon, Amersham, UK). In some cases (cand181 and cand707), mature bands were detected only after a weeklong exposure of a blot, indicating the sensitivity limits of Northern blot analysis.

### RAKE Microarray Design and Analysis

The microarray for verification of candidate microRNAs using the RAKE assay was designed as a 44K custom microarray (Agilent Technologies, Palo Alto Calif., USA). 60-mer probes that are attached to the glass surface with their 3′-end were designed to include a fully matching probe sequence of 25 nucleotides complementary to the predicted microRNA with universal spacers on each side (5′-end, 5′-spacer: CGATCTTT, sequence of 21 nt complementary to the microRNA candidate region (tiling path), 3′-spacer: TAGGGTCCGATAAGGGTCAGTGCTCGCTCTA, 3′-end attached to glass surface). The three T's in the 5′-spacer function as a template for Klenow-mediated microRNA extension using biotin-dATP. A tiling path of 11 nucleotides was designed to cover the most likely Dicer/Drosha cleavage site determined at 22 nt upstream and downstream from the terminal loop extended to contain at least 11 unpaired nucleotides. For all cases, probes were designed for both arms of the hairpin sequence and for 648 candidates an additional set of 2×11 probes was designed as the transcript originating from the antisense genomic sequence can also efficiently fold into a stable hairpin structure. All 22/44 probes for a candidate microRNA were located in clusters on the array to exclude regional background effects. 10 different hybridization controls complementary to plant microRNAs (miR-402, UUCGAGGCCUAUUAAACCUCUG; miR-418, UAAUGUGAUGAUGAACUGACCU; miR-167, UGAAGCUGCCAGCAUGATCUGG; miR-416, GGUUCGUACGUACACUGUUCAU; miR-173, UUCGCUUGCAGAGAGAAAUCAC; miR-417, GAAGGUAGUGAAUUUGUUCGAC; miR-163, GAAGAGGACUUGGAACUUCGAU; miR-419, UUAUGAAUGCUGAGGAUGUUGU; miR-405, GAGUUGGGUCUAACCCAUAACU; miR-420, UAAACUAAUCACGGAAAUGCAC) were represented 10 times randomly distributed on the array. Microarrays were scanned on an Agilent scanner model G2565B at 10 μm resolution and spot identification and intensity determination was done using Agilent Feature Extraction software (Image Analysis version A. 7.5.1) with standard settings. To permit manual inspection and annotation of mature microRNA sequences, the raw images and spot intensity data were processed using custom scripts and visualized together with tiling path sequence information. Web-based interfaces were designed for annotation of single experiments and for summarizing all experiments. After manual inspection, all novel mature microRNA sequences that were positive were fed into the bioinformatic analysis pipeline set up for the evaluation of the cloned small RNAs, to filter out signal originating from repetitive elements and structural RNAs and to find homologous miRNAs in other species.

### Modified RAKE Assay

The original RAKE assay (Nelson et al., 2004) was modified for use with high-density custom-printed microarrays in the Agilent platform. Most importantly, in contrast to most custom-spotted micro-arrays, custom-printed probes are attached with their 3′-end to the glass surface. This excludes the need for the exonuclease that was included in the original protocol to reduce background signal from fold-backs of the free 3-ends of the probes that result in double-stranded DNA structures that can function as a template for the Klenow extension, resulting in aspecific background signal. Furthermore, hybridization, washing, and incubation conditions were adapted. All hybridization and wash buffers were made fresh from autoclaved stock solutions using DEPC-treated water, filter-sterilized and pre-heated. Microarray slides and coverslips were pre-washed two times for 2 minutes at 37° C. with preheated wash buffer (2×SSPE, 0.025% N-lauroylsarcosine), followed by 5 minute incubation with pre-hybridization buffer (5×SSPE, 40% formamide, 0.025% N-lauroylsarcosine). Next, the Agilent hybridization chamber was completely filled with hybridization mix, leaving no air-bubbles, as the usual air-bubble for mixing does not move around at low temperature and with the hybridization mix used. The hybridization mix (750 μl total per slide) consists of 500 μl 1.5× hybridization buffer (7.5×SSPE, 60% formamide, 0.0375% N-lauroylsarcosine), 10 μl spike—in RNA (control plant microRNAs stock: miR-402, 1×10⁻⁶ M; miR-418, 3.3×10⁻⁷ M; miR-167, 1×10⁻⁷ M; miR-416, 3.3×10⁻⁸ M; miR-173, 1×10⁻⁸ M; miR-417, 3.3×10⁻⁹ M; miR-163, 1×10⁻⁹ M; miR-419, 3.3×10⁻¹⁰ M; miR-405, 1×10⁻¹⁰ M; miR-420, 3.3×10⁻¹¹ M), and 20 μg small RNA sample (8.5 dpc and 16.5 dpc mouse embryo, mouse embryonic stem (ES) cells and total brain), isolated using the MirVana microRNA isolation kit (Ambion, Austin Tex., USA) and supplemented with DEPC-treated water up to 240 μl. The hybridization mix was heated to 75° C. for 5 minutes and cooled on ice before application to the array. The array was incubated overnight at 37° C., followed by 4 washes of 2 minutes in wash buffer and 1 wash for 2 minutes in 1× Klenow buffer (10 mM Tris pH7.9, 50 mM NaCl, 10 mM MgCl₂, 1 mM DTT, 0.025% N-lauroylsarcosine). For the Klenow extension, an enzyme mix (750 μl total per slide) containing 375 μl 2× Klenow buffer, 365 μl DEPC-treated water, 2.5 μl Klenow Exo—(50,000 U/μl, NEB, Ipswich Mass., USA), and 7.5 μl biotin-14-dATP (4 μM stock, Perkin Elmer, Wellesley Mass., USA) was applied to the array in a clean incubation chamber and incubated for 1 hour at 37° C. Next, the array was washed four times for 2 minutes with wash buffer and once for 2 minutes with 1× Klenow buffer. Next, the dye conjugation mix (total volume 750 μl) consisting of 375 μl 2× Klenow buffer, 368 μl DEPC-treated water and 20 μl streptavidin-conjugated Alexa fluor-647 (2 mg/ml stock, Invitrogen, Carlsbad Calif., USA) was applied in a new incubation chamber for 30 minutes at 37° C., followed by four washes of 2 minutes at 37° C. with wash buffer and 5 brief dips in DEPC water to remove salts. Slides were dried by centrifugation in a 50 ml tube by spinning for 5 minutes at 1000 rpm (180×g).

### Small RNA Library Construction and Sequencing.

Seven high-titer small RNA libraries were made. Briefly, the small RNA fraction from adult mouse brain (12 weeks) and various human fetal tissues (17 weeks of development: brain; heart; skin; lung; mix 1: multiple fetal tissues; mix 2: liver, stomach, bowel) was isolated using the mirVana microRNA isolation kit (Ambion), followed by an additional enrichment by excision of the 15 to 30 nt fraction from a polyacrylamide gel. For cDNA synthesis the RNA molecules in this fraction were first poly A-tailed using yeast poly(A)polymerase followed by ligation of a RNA linker oligo to the 5′ phosphate of the miRNAs. First strand cDNA synthesis was then performed using an oligo(dT)-linker primer and M-MLV-RNase H-reverse transcriptase. The resulting cDNA was then PCR amplified for 15 to 22 cycles (depending on the start material quality and quantity), followed by restriction nuclease treatment, gel purification of the 95-110 bp fraction, and cloning in the EcoRI and BamHI sites of the pBSII SK+plasmid vector. Ligations were electroporated into T1 Phage resistant TransforMaxTMEC100TM electrocompetent cells (Epicentre), resulting in titers between 1.2 and 3.3×10⁶ recombinant clones per library. A total of 83,328 colonies were automatically picked into 384-well plates (Genetix QPix2, New Milton Hampshire, UK) containing 75 μl LB-Amp and grown overnight at 37° C. with continuous shaking. All following pipetting steps were performed using liquid handling robots (Tecan (Mannedorf, Switzerland) Genesis RSP200 with integrated TeMo96 and Velocity11 (Menlo Park Calif., USA) Vprep with BenchCell 4×). 5 μl of culture was transferred to a 384-well PCR plate (Greiner, Mannheim, Germany) containing 20 μl water, and cells were lysed by heating for 15 minutes at 95° C. in a PCR machine. 1 μl of lysed suspension was transferred to a fresh 384-wells plate containing 4 μl PCR mix (final concentrations: 0.2 μM M13forward, TGTAAAACGACGGCCAGT; 0.2 μM M13reverse, AGGAAACAGCTATGACCAT, 400 μM of each dNTP, 25 mM tricine, 7.0% glycerol (w/v), 1.6% DMSO (w/v), 2 mM MgCl₂, 85 mM ammonium acetate pH 8.7 and 0.2 U Taq Polymerase in a total volume of 10 μl) and the insert was amplified by 35 cycles of 20″ 94° C., 10″ 58° C., 30″ 72° C. After adding 30 μl water, 1 μl of PCR product was directly used for dideoxy sequencing by transferring to a new 384-well PCR plate containing 4 μl sequencing mix (0.027 μl BigDye terminator mix v3.1 (Applied Biosystems, Foster City, Calif., USA), 1.96 μl 2.5× dilution buffer (Applied Biosystems), 0.01 μl sequencing oligo (100 μM stock T7, GTAATACGACTCACTATAGGGC), and 2 μl water). Thermocycling was performed for 35 cycles of 10″ 94° C., 10″ 50° C., 20″ 60° C. and final products were purified by ethanol precipitation in 384-well plates as recommended by the manufacturer (Applied Biosystems) and analyzed on ABI3730XL sequencers with a modified protocol for generating approximately 100 nt sequencing reads.
Computational analysis of cloned small RNAs sequencing reads
Base calling and quality trimming of sequence chromatograms was done by phred software (Ewing et al., 1998). After masking of vector and adapter sequences, and removing redundancy, inserts of length 18 bases and longer were mapped to genomes (ncbi35 assembly for human and ncbim34 assembly for mouse) using megablast software (ftp://ftp.ncbi.nlm.nih.gov/blast/). Not all inserts matched perfectly to a genome, and detailed analysis of non-matching sequences indicated that many of them represent known microRNAs with several additional nucleotides added to one of the ends. These non-genomic sequences may be artifacts of the cloning procedure or a result of non-templated modification of mature microRNAs (Aravin et al., 2005). Such sequences were corrected according to the best blast hit to a genome. Next, for every genomic locus matching to an insert, repeat annotations were retrieved from the Ensembl database (http://www.ensembl.org) and repetitive regions were discarded from further analysis, with the exception of the following repeats: MIR, MER, L2, MARNA, MON, Arthur and trf, since these repeat annotations overlap with some known microRNAs. Genomic regions containing inserts with 100 nt flanks were retrieved from Ensembl and a sliding window of 100 nt was used to calculate RNA secondary structures by RNAfold (Hofacker, 2003). Only regions that folded into hairpins and contained an insert in one of the hairpin arms, we used in further analysis. Since every non-redundant insert produced independent hits at this stage, hairpins with overlapping genomic coordinates were merged into one region, tracing locations of matching inserts. In cases when several inserts overlapped, the complete region covered by overlapping inserts was used in downstream calculations as a mature sequence. Next, gene and repeat annotations for hairpin genomic regions were retrieved from Ensembl, and repetitive regions (with above mentioned exceptions) as well as ribosomal RNAs, tRNAs and snoRNAs were discarded. To find homologous hairpins in other genomes, mature regions were blasted against human, mouse, rat, dog, cow, opossum, chicken, zebrafish and fugu genomes. Hits with length of at least 20 nt and identity of at least 70% were extracted from genomes along with flanking sequences of length similar to that observed in original hairpins to which a certain mature query sequence belonged. Extracted sequences were checked for hairpin structures using RNAfold, and positive hairpins were aligned with the original hairpin using clustalw (Thompson et al., 1994). Only homologs with at least 70% overall identity and 90% identity within mature sequence were considered. In cases were several homologous hairpins in a species were identified, the best clustalw-scoring hairpin was retained. Next, homologs from different organisms were aligned with the original hairpin by clustalw to produce a final multiple alignment of the hairpin region. Chromosomal location of homologous sequences were used to retrieve gene and repeat annotations from respective species Ensembl databases. Hairpins that contained repeat/RNA annotations in one of the species, as well as hairpins containing mature regions longer that 25 nt or with GC-content higher than 85% were discarded. For remaining hairpins, randfold values were calculated for every sequence in an alignment using mononucleotide shuffling and 1000 iterations. The cut-off of 0.01 was used for randfold and only regions that contained a hairpin below this cut-off for at least one species in an alignment, were considered as microRNA genes. Finally, positive hairpins were split into known and novel microRNAs according annotations. To facilitate these annotations and also to track performance of the pipeline, mature sequences of known microRNAs from miRBase (Griffiths-Jones, 2004) were included into the analysis.
Expression of miRNA in Tissue Samples
Custom microarrays (Amersham Codelink) were made by spotting 3′-aminolinked oligonucleotides (60-mers, as described above for the custom Agilent microarrays) for detection of all known and novel mature microRNAs. At this point, no tiling path is needed anymore, resulting in a slide with about 15,000 spots that represent the full human, mouse and rat miRNA reportoire in 8-fold. These slides were hybridized with small RNA from mouse heart and mouse thymus (isolated using the Ambion MirVana small RNA isolation kit) as described above for the custom Agilent microarrays. In the table below, normalized intensities (arbitrary values, average of 8 spots, normalized by assuming a constant total amount of microRNA molecules per sample) for thymus and heart are shown for the those miRNAs that are more than two-fold differentially expressed. It should be noted that low values may indicate background signal and absense of this particular miRNA in a sample. Clearly, eight out of the 24 miRNAs that are differentially expressed between thymus and heart and hence provide a characteristic signature of the respective tissues, are novel miRNAs as described in FIG. 1.

TABLE
expression of miRNAs as detected by microarray analysis

| | |
|---|---|
| signal intensity | fold |

| rank | miRNA | thymus | heart | difference |
|---|---|---|---|---|
| 1 | mmu-mir-133b | 0.2767 | 5.3531 | 19.3 |
| 2 | novel Mmd_532 | 3.5050 | 0.2970 | −11.8 |
| 3 | mmu-mir-125b | 1.3814 | 11.9810 | 8.7 |
| 4 | mmu-mir-99a | 0.8470 | 6.1479 | 7.3 |
| 5 | novel Mmd_524 | 0.0117 | 0.0527 | 4.5 |
| 6 | novel Mmd_124 | 0.0094 | 0.0412 | 4.4 |
| 7 | mmu-mir-126 | 4.2831 | 16.3321 | 3.8 |
| 8 | mmu-mir-145 | 1.1160 | 4.1833 | 3.7 |
| 9 | mmu-mir-30a | 2.1039 | 7.3289 | 3.5 |
| 10 | mmu-mir-150 | 4.5540 | 1.4430 | −3.2 |
| 11 | mmu-mir-106a | 0.6968 | 0.2245 | −3.1 |
| 12 | mmu-mir-30e | 2.6240 | 7.6983 | 2.9 |
| 13 | novel Mmd_297 | 0.2878 | 0.8431 | 2.9 |
| 14 | mmu-mir-145 | 0.5578 | 1.5293 | 2.7 |
| 15 | mmu-mir-21 | 4.1493 | 1.5676 | −2.6 |
| 16 | novel Mmd_254 | 0.0178 | 0.0461 | 2.6 |
| 17 | novel Mmd_120 | 0.3228 | 0.1308 | −2.5 |
| 18 | mmu-mir-26a | 2.4855 | 5.9199 | 2.4 |
| 19 | mmu-let-7e | 1.1802 | 2.7889 | 2.4 |
| 20 | novel Mmd_45 | 0.3750 | 0.1599 | −2.3 |
| 21 | novel Mmd_93 | 0.0239 | 0.0558 | 2.3 |
| 22 | mmu-mir-185 | 0.6790 | 1.5214 | 2.2 |
| 23 | mmu-mir-149 | 0.1115 | 0.2333 | 2.1 |
| 24 | mmu-mir-18 | 1.9616 | 0.9721 | −2.0 |

### REFERENCES

Abbott, A. L., Alvarez-Saavedra, E., Miska, E. A., Lau, N. C., Bartel, D. P., Horvitz, H. R., Ambros, V. (2005). The let-7 microRNA family members mir-48, mir-84 and mir-241 function together to regulate developmental timing in Caenorhabditis elegans. Dev. Cell 9, 403-414.
Alvarez-Garcia, I. & Miska, E. A. MicroRNA functions in animal development and human disease. Development 132, 4653-62 (2005).
Ambros, V. (2004). The functions of animal microRNAs. Nature 431, 350-355.
Ambros, V., Lee, R. C., Lavanway, A., Williams, P. T. and Jewell, D. (2003). MicroRNAs and Other Tiny Endogenous RNAs in C. elegans. Curr Biol 13: 807-18.
Aravin, A. & Tuschl, T. Identification and characterization of small RNAs involved in RNA silencing. FEBS Lett 579, 5830-40 (2005).
Aravin, A. A., Naumova, N. M., Tulin, A. V., Vagin, V. V., Rozovsky, Y. M. and Gvozdev, V. A. (2001). Double-stranded RNA-mediated silencing of genomic tandem repeats and transposable elements in the D. melanogaster germline. Curr Biol 11: 1017-27.
Bagga, S., Bracht, J., Hunter, S., Massirer, K., Holtz, J., Eachus, R., Pasquinelli, A. E. (2005). Regulation by let-7 and lin-4 miRNAs results in target mRNA degradation. Cell 122, 553-563.
Bartel, D. P. (2004). MicroRNAs: genomics, biogenesis, mechanism, and function. Cell 116, 281-297.
Bentwich I, Avniel A, Karov Y, Aharonov R, Gilad S, Barad O, Barzilai A, Einat P, Einav U, Meiri E, Sharon E, Spector Y, Bentwich Z. (2005) Identification of hundreds of conserved and nonconserved human microRNAs. Nature Genet. 37, 766-770.
Berezikov, E., Plasterk, R. H. and Cuppen, E. (2002). GENOTRACE: cDNA-based local GENOme assembly from TRACE archives. Bioinformatics 18, 1396-1397.
Berezikov, E., Guryev, V., van de Belt, J., Wienholds, E., Plasterk, R. H., Cuppen, E. (2005). Phylogenetic shadowing and computational identification of human microRNA genes. Cell 120, 21-24.
Bernstein, E., Caudy, A. A., Hammond, S. M. and Hannon, G. J. (2001). Role for a bidentate ribonuclease in the initiation step of RNA interference. Nature 409: 363-6.
Boffelli, D., McAuliffe, J., Oveharenko, D., Lewis, K. D., Oveharenko, I., Pachter, L. and Rubin, E. M. (2003). Phylogenetic shadowing of primate sequences to find functional regions of the human genome. Science 299, 1391-1394.
Bohnsack, M. T., Czaplinski, K. and Gorlich, D. (2004). Exportin 5 is a RanGTP-dependent dsRNA-binding-protein that mediates nuclear export of pre-miRNAs. Rna 10: 185-91.
Bonnet, E., Wuyts, J., Rouze, P. and Van De, P.e.Y. (2004). Evidence that microRNA precursors, unlike other non-coding RNAs, have lower folding free energies than random sequences. Bioinformatics 20, 2911-2917.
Brennecke, J., Hipfner, D. R., Stark, A., Russell R. B. and Cohen, S. M. (2003). bantam encodes a developmentally regulated microRNA that controls cell proliferation and regulates the proapoptotic gene hid in Drosophila. Cell 113: 25-36.
Cai, X., Hagedorn, C. H. and Cullen, B. R. (2004). Human microRNAs are processed from capped, polyadenylated transcripts that can also function as mRNAs. RNA 10, 1957-1966.
Calin, G. A. et al. A MicroRNA signature associated with prognosis and progression in chronic lymphocytic leukemia. N Engl J Med 353, 1793-801 (2005).
Chen, X. (2004). A microRNA as a translational repressor of APETALA2 in Arabidopsis flower development. Science 303: 2022-5.
Ewing, B., Hillier, L., Wendl, M. C. & Green, P. Base-calling of automated sequencer traces using phred. I. Accuracy assessment. Genome Res 8, 175-85 (1998).
Farh, K. K., Grimson, A., Jan, C., Lewis, B. P., Johnston, W. K., Lim, L. P., Burge, C. B., Bartel, D. P. (2005). The widespread impact of mammalian microRNAs on mRNA repression and evolution. Science 310, 1817-1821.
Fire, A., Xu, S., Montgomery, M. K., Kostas, SA., Driver, S. E. and Mello, C. C. (1998). Potent and specific genetic interference by double-stranded RNA in Caenorhabditis elegans. Nature 391: 806-11.
Giraldez, A. J., Cinalli, R. M., Glasner, M. E., Enright, A. J., Thomson, J. M., Baskerville, S., Hammond, S. M., Bartel, D. P., Schier, A. F. (2005). MicroRNAs regulate brain morphogenesis in zebrafish. Science 308, 833-838.
Gordon, D., Abajian, C. and Green, P. (1998). Consed: a graphical tool for sequence finishing. Genome Res 8, 195-202.
Griffiths-Jones, S. (2004). The microRNA Registry. Nucleic Acids Res 32 Database issue, D109-11.
Grishok, A., Pasquinelli, A. E., Conte, D., Li, N., Parrish, S., Ha, I., Baillie, D. L., Fire, A., Ruvkun, G. and Mello, C. C. (2001). Genes and mechanisms related to RNA interference regulate expression of the small temporal RNAs that control C. elegans developmental timing. Cell 106: 23-34.
Hamilton, A. J. and Baulcombe, D. C. (1999). A species of small antisense RNA in posttranscriptional gene silencing in plants. Science 286: 950-2.
Hammond, S. M. MicroRNAs as oncogenes. Curr Opin Genet Dev (2005). He L, Thomson J M, Hemann M T, Hernando-Monge E, Mu D, Goodson S, Powers S, Cordon-Cardo C, Lowe S W, Hannon G J, Hammond S M. (2005) A microRNA polycistron as a potential human oncogene. Nature 435, 828-33.
Hofacker, I. L. Vienna RNA secondary structure server. Nucleic Acids Res 31, 3429-31 (2003).
Hornstein, E., Mansfield, J. H., Yekta, S., Kuang-Hsien Hu, J., Harfe, B. D., McManus, M. T., Baskerville, S., Bartel, D. P., Tabin, C. J. (2005). The microRNA miR-196 acts upstream of Hoxb8 and Shh in limb development. Nature 438, 671-674.
Hutvagner, G., Mclachlan, J., Pasquinelli, A. E., Balint, E., Tuschl, T. and Zamore, P. D. (2001). A cellular function for the RNA-interference enzyme Dicer in the maturation of the let-7 small temporal RNA. Science 293: 8348.
Johnson, S. M., Lin, S. Y. and Slack, F. J. (2003). The time of appearance of the C. elegans let-7 microRNA is transcriptionally controlled utilizing a temporal regulatory element in its promoter. Dev Biol 259: 364-79.
Johnston, R. J. and Hobert, O. (2003). A microRNA controlling left/right neuronal asymmetry in Caenorhabditis elegans. Nature 426: 845-9.
Kapranov, P., Cawley, S. E., Drenkow, J., Bekiranov, S., Strausberg, R. L., Fodor, S. P. and Gingeras, T. R. (2002). Large-scale transcriptional activity in chromosomes 21 and 22. Science 296, 916-919.
Ketting, R. F., Fischer, S. E., Bernstein, E., Sijen, T., Hannon, G. J. and Plasterk, R. H. (2001). Dicer functions in RNA interference and in synthesis of small RNA involved in developmental timing in C. elegans. Genes Dev. 15: 2654-9.
Khvorova, A., Reynolds, A. and Jayasena, S. D. (2003). Functional siRNAs and miRNAs exhibit strand bias. Cell 115: 209-16.
Lagos-Quintana, M., Rauhut, R., Lendeckel, W. & Tuschl, T. Identification of novel genes coding for small expressed RNAs. Science 294, 853-8 (2001).
Lai, E. C., Tam, B., Rubin, G. M. (2005). Pervasive regulation of Drosophila Notch target genes by GY-box-, Brd-Box-, and K-box-class microRNAs. Genes Dev. 19, 1067-1080.
Lee, R. C., Feinbaum, R. L., Ambros, V. (1993). The C. elegans heterochronic gene lin-4 encodes small RNAs with antisense complementarity to lin-14. Cell 75, 843-854.
Lee, C., Grasso, C. and Sharlow, M. F. (2002). Multiple sequence alignment using partial order graphs. Bioinformatics 18, 452-464.
Lee, Y., Ahn, C., Han, J., Choi, H., Kim, J., Yim, J., Lee, J., Provost, P., Radmark, O., Kim, S. and Kim, V. N. (2003). The nuclear RNase III Drosha initiates microRNA processing. Nature 425: 415-9.
Lee, Y., Kim, M., Han, J., Yeom, K. H., Lee, S., Baek, S. H. and Kim, V. N. (2004). MicroRNA genes are transcribed by RNA polymerase II. EMBO J 23, 4051-4060.
Lee, Y. S., Nakahara, K., Pham, J. W., Kim, K., He, Z., Sontheimer, E. J. and Carthew, R. W. (2004). Distinct roles for Drosophila Dicer-1 and Dicer-2 in the siRNA/miRNA silencing pathways. Cell 117: 69-81.
Lewis, B. P., Burge, C. B., Bartel, D. P. (2005). Conserved seed pairing, often flanked by adenosines, indicates that thousands of human genes are microRNA targets. Cell 120, 15-20.
Lim, L. P., Glasner, M. E., Yekta, S., Burge, C. B. and Bartel, D. P. (2003). Vertebrate microRNA genes. Science 299, 1540.
Lim, L. P., Lau, N. C., Garrett-Engele, P., Grimson, A., Schelter, J. M., Castle, J., Bartel, D. P., Linsley, P. S., Johnson, J. M. (2005). Microarray analysis shows that some microRNAs downregulate large numbers of target mRNAs. Nature 433, 769-773.
Lingel, A., Simon, B., Izaurralde, E. and Sattler, M. (2003). Structure and nucleic-acid binding of the Drosophila Argonaute 2 PAZ domain. Nature 426: 465-9.
Lu, J. et al. MicroRNA expression profiles classify human cancers. Nature 435, 834-8 (2005).
Lund, E., Guttinger, S., Calado, A., Dahlberg, J. E. and Kutay, U. (2004). Nuclear export of microRNA precursors. Science 303: 95-8.
Ma, J., Ye, K. and Patel, D. (2004). Structural basis for overhang-specific small interfering RNA recognition by the PAZ domain. Nature in press.
Martinez, J. and Tuschl, T. (2004). RISC is a 5′ phosphomonoester-producing RNA endonuclease. Genes Dev.
Nelson, P. T. et al. Microarray-based, high-throughput gene expression profiling of microRNAs. Nat Methods 1, 155-61 (2004).
O'Donnell K A, Wentzel E A, Zeller K I, Dang C V, Mendell J T. (2005) c-Myc-regulated microRNAs modulate E2F1 expression. Nature 435:839-43
Ohler, U., Yekta, S., Lim, L. P., Bartel, D. P. and Burge, C. B. (2004). Patterns of flanking sequence conservation and a characteristic upstream motif for microRNA gene identification. RNA 10, 1309-1322.
Okazaki, Y., Furuno, M., Kasukawa, T., Adachi, J., Bono, H., Kondo, S., Nikaido, I., Osato, N., Saito, R., Suzuki, H., Yamanaka, I., Kiyosawa, H., Yagi, K, Tomaru, Y., Hasegawa, Y., Nogami, A., Schonbach, C., Gojobori, T., Baldarelli, R. and Hill, D. P. (2002). Analysis of the mouse transcriptome based on functional annotation of 60,770 full-length cDNAs. Nature 420, 563-573.
Park, W., Li, J., Song, R., Messing, J. and Chen, X. (2002). CARPEL FACTORY, a Dicer homolog, and HEN1, a novel protein, act in microRNA metabolism in Arabidopsis thaliana. Curr Biol 12: 1484-95.
Pham, J. W., Pellino, J. L., Lee, Y. S., Carthew, R. W. and Sontheimer, E. J. (2004). A Dicer-2-dependent 80s complex cleaves targeted mRNAs during RNAI in Drosophila. Cell 117: 83-94.
Pillai, R. S., Bhattacharyya, S. N., Artus, C. G., Zoller, T., Cougot, N., Basyuk, E., Bertrand, E., Filipowicz, W. (2005). Inhibition of translational initiation by let-7 microRNA in human cells. Science 309, 1573-1576.
Poy, M. N., Eliasson, L., Krutzfeldt, J., Kuwajima, S., Ma, X., Macdonald, P. E., Pfeffer, S., Tuschl, T., Rajewsky, N., Rorsman, P. and Stoffel, M. (2004). A pancreatic islet-specific microRNA regulates insulin secretion. Nature 432, 226-230.
Reinhart, B. J. and Bartel, D. P. (2002). Small RNAs correspond to centromere heterochromatic repeats. Science 297: 1831.
Reinhart, B. J., Slack, F. J., Basson, M., Pasquinelli, A. E., Bettinger, J. C., Rougvie, A. E., Horvitz, H. R., Ruvkun, G. (2000). The 21-nucleotide let-7 RNA regulates developmental timing in Caenorhabditis elegans. Nature 403, 901-906.
Reinhart, B. J., Weinstein, E. G., Rhoades, M. W., Bartel, B. and Bartel, D. P. (2002). MicroRNAs in plants. Genes Dev 16: 1616-26.
Rodriguez, A., Griffiths-Jones, S., Ashurst, J. L. and Bradley, A. (2004). Identification of Mammalian microRNA Host Genes and Transcription Units. Genome Res 14, 1902-1910.
Schwarz, D. S., Hutvagner, G., Du, T., Xu, Z., Aronin, N. and Zamore, P. D. (2003). Asymmetry in the assembly of the RNAi enzyme complex. Cell 115: 199-208.
Schwarz, D. S., Tomari, Y. and Zamore, P. D. (2004). The RNA-Induced Silencing Complex Is a Mg(2+)-Dependent Endonuclease. Curr Biol 14: 787-91.
Song, J. J., Liu, J., Tolia, N. H., Schneiderman, J., Smith, S. C, Martienssen, R. A., Hannon, G. J. and Joshua-Tor, L. (2003). The crystal structure of the Argonaute2 PAZ domain reveals an RNA binding motif in RNAi effector complexes. Nat Struct Biol 10: 1026-1032.
Stark, A., Brennecke, J., Bushati, N., Russell, R. B., Cohen, S. M. (2005). Animal microRNAs confer robustness to gene expression and have a significant impact on 3′-UTR evolution. Cell 123, 1133-1146.
Thompson, J. D., Higgins, D. G. & Gibson, T. J. CLUSTAL W: improving the sensitivity of progressive multiple sequence alignment through sequence weighting, position-specific gap penalties and weight matrix choice. Nucleic Acids Res 22, 4673-80 (1994).
Tomari, Y., Du, T., Haley, B., Schwarz, D. S., Bennett, R., Cook, H. A., Koppetsch, B. S., Theurkauf, W. E. and Zamore, P. D. (2004). RISC assembly defects in the Drosophila RNAi mutant armitage. Cell 116: 831-41.
Wienholds, E., Kloosterman, W. P., Miska, E., Alvarez-Saavedra, E., Berezikov, E., de Bruijn, E., Horvitz, H. R., Kauppinen, S., Plasterk, R. H. (2005). MicroRNA expression in zebrafish embryonic development. Science 309, 310-311.
Wienholds, E., Koudijs, M. J., van Eeden, F. J., Cuppen, E., Plasterk, R. H. (2003). The microRNA-producing enzyme Dicer 1 is essential for zebrafish development. Nat. Genet. 35, 217-218.
Xie, Z., Johansen, L. K, Gustafson, A. M., Kasschau, K. D., Lellis, A. D., Zilberman, D., Jacobsen, S. E. and Carrington, J. C. (2004). Genetic and Functional Diversification of Small RNA Pathways in Plants. PLoS Biol 2: E104.
Yan, K. S., Yan, S., Farooq, A., Han, A., Zeng, L. and Zhou, M. M. (2003). Structure and conserved RNA binding of the PAZ domain. Nature 426: 468-74.
Yekta, S., Shih, I. H. and Bartel, D. P. (2004). MicroRNA-directed cleavage of HOXB8 mRNA. Science 304: 594-6.
Yi, R., Qin, Y., Macara, I. G. and Cullen, B. R. (2003). Exportin-5 mediates the nuclear export of pre-microRNAs and short hairpin RNAs. Genes Dev 17: 3011-6.
Yoo, A. S., Greenwald, I. (2005). Lin-121Notch activation leads to microRNA-mediated down-regulation of Vav in C. elegans. Science 310, 1330-1333.
Zhang, H., Kolb, F. A., Jaskiewisz, L., Westhof, E. and Rilipowicz, W. (2004). Single processing center models for human Dicer and bacterial RNase III. Cell in press.

1. A method for characterising a sample comprising nucleic acid derived from a cell, said method comprising:
determining whether said sample comprises at least a minimal sequence of at least one microRNA (miRNA) of FIG. 1 or a mammalian homologue thereof or whether said sample comprises a hairpin RNA of FIG. 1 or a mammalian homologue thereof; and
characterizing said sample on the basis of the presence or absence of said miRNA or hairpin RNA.

2. The method according to claim 1, further comprising determining whether said sample comprises at least a minimal sequence of at least five miRNAs of FIG. 1 or mammalian homologues thereof.

3. The method according to claim 2, wherein said at least five miRNA are each derived from a different precursor miRNA (pre-miRNA) and wherein said method further comprises characterizing said sample on the basis of the presence or absence of said miRNAs.

4. The method according to claim 1, wherein said sample comprises nucleic acid of a cell selected from the group consisting of a differentiated cell, an embryonic cell, a cell derived from an embryonic cell line, a stem cell, a cell with an aberrant proliferation phenotype, a tumor cell, a cell derived from a tumor cell line, a lung cell, a skin cell, a brain cell, a liver cell, and a heart cell.

5-8. (canceled)

9. A method for determining whether a cell in a sample is different from a reference cell, said method comprising:
determining whether miRNA expression of at least one miRNA (miRNA) of FIG. 1 or a mammalian homologue thereof, or a hairpin RNA of FIG. 1 or a mammalian homologue thereof, in said cell is different when compared to the expression thereof in said reference cell.

10. The method according to claim 9, wherein said cell is selected from the group consisting of a differentiated cell, an embryonic cell, a cell derived from an embryonic cell line, a stem cell, a cell with an aberrant phenotype, a tumor cell, a cell derived from a tumor cell line, a lung cell, a skin cell, a liver cell, and a heart cell.

11-14. (canceled)

15. An isolated nucleic acid molecule comprising:
a) a nucleotide sequence as shown in FIG. 1,
b) a nucleotide sequence which is a complement of a),
c) a nucleotide sequence which has an identity of at least 80% to a miRNA sequence of FIG. 1 or 70% to a hairpin RNA of FIG. 1 or
d) a nucleotide sequence which hybridizes under stringent conditions to a sequence of a), b) or c).

16. The nucleic acid molecule of claim 15, wherein the identity of sequence c) to a sequence of FIG. 1 is at least 90%.

17. The nucleic acid molecule of claim 15 wherein the nucleic acid molecule is a miRNA molecule or an analogue thereof.

18. The nucleic acid molecule of claim 17, wherein the nucleic acid molecule has a length of from 18 to 26 nucleotides.

19. The nucleic acid molecule of claim 15, wherein the nucleic acid molecule is a pre-miRNA or a DNA molecule coding therefore.

20. The nucleic acid molecule of claim 19, wherein the nucleic acid molecule has a length of 60-110 nucleotides.

21. The nucleic acid molecule of claim 15, wherein the nucleic acid molecule is single-stranded.

22. The nucleic acid molecule of claim 15, wherein the nucleic acid molecule is at least partially double-stranded.

23. The nucleic acid molecule of claim 15, wherein the nucleic acid molecule comprises RNA, DNA, nucleic acid analogue molecules or combinations thereof.

24. The nucleic acid molecule of claim 23, wherein the nucleic acid molecule comprises at least one modified nucleotide analogue.

25. A method of performing a diagnosis, the method comprising:
obtaining a sample comprising nucleic acid derived from a cell;
determining whether said sample comprises the nucleic acid molecule of claim 15; and
characterizing said sample on the basis of the presence or absence of said nucleic acid molecule of claim 15; and
providing a diagnosis based on said characterization.

26. A collection of nucleic acid molecules, comprising at least 5 nucleic acid molecules comprising a nucleotide sequence as shown in FIG. 1.

27. A collection of nucleic acid molecules, comprising at least 5 nucleic acid molecules with a nucleotide sequence which is a complement of a nucleotide sequence as shown in FIG. 1, or with a nucleotide sequence which hybridizes under stringent conditions to a nucleotide sequence as shown in FIG. 1.

28. A method of diagnosing of cancer, heart disease, viral infection, or disease susceptibility, the method comprising:
obtaining a sample comprising nucleic acid derived from a cell;
determining whether said sample comprises one or more of the nucleic acid molecules in the collection of nucleic acid molecules of claim 26;
characterizing said sample on the basis of the presence or absence of one or more of the nucleic acid molecules in the collection of nucleic acid molecules of claim 26; and
providing a diagnosis of cancer, heart disease, viral infection, or disease susceptibility based on said characterization.

29. A method of diagnosing of cancer, heart disease, or viral infection, the method comprising:
obtaining a sample comprising nucleic acid derived from a cell;
determining whether said sample comprises one or more of the nucleic acid molecules in the collection of nucleic acid molecules of claim 27;
characterizing said sample on the basis of the presence or absence of one or more of the nucleic acid molecules in the collection of nucleic acid molecules of claim 27; and
providing a diagnosis of cancer, heart disease, or viral infection based on said characterization.

30. A recombinant expression vector, comprising a nucleic acid molecule according to claim 15.

31. A set of probes comprising the collection of nucleic acid molecules of claim 28.

32. An array comprising the set of probes according to claim 31.

33. (canceled)

34. A method of performing a diagnostic test, the method comprising:
obtaining a sample comprising nucleic acid derived from a cell;
determining whether said sample comprises one or more nucleic acid molecules which hybridize to one or more of the nucleic acid molecules of the array of claim 32;
characterizing said sample on the basis of the presence or absence of one or more nucleic acid molecules which hybridize to one or more of the nucleic acid molecules of the array of claim 32; and
providing a diagnosis based on said characterization.

35. A pharmaceutical composition comprising, as an active agent, at least one nucleic acid molecule according to claim 15.

36. A method of modulating a developmental or pathogenic disorder, the method comprising administering the pharmaceutical composition of claim 35 to a cell.

37. The method according to claim 36, wherein said disorder is cancer.

38. (canceled)

39. The pharmaceutical composition of claim 35 further comprising a pharmaceutically acceptable carrier.

40. A method of performing a therapeutic application, the method comprising;
administering the nucleic acid molecule of claim 15 to a cell.
